SEQUENCE LISTING (37 sequences)

SEQ ID NO: 1 - DNA, 41 bp, Artificial
  PCR primer
aatggactatcatatgcttaccgtaacttgaaagtatttcg

SEQ ID NO: 2 - DNA, 45 bp, Artificial
  PCR primer
ctttagtttgtatgtctgttgctattatgtctactattctttccc

SEQ ID NO: 3 - DNA, 67 bp, Artificial
  PCR primer
aatgatacggcgaccaccgagaaagtatttcgatttcttggctttatatatcttgtggannnnacga

SEQ ID NO: 4 - DNA, 66 bp, Artificial
  PCR primer
caagcagaagacggcatacgagctcttccgatcttgtggatgaatactgccatttgtctcgaggtc

SEQ ID NO: 5 - DNA, 41 bp, Artificial
  PCR primer
gagaaagtatttcgatttcttggctttatatatcttgtgga

SEQ ID NO: 6 - DNA, 40 bp, Artificial
  PCR primer
ttttagcactgccnnnnnnnctcgcgggccgcaggtccat

SEQ ID NO: 7 - DNA, 45 bp, Artificial
  PCR primer
ccggtttttagcatcgccnnnnnnnctcgcggccgcaggtccatg

SEQ ID NO: 8 - DNA, 45 bp, Artificial
  PCR primer
aattcatggacctgcggccgcgagnnnnnnnggcgatgctaaaaa

SEQ ID NO: 9 - DNA, 20 bp, Artificial
  ND1 forward primer
ccctaaaacccgccacatct

SEQ ID NO: 10 - DNA, 22 bp, Artificial
  ND1 reverse primer
gagcgatggtgagagctaaggt

SEQ ID NO: 11 - DNA, 25 bp, Artificial
  ND2 forward primer
tgttggttatacccttcccgtacta

SEQ ID NO: 12 - DNA, 25 bp, Artificial
  ND2 reverse primer
cctgcaaagatggtagagtagatga

SEQ ID NO: 13 - DNA, 19 bp, Artificial
  Alu forward primer
cttgcagtgagccgagatt

SEQ ID NO: 14 - DNA, 21 bp, Artificial
  Alu reverse primer
gagacggagtctcgctctgtc

SEQ ID NO: 15 - DNA, 20 bp, Artificial
  GLUT1 forward primer
tcgtcggcatcctcatcgcc

SEQ ID NO: 16 - DNA, 20 bp, Artificial
  GLUT1 reverse primer
ccggttctcctcgttgcggt

SEQ ID NO: 17 - DNA, 20 bp, Artificial
  GLUT3 forward primer
ttgctcttcccctccgctgc

SEQ ID NO: 18 - DNA, 20 bp, Artificial
  GLUT3 reverse primer
accgtgtgcctgcccttcaa

SEQ ID NO: 19 - DNA, 33 bp, Artificial
  SLC2A3 forward primer
gcatggatccaccatgggcacacagaaggtcac

SEQ ID NO: 20 - DNA, 32 bp, Artificial
  SLC2A3 reverse primer
gcatcaattgttagacattggtggtggtctcc

SEQ ID NO: 21 - DNA, 23 bp, Artificial
  MT-ND1 forward primer
ggtttgttaagatggcagagccc

SEQ ID NO: 22 - DNA, 26 bp, Artificial
  MT-ND1 reverse primer
gatgggttcgattctcatagtcctag

SEQ ID NO: 23 - DNA, 27 bp, Artificial
  MT-ND2 forward primer
taaggtcagctaaataagctatcgggc

SEQ ID NO: 24 - DNA, 31 bp, Artificial
  MT-ND2 reverse primer
cttagctgttacagaaattaagtattgcaac

SEQ ID NO: 25 - DNA, 31 bp, Artificial
  MT-ND3/4 forward primer
ttgatgagggtcttactcttttagtataaat

SEQ ID NO: 26 - DNA, 23 bp, Artificial
  MT-ND3/4 reverse primer
gataagtggcgttggcttgccat

SEQ ID NO: 27 - DNA, 23 bp, Artificial
  MT-ND4 forward primer
ccttttcctccgaccccctaaca

SEQ ID NO: 28 - DNA, 26 bp, Artificial
  MT-ND4 reverse primer
tagcagttcttgtgagctttctcggt

SEQ ID NO: 29 - DNA, 31 bp, Artificial
  MT-ND5 forward primer
aacatggctttctcaacttttaaaggataac

SEQ ID NO: 30 - DNA, 28 bp, Artificial
  MT-ND5 reverse primer
cgtttgtgtatgatatgtttgcggtttc

SEQ ID NO: 31 - DNA, 23 bp, Artificial
  MT-ND5/6 forward primer
acttcaacctccctcaccattgg

SEQ ID NO: 32 - DNA, 30 bp, Artificial
  MT-ND5/6 reverse primer
tcattggtgttcttgtagttgaaatacaac

SEQ ID NO: 33 - DNA, 27 bp, Artificial
  Ndi EcoRI forward primer
atgaattccatcacatcatcgaattac

SEQ ID NO: 34 - DNA, 36 bp, Artificial
  Ndi Xhol reverse primer
atctcgagaaaagggcatgttaatttcatctataat

SEQ ID NO: 35 - DNA, 25 bp, Artificial
  primer targeting vector
agtagacggcatcgcagcttggata

SEQ ID NO: 36 - DNA, 21 bp, Artificial
  primer targeting vector
ggcggaatttacgtagcggcc

SEQ ID NO: 37 - DNA, 25 bp, Artificial
  GLUT3 primer
gagccgattgtagcaactgtgatgg

(19) United States
(12) Patent Application Publication
(10) Pub. No.: US 2014/0348749 A1
(43) Pub. Date: Nov. 27, 2014

(54) IDENTIFICATION AND TREATMENT OF TUMORS SENSITIVE TO GLUCOSE LIMITATION

(71) Applicant: Whitehead Institute for Biomedical Research, Cambridge, MA (US)

(72) Inventors: Kivanc Birsoy, Cambridge, MA (US); Richard Possemato, Brighton, MA (US); David M. Sabatini, Cambridge, MA (US)

(73) Assignee: Whitehead Institute for Biomedical Research, Cambridge, MA (US)

(21) Appl. No.: 14/190,069

(22) Filed: Feb. 25, 2014

Related U.S. Application Data
(60) Provisional application No. 61/769,185, filed on Feb. 25, 2013.

(51) Int. Cl. G01N 33/50

(52) U.S. Cl. CPC G01N 33/5011
USPC 424/9.2; 435/32; 435/375; 435/6.12

(57) ABSTRACT

In some aspects, compositions and methods useful for classifying tumor cells, tumor cell lines, or tumors according to predicted sensitivity to glucose restriction are provided. In some aspects, compositions and methods useful for classifying tumor cells, tumor cell lines, or tumors according to predicted sensitivity to OXPHOS inhibitors are provided. In some aspects, compositions and methods useful for classifying tumor cells, tumor cell lines, or tumors according to predicted sensitivity to biguanides are provided. In some aspects, methods of identifying subjects with cancer who are candidates for treatment with an OXPHOS inhibitor are provided. In some aspects, methods of identifying subjects with cancer who are candidates for treatment with a biguanide are provided. In some aspects, methods of treating subjects with cancers that are sensitive to glucose restriction are provided.

### CROSS-REFERENCE TO RELATED APPLICATION

This application claims the benefit of U.S. Provisional Application No. 61/769,185, filed Feb. 25, 2013. The entire teachings of the above application are incorporated herein by reference.

### GOVERNMENT SUPPORT

The invention was made with government support under R01-CA 103866-06 awarded by the National Institutes of Health. The government has certain rights in the invention.

### BACKGROUND

Cancer is a major cause of death worldwide. The observation that most tumors have an elevated rate of glucose consumption compared to normal tissues, first made several decades ago, has recently received renewed attention, and the role of altered cell metabolism in cancer is becoming increasingly appreciated. A number of compounds that target various aspects of the metabolic machinery are currently undergoing preclinical or clinical evaluation as potential therapeutic agents for cancer. Metformin is a drug of the biguanide class that is widely used in the treatment of Type II diabetes. Metformin functions as an oral antihyperglycemic agent, effectively lowering blood glucose levels by mechanisms that are incompletely understood. Recently, retrospective studies have shown that Type II diabetic patients with cancer who are taking metformin for treatment of their diabetes have better outcomes as a group than patients not taking metformin. These observations have prompted considerable interest in metformin as a chemotherapeutic agent.

### SUMMARY

In some aspects, the present disclosure relates to large scale approaches to study tumor metabolism. In some embodiments, the present disclosure relates to products and methods useful for discovering which genes, e.g., which metabolic genes, are required for cancer-relevant processes. For example, in some embodiments the disclosure relates to products and methods useful for identifying metabolic genes required proliferation, survival, and/or cell state in context of environment and genotype. In some embodiments the disclosure relates to determining why certain metabolic genes are required for such processes, e.g., under certain environmental conditions. In some embodiments, metabolic genes, genetic liabilities, and/or metabolic liabilities described herein are of use to identify tumors that are more likely to respond to particular therapeutic approaches and/or agents.

In some aspects, the present disclosure provides the insight that the survival and/or proliferation of tumor cells of diverse origin are differentially affected by glucose concentration, e.g., glucose restriction. For example, analysis of more than two dozen tumor cell lines revealed that certain cell lines proliferated significantly less rapidly in medium containing a low concentration of glucose (e.g., about 0.75 mM-1 mM glucose) than in medium containing a standard glucose concentration (about 10 mM glucose). Other cell lines proliferated more rapidly in medium containing a low glucose concentration (e.g., about 0.75 mM-1 mM glucose) than in medium containing 10 mM glucose. Some cell lines exhibited little of no difference in proliferation rate between these conditions. The disclosure further provides the insight that that the relative sensitivity or resistance of tumor cells to glucose restriction has significant implications with regard to the response of such cells to agents that modulate aspects of cellular metabolism, such as agents that target pathways used by cells to produce ATP or that otherwise affect cellular energy status. For example, in some aspects, tumor cells that are sensitive to glucose restriction display increased sensitivity to agents that target pathways used by cells to produce ATP as compared with tumor cells that are not sensitive to glucose restriction.

In some aspects, the invention relates to the recognition that tumor cells, tumor cell lines, and tumors may exhibit variable degrees of sensitivity to OXPHOS inhibitors. In some aspects, methods of identifying tumors or tumor cells that have an increased likelihood of sensitivity to OXPHOS inhibitors are provided herein. In some embodiments, such methods may be used to identify patients with cancer who would be likely to benefit from treatment with an OXPHOS inhibitor, e.g., patients who are likely to respond or who are likely to exhibit a robust response.

In some aspects, the invention relates to the recognition that tumor cells, tumor cell lines, and tumors may exhibit variable degrees of sensitivity to biguanides. In some aspects, methods of identifying tumors or tumor cells that have an increased likelihood of biguanide sensitivity are provided herein. In some embodiments, such methods may be used to identify patients with cancer who would be likely to benefit from treatment with a biguanide, e.g., patients who are likely to respond or who are likely to exhibit a robust response.

In some aspects, the invention provides a method of classifying a tumor cell or tumor according to predicted sensitivity to OXPHOS inhibition, the method comprising: assessing expression of at least one gene listed in Table 1 in the tumor or in a sample obtained from the tumor, wherein an decreased level of expression is correlated with increased likelihood of sensitivity to OXPHOS inhibition; and classifying the tumor with respect to predicted sensitivity to OXPHOS inhibition based at least in part on the level of expression of the gene(s) in the tumor or sample. In some embodiments the method comprises: (a) determining the level of a gene product of a gene listed in Table 1 in the tumor or sample; (b) comparing the level of the gene product with a reference level, and (c) classifying the tumor as having or not having increased likelihood of sensitivity to OXPHOS inhibition based at least in part on the result of step (b). In some embodiments reduced expression of the gene(s) as compared with average expression in a diverse set of tumors is indicative of increased likelihood of sensitivity to OXPHOS inhibition. In some embodiments reduced expression of the gene(s) as compared with average expression in tumors of the same type is indicative of increased likelihood of sensitivity to OXPHOS inhibition. In some embodiments expression of the gene(s) at or below the average level of expression of the gene in tumors that are sensitive to glucose limitation is indicative of increased likelihood of sensitivity to OXPHOS inhibition. In some embodiments the gene is CYC1, UQCRC1, or SLC2A3 (GLUT3). The NCBI Gene ID of human SLC2A3 is 6515. The NCBI RefSeq mRNA and protein accession numbers are NM_{—}006931 and NP_{—}008862, respectively. In some embodiments expression level of one or more genes listed in Table 3 is used to assess likelihood of sensitivity to low glucose, likelihood of sensitivity to OXPHOS inhibition, or likelihood of sensitivity to biguanides.

In some aspects, the invention provides a method of classifying a tumor cell or tumor according to predicted biguanide sensitivity, the method comprising: assessing expression of at least one gene listed in Table 1 in the tumor or in a sample obtained from the tumor, wherein an decreased level of expression is correlated with increased biguanide sensitivity; and classifying the tumor with respect to predicted sensitivity to the compound based at least in part on the level of expression of the gene(s) in the tumor or sample. In some embodiments the method comprises: (a) determining the level of a gene product of a gene listed in Table 1 in the tumor or sample; (b) comparing the level of the gene product with a reference level, and (c) classifying the tumor as having or not having an increased likelihood of biguanide sensitivity based at least in part on the result of step (b). In some embodiments reduced expression of the gene(s) as compared with average expression in a diverse set of tumors is indicative of increased likelihood of biguanide sensitivity. In some embodiments reduced expression of the gene(s) as compared with average expression in tumors of the same type is indicative of increased likelihood of biguanide sensitivity. In some embodiments expression of the gene(s) at or below the average level of expression of the gene in tumors that are sensitive to glucose limitation is indicative of increased likelihood of biguanide sensitivity. In some embodiments the gene is CYC1, UQCRC1, or SLC2A3 (GLUT3).

In some aspects, the invention provides a method of classifying a tumor cell or tumor according to predicted sensitivity to OXPHOS inhibition, the method comprising: assessing expression of at least one gene listed in Table 4 in the tumor or in a sample obtained from the tumor, wherein an decreased level of expression is correlated with increased likelihood of sensitivity to OXPHOS inhibition; and classifying the tumor with respect to predicted sensitivity to OXPHOS inhibition based at least in part on the level of expression of the gene(s) in the tumor or sample. In some embodiments the method comprises: (a) determining the level of a gene product of a gene listed in Table 4 in the tumor or sample; (b) comparing the level of the gene product with a reference level, and (c) classifying the tumor as having or not having increased likelihood of sensitivity to OXPHOS inhibition based at least in part on the result of step (b). In some embodiments expression of at least 2, 3, 4, 5, 6, 7, 8, 9, 10, or more the genes is assessed in step (b). In some embodiments reduced expression of one or more of the gene(s) as compared with average expression in a diverse set of tumors is indicative of increased likelihood of sensitivity to OXPHOS inhibition. In some embodiments reduced expression of one or more of the gene(s) as compared with average expression in tumors of the same type is indicative of increased likelihood of sensitivity to OXPHOS inhibition. In some embodiments expression of one or more of the gene(s) at or below the average level of expression of the gene in tumors that are sensitive to glucose limitation is indicative of increased likelihood of sensitivity to OXPHOS inhibition. In some embodiments expression of one or more of the gene(s) at or below the average level of expression of the gene in tumors that are sensitive to glucose limitation is indicative of increased likelihood of sensitivity to biguanides. In some embodiments expression of one or more of the gene(s) at or below the average level of expression of the gene in tumors that are sensitive to glucose limitation is indicative of increased likelihood of sensitivity to glucose limitation.

In some aspects, the invention provides a method of classifying a tumor cell or tumor according to predicted biguanide sensitivity, the method comprising: assessing expression of at least one gene listed in Table 4 in the tumor or in a sample obtained from the tumor, wherein an decreased level of expression is correlated with increased biguanide sensitivity; and classifying the tumor with respect to predicted sensitivity to biguanides based at least in part on the level of expression of the gene(s) in the tumor or sample. In some embodiments the method comprises: (a) determining the level of a gene product of a gene listed in Table 4 in the tumor or sample; (b) comparing the level of the gene product with a reference level, and (c) classifying the tumor as having or not having an increased likelihood of biguanide sensitivity based at least in part on the result of step (b). In some embodiments reduced expression of the gene(s) as compared with average expression in a diverse set of tumors is indicative of increased likelihood of biguanide sensitivity. In some embodiments reduced expression of the gene(s) as compared with average expression in tumors of the same type is indicative of increased likelihood of biguanide sensitivity. In some embodiments expression of the gene(s) at or below the average level of expression of the gene in tumors that are sensitive to glucose limitation is indicative of increased likelihood of biguanide sensitivity. In some embodiments the gene is ENO1, GAPDH, GPI, HK1, PKM, TPI1, ALDOA, PFKP, or PGI1 or any combination thereof. In some embodiments expression of at least 2, 3, 4, 5, 6, 7, 8, 9, 10, or more the genes is decreased.

In some aspects, the disclosure provides a method of predicting the likelihood that a tumor cell, tumor cell line, or tumor, is sensitive to OXPHOS inhibition, the method comprising: assessing expression of at least one gene listed in Table 1 by the tumor cell, tumor cell line, or tumor; and generating a prediction of the likelihood that the tumor cell, tumor cell line, or tumor, is sensitive to the OXPHOS inhibition based at least in part on the assessment. In some embodiments assessing expression of the gene comprises (a) determining the level of a gene product of the gene in the tumor cell, tumor cell line, tumor, or a sample obtained therefrom; and (b) comparing the level with a reference level of the gene product.

In some aspects, the disclosure provides a method of predicting the likelihood that a tumor cell, tumor cell line, or tumor, is sensitive to OXPHOS inhibition, the method comprising: assessing expression of at least one gene listed in Table 4 by the tumor cell, tumor cell line, or tumor; and generating a prediction of the likelihood that the tumor cell, tumor cell line, or tumor, is sensitive to the OXPHOS inhibition based at least in part on the assessment. In some embodiments assessing expression of the gene comprises (a) determining the level of a gene product of the gene in the tumor cell, tumor cell line, tumor, or a sample obtained therefrom; and (b) comparing the level with a reference level of the gene product.

In some aspects, the disclosure provides a method of predicting the likelihood that a tumor cell, tumor cell line, or tumor, is sensitive to biguanides, the method comprising: assessing expression of at least one gene listed in Table 1 by the tumor cell, tumor cell line, or tumor; and generating a prediction of the likelihood that the tumor cell, tumor cell line, or tumor, is sensitive to biguanides based at least in part on the assessment. In some embodiments assessing expression of the gene comprises (a) determining the level of a gene product of the gene in the tumor cell, tumor cell line, tumor, or a sample obtained therefrom; and (b) comparing the level with a reference level of the gene product. In some embodiments the gene is CYC1, UQCRC1, or SLC2A3 (GLUT3). In some embodiments a low level of expression of a gene listed in Table 1, e.g., CYC1 and/or UQCRC1, is a level at or below twice the level of expression in a tumor cell line selected from the group consisting of: Jurkat, MC116, U927, NCI-H929 or selected from the group consisting of Jurkat, MC116, KMS-26, NCI-H929, LP-1, L-363, MOLP-8, D341 Med, and KMS-28BM. In some embodiments a low level of expression of a gene listed in Table 1, e.g., CYC1 and/or UQCRC1, is a level at or below twice the average level of expression in the afore-mentioned cell lines. In some embodiments a low level of expression of a gene listed in Table 1, e.g., CYC1 and/or UQCRC1, is a level at or below the level of expression in a tumor cell line selected from the group consisting of: Jurkat, MC116, U927, NCI-H929 or selected from the group consisting of Jurkat, MC116, KMS-26, NCI-H929, LP-1, L-363, MOLP-8, D341 Med, and KMS-28BM. In some embodiments a low level of expression of a gene listed in Table 1, e.g., CYC1 and/or UQCRC1, is a level at or below the average level of expression in the afore-mentioned cell lines. In some embodiments a low level of expression of SLC2A3 is a level at or below twice the level of expression in a tumor cell line selected from the group consisting of: KMS-26 and NCI-H929. In some embodiments a low level of expression of SLC2A3 is a level at or below the level of expression in a tumor cell line selected from the group consisting of: KMS-26 and NCI-H929. In some embodiments a low level of expression of SLC2A3 is a level at or below twice the level of expression in KMS-26 and NCI-H929 cells. In some embodiments a low level of expression of SLC2A3 is a level at or below the level of expression in KMS-26 and NCI-H929 cells. An expression level may be measured using any suitable expression level determining system or method in various embodiments. In some embodiments expression level is determined using, e.g., IHC, western blotting, qPCR, etc. In some embodiments an activity of a gene product of a gene listed in Table 1 or a complex comprising such gene product is measured instead of or in addition to measuring the level of a gene product. In some embodiments an activity of SLC2A3 is measured instead of or in addition to measuring the level of a SLC2A3 gene product. In some embodiments an activity is glucose import.

In some aspects, the disclosure provides a method of predicting the likelihood that a tumor cell, tumor cell line, or tumor, is sensitive to biguanides, the method comprising: assessing expression of at least one gene listed in Table 4 by the tumor cell, tumor cell line, or tumor; and generating a prediction of the likelihood that the tumor cell, tumor cell line, or tumor, is sensitive to biguanides based at least in part on the assessment. In some embodiments assessing expression of the gene comprises (a) determining the level of a gene product of the gene in the tumor cell, tumor cell line, tumor, or a sample obtained therefrom; and (b) comparing the level with a reference level of the gene product. In some embodiments a low level of expression of a gene listed in Table 4, is a level at or below twice the level of expression in a tumor cell line selected from the group consisting of: Jurkat, MC116, U927, NCI-H929, KMS-26, LP-1, L-363, MOLP-8, D341 Med, and KMS-28BM. In some embodiments a low level of expression of a gene listed in Table 4 is a level at or below twice the average level of expression in the afore-mentioned cell lines. In some embodiments the cell line is KMS-26 or NCI-H929. In some embodiments a low level of expression of SLC2A3 is a level at or below the level of expression in KMS-26 and NCI-H929 cells.

In some aspects, the disclosure provides method of determining whether a subject in need of treatment for a tumor is a candidate for treatment with an OXPHOS inhibitor, the method comprising assessing expression of at least one gene listed in Table 1; and identifying the subject as a candidate for treatment with an OXPHOS inhibitor based at least in part on the assessment. In some embodiments the method comprises (a) determining the level of an gene product of a gene listed in Table 1 in the tumor or a sample obtained therefrom; and (b) comparing the level with a reference level of the gene product. In some embodiments the gene is CYC1 and/or UQCRC1.

In some aspects, the disclosure provides method of determining whether a subject in need of treatment for a tumor is a candidate for treatment with an OXPHOS inhibitor, the method comprising assessing expression of at least one gene listed in Table 4; and identifying the subject as a candidate for treatment with an OXPHOS inhibitor based at least in part on the assessment. In some embodiments the method comprises (a) determining the level of an gene product of a gene listed in Table 4 in the tumor or a sample obtained therefrom; and (b) comparing the level with a reference level of the gene product.

In some aspects, the disclosure provides a method of treating a subject in need of treatment for a tumor, the method comprising: (a) determining that the subject's tumor has one or more genotypic or phenotypic characteristics indicative of increased likelihood of sensitivity to glucose limitation; and (b) treating the subject with an OXPHOS inhibitor. In some aspects, the disclosure provides a method of treating a subject in need of treatment for a tumor, the method comprising: (a) determining that the subject's tumor has one or more genotypic or phenotypic characteristics indicative of increased likelihood of sensitivity to OXPHOS inhibition; and (b) treating the subject with an OXPHOS inhibitor. In some aspects, the disclosure provides a method of treating a subject in need of treatment for a tumor, the method comprising: (a) determining that the subject's tumor has one or more genotypic or phenotypic characteristics indicative of increased likelihood of sensitivity to glucose limitation; and (b) treating the subject with a biguanide. In some aspects, the disclosure provides a method of treating a subject in need of treatment for a tumor, the method comprising: (a) determining that the subject's tumor has one or more genotypic or phenotypic characteristics indicative of increased likelihood of sensitivity to OXPHOS inhibition; and (b) treating the subject with a biguanide. In some embodiments a genotypic characteristic is presence of a mutation in a gene encoding an OXPHOS component, e.g., a complex I component. In some embodiments the gene is a mitochondrial gene. In some embodiments the gene is ND1 or ND5 or ND4. In some embodiments a phenotypic characteristic is a defect in OXPHOS. In some embodiments a phenotypic characteristic is decreased ability to take up glucose. In some embodiments a phenotypic characteristic is decreased expression or activity of SLC2A3, e.g., as compared with average SLC2A3 expression in tumors. In some embodiments a phenotypic characteristic is an inability to upregulate OCR in response to glucose limitation. In some embodiments a phenotypic characteristic is decreased expression or activity of one or more genes listed in Table 4, e.g., as compared with the average expression of such gene in tumors. In some embodiments a phenotypic characteristic is increased basal AMPK phosphorylation.

In some embodiments a reference level in a method disclosed herein is a level of the gene product in tumors or tumor cell lines that are sensitive to glucose limitation. In some embodiments if a gene in Table 1 and/or in Table 4 is expressed in a tumor or tumor cell line at or below twice the level of its expression in tumors or tumor cell lines that are sensitive to glucose limitation, the tumor or tumor cell line is predicted to be sensitive to glucose limitation, OXPHOS inhibition, or both. In some embodiments if a gene in Table 1 and/or in Table 4 is expressed in a tumor or tumor cell line at or below the level of its expression in tumors or tumor cell lines that are sensitive to glucose limitation, the tumor or tumor cell line is predicted to be sensitive to glucose limitation, OXPHOS inhibition, or both. In some embodiments if a gene in Table 1 and/or in Table 4 is expressed in a tumor or tumor cell line at or below twice the level of its expression in tumors or tumor cell lines that are sensitive to glucose limitation, the tumor or tumor cell line is predicted to be sensitive to biguanides. In some embodiments if a gene in Table 1 and/or in Table 4 is expressed in a tumor or tumor cell line at or below the level of its expression in tumors or tumor cell lines that are sensitive to glucose limitation, the tumor or tumor cell line is predicted to be sensitive to biguanides. In some embodiments a tumor or tumor cell line having an expression level of a gene or gene product falling within the lowest 25% of tumors or tumor cell lines of that type is considered to have low expression of the gene or gene product. In some embodiments a tumor or tumor cell line having an expression level of a gene or gene product falling within the lowest 20% of tumors or tumor cell lines of that type is considered to have low expression of the gene or gene product. In some embodiments a tumor or tumor cell line having an expression level of a gene or gene product falling within the lowest 15% of tumors or tumor cell lines of that type is considered to have low expression of the gene or gene product. In some embodiments a tumor having an expression level of a gene or gene product falling within the lowest 10% of tumors or tumor cell lines of that type is considered to have low expression of the gene or gene product.

The passage of glucose across cell membranes is facilitated by a family of integral membrane transporter proteins, the GLUTs. There are currently 14 members of the SLC2 family of GLUTs. In some embodiments low expression of a glucose transporter, e.g., SLC2A3 (GLUT3), results in sensitivity to glucose limitation. Further information regarding SLC2A3 (GLUT3) may be found in Simpson, I A, et al., The facilitative glucose transporter GLUT3: 20 years of distinction. Am J Physiol Endocrinol Metab. 2008; 295(2):E242-53, and references therein. In some embodiments if SLC2A3 is expressed in a tumor or tumor cell line at or below twice the level of its expression in tumors or tumor cell lines that are sensitive to glucose limitation and have low SLC2A3 expression (e.g., KMS26 or NCI-H929 cells), the tumor or tumor cell line is predicted to be sensitive to glucose limitation, OXPHOS inhibition, or both. In some embodiments if SLC2A3 is expressed in a tumor or tumor cell line at or below the level of its expression in tumors or tumor cell lines that are sensitive to glucose limitation and have low SLC2A3 expression (e.g., KMS26 or NCI-H929 cells), the tumor or tumor cell line is predicted to be sensitive to glucose limitation, OXPHOS inhibition, or both. In some embodiments if SLC2A3 is expressed in a tumor or tumor cell line at or below twice the level of its expression in tumors or tumor cell lines that are sensitive to glucose limitation and have low SLC2A3 expression (e.g., KMS26 or NCI-H929 cells), the tumor or tumor cell line is predicted to be sensitive to biguanides. In some embodiments if SLC2A3 is expressed in a tumor or tumor cell line at or below the level of its expression in tumors or tumor cell lines that are sensitive to glucose limitation and have low SLC2A3 expression (e.g., KMS26 or NCI-H929 cells), the tumor or tumor cell line is predicted to be sensitive to biguanides. In some embodiments a tumor or tumor cell line tested for SLC2A3 expression is a prostate, esophagus, breast, stomach, lung, and pancreas tumor or tumor cell line. In some embodiments a tumor having an expression level of SLC2A3 falling within the lowest 25% of tumors of that type is considered to have low SLC2A3 expression. In some embodiments a tumor or tumor cell line having an expression level of SLC2A3 falling within the lowest 20% of tumors or tumor cell lines of that type is considered to have low SLC2A3 expression. In some embodiments a tumor having an expression level of SLC2A3 falling within the lowest 15% of tumors or tumor cell lines of that type is considered to have low SLC2A3 expression. In some embodiments a tumor or tumor cell line having an expression level of SLC2A3 falling within the lowest 10% of tumors or tumor cell lines of that type is considered to have low SLC2A3 expression. As described herein, low expression of SLC2A3 is part of a gene expression signature indicative of low glucose utilization. Other genes whose low expression is associated with low glucose utilization include ENO1, GAPDH, GPI, HK1, PKM, TPI1, ALDOA, PFKP, and PGI1. Expression levels constituting low levels of expression of such genes may be determined as described for SLC3A2.

In some embodiments of any of the above methods, the method further comprises treating a subject in need of treatment for the tumor with an OXPHOS inhibitor at least in part on the classification, prediction, or determination. In some embodiments of any of the above methods, the method further comprises treating a subject in need of treatment for the tumor with a biguanide, e.g., metformin, at least in part on the classification, prediction, or determination. In some embodiments any such methods may further comprise treating the subject with a second anti-tumor therapy.

In some embodiments of any of the above methods, the method further comprises storing the result of the assessment, classification, determination, or prediction in a database, optionally in association with a sample identifier or subject identifier.

In some embodiments of any of the above methods, the method further comprises providing the result of an assessment, classification, determination, or prediction to a health care provider. In some embodiments of any of the above methods, the method further comprises providing the result of an assessment, classification, determination, or prediction to a subject, e.g., a subject in need of treatment for the tumor.

In some aspects, the disclosure provides a method of treating a subject in need of treatment for a tumor the method comprising: treating the subject with an OXPHOS inhibitor, wherein the tumor has been determined to have one or more genetic or phenotypic characteristics indicative of increased likelihood of sensitivity to OXPHOS inhibition. In some aspects, the disclosure provides a method of treating a subject in need of treatment for a tumor the method comprising: treating the subject with a biguanide, wherein the tumor has been determined to have one or more genetic or phenotypic characteristics indicative of increased likelihood of sensitivity to biguanides.

In some aspects, the disclosure provides a kit comprising: a detection reagent suitable for detecting a gene product of a gene listed in Table 1 or Table 4. In some embodiments the detection reagent is suitable for detecting a CYC1, UQCRC1, or SLC2A3 (GLUT3) gene product in a tumor sample. In some embodiments the detection reagent is suitable for detecting a mutation in a gene encoding an OXPHOS component, e.g., a component of complex I, e.g., ND1 or ND5 or ND4. In some embodiments the detection reagent is suitable for performing a method set forth herein. In some embodiments, the agent has been validated for use in a method set forth above or elsewhere herein. In some embodiments the detection reagent comprises an antibody that binds to polypeptide encoded by the gene. In some embodiments the detection reagent comprises a probe or primer that hybridizes to mRNA of the gene or a complement thereof. In some embodiments a kit further comprises (i) instructions for using the kit for tumor classification, prediction, or treatment selection; (ii) a substrate or secondary antibody; and/or (iii) a control substance. In some embodiments a kit comprises a label or package insert indicating that the kit is approved by a government regulatory agency for use in tumor classification, prediction, or treatment selection. In some embodiments a kit comprises a label or package insert indicating that the kit is approved by a government regulatory agency for use as a companion diagnostic for identifying patients who are candidates for treatment with an OXPHOS inhibitor. In some embodiments a kit comprises a label or package insert indicating that the kit is approved by a government regulatory agency for use as a companion diagnostic for identifying patients who are candidates for treatment with a biguanide.

In some aspects, the disclosure provides a method of determining whether a subject in need of treatment for a tumor is a candidate for treatment with an OXPHOS inhibitor, the method comprising determining whether the tumor has one or more genetic or phenotypic characteristics indicative of increased likelihood of sensitivity to OXPHOS inhibition; and, if so, identifying the subject as a candidate for treatment with an OXPHOS inhibitor. In some aspects, the disclosure provides a method of determining whether a subject in need of treatment for a tumor is a candidate for treatment with a biguanide, the method comprising determining whether the tumor has one or more genetic or phenotypic characteristics indicative of increased likelihood of sensitivity to a biguanide and, if so, identifying the subject as a candidate for treatment with a biguanide, e.g., metformin.

In some aspects, the disclosure provides method of identifying a candidate anti-cancer agent the method comprising: (a) providing a test agent; and (b) determining whether the test agent inhibits expression or activity of a gene product encoded by a gene listed in Table 1 or Table 4, wherein the test agent is identified as a candidate anti-cancer agent if the test agent inhibits expression or activity of the gene product. In some embodiments the method comprising determining whether the test agent inhibits expression or activity of a gene product comprises (i) contacting the test agent with one or more cells that express the gene product; and (ii) measuring the level of expression or activity of the gene product; wherein a decrease in expression or activity of the gene product relative to control cell(s) not exposed to the test agent is indicative that the test agent inhibits expression or activity of the gene product. In some embodiments a method comprises testing the effect of an identified candidate agent on cancer cells. In some embodiments the cancer cells have at least one genetic or phenotypic characteristic indicative of increased likelihood of sensitivity to OXPHOS inhibition. In some embodiments a method comprises preparing a composition comprising an identified candidate agent and a pharmaceutically acceptable carrier. In some embodiments a method comprises testing the effect of an identified candidate agent on tumor cell survival or proliferation. In some embodiments a method comprises testing the effect of an identified candidate agent on a tumor in vivo, e.g., in a non-human animal that serves as a tumor model. In some embodiments an identified candidate agent is tested in combination with an OXPHOS inhibitor. In some embodiments an identified candidate agent is tested in combination with a biguanide.

In some aspects, the disclosure provides method of identifying a candidate anti-cancer agent the method comprising: (a) providing a test agent; and (b) determining whether the test agent inhibits expression or activity of a gene product encoded by a glucose utilization signature gene listed in Table 4, wherein the test agent is identified as a candidate anti-cancer agent if the test agent inhibits expression or activity of the gene product. In some embodiments the method comprising determining whether the test agent inhibits expression or activity of a gene product comprises (i) contacting the test agent with one or more cells that express the gene product; and (ii) measuring the level of expression or activity of the gene product; wherein a decrease in expression or activity of the gene product relative to control cell(s) not exposed to the test agent is indicative that the test agent inhibits expression or activity of the gene product. In some embodiments a method comprises testing the effect of an identified candidate agent on cancer cells. In some embodiments the cancer cells have at least one genetic or phenotypic characteristic indicative of increased likelihood of sensitivity to OXPHOS inhibition. In some embodiments a method comprises preparing a composition comprising an identified candidate agent and a pharmaceutically acceptable carrier. In some embodiments a method comprises testing the effect of an identified candidate agent on tumor cell survival or proliferation. In some embodiments a method comprises testing the effect of an identified candidate agent on a tumor in vivo, e.g., in a non-human animal that serves as a tumor model. In some embodiments an identified candidate agent is tested in combination with an OXPHOS inhibitor. In some embodiments an identified candidate agent is tested in combination with a biguanide.

In some aspects, the disclosure provides a method of inhibiting survival or proliferation of a tumor cell comprising: (a) determining that the tumor cell expresses a decreased level of GLUT3; and (b) contacting the tumor cell with an OXPHOS inhibitor In some embodiments the tumor cell is contacted with the OXPHOS inhibitor in culture. In some embodiments the tumor cell is contacted with the OXPHOS inhibitor by administering the OXPHOS inhibitor to a subject having a tumor.

In some aspects, the disclosure provides a method of inhibiting survival or proliferation of a tumor cell comprising: (a) determining that the tumor cell expresses a decreased level of GLUT3; and (b) contacting the tumor cell with a biguanide. In some embodiments the tumor cell is contacted with the biguanide by administering the biguanide to a subject having a tumor. In some embodiments of any aspect described herein, a tumor may be a tumor that has a defect in OXPHOS. In some embodiments of any aspect described herein, a tumor may be a tumor that has a defect in glucose uptake.

In some embodiments of any aspect described herein a tumor may be of any tumor type. In some embodiments a tumor may be a carcinoma. In some embodiments of any aspect described herein, a tumor may be a multiple myeloma or small cell lung cancer.

The practice of certain aspects of the present invention may employ conventional techniques of molecular biology, cell culture, recombinant nucleic acid (e.g., DNA) technology, immunology, transgenic biology, microbiology, nucleic acid and polypeptide synthesis, detection, manipulation, and quantification, and RNA interference that are within the ordinary skill of the art. See, e.g., Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual. ^{3rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988. Information regarding diagnosis and treatments of various diseases, including cancer, is found in Longo, D., et al. (eds.), Harrison's Principles of Internal Medicine, 18th Edition; McGraw-Hill Professional, 2011. Information regarding various therapeutic agents and human diseases, including cancer, is found in Brunton, L., et al. (eds.) Goodman and Gilman's The Pharmacological Basis of Therapeutics, 12^{th} Ed., McGraw Hill, 2010 and/or Katzung, B. (ed.) Basic and Clinical Pharmacology, McGraw-Hill/Appleton & Lange; 11th edition (July 2009). All patents, patent applications, books, articles, documents, databases, websites, publications, references, etc., mentioned herein are incorporated by reference in their entirety. In case of a conflict between the specification and any of the incorporated references, the specification (including any amendments thereof), shall control. Applicants reserve the right to amend the specification based, e.g., on any of the incorporated material and/or to correct obvious errors. None of the content of the incorporated material shall limit the invention. Standard art-accepted meanings of terms are used herein unless indicated otherwise.

Standard abbreviations for various terms are used herein.

### BRIEF DESCRIPTION OF THE DRAWINGS

FIG. 1. Left: Schematic overview of metabolic genes, transporters, and metabolic pathways. Right: Overview of various approaches to study metabolism.

FIG. 2. Schematic diagram of tumor development, illustrating that tumor cells often exist in a nutrient poor environment.

FIG. 3. Schematic diagram illustrating that tumor cells typically exhibit a viability threshold as distance from microvessels increases.

FIG. 4. Micrographs showing cuffs of viable cancer cells around tumor vessels.

FIG. 5. Plots illustrating that glucose is highly consumed by cancer cells, and its concentration low in tumors.

FIG. 6. Schematic diagram illustrating that nutrient levels are low in tumors compared to normal tissues but are not zero.

FIG. 7. (A) Schematic diagram illustrating some of the challenges of modeling continuous long term glucose limitation in culture. The glucose concentrations in cell culture medium changes at a variable rate depending, in part, on the starting glucose concentration and the number and proliferation rate of the cells. (B) Proliferation and media glucose levels in standard culture conditions. a, Jurkat cell proliferation under 10 mM (black) versus 1 mM (blue) glucose in standard culture conditions. b, Media glucose concentrations over time from cultures in (a). Error bars are SEM, n=3. Replicates are biological, means reported. Asterisks indicate significance p<0.05 by two-sided student's t-test.

FIG. 8. Schematic diagrams of a Nutrostat.

FIG. 9. Plots showing Jurkat cell proliferation in a Nutrostat that maintains constant glucose concentration. The upper plot shows that the glucose concentration remains approximately constant over time whether starting at 10 mM glucose (squares) or 0.75 mM glucose (circles). 10 mM represents a standard glucose concentration for culturing this cell type.

FIG. 10. Plots and heatmap showing various metabolic effects of long term glucose limitation. Upper panel shows indicated metabolite levels in Nutrostats at 10 mM (black) or 0.75 mM (blue) glucose. Lower panel shows differential intracellular metabolite abundances (p<0.05) from cells in Nutrostats at 10 mM (bottom three rows) or 0.75 mM (top three rows) glucose. Color bar indicates scale (Log 2 transformed). Error bars are SEM (n=2 (glucose and lactate), 3 (NAD(H) ratio) and 8 for ATP levels). Replicates are biological, means reported. Asterisks indicate significance p<0.05 by two-sided student's t-test.

FIG. 11. Schematic diagram of a screen for identification of metabolic genes required for proliferation under glucose limitation. (Screen also identifies metabolic genes required for proliferation under high glucose).

FIG. 12. Summary of results of screen for identification of metabolic genes required for proliferation under low or high glucose conditions.

FIG. 13. Lists of hits from screen for identification of metabolic genes required for proliferation under low or high glucose conditions.

FIG. 14. (A) Schematic diagram of electron transport chain showing the distribution of electron transport chain hits identified in the screen as differentially required for proliferation under glucose limitation. Number of mitochondria- or nuclear-encoded components and number of nuclear-encoded genes that scored indicated (red text). Significance of gene classes by complex is as follows: Complex I (p<9.3×10-49), III (p<6.6×10-20), IV (p<8.3×10-10) and V (p<5.6×10-19) by chi-squared test. (B) Nuclearly encoded core Complex I genes are written in the grey box indicating those which score (right, red text). Dot plot reports differential essentiality in 10 mM versus 0.75 mM glucose of individual shRNAs targeting non-core Complex I genes, core Complex I genes, or non-targeting controls. Red bar is the population median. (C) Gene suppression of cells expressing indicated shRNAs (top) and proliferation (bottom) in 0.75 mM (blue) relative to 10 mM glucose (black). Asterisks indicate significance (p<0.05) relative to shRFP, 0.75 mM glucose. Error bars are SEM (n=3). Replicates are biological, means reported. Asterisks in f indicate significance p<0.05 by two-sided student's t-test. (D) Validation of top hit identified as differentially required in high glucose conditions. Immunoblots depict suppression of PKM by shRNAs (PKM_{—}1, PKM_{—}2) compared to control (RFP). Bottom, proliferation of cells in 0.75 mM (blue) relative to 10 mM glucose (black) harboring shRNAs targeting PKM or control. Asterisks indicate probability value (p)<0.05 relative to RFP 0.75 mM glucose.

FIG. 15. Only a subset of OXPHOS genes scores as hits in the screen despite similar levels of knockdown by the shRNAs used in the screen. The upper plot shows that similar levels of knockdown of the indicated genes was achieved. The lower plot shows that COX5A scored as a hit while the other genes indicated did not.

FIG. 16. Schematic diagram of electron transport chain noting that differential requirement of electron transport chain components for proliferation under glucose limitation was confirmed using mitochondrial toxins.

FIG. 17. Schematic diagram of an experiment in which the ability of 30 cancer cell lines of diverse cancer types, each harboring distinct stable DNA barcodes to allow identification, to proliferate in conditions of low or high glucose was evaluated.

FIG. 18. Plot showing that cancer cells exhibit diverse responses to glucose limitation. Certain cancer cells show unchanged or increased ability to proliferate in low glucose (i.e., are resistant to glucose limitation) while others show decreased ability to proliferate in low glucose (i.e., are sensitive to glucose limitation).

FIG. 19. Left panel shows a schematic summary of results of transcriptome-wide correlation analysis for sensitivity to glucose limitation. Low CYC1 expression was highly correlated with sensitivity to glucose limitation. Right side shows Western blot confirming that CYC1 is expressed at only low levels in most glucose limitation sensitive cell lines and expressed at much higher levels in most glucose resistant cell lines. Inset at upper right indicates that CYC1 was the top hit in the screen for genes differentially required for proliferation under low glucose conditions.

FIG. 20. Investigation of potential reasons why certain cell lines are sensitive to glucose limitation. Plots showing measurement of mtDNA amount (left) and mitochondrial mass (right) in various glucose limitation sensitive and glucose limitation resistant cell lines.

FIG. 21. Plots of OCR (left) and OCR/ECAR (right) in various glucose limitation resistant (black bars) and glucose limitation sensitive (red bars) cancer cell lines cultured in conditions of 10 mM glucose. OCR=oxygen consumption rate. ECAR=ExtraCellular Acidification Rate. OCR or OCR/ECAR serves as an approximate measure of OXPHOS activity. ECAR serves as an approximate measure of glycolytic activity.

FIG. 22. Metabolic responses of cancer cells to glucose addition: Crabtree Effect. Plot showing fold increase in OCR (left panel) and ECAR (lower panel) when Jurkat cells cultured in media with 0.75 mM glucose are either maintained in media with 0.75 mM glucose or subjected to increasing concentrations of glucose up to 10 mM. The data show that glycolysis increases as glucose concentration is increased.

FIG. 23. (A) Metabolic responses of cell lines to glucose limitation. Plot showing fold increase in OCR (left panel) when cells of various glucose limitation resistant (black bars) and glucose limitation sensitive (red bars) are shifted from culture in media with 10 mM glucose to culture in 0.75 mM glucose. The right panel shows average fold change in OCR for the glucose limitation resistant (black) and glucose limitation sensitive (red) cell lines. The data show that glucose limitation sensitive cell lines exhibit a much lower increase in their OCR upon glucose limitation than do glucose limitation resistant cell lines. (B) Fold increase in OCR of indicated cell lines in 0.75 mM (blue) relative to 10 mM glucose (black). Error bars are SEM (n=5-6 for a, b, c, e, f, h, k; n=3 for d, g). Replicates are biological, means reported. Asterisks indicate significance p<0.05 by two-sided student's t-test.

FIG. 24. Metabolic responses of various glucose limitation resistant (black) and glucose limitation sensitive (red) cancer cell lines to mitochondrial uncoupling. Figure shows percent change in OCR relative to third basal measurement and upon addition of FCCP (measurements 4-6) in low glucose resistant (black) or sensitive lines (grey).

FIG. 25. Glucose consumption rate in 10 mM (black) or 0.75 mM glucose (blue) of indicated cell lines.

FIG. 26. The plot on the left shows fold increase in OCR (left panel) when cells of various glucose limitation resistant (black bars) and glucose limitation sensitive (red bars) are shifted from culture in media with 10 mM glucose to culture in 0.75 mM glucose. The plot is the same as shown in FIG. 23 and highlights the fact that KMS26 and NCI-H929 cells exhibit essentially no change in OCR upon shift to low glucose. The plot on the left shows that KMS26 and NCI-H929 cells have high basal OCR, indicating that these cell lines do not have a defect in mitochondrial activity.

FIG. 27. (A) Plot showing that KMS26 and NCI-H929 cells have low GLUT3 (SLC2A3) expression. (B) Expression (qPCR) of SLC2A1 (black) or SLC2A3 (grey) of indicated cell lines (log₂ scale relative to NCI-H929). (C) and (D) Glucose consumption rate of indicated cell lines under 0.75 mM glucose. (E) Proliferation (4 days) of control (Vector) or GLUT3 over-expressing (GLUT3) cell lines in 10 mM (black) or 0.75 mM glucose (blue).

FIG. 28. Plot showing that KMS-26 and NCI-H929 cells do not take up glucose effectively, particularly upon glucose limitation. The black bars (left bar in each pair of bars for each cell line) represents glucose uptake at 10 mM. The blue bars (right bar in each pair of bars) represents glucose uptake at 0.75 mM.

FIG. 29. Increased GLUT1 expression rescues proliferative defect of KMS-26 cells under glucose limitation. Left: Western blot showing expression of SLC2A1 (GLUT1) by KMS-26 cells after introduction of SLC2A1 (left) or control GFP (right) cDNA. Plots show increase in glucose uptake (left plot) and rescue of proliferation defect (right plot) by expression of SLC2A1.

FIG. 30. (A) Plot showing increase in glucose uptake by KMS-26 cells resulting from expression of SLC2A3 from introduced cDNA. (B) Plot showing rescue of proliferation defect in KMS-26 cells by expression of SLC2A3 from introduced cDNA.

FIG. 31. Same plots as shown in FIG. 26, highlighting certain cell lines whose low ability to increase OCR in response to glucose limitation is not explained by defects in glucose uptake.

FIG. 32. Plot showing that U937 cells have defective complex I activity and partial complex II activity.

FIG. 33. Sequencing reveals that U937 cells have mutations in various mtDNA genes that encode complex I components. Mutations identified in genes encoding ND1 and ND5 are shown.

FIG. 34. Diagram of mammalian mitochondrial DNA (mtDNA). Human mtDNA is a 16,569 bp circular DNA that encodes 13 of the ˜90 OXPHOS subunits. It exists in multiple copies within mitochondria. These copies may be identical (homoplasmy) or different (heteroplasmy). Somatic mutations in mtDNA have been identified in a variety of cancers, both in primary tumors as well as tumor cell lines.

FIG. 35. Diagram illustrating that damaging somatic mtDNA mutations occur frequently in tumors (13-63%) and showing the approximate distribution of various types of mutation.

FIG. 36. Left: Schematic diagram of experiment designed to examine sensitivity of glucose limitation sensitive and glucose limitation resistant cell lines to inhibition of OXPHOS brought about by shRNA-mediated inhibition of various genes encoding OXPHOS components. Right: Results (right) show that glucose limitation-sensitive cell lines are sensitive to OXPHOS inhibition.

FIG. 37. Plot showing response to metformin of various glucose limitation resistant (black bars, left) and glucose limitation sensitive (red bars, right) cell lines cultured in media with 0.75 mM glucose. Metformin has greater inhibitory effects on proliferation of glucose limitation sensitive cell lines than glucose limitation resistant cell lines.

FIG. 38. (A) Plot showing correlation of UQCRC1+CYC1 expression levels with metformin sensitivity. Cell lines with low expression of UQCRC1+CYC1 exhibit increased sensitivity (decreased proliferation) when exposed to metformin relative to cells with higher expression. (B) Plot showing that the sensitivity of cell lines to low glucose correlated with the combined sensitivity to metformin and low glucose.

FIG. 39. Tumors from glucose-limitation sensitive cell line are sensitive to metformin. Results of in vivo experiment in which metformin was administered to mice harboring tumors from cells of the indicated cell lines. Metformin treatment did not affect size of tumors from glucose limitation resistant cell line NCI-H82 but caused an approximately 50% reduction in size of tumors from glucose limitation sensitive cell line NCI-H929 as compared with the size of tumors in mice treated with vehicle (PBS). Micrographs on the right show increased level of cleaved caspase 3 in tumors from glucose limitation sensitive cell line NCI-H929 in mice treated with metformin as compared with vehicle. This effect was not observed in tumors from glucose limitation resistant cell line NCI-H82.

FIG. 40. Model of the metabolic determinants of sensitivity to low glucose and biguanides. This diagram outlines the interplay between reserve oxidative phosphorylation (OXPHOS) capacity, sensitivity to biguanides, and sensitivity to culture in low glucose. Most cancer cell lines and normal cells tested exhibited an ability to respond to glucose limitation by upregulating OXPHOS, rendering them less sensitive to biguanides and low glucose conditions. In contrast, cell lines harboring mutations in mtDNA encoded Complex I subunits or exhibiting impaired glucose utilization have a limited reserve OXPHOS capacity and are therefore unable to properly respond to biguanides and low glucose, rendering them sensitive to these perturbations. At the extreme, cells artificially engineered to have no OXPHOS (Rho cells) exhibit extreme low glucose sensitivity, but resistance to further inhibition of OXPHOS. Thus, mtDNA mutant cancer cells exist at an intermediate state of OXPHOS functionality that renders them sensitive to treatment with biguanides in vitro and in vivo. Similarly, cell lines with impaired glucose utilization exhibit biguanide sensitivity specifically under the low glucose conditions seen in the tumor microenvironment.

FIG. 41. Additional data characterizing mitochondrial dysfunction and impaired glucose utilization in cancer cell lines, a, Oxygen consumption rate (OCR) to extracellular acidification rate (ECAR) ratio (left) or OCR normalized to protein content (right) for glucose limitation resistant (black) or sensitive (blue) cell lines. b, Left, mitochondrial DNA content for indicated cell lines by qPCR using primers targeting ND1 (black) or ND2 (grey) normalized to gDNA repetitive element (Alu) relative to KMS-12BM. Right, mitochondrial mass measured by fluorescence intensity of mitotracker green dye for indicated cell lines. c, Percent change from baseline (second measurement) of ECAR or OCR in Jurkat cells where glucose concentration was maintained at 0.75 mM (blue) or increased to indicated concentrations (black). d, Uptake of 3H-labeled 2-DG (counts per minute per ng protein) in 0.75 mM glucose at indicated timepoints in GLUT3 high (grey) or low (blue) cell lines. e, Heatmap of gene expression values for genes indicated at top and cell lines indicated at left. Genes organized by p-value with lowest expressed genes in NCI-H929 and KMS-26 at left, those significantly lower are colored red. Expression values reported are Log 2 transformed fold difference from the median (scale color bar at right). f, Immunoblots for GLUT3 and NDI1 expression in indicated cell lines (beta-actin loading control). g,i, Proliferation of cell number in cells over-expressing GLUT3 or NDI1 relative to control vector (4 days). h, OCR of permeabilized cell indicated upon addition of indicated metabolic toxins and substrates. j, Fold change in OCR in indicated cells expressing NDI1 relative to control vector. k-l, Proliferation for 4 days of control (Vector) or NDI1 expressing cell lines indicated (NDI1) under 10 mM (black) and 0.75 mM glucose (blue). Error bars are SEM, n=4 for a-c, h, j; n=3 for d, g, i, k, l. Replicates are biological, means reported. Asterisks indicate significance p<0.05 by two-sided student's t-test.

FIG. 42. Gene expression signature for identifying cell lines with impaired glucose utilization. Heatmap of gene expression values for the genes indicated on the right for the cell lines in the CCLE set. Gene expression values are reported as the difference from the median across the entire sample set according to the scale color bar on the upper right. Genes 1-8 comprised the gene expression signature used to identify samples with impaired glucose utilization. Samples are sorted based upon this signature with those predicted to exhibit impaired glucose utilization at the top. The order of samples and all values are reported in Table 6.

FIG. 43. a, Viability of indicated lines, as measured by ATP levels on Day 3 at phenformin concentrations indicated by black-blue scale, in 0.75 mM glucose, compared to ATP levels on Day 0. Value of 1 indicates fully viable cells (untreated). Value of 0 indicates no change in ATP level compared to Day 0 (cytostatic). Negative values indicate decrease in ATP levels (−1 indicates no ATP). b, Viability as in a of NCI-H2171 and NCI-H929 cell lines under 0.75 and 10 mM glucose. c, Relative increase in cell number (top) and viability as in a (bottom) of control (Vector) or GLUT3 over-expressing (GLUT3) cell lines in 10 mM or 0.75 mM glucose at indicated phenformin concentrations relative to untreated cells in 10 mM glucose. d, Relative increase in cell number (top) and viability as in a (bottom) of vector control (black) or NDI1 (grey) expressing lines in 0.75 mM glucose at indicated phenformin concentrations relative to untreated cells in 0.75 mM glucose. e. Percent change in oxygen consumption rate (OCR) of control (Vector) or NDI1-expressing lines (NDI1) relative to the second basal measurement at indicated phenformin concentrations. f, Average volume (relative to Day 0) of established xenografted tumours derived from control (NCI-H2171, NCI-H82), mtDNA Complex I mutant (U-937), or impaired glucose utilization (NCI-H929) cell lines in mice treated with vehicle (black) or phenformin (blue) in drinking water starting at Day 0. g, Average tumor volume as in f of indicated cell lines infected with control, NDI1- or GLUT3-expressing vectors. Error bars are SEM (n=5 for a, b, c (bottom), d (bottom) and e; n=4-5 for f; n=6-8 for g; n=3 for c (top) and d (top)). Replicates are biological, means reported. Asterisks indicate significance p<0.05 by two-sided student's t-test.

FIG. 44. GLUT3 over-expression increases tumor xenograft growth and cell proliferation in low glucose media. a, KMS-26 cell lines infected with GLUT3 overexpressing vector or infected with control vector were mixed in equal proportions and cultured under different glucose concentrations. Additionally, these mixed cell lines were injected into NOD/SCID mice subcutaneously. 2.5 weeks later, genomic DNA was isolated from tumors as well as cells grown in vitro under the indicated glucose concentrations. Using qPCR, relative abundance of control vector and GLUT3 vector were determined and plotted relative to 10 mM glucose in culture (n=9). b, Average volume of unmixed tumor xenografts from KMS-26 cell lines infected with GLUT3 overexpressing vector relative to control vector (2.5 weeks) (n=6). Replicates are biological, means reported. Asterisks indicate significance p<0.05 by two-sided student's t-test.

FIG. 45. Sanger sequencing traces validating mtDNA mutations. Traces for each cell line (left) are shown in the order indicated by the table. “Reverse str” indicates instances when the sequence shown is in the reverse orientation to the revised Cambridge Reference Sequence. For each trace, the gene sequenced is at the bottom left, the DNA sequence is at the top, and the nucleotide alteration is in red text.

FIG. 46. Additional data supporting the hypersensitivity of cell lines with the identified biomarkers to biguanides. a-b, Viability (a, 10 mM glucose) or relative change in cell number (b, 4 days, glucose concentration indicated in key) of indicated cell lines at phenformin concentrations indicated. Viability measured by ATP levels on Day 3 at phenformin concentrations indicated by black-blue scale, compared to ATP levels on Day 0. Value of 1 indicates fully viable cells (untreated). Value of 0 indicates no change in ATP level compared to Day 0 (cytostatic). Negative values indicate decrease in ATP levels (−1 indicates no ATP). c, Viability as in (a) of indicated cell lines under 0.75 mM and 10 mM glucose at indicated phenformin concentrations. d, Left, relative change in cell number in 0.75 mM glucose, 2 mM metformin relative to untreated in glucose limitation resistant (black) and sensitive (blue) cell lines. Right, relative size of tumor xenografts derived from the indicated cell lines in mice injected with PBS or metformin (IP, 300 mg/kg/day). e, Viability as in (a) of NCI-H929 cells at the indicated concentrations of phenformin and glucose. f, Relative size of indicated cell line xenografts in mice treated with PBS or phenformin (1.7 mg/ml in drinking water). g, Percent change in oxygen consumption rate (OCR) of control (Vector) or NDI1-expressing lines (NDI1) relative to the second basal measurement and at indicated phenformin concentrations. h, Proliferation of 143B wild type or 143B rho (no mtDNA) cell lines under 0.75 mM or 10 mM glucose with or without phenformin treatment. Error bars are SEM (n=4 for a, c, e, g; n=3 for b, d, and h (left); n=5 for d (right) and f). Replicates are biological, means reported. Asterisks indicate significance p<0.05 by two-sided student's t-test.

FIG. 47. Long term treatment of mtDNA mutant cells with phenformin. a, Sanger-sequencing traces of mtDNA encoded ND1 and ND4 genes from Cal-62 cells expressing NDI1 or control vector cultured under 5-20 uM phenformin or no phenformin for 1.5 months. Regions containing mutant sequence indicated by red box. b, Heteroplasmy levels for mutation in ND1 or ND4 were assessed by measuring the relative areas under the curve from Sanger-sequencing and plotted. c, Cal-62 cell lines cultured with or without phenformin for 1.5 months assessed for their ability to proliferate in 0.75 mM glucose (blue) relative to 10 mM glucose (black). The proliferation assay was for 4 days in the absence of phenformin. d, Heteroplasmy levels of ND1 and ND4 as in b of Cal-62 tumor xenografts in mice treated with or without phenformin for 28 days. Error bars are SEM, n=3. Replicates are biological (c) or technical (b,d), means reported. Asterisks indicate significance p<0.05 by two-sided student's t-test.

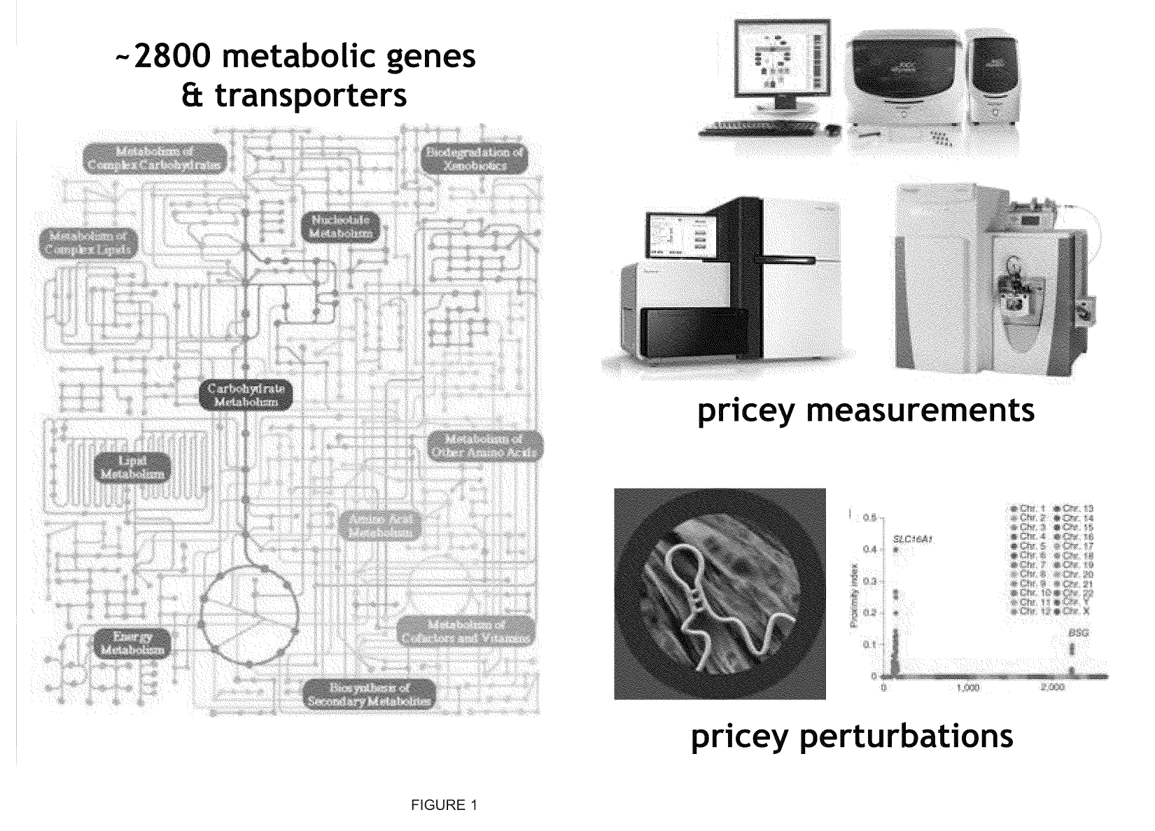

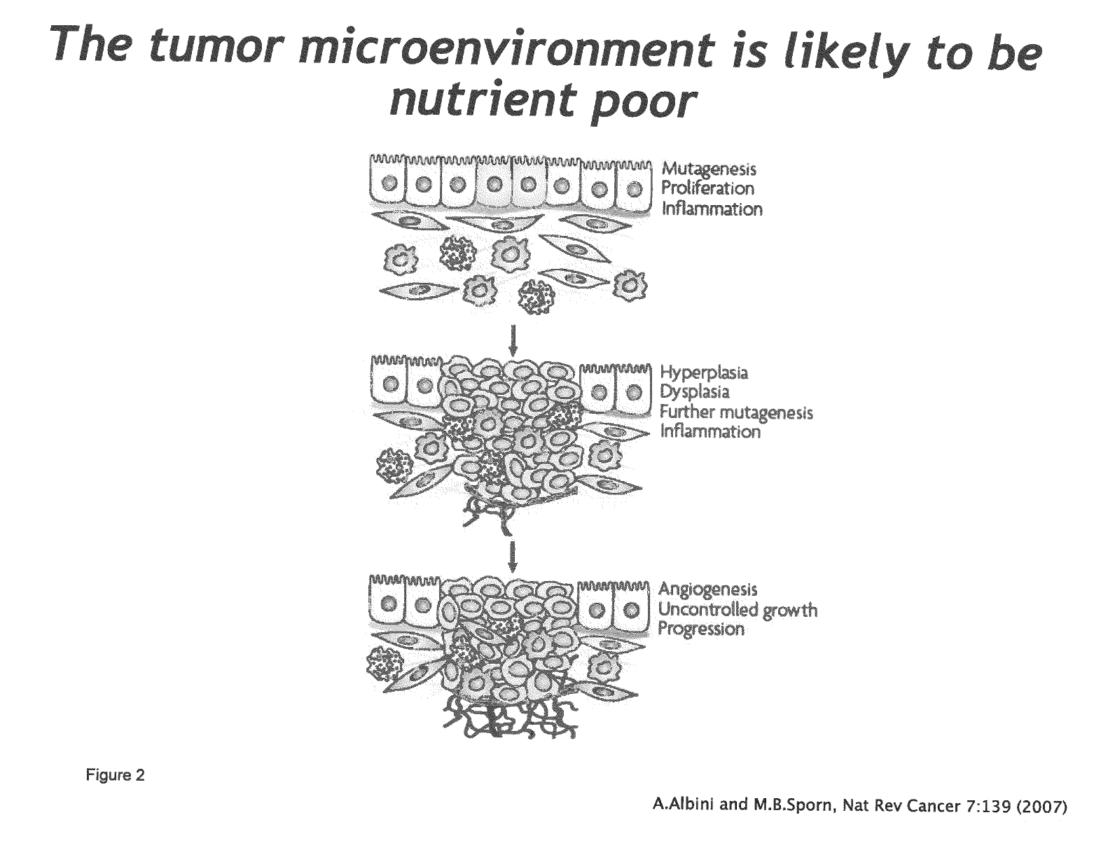

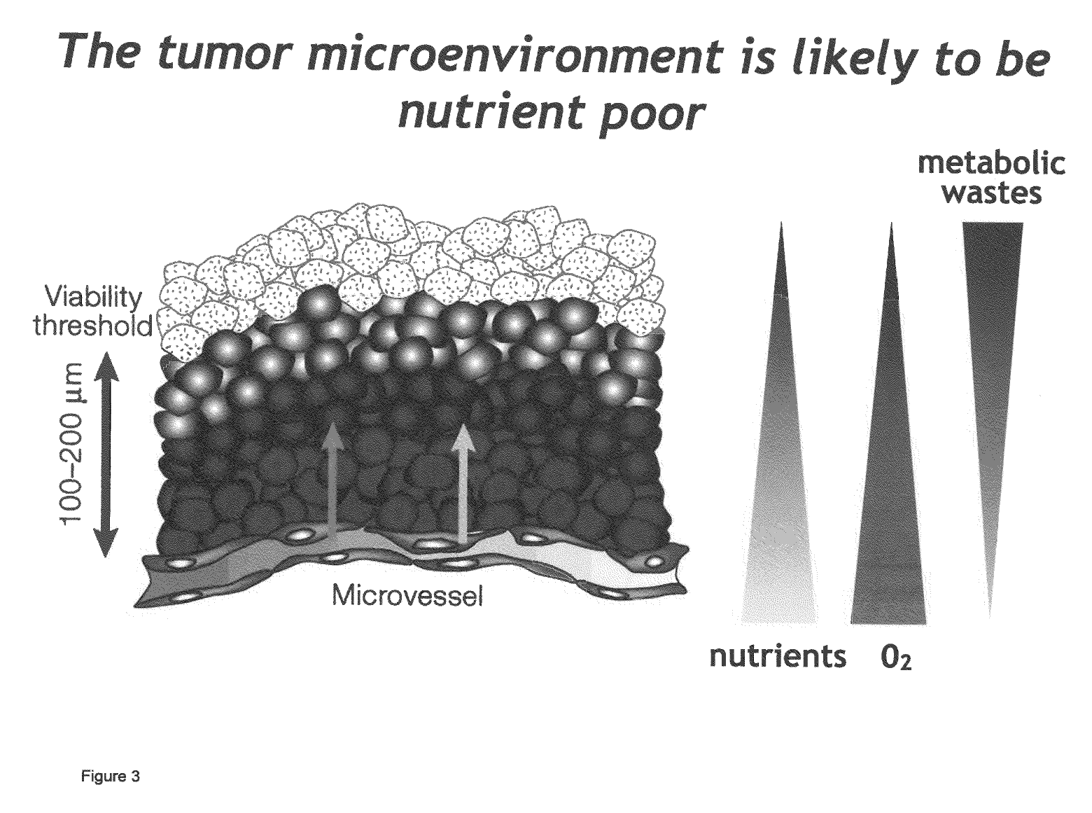

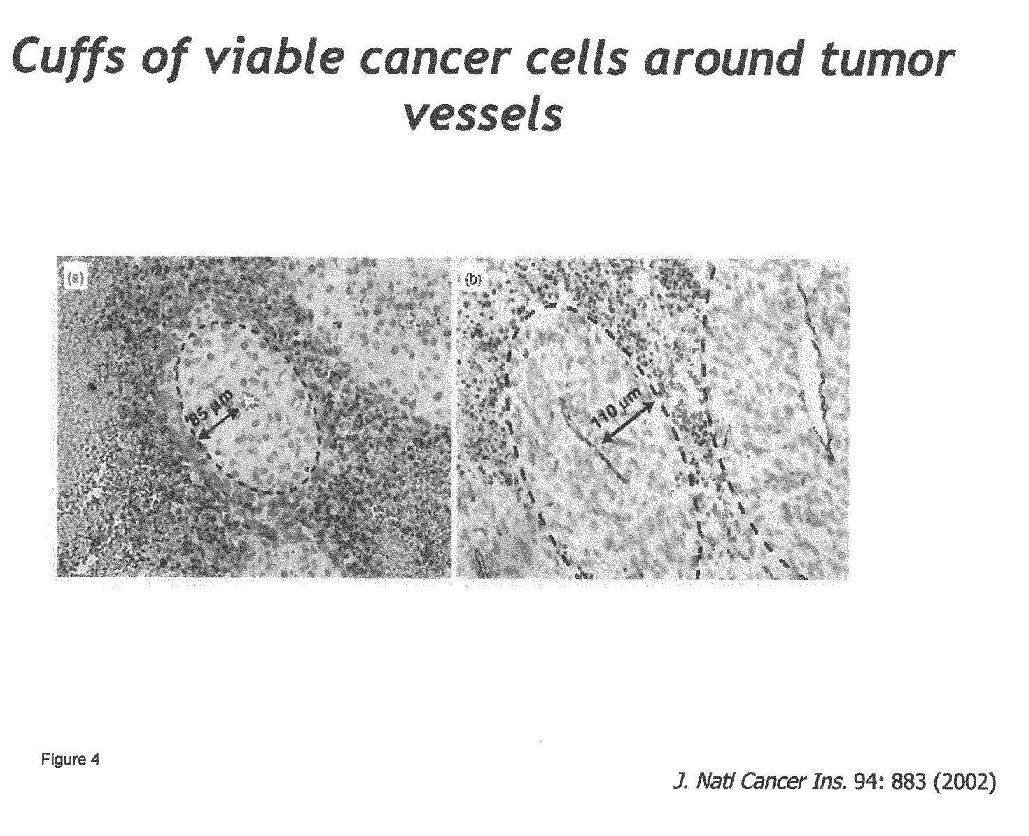

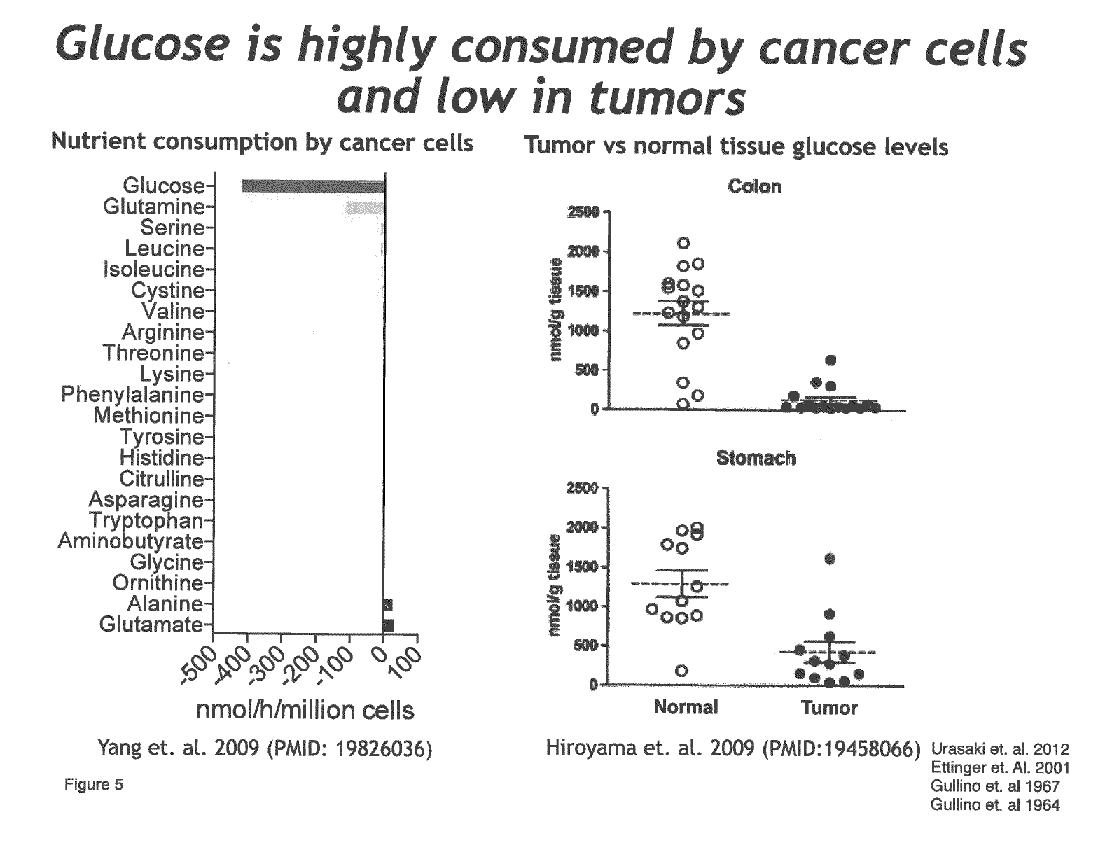

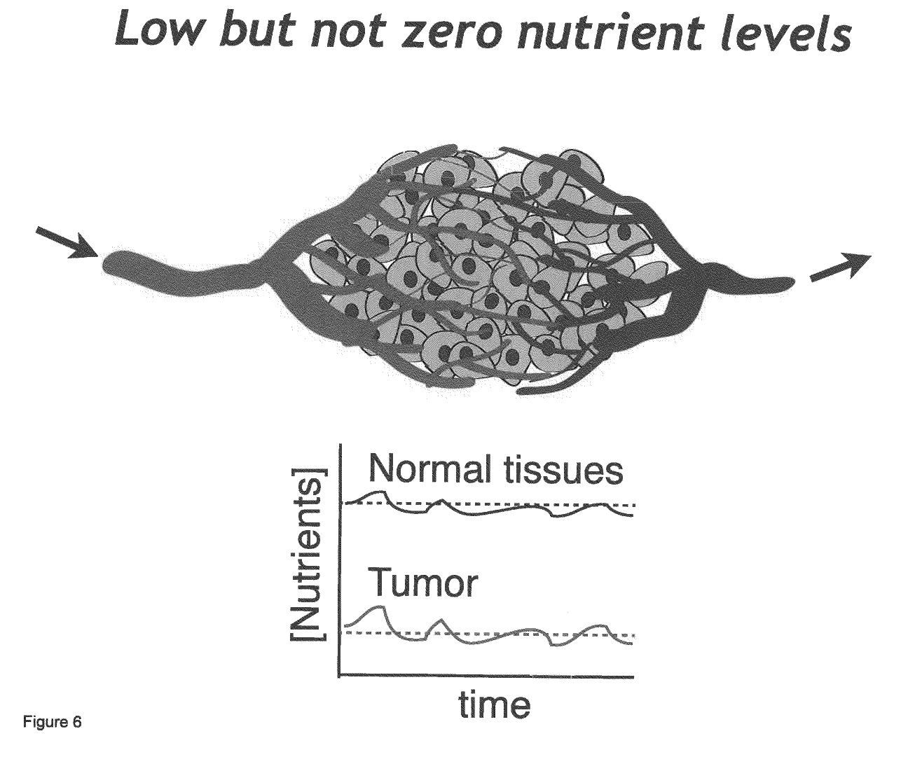

### DETAILED DESCRIPTION OF CERTAIN EMBODIMENTS

### I. Glossary

Descriptions and certain information relating to various terms used in the present disclosure are collected here for convenience.

“Agent” is used herein to refer to any substance, compound (e.g., molecule), supramolecular complex, material, or combination or mixture thereof. A compound may be any agent that can be represented by a chemical formula, chemical structure, or sequence. Example of agents, include, e.g., small molecules, polypeptides, nucleic acids (e.g., RNAi agents, antisense oligonucleotide, aptamers), lipids, polysaccharides, etc. In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

An “analog” of a first agent refers to a second agent that is structurally and/or functionally similar to the first agent. A “structural analog” of a first agent is an analog that is structurally similar to the first agent. A structural analog of an agent may have substantially similar physical, chemical, biological, and/or pharmacological propert(ies) as the agent or may differ in at least one physical, chemical, biological, or pharmacological property. In some embodiments at least one such property may be altered in a manner that renders the analog more suitable for a purpose of interest. In some embodiments a structural analog of an agent differs from the agent in that at least one atom, functional group, or substructure of the agent is replaced by a different atom, functional group, or substructure in the analog. In some embodiments, a structural analog of an agent differs from the agent in that at least one hydrogen or substituent present in the agent is replaced by a different moiety (e.g., a different substituent) in the analog. In some embodiments an analog may comprise a moiety that reacts with a target to form a covalent bond. In some embodiments an analog of an agent described herein may be used for the same purpose, e.g., a structural analog.

The terms “assessing”, “determining”, “evaluating”, “assaying” are used interchangeably herein to refer to any form of detection or measurement, and include determining whether a substance, signal, disease, condition, etc., is present or not. The result of an assessment may be expressed in qualitative and/or quantitative terms. Assessing may be relative or absolute. “Assessing the presence of” includes determining the amount of something that is present or determining whether it is present or absent.

“Cellular marker” refers to a molecule (e.g., a protein, RNA, DNA, lipid, carbohydrate), complex, or portion thereof, the presence, absence, or level of which in or on a cell (e.g., at least partly exposed at the cell surface) characterizes, indicates, or identifies one or more cell type(s), cell lineage(s), or tissue type(s) or characterizes, indicates, or identifies a particular state (e.g., a diseased or physiological state such as apoptotic or non-apoptotic, a differentiation state, a stem cell state). In some embodiments a cellular marker comprises the presence, absence, or level of a particular modification of a molecule or complex, e.g., a co- or post-translational modification of a protein. A level may be reported in a variety of different ways, e.g., high/low; +/−; numerically, etc. The presence, absence, or level of certain cellular marker(s) may indicate a particular physiological or diseased state of a patient, organ, tissue, or cell. It will be understood that multiple cellular markers may be assessed to, e.g., identify or isolate a cell type of interest, diagnose a disease, etc. In some embodiments between 2 and 10 cellular markers may be assessed. A cellular marker present on or at the surface of cells may be referred to as a “cell surface marker” (CSM). It will be understood that a CSM may be only partially exposed at the cell surface. In some embodiments a CSM or portion thereof is accessible to a specific binding agent present in the environment in which such cell is located, so that the binding agent may be used to, e.g., identify, label, isolate, or target the cell. In some embodiments a CSM is a protein at least part of which is located outside the plasma membrane of a cell. Examples of CSMs include CD molecules, receptors with an extracellular domain, channels, and cell adhesion molecules. In some embodiments, a receptor is a growth factor receptor, hormone receptor, integrin receptor, folate receptor, or transferrin receptor. A cellular marker may be cell type specific. A cell type specific marker is generally expressed or present at a higher level in or on (at the surface of) a particular cell type or cell types than in or on many or most other cell types (e.g., other cell types in the body or in an artificial environment). In some cases a cell type specific marker is present at detectable levels only in or on a particular cell type of interest and not on other cell types. However, useful cell type specific markers may not be and often are not absolutely specific for the cell type of interest. A cellular marker, e.g., a cell type specific marker, may be present at levels at least 1.5-fold, at least 2-fold or at least 3-fold greater in or on the surface of a particular cell type than in a reference population of cells which may consist, for example, of a mixture containing cells from multiple (e.g., 5-10; 10-20, or more) of different tissues or organs in approximately equal amounts. In some embodiments a cellular marker, e.g., a cell type specific marker, may be present at levels at least 4-5 fold, between 5-10 fold, between 10-fold and 20-fold, between 20-fold and 50-fold, between 50-fold and 100-fold, or more than 100-fold greater than its average expression in a reference population. It will be understood that a cellular marker, e.g., a CSM, may be present in a cell fraction, organelle, cell fragment, or other material originating from a cell in which it is present and may be used to identify, detect, or isolate such material. In general, the level of a cellular marker may be determined using standard techniques such as Northern blotting, in situ hybridization, RT-PCR, sequencing, immunological methods such as immunoblotting, immunohistochemistry, fluorescence detection following staining with fluorescently labeled antibodies (e.g., flow cytometry, fluorescence microscopy), similar methods using non-antibody ligands that specifically bind to the marker, oligonucleotide or cDNA microarray, protein microarray analysis, mass spectrometry, etc. A CSM, e.g., a cell type specific CSM, may be used to detect or isolate cells or as a target in order to deliver an agent to cells. For example, the agent may be linked to a moiety that binds to a CSM. Suitable binding moieties include, e.g., antibodies or ligands, e.g., small molecules, aptamers, or polypeptides. Methods known in the art can be used to separate cells that express a cellular marker, e.g., a CSM, from cells that do not, if desired. In some embodiments a specific binding agent can be used to physically separate cells that express a CSM from cells that do not. In some embodiments, flow cytometry is used to quantify cells that express a cellular marker, e.g., a CSM, or to separate cells that express a cellular marker, e.g., a CSM, from cells that do not. For example, in some embodiments cells are contacted with a fluorescently labeled antibody that binds to the CSM. Fluorescence activated cell sorting (FACS) is then used to separate cells based on fluorescence.

“Computer-assisted” as used herein encompasses methods in which a computer is used to gather, process, manipulate, display, visualize, receive, transmit, store, or in any way handle or analyze information (e.g., data, results, structures, sequences, etc.). A method may comprise causing the processor of a computer to execute instructions to gather, process, manipulate, display, receive, transmit, or store data or other information. The instructions may be embodied in a computer program product comprising a computer-readable medium. A computer-readable medium may be any tangible medium (e.g., a non-transitory storage medium) having computer usable program instructions embodied in the medium. Any combination of one or more computer usable or computer readable medium(s) may be utilized in various embodiments. A computer-usable or computer-readable medium may be or may be part of, for example but not limited to, an electronic, magnetic, optical, electromagnetic, infrared, or semiconductor system, apparatus, or device. Examples of a computer-readable medium include, e.g., a hard disk, a random access memory (RAM), a read-only memory (ROM), an erasable programmable read-only memory (e.g., EPROM or Flash memory), a portable compact disc read-only memory (CDROM), a floppy disk, an optical storage device, or a magnetic storage device. In some embodiments a method comprises transmitting or receiving data or other information over a communication network. The data or information may be generated at or stored on a first computer-readable medium at a first location, transmitted over the communication network, and received at a second location, where it may be stored on a second computer-readable medium. A communication network may, for example, comprise one or more intranets or the Internet.

“Detection reagent” refers to an agent that is useful to specifically detect a gene product or other analyte of interest, e.g., an agent that specifically binds to the gene product or other analyte. Examples of agents useful as detection reagents include, e.g., nucleic acid probes or primers that hybridize to RNA or DNA to be detected, antibodies, aptamers, or small molecule ligands that bind to polypeptides to be detected, and the like. In some embodiments a detection reagent comprises a label. In some embodiments a detection reagent is attached to a support. Such attachment may be covalent or noncovalent in various embodiments. Methods suitable for attaching detection reagents or analytes to supports will be apparent to those of ordinary skill in the art. A support may be a substantially planar or flat support or may be a particulate support, e.g., an approximately spherical support such as a microparticle (also referred to as a “bead”, “microsphere”), nanoparticle (or like terms), or population of microparticles. In some embodiments a support is a slide, chip, or filter. In some embodiments a support is at least a portion of an inner surface of a well or other vessel, channel, flow cell, or the like. A support may be rigid, flexible, solid, or semi-solid (e.g., gel). A support may be comprised of a variety of materials such as, for example, glass, quartz, plastic, metal, silicon, agarose, nylon, or paper. A support may be at least in part coated, e.g., with a polymer or substance comprising a reactive functional group suitable for attaching a detection reagent or analyte thereto.

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered to a subject in a single dose, or through use of multiple doses, in various embodiments.

The term “expression” encompasses the processes by which nucleic acids (e.g., DNA) are transcribed to produce RNA, and (where applicable) RNA transcripts are processed and translated into polypeptides.

The term “gene product” (also referred to herein as “gene expression product” or “expression product”) encompasses products resulting from expression of a gene, such as RNA transcribed from a gene and polypeptides arising from translation of such RNA. It will be appreciated that certain gene products may undergo processing or modification, e.g., in a cell. For example, RNA transcripts may be spliced, polyadenylated, etc., prior to mRNA translation, and/or polypeptides may undergo co-translational or post-translational processing such as removal of secretion signal sequences, removal of organelle targeting sequences, or modifications such as phosphorylation, fatty acylation, etc. The term “gene product” encompasses such processed or modified forms. Genomic, mRNA, polypeptide sequences from a variety of species, including human, are known in the art and are available in publicly accessible databases such as those available at the National Center for Biotechnology Information (www.ncbi.nih.gov) or Universal Protein Resource (www.uniprot.org). Databases include, e.g., GenBank, RefSeq, Gene, UniProtKB/SwissProt. UniProtKBiTrembl, and the like. In general, sequences, e.g., mRNA and polypeptide sequences, in the NCBI Reference Sequence database may be used as gene product sequences for a gene of interest. It will be appreciated that multiple alleles of a gene may exist among individuals of the same species. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations often do not alter the encoded amino acid sequence, although DNA polymorphisms that lead to changes in the sequence of the encoded proteins can exist. Examples of polymorphic variants can be found in, e.g., the Single Nucleotide Polymorphism Database (dbSNP), available at the NCBI website at www.ncbi.nlm.nih.gov/projects/SNP/. (Sherry S T, et al. (2001). “dbSNP: the NCBI database of genetic variation”. Nucleic Acids Res. 29 (1): 308-311; Kitts A, and Sherry S, (2009). The single nucleotide polymorphism database (dbSNP) of nucleotide sequence variation in The NCBI Handbook [Internet]. McEntyre J, Ostell J, editors. Bethesda (Md.): National Center for Biotechnology Information (US); 2002 (www.ncbi.nlm.nih.gov/bookshelf/br.fcgi?book=handbook&part=ch5). Multiple isoforms of certain proteins may exist, e.g., as a result of alternative RNA splicing or editing. In general, where aspects of this disclosure pertain to a gene or gene product, embodiments pertaining to allelic variants or isoforms are encompassed, if applicable, unless indicated otherwise. Certain embodiments may be directed to particular sequence(s), e.g., particular allele(s) or isoform(s).

“Identity” or “percent identity” is a measure of the extent to which the sequence of two or more nucleic acids or polypeptides is the same. The percent identity between a sequence of interest A and a second sequence B may be computed by aligning the sequences, allowing the introduction of gaps to maximize identity, determining the number of residues (nucleotides or amino acids) that are opposite an identical residue, dividing by the minimum of TG_{A} and TG_{B} (here TG_{A} and TG_{B} are the sum of the number of residues and internal gap positions in sequences A and B in the alignment), and multiplying by 100. When computing the number of identical residues needed to achieve a particular percent identity, fractions are to be rounded to the nearest whole number. Sequences can be aligned with the use of a variety of computer programs known in the art. For example, computer programs such as BLAST2, BLASTN, BLASTP, Gapped BLAST, etc., may be used to generate alignments and/or to obtain a percent identity. The algorithm of Karlin and Altschul (Karlin and Altschul, Proc. Natl. Acad. Sci. USA 87:22264-2268, 1990) modified as in Karlin and Altschul, Proc. Natl. Acad Sci. USA 90:5873-5877, 1993 is incorporated into the NBLAST and XBLAST programs of Altschul et al. (Altschul. et al., J. Mol. Biol. 215:403-410, 1990). In some embodiments, to obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. See the Web site having URL www.ncbi.nlm.nih.gov and/or McGinnis, S. and Madden, T L, W20-W25 Nucleic Acids Research, 2004, Vol. 32, Web server issue. Other suitable programs include CLUSTALW (Thompson J D, Higgins D G, Gibson T J, Nuc Ac Res, 22:4673-4680, 1994) and GAP (GCG Version 9.1; which implements the Needleman & Wunsch, 1970 algorithm (Needleman S B, Wunsch C D, J Mol Biol, 48:443-453, 1970.) Percent identity may be evaluated over a window of evaluation. In some embodiments a window of evaluation may have a length of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or more, e.g., 100%, of the length of the shortest of the sequences being compared. In some embodiments a window of evaluation is at least 100; 200; 300; 400; 500; 600; 700; 800; 900; 1,000; 1,200; 1,500; 2,000; 2,500; 3,000; 3,500; 4,000; 4,500; or 5,000 amino acids. In some embodiments no more than 20%, 10%, 5%, or 1% of positions in either sequence or in both sequences over a window of evaluation are occupied by a gap. In some embodiments no more than 20%, 10%, 5%, or 1% of positions in either sequence or in both sequences are occupied by a gap.

“Inhibit” may be used interchangeably with terms such as “suppress”, “decrease”, “reduce” and like terms, as appropriate in the context. It will be understood that the extent of inhibition may vary. For example, inhibition may refer to a reduction of the relevant level by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99%. In some embodiments inhibition refers to a decrease of 100%, e.g., to background levels or undetectable levels. In some embodiments inhibition is statistically significant. The term “inhibitor” generally refers to an agent that inhibits a target, e.g., a target molecule, pathway, or process. For example, an inhibitor may inhibit expression of a gene. In some embodiments an inhibitor inhibits expression or activity of a gene product.

“Isolated” means 1) separated from at least some of the components with which it is usually associated in nature; 2) prepared or purified by a process that involves the hand of man; and/or 3) not occurring in nature, e.g., present in an artificial environment. In some embodiments an isolated cell is a cell that has been removed from a subject, separated from at least some other cells in a cell population, or a cell that remains after at least some other cells in a cell population have been removed or eliminated.

The term “label” (also referred to as “detectable label”) refers to any moiety that facilitates detection and, optionally, quantification, of an entity that comprises it or to which it is attached. In general, a label may be detectable by, e.g., spectroscopic, photochemical, biochemical, immunochemical, electrical, optical, chemical or other means. In some embodiments a detectable label produces an optically detectable signal (e.g., emission and/or absorption of light), which can be detected e.g., visually or using suitable instrumentation such as a light microscope, a spectrophotometer, a fluorescence microscope, a fluorescent sample reader, a fluorescence activated cell sorter, a camera, or any device containing a photodetector. Labels that may be used in various embodiments include, e.g., organic materials (including organic small molecule fluorophores (sometimes termed “dyes”), quenchers (e.g., dark quenchers), polymers, fluorescent proteins); enzymes; inorganic materials such as metal chelates, metal particles, colloidal metal, metal and semiconductor nanocrystals (e.g., quantum dots); compounds that exhibit luminescensce upon enzyme-catalyzed oxidation such as naturally occurring or synthetic luciferins (e.g., firefly luciferin or coelenterazine and structurally related compounds); haptens (e.g., biotin, dinitrophenyl, digoxigenin); radioactive atoms (e.g., radioisotopes such as ³H, ¹⁴C, ³²P ³³P, ³⁵S, ¹²⁵I), stable isotopes (e.g., ¹³C, ²H); magnetic or paramagnetic molecules or particles, etc. Fluorescent dyes include, e.g., acridine dyes; BODIPY, coumarins, cyanine dyes, napthalenes (e.g., dansyl chloride, dansyl amide), xanthene dyes (e.g., fluorescein, rhodamines), and derivatives of any of the foregoing. Examples of fluorescent dyes include Cy3, Cy3.5, Cy5. Cy5.5, Cy7, Alexa® Fluor dyes, DyLight® Fluor dyes, FITC, TAMRA, Oregon Green dyes, Texas Red, to name but a few. Fluorescent proteins include green fluorescent protein (GFP), blue, sapphire, yellow, red, orange, and cyan fluorescent proteins and fluorescent variants such as enhanced GFP (eGFP), mFruits such as mCherry, mTomato, mStrawberry; R-Phycoerythrin, etc. Enzymes useful as labels include, e.g., enzymes that act on a substrate to produce a colored, fluorescent, or luminescent substance. Examples include luciferases, beta-galactosidase, horseradish peroxidase, and alkaline phosphatase. Luciferases include those from various insects (e.g., fireflies, beetles) and marine organisms (e.g., cnidaria such as Renilla (e.g., Renilla reniformis, copepods such as Gaussia(e.g., Gaussia princeps) or Metridia (e.g., Metridia longa, Metridia pacifica), and modified versions of the naturally occurring proteins. A wide variety of systems for labeling and/or detecting labels or labeled entities are known in the art. Numerous detectable labels and methods for their use, detection, modification, and/or incorporation into or conjugation (e.g., covalent or noncovalent attachment) to biomolecules such as nucleic acids or proteins, etc., are described in Iain Johnson, I., and Spence, M. T. Z. (Eds.), The Molecular Probes® Handbook—A Guide to Fluorescent Probes and Labeling Technologies. 11th edition (Life Technologies/Invitrogen Corp.) available online on the Life Technologies website at http://www.invitrogen.com/site/us/en/home/References/Molecular-Probes-The-Handbook.html and Hermanson, G T., Bioconjugate Techniques, 2^{nd} ed., Academic Press (2008). Many labels are available as derivatives that are attached to or incorporate a reactive functional group so that the label can be conveniently conjugated to a biomolecule or other entity of interest that comprises an appropriate second functional group (which second functional group may either occur naturally in the biomolecule or may be introduced during or after synthesis). For example, an active ester (e.g., a succinimidyl ester), carboxylate, isothiocyanate, or hydrazine group can be reacted with an amino group; a carbodiimide can be reacted with a carboxyl group; a maleimide, iodoacetamide, or alkyl bromide (e.g., methyl bromide) can be reacted with a thiol (sulfhydryl); an alkyne can be reacted with an azide (via a click chemistry reaction such as a copper-catalyzed or copper-free azide-alkyne cycloaddition). Thus, for example, an N-hydroxysuccinide (NHS)-functionalized derivative of a fluorophore or hapten (such as biotin) can be reacted with a primary amine such as that present in a lysine side chain in a protein or in an aminoallyl-modified nucleotide incorporated into a nucleic acid during synthesis. A label may be directly attached to an entity or may be attached to an entity via a spacer or linking group, e.g., an alkyl, alkylene, aminoallyl, aminoalkynyl, or oligoethylene glycol spacer or linking group, which may have a length of, e.g., between 1 and 4, 4-8, 8-12, 12-20 atoms, or more in various embodiments. A label or labeled entity may be directly detectable or indirectly detectable in various embodiments. A label or labeling moiety may be directly detectable (i.e., it does not require any further reaction or reagent to be detectable, e.g., a fluorophore is directly detectable) or it may be indirectly detectable (e.g., it is rendered detectable through reaction or binding with another entity that is detectable, e.g., a hapten is detectable by immunostaining after reaction with an appropriate antibody comprising a reporter such as a fluorophore or enzyme; an enzyme acts on a substrate to generate a directly detectable signal). A label may be used for a variety of purposes in addition to or instead of detecting a label or labeled entity. For example, a label can be used to isolate or purify a substance comprising the label or having the label attached thereto. The term “labeled” is used herein to indicate that an entity (e.g., a molecule, probe, cell, tissue, etc.) comprises or is physically associated with (e.g., via a covalent bond or noncovalent association) a label, such that the entity can be detected. In some embodiments a detectable label is selected such that it generates a signal that can be measured and whose intensity is related to (e.g., proportional to) the amount of the label. In some embodiments two or more different labels or labeled entities are used or present in a composition. In some embodiments the labels may be selected to be distinguishable from each other. For example, they may absorb or emit light of different wavelengths. In some embodiments the labels may be selected to interact with each other. For example, a first label may be a donor molecule that transfers energy to a second label, which serves as an acceptor molecule through nonradiative dipole-dipole coupling as in resonance energy transfer (RET), e.g., Förster resonance energy transfer (FRET, also commonly nfluorescence resonance energy transfer), etc.

“Modulate” as used herein means to decrease (e.g., inhibit, reduce) or increase (e.g., stimulate, activate) a level, response, property, activity, pathway, or process. A “modulator” is an agent capable of modulating a level, response, property, activity, pathway, or process. A modulator may be an inhibitor, antagonist, activator, or agonist.

“Nucleic acid” is used interchangeably with “polynucleotide” and encompasses polymers of nucleotides. “Oligonucleotide” refers to a relatively short nucleic acid, e.g., typically between about 4 and about 100 nucleotides (nt) long, e.g., between 8-60 nt or between 10-40 nt long. Nucleotides include, e.g., ribonucleotides or deoxyribonucleotides. In some embodiments a nucleic acid comprises or consists of DNA or RNA. In some embodiments a nucleic acid comprises or includes only standard nucleobases (often referred to as “bases”). The standard bases are cytosine, guanine, adenine (which are found in DNA and RNA), thymine (which is found in DNA) and uracil (which is found in RNA), abbreviated as C, G, A, T, and U, respectively. In some embodiments a nucleic acid may comprise one or more non-standard nucleobases, which may be naturally occurring or non-naturally occurring (i.e., artificial; not found in nature) in various embodiments. In some embodiments a nucleic acid may comprise one or more chemically or biologically modified bases (e.g., alkylated (e.g., methylated) bases), modified sugars (e.g., 2′-O-alkyribose (e.g., 2′-O methylribose), 2′-fluororibose, arabinose, or hexose), modified phosphate groups or modified internucleoside linkages (i.e., a linkage other than a phosphodiester linkage between consecutive nucleosides, e.g., between the 3′ carbon atom of one sugar molecule and the 5′ carbon atom of another), such as phosphorothioates, 5′-N-phosphoramidites, alkylphosphonates, phosphorodithioates, phosphate esters, alkylphosphonothioates, phosphoramidates, carbamates, carbonates, phosphate triesters, acetamidates, carboxymethyl esters and peptide bonds). In some embodiments a modified base has a label (e.g., a small organic molecule such as a fluorophore dye) covalently attached thereto. In some embodiments the label or a functional group to which a label can be attached is incorporated or attached at a position that is not involved in Watson-Crick base pairing such that a modification at that position will not significantly interfere with hybridization. For example the C-5 position of UTP and dUTP is not involved in Watson-Crick base-pairing and is a useful site for modification or attachment of a label. In some embodiments a “modified nucleic acid” is a nucleic acid characterized in that (1) at least two of its nucleosides are covalently linked via a non-standard internucleoside linkage (i.e., a linkage other than a phosphodiester linkage between the 5′ end of one nucleotide and the 3′ end of another nucleotide); (2) it incorporates one or more modified nucleotides (which may comprise a modified base, sugar, or phosphate); and/or (3) a chemical group not normally associated with nucleic acids in nature has been covalently attached to the nucleic acid. Modified nucleic acids include, e.g., locked nucleic acids (in which one or more nucleotides is modified with an extra bridge connecting the 2′ oxygen and 4′ carbon i.e., at least one 2′-O,4′-C-methylene-β-D-ribofuranosyl nucleotide), morpholinos (nucleic acids in which at least some of the nucleobases are bound to morpholine rings instead of deoxyribose or ribose rings and linked through phosphorodiamidate groups instead of phosphates), and peptide nucleic acids (in which the backbone is composed of repeating N-(2-aminoethyl)-glycine units linked by peptide bonds and the nucleobases are linked to the backbone by methylene carbonyl bonds). Modifications may occur anywhere in a nucleic acid. A modified nucleic acid may be modified throughout part or all of its length, may contain alternating modified and unmodified nucleotides or internucleoside linkages, or may contain one or more segments of unmodified nucleic acid and one or more segments of modified nucleic acid. A modified nucleic acid may contain multiple different modifications, which may be of different types. A modified nucleic acid may have increased stability (e.g., decreased susceptibility to spontaneous or nuclease-catalyzed hydrolysis) or altered hybridization properties (e.g., increased affinity or specificity for a target, e.g., a complementary nucleic acid), relative to an unmodified counterpart having the same nucleobase sequence. In some embodiments a modified nucleic acid comprises a modified nucleobase having a label covalently attached thereto. Non-standard nucleotides and other nucleic acid modifications known in the art as being useful in the context of nucleic acid detection reagents, RNA interference (RNAi), aptamer, or antisense-based molecules for research or therapeutic purposes are contemplated for use in various embodiments of the instant invention. See, e.g., The Molecular Probes® Handbook—A Guide to Fluorescent Probes and Labeling Technologies (cited above), Bioconjugate Techniques (cited above), Crooke, S T (ed.) Antisense drug technology: principles, strategies, and applications, Boca Raton: CRC Press, 2008; Kurrcek. J. (ed.) Therapeutic oligonucleotides, RSC biomolecular sciences. Cambridge: Royal Society of Chemistry, 2008. A nucleic acid can be single-stranded, double-stranded, or partially double-stranded. An at least partially double-stranded nucleic acid can have one or more overhangs, e.g., 5′ and/or 3′ overhang(s). Where a nucleic acid sequence is disclosed herein, it should be understood that its complement and double-stranded form is also disclosed.

A “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. In some embodiments, a non-standard, naturally occurring amino acid is found in mammals. For example, omithine, citrulline, and homocysteine are naturally occurring non-standard amino acids that have important roles in mammalian metabolism. Examples of non-standard amino acids include, e.g., singly or multiply halogenated (e.g., fluorinated) amino acids, D-amino acids, homo-amino acids, N-alkyl amino acids (other than proline), dehydroamino acids, aromatic amino acids (other than histidine, phenylalanine, tyrosine and tryptophan), and α,α disubstituted amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, etc. Modifications may occur anywhere in a polypeptide, e.g., the peptide backbone, the amino acid side-chains and the amino or carboxyl termini. A given polypeptide may contain many types of modifications. Polypeptides may be branched or they may be cyclic, with or without branching. Polypeptides may be conjugated with, encapsulated by, or embedded within a polymer or polymeric matrix, dendrimer, nanoparticle, microparticle, liposome, or the like. Modification may occur prior to or after an amino acid is incorporated into a polypeptide in various embodiments. Polypeptides may, for example, be purified from natural sources, produced in vitro or in vivo in suitable expression systems using recombinant DNA technology (e.g., by recombinant host cells or in transgenic animals or plants), synthesized through chemical means such as conventional solid phase peptide synthesis, and/or methods involving chemical ligation of synthesized peptides (see, e.g., Kent, S., J Pept Sci., 9(9):574-93, 2003 or U.S. Pub. No. 20040115774), or any combination of the foregoing.

As used herein, the term “purified” refers to agents that have been separated from most of the components with which they are associated in nature or when originally generated or with which they were associated prior to purification. In general, such purification involves action of the hand of man. Purified agents may be partially purified, substantially purified, or pure. Such agents may be, for example, at least 50%, 60%, 70%, 75%, 80%, 85%, 90%, 95%, 96%, 97%, 98%, 99%, or more than 99% pure. In some embodiments, a nucleic acid, polypeptide, or small molecule is purified such that it constitutes at least 75%, 80%, 85%, 90%, 95%, 96%, 97%, 98%, 99%, or more, of the total nucleic acid, polypeptide, or small molecule material, respectively, present in a preparation. In some embodiments, an organic substance, e.g., a nucleic acid, polypeptide, or small molecule, is purified such that it constitutes at least 75%, 80%, 85%, 90%, 95%, 96%, 97%, 98%, 99%, or more, of the total organic material present in a preparation. Purity may be based on, e.g., dry weight, size of peaks on a chromatography tracing (GC, HPLC, etc.), molecular abundance, electrophoretic methods, intensity of bands on a gel, spectroscopic data (e.g., NMR), elemental analysis, high throughput sequencing, mass spectrometry, or any art-accepted quantification method. In some embodiments, water, buffer substances, ions, and/or small molecules (e.g., synthetic precursors such as nucleotides or amino acids), can optionally be present in a purified preparation. A purified agent may be prepared by separating it from other substances (e.g., other cellular materials), or by producing it in such a manner to achieve a desired degree of purity. In some embodiments “partially purified” with respect to a molecule produced by a cell means that a molecule produced by a cell is no longer present within the cell, e.g., the cell has been lysed and, optionally, at least some of the cellular material (e.g., cell wall, cell membrane(s), cell organelle(s)) has been removed and/or the molecule has been separated or segregated from at least some molecules of the same type (protein, RNA, DNA, etc.) that were present in the lysate.

The term “RNA interference” (RNAi) encompasses processes in which a molecular complex known as an RNA-induced silencing complex (RISC) silences or “knocks down” gene expression in a sequence-specific manner in, e.g., eukaryotic cells, e.g., vertebrate cells, or in an appropriate in vitro system. RISC may incorporate a short nucleic acid strand (e.g., about 16-about 30 nucleotides (nt) in length) that pairs with and directs or “guides” sequence-specific degradation or translational repression of RNA (e.g., mRNA) to which the strand has complementarity. The short nucleic acid strand may be referred to as a “guide strand” or “antisense strand”. An RNA strand to which the guide strand has complementarity may be referred to as a “target RNA”. A guide strand may initially become associated with RISC components (in a complex sometimes termed the RISC loading complex) as part of a short double-stranded RNA (dsRNA), e.g., a short interfering RNA (siRNA).

As used herein, the term “RNAi agent” encompasses nucleic acids that can be used to achieve RNAi in eukaryotic cells. Short interfering RNA (siRNA), short hairpin RNA (shRNA), and microRNA (miRNA) are examples of RNAi agents. siRNAs typically comprise two separate nucleic acid strands that are hybridized to each other to form a structure that contains a double stranded (duplex) portion at least 15 nt in length, e.g., about 15-about 30 nt long, e.g., between 17-27 nt long, e.g., between 18-25 nt long, e.g., between 19-23 nt long, e.g., 15, 16, 17, 18, 19, 20, 21, 22, 23, 24, 25, 26, 27, 28, 29, or 30 nucleotides. In some embodiments the strands of an siRNA are perfectly complementary to each other within the duplex portion. In some embodiments the duplex portion may contain one or more unmatched nucleotides, e.g., one or more mismatched (non-complementary) nucleotide pairs or bulged nucleotides. In some embodiments either or both strands of an siRNA may contain up to about 1, 2, 3, or 4 unmatched nucleotides within the duplex portion. In some embodiments a strand may have a length of between 15-35 nt, e.g., between 17-29 nt, e.g., 19-25 nt, e.g., 21-23 nt. Strands may be equal in length or may have different lengths in various embodiments. In some embodiments strands may differ by between 1-10 nt in length. A strand may have a 5′ phosphate group and/or a 3′ hydroxyl (—OH) group. Either or both strands of an siRNA may comprise a 3′ overhang of, e.g., about 1-10 nt (e.g., 1-5 nt, e.g., 2 nt). Overhangs may be the same length or different in lengths in various embodiments. In some embodiments an overhang may comprise or consist of deoxyribonucleotides, ribonucleotides, or modified nucleotides or modified ribonucleotides such as 2′-O-methylated nucleotides, or 2′-O-methyl-uridine. An overhang may be perfectly complementary, partly complementary, or not complementary to a target RNA in a hybrid formed by the guide strand and the target RNA in various embodiments. shRNAs are nucleic acid molecules that comprise a stem-loop structure and a length typically between about 40-150 nt, e.g., about 50-100 nt, e.g., 60-80 nt. A “stem-loop structure” (also referred to as a “hairpin” structure) refers to a nucleic acid having a secondary structure that includes a region of nucleotides which are known or predicted to form a double strand (stem portion; duplex) that is linked on one side by a region of (usually) predominantly single-stranded nucleotides (loop portion). Such structures are well known in the art and the term is used consistently with its meaning in the art. A guide strand sequence may be positioned in either arm of the stem, i.e., 5′ with respect to the loop or 3′ with respect to the loop in various embodiments. As is known in the art, the stem structure does not require exact base-pairing (perfect complementarity). Thus, the stem may include one or more unmatched residues or the base-pairing may be exact, i.e., it may not include any mismatches or bulges. In some embodiments the stem is between 15-30 nt, e.g., between 17-29 nt, e.g., 19-25 nt. In some embodiments the stem is between 15-19 nt. In some embodiments a loop sequence may be absent (in which case the termini of the duplex portion may be directly linked). In some embodiments a loop sequence may be at least partly self-complementary. In some embodiments the loop is between 1 and 20 nt in length, e.g., 1-15 nt, e.g., 4-9 nt. The shRNA structure may comprise a 5′ or 3′ overhang. As known in the art, an shRNA may undergo intracellular processing, e.g., by the ribonuclease (RNase) III family enzyme known as Dicer, to remove the loop and generate an siRNA.

Mature endogenous miRNAs are short (typically 18-24 nt, e.g., about 22 nt), single-stranded RNAs that are generated by intracellular processing from larger, endogenously encoded precursor RNA molecules termed miRNA precursors (see, e.g., Bartel, D., Cell. 116(2):281-97 (2004); Bartel DP. Cell. 136(2):215-33 (2009); Winter, J., et al., Nature Cell Biology 11: 228-234 (2009). Artificial miRNA may be designed to take advantage of the endogenous RNAi pathway in order to silence a target RNA of interest.

In some embodiments an RNAi agent is a vector (e.g., an expression vector) suitable for causing intracellular expression of one or more transcripts that give rise to a siRNA, shRNA, or miRNA in the cell. Such a vector may be referred to as an “RNAi vector”. An RNAi vector may comprise a template that, when transcribed, yields transcripts that may form a siRNA (e.g., as two separate strands that hybridize to each other), shRNA, or miRNA precursor (e.g., pri-miRNA or pre-mRNA).

An RNAi agent that contains a strand sufficiently complementary to an RNA of interest so as to result in reduced expression of the RNA of interest (e.g., as a result of degradation or repression of translation of the RNA) in a cell or in an in vitro system capable of mediating RNAi and/or that comprises a sequence that is at least 80%, 90%, 95%, or more (e.g., 100%) complementary to a sequence comprising at least 10, 12, 15, 17, or 19 consecutive nucleotides of an RNA of interest may be referred to as being “targeted to” the RNA of interest. An RNAi agent targeted to an RNA transcript may also considered to be targeted to a gene from which the transcript is transcribed. An RNAi agent may be produced in any of variety of ways in various embodiments. For example, nucleic acid strands may be chemically synthesized (e.g., using standard nucleic acid synthesis techniques) or may be produced in cells or using an in vitro transcription system. Strands may be allowed to hybridize (anneal) in an appropriate liquid composition (sometimes termed an “annealing buffer”). An RNAi vector may be produced using standard recombinant nucleic acid techniques.

The term “sample” may be used to generally refer to an amount or portion of something. A sample may be a smaller quantity taken from a larger amount or entity; however, a complete specimen may also be referred to as a sample where appropriate. A sample is often intended to be similar to and representative of a larger amount of the entity of which it is a sample. In some embodiments a sample is a quantity of a substance that is or has been or is to be provided for assessment (e.g., testing, analysis, measurement) or use. A sample may be any biological specimen. In some embodiments a sample comprises a body fluid such as blood, cerebrospinal fluid, (CSF), sputum, lymph, mucus, saliva, a glandular secretion, or urine. In some embodiments a sample comprises cells, tissue, or cellular material (e.g., material derived from cells, such as a cell lysate or fraction thereof). A sample may be obtained from (i.e., originates from, was initially removed from) a subject. Methods of obtaining biological samples from subjects are known in the art and include, e.g., tissue biopsy, such as excisional biopsy, incisional biopsy, core biopsy; fine needle aspiration biopsy; surgical excision, brushings; lavage; or collecting body fluids that may contain cells, such as blood, sputum, lymph, mucus, saliva, or urine. A sample is often intended to be similar to and representative of a larger amount of the entity of which it is a sample. A sample of a cell line comprises a limited number of cells of that cell line. A tumor sample is a sample that comprises at least some tumor cells, e.g., at least some tumor tissue. In some embodiments a sample may be obtained from an individual who has been diagnosed with or is suspected of having cancer. In some embodiments a sample is obtained from a tumor, e.g., a solid tumor. In some embodiments a tumor sample is obtained from the interior of a tumor. In some embodiments a tumor sample may comprise some non-tumor tissue or non-tumor cells, in addition to tumor tissue or tumor cells. For example a sample from the edge of a tumor may include some tumor tissue and some non-tumor tissue. A tumor sample may be obtained from a tumor prior to, during, or following removal of the tumor from a subject, or without removing the tumor from the subject. In some embodiments a sample contains at least some intact cells. In some embodiments a sample retains at least some of the microarchitecture of a tissue from which it was removed. A sample may be subjected to one or more processing steps, e.g., after having been obtained from a subject, and/or may be split into one or more portions. For example, in some embodiments a sample comprises plasma or serum obtained from a blood sample that has been processed to obtain such plasma or serum. The term sample encompasses processed samples, portions of samples, etc., and such samples are, where applicable, considered to have been obtained from the subject from whom the initial sample was removed. A sample may be procured directly from a subject, or indirectly, e.g., by receiving the sample from one or more persons who procured the sample directly from the subject, e.g., by performing a biopsy, surgery, or other procedure on the subject. In some embodiments a sample may be assigned an identifier (ID), which may be used to identify the sample as it is transported, processed, analyzed, and/or stored. In some embodiments the sample ID corresponds to the subject from whom the sample originated and allows the sample and/or results obtained by assessing the sample to be matched with the subject. In some embodiments the sample has an identifier affixed thereto. In some embodiments the identifier comprises a bar code.

A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

“Specific binding” generally refers to a physical association between a target molecule (e.g., a polypeptide) or complex and a binding agent such as an antibody, aptamer or ligand. The association is typically dependent upon the presence of a particular structural feature of the target such as an antigenic determinant, epitope, binding pocket or cleft, recognized by the binding agent. For example, if an antibody is specific for epitope A, the presence of a polypeptide containing epitope A or the presence of free unlabeled A in a reaction containing both free labeled A and the binding agent that binds thereto, will typically reduce the amount of labeled A that binds to the binding agent. It is to be understood that specificity need not be absolute but generally refers to the context in which the binding occurs. For example, it is well known in the art that antibodies may in some instances cross-react with other epitopes in addition to those present in the target. Such cross-reactivity may be acceptable depending upon the application for which the antibody is to be used. One of ordinary skill in the art will be able to select binding agents, e.g., antibodies, aptamers, or ligands, having a sufficient degree of specificity to perform appropriately in any given application (e.g., for detection of a target molecule). It is also to be understood that specificity may be evaluated in the context of additional factors such as the affinity of the binding agent for the target versus the affinity of the binding agent for other targets, e.g., competitors. If a binding agent exhibits a high affinity for a target molecule that it is desired to detect and low affinity for nontarget molecules, the binding agent will likely be an acceptable reagent. Once the specificity of a binding agent is established in one or more contexts, it may be employed in other contexts, e.g., similar contexts such as similar assays or assay conditions, without necessarily re-evaluating its specificity. In some embodiments specificity of a binding agent can be tested by performing an appropriate assay on a sample expected to lack the target (e.g., a sample from cells in which the gene encoding the target has been disabled or effectively inhibited) and showing that the assay does not result in a signal significantly different to background. In some embodiments, a first entity (e.g., molecule, complex) is said to “specifically bind” to a second entity if it binds to the second entity with substantially greater affinity than to most or all other entities present in the environment where such binding takes place and/or if the two entities bind with an equilibrium dissociation constant, K_{d}, of 10⁻⁴ or less, e.g., 10⁻⁵ M or less, e.g., 10⁻⁶ M or less, 10⁻⁷ M or less, 10⁻⁸ M or less, 10⁻⁹ M or less, or 10⁻¹⁰ M or less. K_{d} can be measured using any suitable method known in the art, e.g., surface plasmon resonance-based methods, isothermal titration calorimetry, differential scanning calorimetry, spectroscopy-based methods, etc. “Specific binding agent” refers to an entity that specifically binds to another entity, e.g., a molecule or molecular complex, which may be referred to as a “target”. “Specific binding pair” refers to two entities (e.g., molecules or molecular complexes) that specifically bind to one another. Examples are biotin-avidin, antibody-antigen, complementary nucleic acids, receptor-ligand, etc.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments a subject is a mammal, e.g. a human, non-human primate, rodent (e.g., mouse, rat, rabbit hamster), ungulate (e.g., ovine, bovine, equine, caprine species), canine, or feline. In some embodiments a subject is an avian. In some embodiments, a human subject is between newborn and 6 months old. In some embodiments, a human subject is between 6 and 24 months old. In some embodiments, a human subject is between 2 and 6, 6 and 12, or 12 and 18 years old. In some embodiments a human subject is between 18 and 30, 30 and 50, 50 and 80, or greater than 80 years old. In some embodiments, a subject is an adult. For purposes hereof a human at least 18 years of age is considered an adult. In some embodiments a subject is an individual who has or may have cancer or is at risk of developing cancer or cancer recurrence.

“Treat”, “treating” and similar terms as used herein in the context of treating a subject refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, or undesirable condition warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject may have been identified as being at risk of developing the disease (e.g., at increased risk relative to the general population or as having a risk factor that increases the likelihood of developing the disease.

The term “tumor” as used herein encompasses abnormal growths comprising aberrantly proliferating cells. As known in the art, tumors are typically characterized by excessive cell proliferation that is not appropriately regulated (e.g., that does not respond normally to physiological influences and signals that would ordinarily constrain proliferation) and may exhibit one or more of the following properties: dysplasia (e.g., lack of normal cell differentiation, resulting in an increased number or proportion of immature cells); anaplasia (e.g., greater loss of differentiation, more loss of structural organization, cellular pleomorphism, abnormalities such as large, hyperchromatic nuclei, high nuclear:cytoplasmic ratio, atypical mitoses, etc.); invasion of adjacent tissues (e.g., breaching a basement membrane); and/or metastasis. In certain embodiments a tumor is a malignant tumor, also referred to herein as a “cancer”. Malignant tumors have a tendency for sustained growth and an ability to spread, e.g., to invade locally and/or metastasize regionally and/or to distant locations, whereas benign tumors often remain localized at the site of origin and are often self-limiting in terms of growth. The term “tumor” includes malignant solid tumors (e.g., carcinomas, sarcomas) and malignant growths in which there may be no detectable solid tumor mass (e.g., certain hematologic malignancies). The term “cancer” is generally used interchangeably with “tumor” herein and/or to refer to a disease characterized by one or more tumors, e.g., one or more malignant or potentially malignant tumors. Cancer includes, but is not limited to: breast cancer; biliary tract cancer; bladder cancer; brain cancer (e.g., glioblastomas, medulloblastomas); cervical cancer; choriocarcinoma; colon cancer; endometrial cancer, esophageal cancer; gastric cancer; hematological neoplasms including acute lymphocytic leukemia and acute myelogenous leukemia; T-cell acute lymphoblastic leukemia/lymphoma; hairy cell leukemia; chronic lymphocytic leukemia, chronic myelogenous leukemia, multiple myeloma; adult T-cell leukemia/lymphoma; intraepithelial neoplasms including Bowen's disease and Paget's disease; liver cancer; lung cancer; lymphomas including Hodgkin's disease and lymphocytic lymphomas; neuroblastoma; melanoma, oral cancer including squamous cell carcinoma; ovarian cancer including ovarian cancer arising from epithelial cells, stromal cells, germ cells and mesenchymal cells; neuroblastoma, pancreatic cancer; prostate cancer; rectal cancer; sarcomas including angiosarcoma, gastrointestinal stromal tumors, leiomyosarcoma, rhabdomyosarcoma, liposarcoma, fibrosarcoma, and osteosarcoma; renal cancer including renal cell carcinoma and Wilms tumor; skin cancer including basal cell carcinoma and squamous cell cancer, testicular cancer including germinal tumors such as seminoma, non-seminoma (teratomas, choriocarcinomas), stromal tumors, and germ cell tumors; thyroid cancer including thyroid adenocarcinoma and medullary carcinoma. It will be appreciated that a variety of different tumor types can arise in certain organs, which may differ with regard to, e.g., clinical and/or pathological features and/or molecular markers. Tumors arising in a variety of different organs are discussed, e.g., in DeVita, supra or in the WHO Classification of Tumours series, 4^{th} ed, or 3^{rd} ed (Pathology and Genetics of Tumours series), by the International Agency for Research on Cancer (IARC), WHO Press, Geneva, Switzerland, all volumes of which are incorporated herein by reference.

A “variant” of a particular polypeptide or polynucleotide has one or more alterations (e.g., additions, substitutions, and/or deletions) with respect to the polypeptide or polynucleotide, which may be referred to as the “original polypeptide” or “original polynucleotide”, respectively. An addition may be an insertion or may be at either terminus. A variant may be shorter or longer than the original polypeptide or polynucleotide. The term “variant” encompasses “fragments”. A “fragment” is a continuous portion of a polypeptide or polynucleotide that is shorter than the original polypeptide. In some embodiments a variant comprises or consists of a fragment. In some embodiments a fragment or variant is at least 20%, 30%, 40%, 50%, 60%, 70%, 80%, 90%, 95%, 96%, 97%, 98%, 99%, or more as long as the original polypeptide or polynucleotide. A fragment may be an N-terminal, C-terminal, or internal fragment. In some embodiments a variant polypeptide comprises or consists of at least one domain of an original polypeptide. In some embodiments a variant polynucleotide hybridizes to an original polynucleotide under stringent conditions, e.g., high stringency conditions, for sequences of the length of the original polypeptide. In some embodiments a variant polypeptide or polynucleotide comprises or consists of a polypeptide or polynucleotide that is at least 50%, 60%, 70%, 80%, 90%, 95%, 96%, 97%, 98%, 99%, or more identical in sequence to the original polypeptide or polynucleotide over at least 20%, 30%, 40%, 50%, 60%, 70%, 80%, 90%, 95%, 96%, 97%, 98%, 99%, or 100% of the original polypeptide or polynucleotide. In some embodiments a variant polypeptide comprises or consists of a polypeptide that is at least 50%, 60%, 70%, 80%, 90%, 95%, 96%, 97%, 98%, 99%, or more identical in sequence to the original polypeptide over at least 20%, 30%, 40%, 50%, 60%, 70%, 80%, 90%, 95%, 96%, 97%, 98%, 99%, or 100% of the original polypeptide, with the proviso that, for purposes of computing percent identity, a conservative amino acid substitution is considered identical to the amino acid it replaces. In some embodiments a variant polypeptide comprises or consists of a polypeptide that is at least 50%, 60%, 70%, 80%, 90%, 95%, 96%, 97%, 98%, 99%, or more identical to the original polypeptide over at least 20%, 30%, 40%, 50%, 60%, 70%, 80%, 90%, 95%, 96%, 97%, 98%, 99%, or 100% of the original polypeptide, with the proviso that any one or more amino acid substitutions (up to the total number of such substitutions) may be restricted to conservative substitutions. In some embodiments a percent identity is measured over at least 100; 200; 300; 400; 500; 600; 700; 800; 900; 1,000; 1,200; 1,500; 2,000; 2,500; 3,000; 3,500; 4,000; 4,500; or 5,000 amino acids. In some embodiments the sequence of a variant polypeptide comprises or consists of a sequence that has N amino acid differences with respect to an original sequence, wherein N is any integer between 1 and 10 or between 1 and 20 or any integer up to 1%, 2%, 5%, or 10% of the number of amino acids in the original polypeptide, where an “amino acid difference” refers to a substitution, insertion, or deletion of an amino acid. In some embodiments a difference is a conservative substitution. Conservative substitutions may be made, e.g., on the basis of similarity in side chain size, polarity, charge, solubility, hydrophobicity, hydrophilicity and/or the amphipathic nature of the residues involved. In some embodiments, conservative substitutions may be made according to Table A, wherein amino acids in the same block in the second column and in the same line in the third column may be substituted for one another other in a conservative substitution. Certain conservative substitutions are substituting an amino acid in one row of the third column corresponding to a block in the second column with an amino acid from another row of the third column within the same block in the second column.

TABLE A

| | | |
|---|---|---|
| Aliphatic | Non-polar | G A P |
| | | I L V |
| | Polar - uncharged | C S T M |
| | | N Q |
| | Polar - charged | D E |
| | | K R |
| Aromatic | | H F W Y |

observed in tumors from glucose limitation resistant cell line NCI-H82.

In some embodiments, proline (P) is considered to be in an individual group. In some embodiments, cysteine (C) is considered to be in an individual group. In some embodiments, proline (P) and cysteine (C) are each considered to be in an individual group. Within a particular group, certain substitutions may be of particular interest in certain embodiments, e.g., replacements of leucine by isoleucine (or vice versa), serine by threonine (or vice versa), or alanine by glycine (or vice versa).

In some embodiments a variant is a functional variant, i.e., the variant at least in part retains at least one activity of the original polypeptide or polynucleotide. In some embodiments a variant at least in part retains more than one or substantially all known biologically significant activities of the original polypeptide or polynucleotide. An activity may be, e.g., a catalytic activity, binding activity, ability to perform or participate in a biological function or process, etc. In some embodiments an activity of a variant may be at least 10%, 20%, 30%, 40%, 50%, 60%, 70%, 80%, 90%, 95%, or more, of the activity of the original polypeptide or polynucleotide, up to approximately 100%, approximately 125%, or approximately 150% of the activity of the original polypeptide or polynucleotide, in various embodiments. In some embodiments a variant, e.g., a functional variant, comprises or consists of a polypeptide at least 95%, 96%, 97%, 98%, 99%, 99.5% or 100% identical to an original polypeptide or polynucleotide over at least 70%, 75%, 80%, 85%, 90%, 95%, 96%, 97%, 98%, or 99% or 100% of the original polypeptide or polynucleotide. In some embodiments an alteration, e.g., a substitution or deletion, e.g., in a functional variant, does not alter or delete an amino acid or nucleotide that is known or predicted to be important for an activity, e.g., a known or predicted catalytic residue or residue involved in binding a substrate or cofactor. In some embodiments nucleotide(s), amino acid(s), or region(s) exhibiting lower degrees of conservation across species as compared with other amino acids or regions may be selected for alteration. Variants may be tested in one or more suitable assays to assess activity.

A “vector” may be any of a number of nucleic acid molecules or viruses or portions thereof that are capable of mediating entry of, e.g., transferring, transporting, etc., a nucleic acid of interest between different genetic environments or into a cell. The nucleic acid of interest may be linked to, e.g., inserted into, the vector using, e.g., restriction and ligation. Vectors include, for example, DNA or RNA plasmids, cosmids, naturally occurring or modified viral genomes or portions thereof, nucleic acids that can be packaged into viral capsids, mini-chromosomes, artificial chromosomes, etc. Plasmid vectors typically include an origin of replication (e.g., for replication in prokaryotic cells). A plasmid may include part or all of a viral genome (e.g., a viral promoter, enhancer, processing or packaging signals, and/or sequences sufficient to give rise to a nucleic acid that can be integrated into the host cell genome and/or to give rise to infectious virus). Viruses or portions thereof that can be used to introduce nucleic acids into cells may be referred to as viral vectors. Viral vectors include, e.g., adenoviruses, adeno-associated viruses, retroviruses (e.g., lentiviruses), vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-competent or replication-defective. In some embodiments, e.g., where sufficient information for production of infectious virus is lacking, it may be supplied by a host cell or by another vector introduced into the cell, e.g., if production of virus is desired. In some embodiments such information is not supplied, e.g., if production of virus is not desired. A nucleic acid to be transferred may be incorporated into a naturally occurring or modified viral genome or a portion thereof or may be present within a viral capsid as a separate nucleic acid molecule. A vector may contain one or more nucleic acids encoding a marker suitable for identifying and/or selecting cells that have taken up the vector. Markers include, for example, various proteins that increase or decrease either resistance or sensitivity to antibiotics or other agents (e.g., a protein that confers resistance to an antibiotic such as puromycin, hygromycin or blasticidin), enzymes whose activities are detectable by assays known in the art (e.g., β-galactosidase or alkaline phosphatase), and proteins or RNAs that detectably affect the phenotype of cells that express them (e.g., fluorescent proteins). Vectors often include one or more appropriately positioned sites for restriction enzymes, which may be used to facilitate insertion into the vector of a nucleic acid, e.g., a nucleic acid to be expressed. An expression vector is a vector into which a desired nucleic acid has been inserted or may be inserted such that it is operably linked to regulatory elements (also termed “regulatory sequences”, “expression control elements”, or “expression control sequences”) and may be expressed as an RNA transcript (e.g., an mRNA that can be translated into protein or a noncoding RNA such as an shRNA or miRNA precursor). Expression vectors include regulatory sequence(s), e.g., expression control sequences, sufficient to direct transcription of an operably linked nucleic acid under at least some conditions; other elements required or helpful for expression may be supplied by, e.g., the host cell or by an in vitro expression system. Such regulatory sequences typically include a promoter and may include enhancer sequences or upstream activator sequences. In some embodiments a vector may include sequences that encode a 5′ untranslated region and/or a 3′ untranslated region, which may comprise a cleavage and/or polyadenylation signal. In general, regulatory elements may be contained in a vector prior to insertion of a nucleic acid whose expression is desired or may be contained in an inserted nucleic acid or may be inserted into a vector following insertion of a nucleic acid whose expression is desired. As used herein, a nucleic acid and regulatory element(s) are said to be “operably linked” when they are covalently linked so as to place the expression or transcription of the nucleic acid under the influence or control of the regulatory element(s). For example, a promoter region would be operably linked to a nucleic acid if the promoter region were capable of effecting transcription of that nucleic acid. One of ordinary skill in the art will be aware that the precise nature of the regulatory sequences useful for gene expression may vary between species or cell types, but may in general include, as appropriate, sequences involved with the initiation of transcription, RNA processing, or initiation of translation. The choice and design of an appropriate vector and regulatory element(s) is within the ability and discretion of one of ordinary skill in the art. For example, one of skill in the art will select an appropriate promoter (or other expression control sequences) for expression in a desired species (e.g., a mammalian species) or cell type. A vector may contain a promoter capable of directing expression in mammalian cells, such as a suitable viral promoter, e.g., from a cytomegalovirus (CMV), retrovirus, simian virus (e.g., SV40), papilloma virus, herpes virus or other virus that infects mammalian cells, or a mammalian promoter from, e.g., a gene such as EF1alpha, ubiquitin (e.g., ubiquitin B or C), globin, actin, phosphoglycerate kinase (PGK), etc., or a composite promoter such as a CAG promoter (combination of the CMV early enhancer element and chicken beta-actin promoter). In some embodiments a human promoter may be used. In some embodiments, a promoter that ordinarily directs transcription by a eukaryotic RNA polymerase I (a “pol I promoter”), e.g., (a U6, H1, 7SK or tRNA promoter or a functional variant thereof) may be used. In some embodiments, a promoter that ordinarily directs transcription by a eukaryotic RNA polymerase II (a “pol II promoter”) or a functional variant thereof is used. In some embodiments, a promoter that ordinarily directs transcription by a eukaryotic RNA polymerase III promoter, e.g., a promoter for transcription of ribosomal RNA (other than 5S rRNA) or a functional variant thereof is used. One of ordinary skill in the art will select an appropriate promoter for directing transcription of a sequence of interest. Examples of expression vectors that may be used in mammalian cells include, e.g., the pcDNA vector series, pSV2 vector series, pCMV vector series, pRSV vector series, pEF1 vector series, Gateway® vectors, etc. Examples of virus vectors that may be used in mammalian cells include, e.g., adenoviruses, adeno-associated viruses, poxviruses such as vaccinia viruses and attenuated poxviruses, retroviruses (e.g., lentiviruses), Semliki Forest virus, Sindbis virus, etc. In some embodiments, regulatable (e.g., inducible or repressible) expression control element(s), e.g., a regulatable promoter, is/are used so that expression can be regulated, e.g., turned on or increased or turned off or decreased. For example, the tetracycline-regulatable gene expression system (Gossen & Bujard, Proc. Natl. Acad. Sci. 89:5547-5551, 1992) or variants thereof (see, e.g., Allen, N, et al. (2000) Mouse Genetics and Transgenics: 259-263; Urlinger, S, et al. (2000). Proc. Natl. Acad. Sci. U.S.A. 97 (14): 7963-8; Zhou, X., et al (2006). Gene Ther. 13 (19): 1382-1390 for examples) can be employed to provide inducible or repressible expression. Other inducible/repressible systems may be used in various embodiments. For example, expression control elements that can be regulated by small molecules such as artificial or naturally occurring hormone receptor ligands (e.g., steroid receptor ligands such as naturally occurring or synthetic estrogen receptor or glucocorticoid receptor ligands), tetracycline or analogs thereof, metal-regulated systems (e.g., metallothionein promoter) may be used in certain embodiments. In some embodiments, tissue-specific or cell type specific regulatory element(s) may be used, e.g., in order to direct expression in one or more selected tissues or cell types. In some embodiments a vector capable of being stably maintained and inherited as an episome in mammalian cells (e.g., an Epstein-Barr virus-based episomal vector) may be used. In some embodiments a vector may comprise a polynucleotide sequence that encodes a polypeptide, wherein the polynucleotide sequence is positioned in frame with a nucleic acid inserted into the vector so that an N- or C-terminal fusion is created. In some embodiments the polypeptide encoded by the polynucleotide sequence may be a targeting peptide. A targeting peptide may comprise a signal sequence (which directs secretion of a protein) or a sequence that directs the expressed protein to a specific organelle or location in the cell such as the nucleus or mitochondria. In some embodiments the polypeptide comprises a tag. A tag may be useful to facilitate detection and/or purification of a protein that contains it. Examples of tags include polyhistidine-tag (e.g., 6×-His tag), glutathione-S-transferase, maltose binding protein, NUS tag, SNUT tag, Strep tag, epitope tags such as V5, HA, Myc, or FLAG. In some embodiments a protease cleavage site is located in the region between the protein encoded by the inserted nucleic acid and the polypeptide, allowing the polypeptide to be removed by exposure to the protease.

### II. Identification of Cancers and Cancer Cell Lines that are Sensitive to OXPHOS Inhibitors

In some aspects, the disclosure provides methods of identifying tumors and tumor cell lines that have increased likelihood of being sensitive to inhibition of oxidative phosphorylation (OXPHOS). In some aspects, the disclosure provides methods of identifying tumors and tumor cell lines that have increased likelihood of being sensitive to inhibitors of oxidative phosphorylation. In some aspects, the disclosure provides methods of identifying tumors and tumor cell lines that have increased likelihood of being sensitive to biguanides. In some embodiments the methods are useful in selecting an appropriate therapy for a subject in need of treatment for cancer. For example, in some embodiments the methods are useful in selecting an OXPHOS inhibitor as an appropriate therapeutic agent. In some embodiments the methods are useful in selecting a biguanide, e.g., metformin, as an appropriate therapeutic agent.

Mitochondria are responsible for producing most of the ATP used by eukaryotic cells as a source of chemical energy. Fuels such as carbohydrates and fats are transported across the inner mitochondrial membrane into the matrix, broken down, and further metabolized in the tricarboxylic acid (TCA) cycle, during which NAD+ and FAD are reduced to NADH and FADH2. Synthesis of ATP occurs via a two stage process. High energy electrons from FADH2 and NADH (from the TCA cycle or glycolysis) are shuttled through a series of protein complexes in the inner mitochondrial membrane to molecular oxygen. The loss of electrons from NADH and FADH2 regenerates the NAD+ and FADH needed for the process to continue. During the electron transport process, protons are pumped out of the mitochondrial matrix to the intermembrane space, resulting in an electrochemical gradient that includes contributions from both a membrane potential (Δψₘ) and a pH difference. The energy released when protons flow back into the matrix across the inner membrane is used by the protein complex termed ATP synthase to synthesize ATP from ADP and inorganic phosphate (Pᵢ). The electrochemical proton gradient drives a variety of other processes in addition to ATP synthesis, such as transport of charged small molecules. The overall process of electron transport and ATP synthesis is referred to as “oxidative phosphorylation” (OXPHOS), and the components responsible for performing these processes are referred to as the “OXPHOS system”. The components involved in OXPHOS include 5 multi-subunit protein complexes (referred to as complexes I, II, III, IV, and V), a small molecule (ubiquinone, also called coenzyme Q), and the protein cytochrome c (Cyt c). The set of proteins and small molecules involved in electron transport is referred to as the “electron transport chain” or “respiratory chain”. Protons are pumped across the inner mitochondrial membrane (i.e., from the matrix to the intermembrane space) by complexes I, III, and IV. Ubiquinone, and cytochrome c function as electron carriers. Electrons from the oxidation of succinate to fumarate are channeled through this complex to ubiquinone. Complex V is ATP synthase (EC 3.6.3.14), which is composed of a head portion, called the F1 ATP synthase (or F1), and a transmembrane proton carrier, called F0. Both F1 and F0 are composed of multiple subunits. ATP synthase can function in reverse mode in which it hydrolyzes ATP. The energy of ATP hydrolysis can be used to pump protons across the inner mitochondrial membrane into the matrix. ATP synthase is also referred to as F0-F1 ATP synthase or F0-F1 ATPase.

Many solid tumors are characterized by nutrient limitation at least in some portions of the tumor, e.g., due to limited blood supply. For example, many tumors exist at least in part in a state of glucose limitation. The present disclosure encompasses the recognition that tumors and tumor cell lines exhibit varying responses to glucose limitation. Certain cancer cell lines were found to exhibit decreased proliferation in response to glucose limitation (low glucose conditions). In some embodiments glucose limitation (also termed “glucose restriction” or “low glucose” herein) refers to a glucose concentration between about 0.50 mM and about 1.0 mM glucose. In some embodiments glucose limitation refers to a glucose concentration between about 0.75 mM and about 1.0 mM glucose, e.g., about 0.75 mM glucose. In some embodiments a “high” glucose concentration refers to a concentration at which the glucose concentration does not limit cell proliferation. In some embodiments a “high” glucose concentration may be a glucose concentration above the mean normal blood glucose level in humans (about 5.5 mM). In some embodiments a “high” glucose concentration refers to a concentration that is standard for culture of certain cancer cell lines. In some embodiments a “high” glucose concentration refers to a concentration between about 5 mM and about 15 mM glucose. In some embodiments a “high” glucose concentration refers to a concentration between about 5 mM and about 10 mM glucose. In some embodiments a “standard” glucose concentration refers to a concentration of about 10 mM glucose. A high glucose concentration may also be referred to herein as a “standard glucose” concentration since it is a standard culture condition for many cell lines. In certain embodiments sensitivity to glucose limitation is a metabolic liability of certain cancers that may be exploited for therapeutic purposes. In certain embodiments methods of identifying cancers that are or are likely to be sensitive to glucose limitation are provided. In some embodiments such methods identify cancers that are or are likely to be sensitive to OXPHOS inhibition, e.g., using OXPHOS inhibitors. In some embodiments such methods identify cancers that are or are likely to be sensitive to biguanides.

In some aspects, the disclosure comprises use of OXPHOS inhibition (which may achieved by administration of an OXPHOS inhibitor) as a therapeutic approach for cancers comprising cancer cells that are sensitive to low glucose. As described herein, cancer cell lines most sensitive to glucose limitation were found to be incapable of inducing OXPHOS upon glucose restriction. Certain cancer cell lines sensitive to glucose limitation were found to have mutations in genes encoding components of the OXPHOS system, e.g., components of complex I. Certain cancer cell lines sensitive to glucose limitation were found to exhibit low glucose uptake, e.g., as a result of decreased expression of a glucose transporter. In particular, decreased expression of SLC2A3 was observed to result in glucose limitation sensitivity. In certain embodiments any of the methods described herein in regard to SLC2A3 may additionally or alternately be applied to a different glucose transporter, e.g., SLC2A1. Certain genes were identified as being differentially required for cancer cell proliferation under low glucose conditions. These genes are listed in Table 1. In some embodiments, low expression or activity of such genes or their gene products is predictive of sensitivity to glucose limitation (e.g., decreased ability to survive or proliferate under low glucose conditions, such as those that may prevail in at least certain portions of solid tumors). According to certain embodiments, tumors characterized by low expression of one or more such genes are amenable to therapy with an OXPHOS inhibitor. A low level of expression of certain genes was identified as constituting a low glucose utilization signature. These genes included glucose transporters SLC2A3 and SLC2A1 as well as several glycolytic enzymes. Low expression of such genes is indicative of a defect in glucose utilization, sensitivity to low glucose conditions, and sensitivity to biguanides. These genes are listed in Table 4. Thus in some aspects, the present disclosure identifies particular glycolytic enzymes and other proteins involved in glucose utilization whose low expression is indicative of sensitivity to low glucose. These genes, and genes encoding Complex I components, are useful, for example, as biomarkers for identifying tumors that are sensitive to glucose limitation and OXPHOS inhibitors, and as targets for development of anti-cancer agents. In some aspects, the disclosure comprises use of biguanides as a therapeutic approach for cancers comprising cancer cells that have low expression of one or more genes listed in Table 4, e.g., low expression of the gene expression signature comprising the genes listed in Table 4. In some embodiments, expression of 2, 3, 4, 5, 6, 7, 8, 9, 10, or all 11 of the genes in Table 4 is assessed. In some embodiments expression of at least ENO1 is assessed. In some embodiments expression of at least ENO1 and GAPDH are assessed. In some embodiments expression of at least SLC2A3 and ENO1 are assessed. In some embodiments expression of SLC2A3 and at least one other gene in Table 4 is assessed. In some aspects, the disclosure comprises use of inhibitors of a gene listed in Table 4 as a therapeutic approach for cancers comprising cancer cells that have low expression of one or more genes listed in Table 4, e.g., low expression of the gene expression signature comprising the genes listed in Table 4. In some aspects, the disclosure comprises use of OXPHOS inhibition (which may achieved by administration of an OXPHOS inhibitor) as a therapeutic approach for cancers comprising cancer cells that have low expression of one or more genes listed in Table 4, e.g., low expression of the gene expression signature comprising the genes listed in Table 4. Certain genes were identified as being differentially required for cancer cell proliferation under high glucose conditions. These genes are listed in Table 2.

Genomic, mRNA, and polypeptide sequences of genes and gene products of interest herein (e.g., genes listed in Table 1, Table 2, Table 3, or Table 4) are known in the art and are available in databases such as the National Center for Biotechnology Information (www.ncbi.nih.gov) or Universal Protein Resource (www.uniprot.org) databases, e.g., GenBank, RefSeq, Gene, UniProtKB/SwissProt, UniProtKB/Trembl, Genome, and the like. For example, Unigene ID of human CYC1 (cytochrome c-1) is Hs.289271; Unigene ID of human UQCRC1 (ubiquinol-cytochrome c reductase core protein 1) is Hs.119251. Unigene ID of human SLC2A3 (solute carrier family 2 (facilitated glucose transporter), member 3) is Hs.419240. Sequence information may be employed, for example, in generating or testing detection reagents or therapeutic agents of use in methods described herein. In some embodiments a sequence listed under a NCBI RefSeq accession number is used. It should be understood that sequences listed under particular accession numbers, e.g., RefSeqs, are exemplary and that different alleles, e.g., polymorphisms, may exist in the population.

Any one or more isoforms or transcript variants may be detected in various embodiments. Detection of particular variants or isoforms may be accomplished using suitable detection reagents and/or by performing an assay under appropriate conditions. For example, antibodies that specifically bind to one, more than one, or all isoforms may be used. Probes, primers, and/or hybridization conditions can be selected such that a probe or primer will hybridize with one, more than one, or all variants. Where multiple isoforms exist, the most widely expressed isoform or an isoform having a particular biological activity (e.g., an emzymatic activity, a nutrient transport activity, and/or involvement in glucose utilization, e.g., glycolysis, glucose transport, or OXPHOS) may be selected.

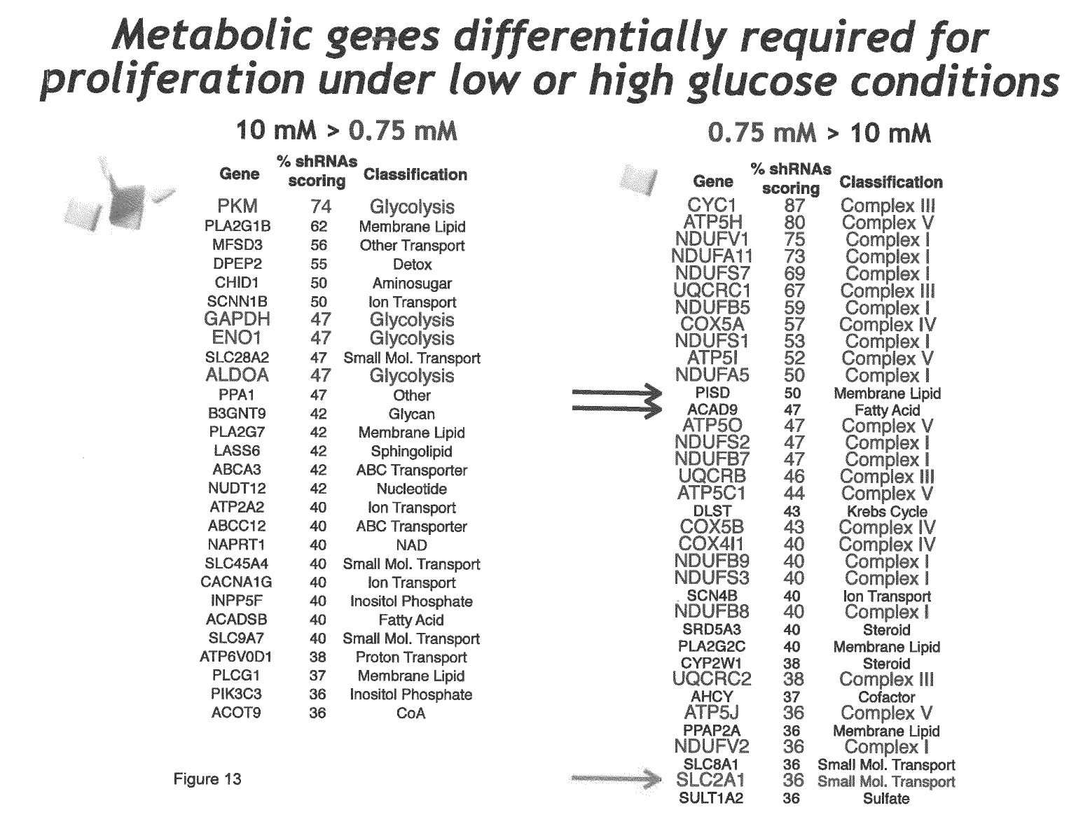

TABLE 1
Human Gene Symbols and Gene IDs for Genes Differentially
Required for Proliferation Under Low Glucose Conditions
(same genes as listed in right column of FIG. 13)

| SYMBOL | NCBI GENE ID | RefSeq mRNA | RefSeq Protein |
|---|---|---|---|
| CYC1 | 1537 | NM_001916 | NP_001907 |
| ATP5H | 10476 | | |
| NDUFV1 | 4723 | | |
| NDUFA11 | 126328 | | |
| NDUFS7 | 374291 | | |
| UQCRC1 | 7384 | NM_003365 | NP_003356 |
| NDUFB5 | 4711 | | |
| COX5A | 9377 | | |
| NDUFS1 | 4719 | | |
| ATP5I | 521 | | |
| NDUFA5 | 4698 | | |
| PISD | 23761 | | |
| ACAD9 | 28976 | | |
| ATP5O | 539 | | |
| NDUFS2 | 4720 | | |
| NDUFB7 | 4713 | | |
| UQCRB | 7381 | | |
| ATP5C1 | 509 | | |
| DLST | 1743 | | |
| COX5B | 1329 | | |
| COX4I1 | 1327 | | |
| NDUFB9 | 4715 | | |
| NDUFS3 | 4722 | | |
| SCN4B | 6330 | | |
| NDUFB8 | 4714 | | |
| SRD5A3 | 79644 | | |
| PLA2G2C | 391013 | | |
| CYP2W1 | 54905 | | |
| UQCRC2 | 7385 | | |
| AHCY | 191 | | |
| ATP5J | 522 | | |
| PPAP2A | 8611 | | |
| NDUFV2 | 4729 | | |
| SLC8A1 | 6546 | | |
| SLC2A1 | 6513 | | |
| SULT1A2 | 6799 | | |

TABLE 2
Human Gene Symbols and Gene IDs for Genes Differentially
Required for Proliferation Under High Glucose Conditions
(same genes as listed in left column of FIG. 13)

| SYMBOL | NCBI GENE ID |
|---|---|
| PKM | 5315 |
| PLA2G1B | 5319 |
| MFSD3 | 113655 |
| DPEP2 | 64174 |
| CHID1 | 66005 |
| SCNN1B | 6338 |
| GAPDH | 2597 |
| ENO1 | 2023 |
| SLC28A2 | 9153 |
| ALDOA | 226 |
| PPA1 | 5464 |
| B3GNT9 | 84752 |
| PLA2G7 | 7941 |
| LASS6 | None currently available |
| ABCA3 | 21 |
| NUDT12 | 83594 |
| ATP2A2 | 488 |
| ABCC12 | 94160 |
| SLC45A4 | 57210 |
| CACNA1G | 8913 |
| INPP5F | 22876 |
| ACADSB | 36 |
| SCL9A7 | None currently available |
| A7P6V0D1 | 9114 |
| PLCG1 | 5335 |
| PIK3C3 | 5289 |
| ACOT9 | 23597 |

TABLE 4
Human Gene Symbols and Gene IDs for Genes
Constituting Low Glucose Utilization Signature

| SYMBOL | NCBI GENE ID |
|---|---|
| ENO1 | 2023 |
| GAPDH | 2597 |
| GPI | 2821 |
| HK1 | 3098 |
| PKM | 5315 |
| SLC2A1 | 6513 |
| SLC2A3 | 6515 |
| TPI1 | 7167 |
| ALDOA | 226 |
| PFKP | 5214 |
| PGK1 | 5230 |

In some aspects, tumors or tumor cell lines that are sensitive to glucose limitation are sensitive to OXPHOS inhibitors.

In some aspects, tumors or tumor cell lines that are sensitive to glucose limitation are sensitive to biguanides. In some embodiments a “biguanide” refers to a compound of the following formula:

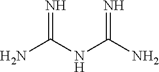

in which any one or more of the hydrogen atoms may be replaced by a substituent, e.g., an acyl, substituted or unsubstituted aliphatic, substituted or unsubstituted heteroaliphatic, substituted or unsubstituted aryl, or substituted or unsubstituted heteroaryl. In some embodiments a substituent is an alkyl (e.g., C1-C4 alkyl, e.g., methyl or ethyl).
In some embodiments, a biguanide is metformin (N,N-dimethylimidodicarbonimidic diamide), shown below:

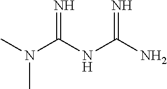

In some embodiments, a biguanide is phenformin (2-(N-phenethylcarbamimidoyl)guanidine), the structure of which is shown below.

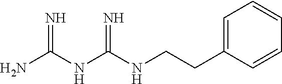 [SMILES: N=C(N)NC(=N)NCCc1ccccc1]

In some embodiments a biguanide is buformin (N-Butylimidocarbonimidic diamide), the structure of which is shown below:

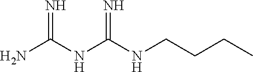

In some embodiments a biguanide is a compound of the following formula:

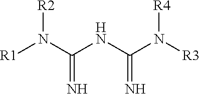

in which R1 and R2, which may be identical or different, represent a branched or unbranched (C1-C6)alkyl chain, or R1 and R2 together form a 3- to 8-membered ring including the nitrogen atom to which they are attached, R3 and R4 together form a ring selected from the group aziridine, pyrrolyl, imidazolyl, pyrazolyl, indolyl, indolinyl, pyrrolidinyl, piperazinyl and piperidyl including the nitrogen atom to which they are attached, e.g., as described in WO/2002/074740.

In some embodiments a biguanide is a compound of the following formula:

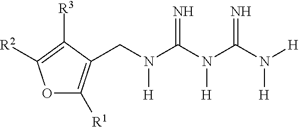

in which R¹, R², and R³ may be the same or different and each represents one member selected from the group consisting of hydrogen, optionally substituted lower alkyls, and optionally substituted lower alkylthios, e.g., as described in WO/2003/091234.

In some embodiments an OXPHOS inhibitor is a compound that inhibits mitochondrial protein synthesis, e.g., by inhibiting mitochondrial translation. In some embodiments an OXPHOS inhibitor is a compound that inhibits bacterial protein synthesis, e.g., by inhibiting bacterial translation. Due to certain similarities between bacteria and mitochondria, such compounds may also inhibit mitochondrial translation. In some embodiments a compound is capable of binding to the 16S part of the 30S ribosomal subunit and prevents the amino-acyl tRNA from binding to the A site of the ribosome. Examples of such compounds include the tetracyclines, a number of which are used clinically as antibacterial therapeutic agents. Tetracyclines are defined as “a subclass of polyketides having an octahydrotetracene-2-carboxamide skeleton” (Nic, M.; Jirat, J.; Kosata, B., eds. (2006-). “tetracyclines”. IUPAC Compendium of Chemical Terminology (Online ed.). doi:10.1351/goldbook. ISBN 0-9678550-9-8. http://goldbook.iupac.org/T06287.html.). They are sometimes collectively known as “derivatives of polycyclic naphthacene carboxamide”. A formula showing the 4 rings of the basic tetracycline structure is shown below.

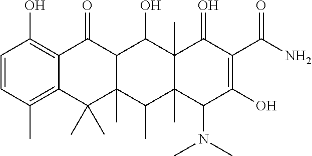 [SMILES: Cc1ccc(O)c2c1C(C)(C)C1(C)C(C2=O)C(O)C2(C)C(=O)C(C(N)=O)=C(O)C(N(C)C)C2(C)C1C]

Naturally occurring tetracyclines include, e.g., tetracycline, chlortetracycline, oxytetracycline, and demeclocycline. Semi-synthetic tetracyclines include, e.g., doxycycline, lymecycline, meclocycline, methacycline, minocycline, and rolitetracycline

It will be appreciated that various tetracyclines have substituents or different substituents at one or more positions of the 4 ring structure shown above. For example, the structure of doxycycline (4S,4aR,5S,5aR,6R,12aS)-4-(dimethylamino)-3,5,10,12,12a-pentahydroxy-6-methyl-1,11-dioxo-1,4,4a,5,5a,6,11,12a-octahydrotetracene-2-carboxamide) is shown below.

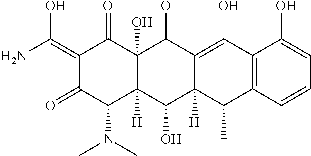

The structure of minocycline ((2E,4S,4aR,5aS,12aR)-2-(amino-hydroxy-methylidene)-4,7-bis(dimethylamino)-10,11,12a-trihydroxy-4a,5,5a,6-tetrahydro-4H-tetracene-1,3,12-trione) is shown below:

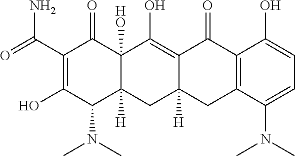 [SMILES: CN(C)c1ccc(O)c2c1C[C@H]1C[C@H]3[C@H](N(C)C)C(O)=C(C(N)=O)C(=O)[C@@]3(O)C(O)=C1C2=O]

Minocycline is among the most lipid-soluble of the tetracycline-class antibiotics, giving it the greatest penetration into certain organs such as the prostate and brain. Doxycycline and minocycline are both classified as long-acting tetracyclines, having a half-life of 16 hours or more. In some embodiments an OXPHOS inhibitor is an aminomethylcycline (AMC) such as PTK 0796 (Paratek). AMCs were evolved from and are structurally related to tetracycline antibiotics.

In some embodiments an OXPHOS inhibitor is a glycylcycline. In some embodiments a glycycycline is a compound having the following formula:

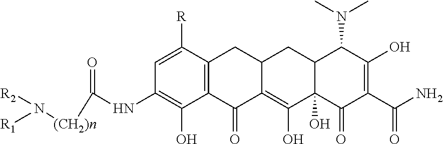

or a pharmaceutically acceptable salt thereof, wherein Ri and R₂ are each independently chosen from hydrogen, straight and branched chain (C1-C6)alkyl, and cycloalkyl, or Ri and R₂, together with N, form a heterocycle; R is —NR₃R₄, where R₃ and R₄ are each independently chosen from hydrogen, and straight and branched chain (C1-C4)alkyl; and n ranges from 1-4.

In some embodiments a glycycycline is tigecycline (N-[(5aR,6aS,7S,9Z,10aS)-9-[amino(hydroxy)methylidene]-4,7-bis(dimethylamino)-1,10a, 12-trihydroxy-8,10,11-trioxo-5,5a,6,6a,7,8,9,10,10a,11-decahydrotetracen-2-yl]-2-(tert-butylamino)acetamide). Tigecycline and other glycylcyclines are structurally similar to the tetracyclines in that they contain a central four-ring carbocyclic skeleton and shares the same mechanism of action. Tigecycline is a derivative of minocycline. Tigecycline has a substitution at the D-9 position which is believed to confer broad spectrum activity. The structure of tigecycline is presented below:

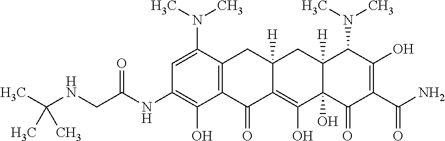 [SMILES: CN(C)c1cc(NC(=O)CNC(C)(C)C)c(O)c2c1C[C@H]1C[C@H]3[C@H](N(C)C)C(O)=C(C(N)=O)C(=O)[C@@]3(O)C(O)=C1C2=O]

Tigecycline and various other glycylcycline antibiotics, methods of preparation and various formulations are described, e.g., in WO/2007/027599-9—AMINOCARBONYLSUBSTITUTED DERIVATIVES OF GLYCYLCYCLINES; WO/2007/127292—TIGEYCLINE CRYSTALLINE FORMS AND PROCESSES FOR PREPARATION THEREOF; WO/2010/017273—TIGECYCLINE FORMULATIONS; WO/2006/130431—METHODS OF PURIFYING TIGECYCLINE; WO/2006/130418—TIGECYCLINE AND METHODS OF PREPARATION; WO/2006/130500—TIGECYCLINE AND METHODS OF PREPARING 9-AMINOMINOCYCLINE; WO2006130431—METHODS OF PURIFYING TIGECYCLINE.

In some embodiments a cancer that may be treated with a mitochondrial protein synthesis inhibitor, e.g., a tetracycline or a glycylcycline, is a hematological cancer, such as a leukemia (e.g., acute myeloid leukemia), lymphoma, or myeloma. In some embodiments a cancer that may be treated with a mitochondrial protein synthesis inhibitor, e.g., a tetracycline or a glycylcycline, is a solid tumor. In some embodiments one or more methods described herein is used to identify the cancer, e.g., a hematological cancer or solid tumor, as being sensitive to glucose limitation. In some embodiments one or more methods described herein is used to identify the cancer, e.g., a hematological cancer or solid tumor, as having increased likelihood of being sensitive to OXPHOS inhibition. A subject in need of treatment for the cancer is then treated with a mitochondrial protein synthesis inhibitor, e.g., a tetracycline or a glycylcycline.

In some aspects, the invention provides methods that comprise assessing expression level of one or more genes listed in Table 1 or expression or activity of a gene product thereof, for purposes of tumor classification, treatment selection, and/or predicting tumor responsiveness to OXPHOS inhibition or biguanides. In some aspects, the invention provides methods that comprise assessing expression level of SLC2A3 (GLUT3) gene or expression or activity of a gene product thereof, for purposes of tumor classification, treatment selection, and/or predicting tumor responsiveness to OXPHOS inhibition or biguanides. In some aspects, described herein are methods of classifying a tumor cell, tumor cell line, or tumor according to predicted sensitivity to OXPHOS inhibition. In some aspects, described herein are methods of classifying a tumor cell, tumor cell line, or tumor according to predicted sensitivity to biguanides. In some embodiments the methods comprise: (a) assessing the level of expression of a gene product of a gene listed in Table 1 in a tumor cell, tumor cell line, or tumor; and (b) classifying the tumor cell, tumor cell line, or tumor with respect to predicted sensitivity to OXPHOS inhibition or biguanides based at least in part on the level of expression of the one or more genes. In some embodiments the methods comprise: (a) assessing the level of expression of a SLC2A3 (GLUT3) gene in a tumor cell, tumor cell line, or tumor; and (b) classifying the tumor cell, tumor cell line, or tumor with respect to predicted sensitivity to OXPHOS inhibition or biguanides based at least in part on the level of expression. In some embodiments assessing expression of a gene in a tumor comprises assessing expression of the gene in one or more samples obtained from the tumor. In certain embodiments low (decreased, reduced) expression of a gene listed in Table 1 or the SLC2A3 (GLUT3) gene identifies tumor cells or tumors that are sensitive to OXPHOS inhibition or biguanides. In certain embodiments low (decreased, reduced) expression is used to identify subjects with cancer who are candidates for treatment with an OXPHOS inhibitor or biguanide. In some embodiments, a measurement of expression is used to establish whether a subject in need of treatment for cancer will likely respond (or not respond) to treatment with an OXPHOS inhibitor or biguanide. In certain embodiments, a tumor is determined to have low expression of a gene and a subject in need of treatment for the tumor is treated with an OXPHOS inhibitor or biguanide.

In some embodiments assessing the level of expression of a gene comprises determining the level of a gene product of the gene in a tumor cell, tumor cell line, tumor or sample obtained from a tumor. In some embodiments the method comprises comparing the level of a gene product in a tumor cell, tumor cell line, tumor, or sample with a reference level, wherein if the level of the gene product in the tumor cell, tumor cell line, tumor, or sample is less than or equal to the reference level, the tumor cell, tumor cell line, or tumor is classified as having an increased likelihood of being sensitive to the compound than if the level is greater than the reference level.

In some aspects, described herein are methods of predicting the likelihood that a tumor cell, tumor cell line, or tumor, is sensitive to an OXPHOS inhibitor, the method comprising: (a) assessing expression of a gene listed in Table 1 or SLC2A3 or another gene listed in Table 4 by the tumor cell, tumor cell line, or tumor; and (b) generating a prediction of the likelihood that the tumor cell, tumor cell line, or tumor, is sensitive to an OXPHOS inhibitor, wherein if the tumor cell, tumor cell line, or tumor, has absent or low expression of the gene listed in Table 1 or SLC2A3, the tumor cell, tumor cell line, or tumor, is predicted to have increased likelihood of being sensitive to an OXPHOS inhibitor. In some embodiments an OXPHOS inhibitor is a complex I inhibitor. In some embodiments an OXPHOS inhibitor is a biguanide. In some embodiments an OXPHOS inhibitor is a tetracycline. In some embodiments an OXPHOS inhibitor is a glyclcycline. In some embodiments, assessing expression of the gene listed in Table 1 or SLC2A3 or another gene listed in Table 4 comprises determining the level of a gene product of a gene listed in Table 1 or SLC2A3 in the tumor cell, tumor cell line, tumor, or a sample obtained therefrom. In some embodiments the method comprises comparing the level of gene product of a gene listed in Table 1 or SLC2A3 or another gene listed in Table 4 with a reference level of the gene product. The reference level may be selected using the teachings herein. Examples of tumor cell lines with expression levels (for a one or more genes in Table 1) correlating with sensitivity to glucose limitation are provided (e.g., Jurkat, MC116, U937, NCI-H929). In some embodiments a level at or below twice the level in one or more such cell lines (or an average thereof) is a low level. In some embodiments a level at or below the level in one or more such cell lines (or an average thereof) is a low level. Examples of tumor cell lines with expression levels (for SLC2A3) correlating with sensitivity to glucose limitation are provided (e.g., KMS-26, NCI-H929). Embodiments that make use of any appropriate cell line are provided. In some embodiments a level of SLC2A3 expression at or below twice the level in one or more such cell lines (or an average thereof) is a low level. In some embodiments a level of SLC2A3 expression at or below the level in one or more such cell lines (or an average thereof) is a low level. Examples of tumor cell lines with expression levels (for one or more genes in Table 4) correlating with sensitivity to glucose limitation are provided (e.g., Jurkat, MC116, KMS-26, NCI-H929, LP-1, L-363, MOLP-8, D341 Med, KMS-28BM). In some embodiments a level at or below twice the level in one or more such cell lines (or an average thereof) is a low level. In some embodiments a level at or below the level in one or more such cell lines (or an average thereof) is a low level. Other tumor cell lines that have a gene expression signature correlating with sensitivity to low glucose are found in Table 6 (about the first 50-55 cell lines listed, e.g., those having a score (SUM) of 1784 or less). One of ordinary skill in the art will appreciate that this is not a precise cutoff. Examples of tumor cell lines with expression levels correlating with resistance to glucose limitation are provided (e.g., Raji, NCI-H82, NCI-H524, H-2171). In some embodiments a level at or above the level in one or more such cell lines (or an average thereof) is not a low level. In some embodiments such a level is a high level.

In some embodiments an expression level of a particular tumor or tumor cell line of interest is determined relative to a mean or median expression level found in a diverse set of tumors and/or tumor cell lines. Such tumors and/or tumor cell lines may be ranked, and the ranking of the particular tumor or tumor cell line of interest may be determined. In some embodiments, a set of tumors and/or tumor cell lines are ranked with respect to expression levels of two or more genes, e.g., two or more genes that constitute a gene signature. The tumors and/or tumor cell lines may be assigned a score for each gene based on their expression level of that gene. A particular tumor or tumor cell line of interest may also be assigned a score for each gene in this manner. Scores may be added for the different genes to arrive at a composite score for each tumor or tumor cell line. Ranking may, for example, be from lowest expression level (lowest score) to highest expression level (highest score) in which case a low composite rank represents a low level of expression of the gene signature. The score for a particular tumor or tumor cell line of interest is compared with the scores for the diverse set of tumors and/or tumor cell lines. In some embodiments, a score falling within the 5%, or in some embodiments within the 10%, of tumors and/or tumor cell lines having the lowest overall scores for expression of a gene signature is considered to exhibit low expression of the gene signature.

In some embodiments a diverse set of tumors or tumor cell lines comprises at least 20, 50, 100, 150, 200, 250, or 300 tumors or tumor cell lines encompassing at least 10, 20, or 30 cell types, selected without regard to their sensitivity or resistance to low glucose, high glucose, biguanides, OXPHOS inhibitors, or glycolysis inhibitors. In some embodiments a diverse set of tumors or tumor cell lines comprises or consists of at least 100, 200, 300, 400, 500, 600, 700, 800, 900, or more tumor cell lines included in the Cancer Cell Line Encyclopedia (CCLE) (see Barretina, J. et al. Nature 483, 603-607 (2012) for description of the original set of 947 CCLE tumor cell lines; see www.broadinstitute.org/ccle for updated list; see also Table 6 herein) selected without regard to their sensitivity or resistance to low glucose, high glucose, biguanides, OXPHOS inhibitors, or glycolysis inhibitors. In some embodiments at least 80%, 90%, 95%, 98%, 99%, or 100% of a diverse set of tumors or tumor cell lines are selected without regard to their sensitivity or resistance to any particular conditions or agents. In some embodiments at least 80%, 90%, 95%, 98%, 99%, or 100% of a diverse set of tumors or tumor cell lines are selected randomly from those included in the CCLE. In some embodiments one or more such cell lines may be substituted for a different tumor cell line of the same type, selected without regard to its sensitivity or resistance to low glucose, high glucose, biguanides, OXPHOS inhibitors, or glycolysis inhibitors or, in some embodiments, any other conditions or agents.

In certain embodiments a method comprises measuring an expression level of at least one gene in Table 1 in a tumor or tumor cell line or sample obtained therefrom; and classifying a tumor or tumor cell line as having an expression level no more than twice the level of expression found in a glucose limitation sensitive cancer cell line, e.g., Jurkat, U937, MC116, or NCI-H292. In some embodiments such classification is predictive that the tumor or tumor cell line is sensitive to glucose limitation, sensitive to OXPHOS inhibition (e.g., sensitive to an OXPHOS inhibitor), sensitive to a biguanide. In some embodiments the method comprises comparing the expression level in a tumor or tumor cell line or sample obtained from the tumor or tumor cell line with the expression level in in a glucose limitation sensitive cancer cell line, e.g., Jurkat, U937, MC116, or NCI-H292. In certain embodiments a method comprises measuring an expression level of at least one gene in Table 1 in a tumor or tumor cell line; and classifying a tumor or tumor cell line as having an expression level at or below the level of expression found in a glucose limitation sensitive cancer cell line, e.g., Jurkat, U937. MC116, or NCI-H292. In some embodiments such classification is predictive that the tumor or tumor cell line is sensitive to glucose limitation, sensitive to OXPHOS inhibition (e.g., sensitive to an OXPHOS inhibitor), sensitive to a biguanide. In some embodiments the method comprises comparing the expression level in a tumor or tumor cell line or sample obtained from the tumor or tumor cell line with the expression level in in a glucose limitation sensitive cancer cell line, e.g., Jurkat, U937, MC116, or NCI-H292.

In certain embodiments a method comprises measuring an expression level of SLC2A3 in a tumor or tumor cell line or sample obtained therefrom; and classifying a tumor or tumor cell line as having an expression level no more than twice the level of expression found in a glucose limitation sensitive cancer cell line that has a defect in glucose import, e.g., KMS-26 or NCI-H929. In some embodiments such classification is predictive that the tumor or tumor cell line is sensitive to glucose limitation, sensitive to OXPHOS inhibition (e.g., sensitive to an OXPHOS inhibitor), sensitive to a biguanide. In certain embodiments a method comprises measuring an expression level of SLC2A3 in a tumor or tumor cell line or sample obtained therefrom; and classifying a tumor or tumor cell line as having an expression level no more than the level of expression found in a glucose limitation sensitive cancer cell line that has a defect in glucose import, e.g., KMS-26 or NCI-H929. In some embodiments such classification is predictive that the tumor or tumor cell line is sensitive to glucose limitation, sensitive to OXPHOS inhibition (e.g., sensitive to an OXPHOS inhibitor), sensitive to a biguanide. In some embodiments the method comprises comparing the expression level in a tumor or tumor cell line or sample obtained from the tumor or tumor cell line with the expression level in in a glucose limitation sensitive cancer cell line that has a defect in glucose import, e.g., KMS-26 or NCI-H929. In some aspects, the afore-mentioned methods may be applied with respect to expression of any one or more genes listed in Table 4. e.g., low expression of the gene expression signature comprising the genes listed in Table 4.

In some aspects, described herein are methods of determining whether a subject in need of treatment for a tumor is a candidate for treatment with an OXPHOS inhibitor, the methods comprising: (a) determining the expression level of one or more genes listed in Table 1 or SLC2A3 by the tumor; and (b) identifying the subject as a candidate for treatment with an OXPHOS inhibitor if the tumor has low expression of at least one of the genes. In some embodiments the method comprises identifying the subject as a candidate for treatment with an OXPHOS inhibitor if the tumor has low expression of at least one of the genes. In some embodiments at least one of the genes is CYC1 or UQCRC1. In general, a subject is a candidate for treatment with an agent if there is sufficient likelihood that the tumor will respond to the agent to justify the risk (e.g., potential side effects) associated with the agent within the judgment of a person of ordinary skill in the art, e.g., a physician such as an oncologist. For example, if a subject has a tumor that lacks expression of the gene the subject is a candidate for treatment with an OXPHOS inhibitor, e.g., a biguanide. It will be understood that expression level may be used together with one or more additional criteria to determine whether the subject should be treated with an OXPHOS inhibitor, e.g., a biguanide Such criteria may include, for example, predicted sensitivity or previous response of the tumor to other therapies. In some embodiments expression level is used in a clinical decision support system (i.e., a computer program product designed to assist physicians and other health professionals with decision making tasks), optionally together with additional information about the tumor and/or subject, to select or assist a health care provider in selecting a treatment for the subject. In some aspects, the afore-mentioned methods may be applied with respect to expression of any one or more genes listed in Table 4, e.g., low expression of the gene expression signature comprising the genes listed in Table 4.

It will be understood that the terms “sensitive” or “resistant” as used herein in regard to sensitivity or resistance to agents or conditions, generally refers to the extent to which a cell, e.g., a tumor cell, or tumor is susceptible to or able to withstand the potential inhibitory effects of an agent or condition to which it is exposed on survival and/or proliferation. For example, tumor cell(s) may be considered sensitive if killed or rendered nonproliferative by an agent, while they may be considered resistant if able to survive and proliferate in the presence of the agent. It will be understood that sensitivity or resistance may at least depend on concentration of an agent, duration of exposure, etc. In some embodiments the level of sensitivity of a cell to an agent may be determined by contacting cells with the agent, e.g., by culturing cells in culture medium containing the agent, and measuring cell survival or proliferation after a suitable time period. Any suitable method of assessing cell survival or proliferation may be used.

In some embodiments tumor cells are classified as sensitive or resistant to an OXPHOS inhibitor or classified as having an increased or decreased likelihood of being sensitive or resistant to an OXPHOS inhibitor. In some embodiments tumor cells are classified as sensitive or resistant to a biguanide or classified as having an increased or decreased likelihood of being sensitive or resistant to a biguanide. In some embodiments tumor cells may be considered sensitive to a compound if the IC₅₀ of the compound is below about 20 μM, e.g., between 1 μM and 5 μM, between 5 M and 10 μM, or between 10 M and 20 μM. In some embodiments tumor cells may be considered sensitive to a compound if the IC₅₀ of the compound is below about 50 μM, 100 μM, 150 μM, 200 μM, 250 μM, 300 μM, 350 μM, 400 μM, or 500 μM, 1 mM, between 1 mm and 2 mM, or between 2 mM and 3 mM, or between 3 mM and 5 mM.

In some embodiments, a method described herein is used to predict in vivo tumor sensitivity to an OXPHOS inhibitor, e.g., to identify a tumor or subject having increased likelihood of responding to treatment with an OXPHOS inhibitor or to predict the likelihood that a tumor or subject will respond to treatment with an OXPHOS inhibitor. Methods and criteria that may be employed for evaluating tumor progression, response to treatment, and outcomes are known in the art and may include objective measurements (e.g., anatomical tumor burden) and criteria, clinical evaluation of symptoms, or combinations thereof. For example, imaging may be used to detect or assess number, size or metabolic activity of tumors (local or metastatic). In some embodiments a classification according to predicted sensitivity correlates with sensitivity as determined by measuring tumor response using such a method. In some embodiments a tumor is considered sensitive to an agent if a response can be obtained when the agent is administered to a subject using dose(s) that can be reasonably tolerated by the subject, while if a response is not obtained within the tolerated dose range, the tumor is considered resistant to the agent.

Expression of the genes of interest herein (e.g., genes listed in Table 1 such as CYC1, UQCRC1, or the SLC2A3 gene or other genes listed in Table 4) can be assessed using any of a variety of methods. In some embodiments gene expression is assessed by determining the level of a gene product. In some embodiments a gene product comprises an RNA, e.g., an mRNA. In some embodiments a gene product comprises a polypeptide. In some embodiments the level of a gene product is detected in a sample obtained from a tumor. In some embodiments a gene product is detected in a lysate or extract prepared from a sample. In some embodiments a gene product is detected using a method that allows detection of the gene product in individual cells that express it. In some embodiments detecting a gene product comprises contacting a sample with an appropriate detection reagent for such gene product and detecting binding of the detection reagent to the gene product by, e.g., detecting the detection reagent bound to the gene product.

In general, any suitable method for measuring RNA can be used to measure the level of an RNA, e.g., mRNA, in a sample. For example, methods based at least in part on hybridization and/or amplification can be used. The sample may comprise RNA that has been isolated from a cell or tissue sample or RNA may be detected within cells. Exemplary methods of use to detect mRNA include, e.g., in situ hybridization, Northern blots, microarray hybridization (e.g., using cDNA or oligonucleotide microarrays), reverse transcription PCR, nanostring technology (see, e.g., Geiss, G., et al., Nature Biotechnology (2008), 26, 317-325; U.S. Ser. No. 09/898,743 (U.S. Pat. Pub. No. 20030013091) for exemplary discussion of nanostring technology and general description of probes of use in nanostring technology). It will be understood that mRNA may be isolated and/or reverse transcribed to cDNA, which may be further copied, e.g., amplified, prior to detection. In some embodiments detecting mRNA comprises reverse transcription of mRNA, followed by PCR amplification with primers specific for a mRNA of interest. Thus it will be understood that in various embodiments detection of mRNA may comprise detecting mRNA molecules and/or detecting a DNA or RNA copy or reverse copy thereof. In some embodiments real-time PCR (also termed quantitative PCR), e.g., reverse transcription real-time PCR is used. Commonly used real time PCR assays include the TaqMan® assay and the SYBR® Green PCR assay. In some embodiments multiplex PCR is used, e.g., to quantify mRNA. It will be understood that certain methods of use to detect mRNA may, in at least some instances, also detect at least some pre-mRNA transcript(s), transcript processing intermediates, and degradation products of sufficient size. In some embodiments a method designed to specifically detect mRNA is used. For example, a polyT primer may be used to reverse transcribe mRNA, which may then be selectively amplified and/or detected.

In some embodiments the level of a target nucleic acid is determined by a method comprising contacting a sample with one or more nucleic acid probe(s) and/or primer(s) comprising a sequence that is substantially or perfectly complementary to the target nucleic acid over at least 10, 12, 15, 20, or 25 nucleotides, maintaining the sample under conditions suitable for hybridization of the probe or primer to its target nucleic acid, and detecting or amplifying a nucleic acid that hybridized to the probe or primer. In some embodiments, “substantially complementary” refers to at least 90% complementarity, e.g., at least 95%, 96%, 97%, 98%, or 99% complementarity. In some embodiments the sequence of a probe or primer is sufficiently long and sufficiently complementary to an mRNA of interest (or its complement) to allow the probe or primer to distinguish between such mRNA (or its complement) and at least 95%, 96%, 97%, 98%, 99%, or 100% of transcripts (or their complements) from other genes in a mammalian cell, e.g., a human cell, under the conditions of an assay. In some embodiments, a probe or primer may also comprise sequences that are not complementary to a mRNA of interest (or its complement). In some embodiments such additional sequences do not significantly hybridize to other nucleic acids in a sample and/or do not interfere with hybridization to a mRNA of interest (or its complement) under conditions of the assay. In some embodiments, an additional sequence may be used, for example, to immobilize a probe or primer to a support or to serve as an identifier or “bar code”. In some embodiments, a probe or primer hybridizes to a target nucleic acid in solution. The probe or primer may subsequently immobilized to a support. In some embodiments a probe or primer is attached to a support prior to hybridization to a target nucleic acid. Methods for attaching probes or primers to a support will be apparent to those of ordinary skill in the art. For example, oligonucleotide probes can be synthesized in situ on a surface or nucleic acids (e.g., cDNAs, PCR products) can be spotted or printed on a surface using, e.g., an array of fine pins or needles often controlled by a robotic arm that is dipped into wells containing the probes and then used to deposit each probe at a designated location on the surface.

In some embodiments a probe or primer is labeled. A probe or primer may be labeled with any of a variety of detectable labels. In some embodiments a label is a radiolabel, fluorescent small molecule (fluorophore), quencher, chromophore, or hapten. Nucleic acid probes or primers may be labeled during synthesis or after synthesis. In some embodiments a nucleic acid to be detected is labeled prior to detection, e.g., prior to or after hybridization to a probe. For example, in microarray-based detection, nucleic acids in a sample may be labeled prior to being contacted with a microarray or after hybridization to the microarray and removal of unhybridized nucleic acids. Methods for labeling nucleic acids and performing hybridization and detection will be apparent to those of ordinary skill in the art. Microarrays are available from various commercial suppliers such as Affymetrix, Inc. (Santa Clara, Calif., USA) and Agilent Technologies, Inc. (Santa Clara, Calif., USA). For example, GeneChips® (Affymetrix) may be used, such as the GeneChip® Human Genome U133 Plus 2.0 Array or successors thereof. Microarrays may comprise one or more probes or probe sets designed to detect each of thousands of different RNAs. In some embodiments a microarray comprises probes designed to detect transcripts from at least 2,500, at least 5,000, at least 10,000, at least 15,000, or at least 20,000 different genes, e.g., human genes.

In some embodiments RNA level is measured using a sequencing-based approach such as serial analysis of gene expression (SAGE) (including modified versions thereof) or RNA-Sequencing (RNA-Seq). RNA-Seq refers to the use of any of a variety of high throughput sequencing techniques to quantify RNA molecules (see, e.g., Wang, Z., et al. Nature Reviews Genetics (2009), 10, 57-63). Other methods of use for detecting RNA include, e.g., electrochemical detection, bioluminescence-based methods, fluorescence-correlation spectroscopy, etc.

In some embodiments a gene product comprises a polypeptide. In general, any suitable method for measuring proteins can be used to measure the level of a polypeptide in a sample. Numerous strategies that may be used for detection of a polypeptide are known in the art. Exemplary detection methods include, e.g., immunohistochemistry (IHC); immunofluorescence, enzyme-linked immunosorbent assay (ELISA), bead-based assays such as the Luminex® assays (Life Technologies/Invitrogen, Carlsbad, Calif.), flow cytometry, protein microarrays, surface plasmon resonance assays (e.g., using BiaCore technology), microcantilevers, immunoprecipitation, immunoblot (Western blot), etc. In some embodiments an immunological method or other affinity-based method is used. In general, immunological detection methods involve detecting specific antibody-antigen interactions in a sample such as a tissue section or cell sample. The sample is contacted with an antibody that binds to the target antigen of interest. The antibody is then detected using any of a variety of techniques. In some embodiments, the antibody that binds to the antigen (primary antibody) or an antibody (secondary antibody) that binds to the primary antibody has a detectable label attached thereto. In general, assays may be performed in any suitable vessel or on any suitable surface. In some embodiments multiwell plates are used.

In some embodiments, a polypeptide is detected using an ELISA assay. Traditional ELISA assays typically involve use of primary or secondary antibodies that are linked to an enzyme, which acts on a substrate to produce a detectable signal (e.g., production of a colored product) to indicate the presence of antigen or other analyte. As used herein, the term “ELISA” also encompasses use of non-enzymatic reporter molecules such as fluorogenic, electrochemiluminescent, or real-time PCR reporter molecules that generate quantifiable signals. It will be appreciated that the term “ELISA” encompasses a number of variations such as “indirect”, “sandwich”, “competitive”, and “reverse” ELISA. Examples of various assays and devices suitable for performing immunoassays or other affinity-based assays are described in U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; 5,480,792; 4,727,022; 4,659,678; and/or 4,376,110.

In some embodiments a polypeptide is detected using immunohistochemistry (IHC). IHC generally refers to the immunological detection of an antigen of interest (e.g., a cellular or tissue constituent) in a tissue or cell sample comprising substantially intact cells, which may be fixed and/or permeabilized. As used herein, IHC encompasses immunocytochemistry (ICC), which term generally refers to the immunological detection of a cellular constituent in isolated cells that essentially lack extracellular matrix components and tissue microarchitecture that would typically be present in a tissue sample. In some embodiments, e.g., where IHC is used for detection, a sample is in the form of a tissue section, which may be a fixed or a fresh (e.g., fresh frozen) tissue section or cell smear in various embodiments. In some embodiments fixation of cells may, for example, be performed by exposing them to 1% paraformaldehyde for 10 minutes at 37 degrees C, which may be followed by permeabilization, e.g., in 90% methanol for about 30 minutes on ice. In some embodiments a sample, e.g., a tissue section, may be embedded, e.g., in paraffin or a synthetic resin or combination thereof. A sample may be fixed using a suitable fixative such as a formalin-based fixative. In some embodiments a tissue section is a paraffin-embedded, formalin-fixed tissue section. A tissue section may be deparaffinized—a process in which paraffin (or other substance in which the tissue section has been embedded) is removed at least sufficiently to allow staining of a portion of the tissue section. To facilitate the immunological reaction of antibodies with antigens in fixed tissue or cells it may be helpful to unmask or “retrieve” the antigens through pretreatment of the sample. A variety of procedures for antigen retrieval (sometimes called antigen recovery) can be used. Such methods can include, for example, applying heat (optionally with pressure) and/or treating with various proteolytic enzymes. Methods can include microwave oven irradiation, combined microwave oven irradiation and proteolytic enzyme digestion, pressure cooker heating, autoclave heating, water bath heating, steamer heating, high temperature incubator, etc. To reduce background staining in IHC, the sample may be incubated with a buffer that blocks the reactive sites to which the primary or secondary antibodies may otherwise bind. Common blocking buffers include, e.g., normal serum, non-fat dry milk, bovine serum albumin (BSA), or gelatin, and various other available blocking buffers. The sample is then contacted with an antibody that specifically binds to the antigen whose detection is desired. After an appropriate period of time, unbound antibody is removed (e.g., by washing), and antibody that remains bound to the sample is detected. After immunohistochemical staining, a second stain may be applied, e.g., to provide contrast that helps the primary stain stand out. Such a stain may be referred to as a “counterstain”. Such stains may show specificity for discrete cellular compartments or antigens or may stain the whole cell. Examples of commonly used counterstains include, e.g., hematoxylin, Hoechst stain, or DAPI. A tissue section can be visualized using appropriate microscopy, e.g., light microscopy, fluorescence microscopy, etc.

Protein microarrays are arrays that comprise a plurality of capture reagents, e.g., detection reagents such as antibodies, immobilized on a support. The array is contacted with a sample under conditions suitable for analytes in the sample to bind to the capture reagents. Unbound material may be removed by washing. Analytes that bound to a capture reagent are detected using any of a variety of approaches. In some embodiments the array is contacted with a second reagent, such as a second detection reagent capable of binding to an analyte of interest. See, e.g., U.S. Patent Pub. Nos. 20030153013 and 20040038428 for examples of protein microarrays and methods of making and using them.

In some embodiments, flow cytometry (optionally including cell sorting) is used to detect expression. Flow cytometry is typically performed on isolated cells suspended in a liquid. For example, a tissue sample may be processed to isolate cells from surrounding tissue. The cells are contacted with a detection reagent that binds to mRNA to be detected (e.g., a nucleic acid probe) or that binds to protein to be detected (e.g., an antibody), washed to remove unbound detection reagent, and subjected to flow cytometry. The detection reagent is appropriately labeled (e.g., with a fluorescent moiety) so as to be detectable by flow cytometry.

In some embodiments an antibody used in an immunological detection method or therapeutic method is monoclonal. In some embodiments an antibody is polyclonal. In some embodiments, an antibody preparation comprises multiple monoclonal antibodies, which may bind to the same epitope or different epitopes of a protein to be detected. Antibodies can be generated using full length protein as an immunogen or binding target or using one or more fragments of a protein as an immunogen or binding target. In some embodiments, an antibody is an anti-peptide antibody. Antibodies capable of detecting various proteins of interest, e.g., human CYC1 protein, are commercially available. One of ordinary skill in the art would be able, using standard methods such as hybridoma technology or phage display, to generate additional antibodies suitable for use to detect proteins of interest herein. An antibody may be of any immunoglobulin class (e.g., IgG, IgA, IgE, IgD, IgM, IgY) or subclass and may be derived from any species (e.g., a mammal such as a mouse, rat, goat, sheep, human), a bird (e.g., a chicken). In some embodiments an antibody is a chimeric antibody, a humanized antibody, or a human antibody. One of ordinary skill in the art would appreciate that useful antibodies may be full size antibodies comprising two heavy and two light chains or may be antibody fragments such as F(ab′)2 fragment, Fab fragment, single chain variable (scFv) fragments, or single domain antibodies, etc.

In some embodiments, a ligand that specifically binds to a protein of interest and that is not an antibody is used as a detection reagent or therapeutic agent. For example, nucleic acid aptamers or various non-naturally occurring polypeptides that are structurally distinct from antibodies may be used. Examples include, e.g., agents referred to in the art as affibodies, anticalins, adnectins, synbodies, etc. See, e.g., Gebauer, M. and Skerra, A., Current Opinion in Chemical Biology, (2009), 13(3): 245-255 PCT/DE1998/002898(published as WO/1999/016873), or PCT/US2009/041570 (published as WO/2009/140039). Such agents may be used to detect a protein in a similar manner to antibodies.

In some embodiments an antibody or other binding agent, e.g., a detection reagent or therapeutic agent, binds to a polypeptide with a K_{d}, of 10⁻⁴ or less, e.g., 10⁻⁵ M or less, e.g., 10⁻⁶ M or less, 10⁻⁷ M or less, 10⁻⁸ M or less, 10⁻⁹ M or less, or 10⁻¹⁰ M or less.

In some embodiments, a non-affinity based method such as mass spectrometry may be used to assess the level of a polypeptide.

In some embodiments expression may be detected in a tumor in vivo by administering an appropriate detection reagent to a subject. In some embodiments the detection reagent binds to a gene product, e.g., a protein, and is then detected by, for example, a suitable detector or imaging method. The amount of detection reagent bound to the tumor provides an indication of the amount of gene product expressed. Useful molecular imaging modalities include molecular MRI (mMRI), magnetic resonance spectroscopy, optical bioluminescence imaging, optical fluorescence imaging, ultrasound, single-photon emission computed tomography (SPECT), positron emission tomography (PET), and combinations thereof. The detection reagent may comprise a label to render it more readily detectable. A label may be a radionuclide such as ¹²³I, ¹¹¹In, ^{99m}Tc, ⁶⁴Cu, or ⁸⁹Zr, a fluorescent moiety, magnetic or paramagnetic particle, microbubble (for ultrasound-based detection), quantum dot (semiconductor nanoparticles), nanocluster, etc. In some embodiments the detection reagent is detected noninvasively. In some embodiments the detection reagent may be detected at the time of surgery to remove a tumor or using a probe or endoscope, which may be equipped with a detector.

A reagent, e.g., detection reagent such as an antibody that binds to a polypeptide or a probe or primer that hybridizes to a mRNA or to a complement thereof, or a procedure for use to detect a gene product may be validated, if desired, by showing that a classification or prediction obtained using such detection reagent or procedure on an appropriate set of test samples correlates with a characteristic of interest such as sensitivity to OXPHOS inhibition or likelihood of therapeutic response to OXPHOS inhibition, or sensitivity to a particular compound or class of compound, e.g., biguanides. A set of test samples may be selected to include, e.g., at least 3, 5, 10, 20, 30, or more samples in each category in a classification system (e.g., high expression, low expression). In some embodiments, a set of test samples comprises samples from tumors of a particular tumor type or tissue of origin. Once a particular reagent or procedure has been validated it can be used to validate additional reagents or procedures.

Suitable controls, normalization procedures, or other types of data processing can be used to accurately quantify expression, where appropriate. In some embodiments measured values are normalized based on total mRNA or total protein or total cell number in a sample. In some embodiments measured values are normalized based on the expression of one or more RNAs or polypeptides whose expression is not correlated with a characteristic of interest such as sensitivity to OXPHOS inhibition and the expression level of which is not expected to vary greatly between tumor cells and non-tumor cells or is not expected to vary greatly among tumors in general or is not expected to vary greatly among tumors of the tumor type to which a particular tumor belongs. In some embodiments the gene used for normalization encodes a ribosomal protein, e.g., ribosomal protein S6. In some embodiments the gene used for normalization encodes an actin, e.g., actin B.

In some embodiments a measured value for the level of a gene product is normalized to account for the fact that different samples may contain different proportions of a cell type of interest, e.g., cancer cells versus non-cancer cells (e.g., stromal cells). Cells may be distinguished by their expression of various cellular markers. For example, in some embodiments the percentage of stromal cells, e.g., fibroblasts, may be assessed by measuring expression of a stromal cell-specific marker, and the result of a measurement of level of an RNA or polypeptide of interest in the sample may be adjusted to accurately reflect such RNA or polypeptide level specifically in the tumor cells. It will be understood that if a sample contains distinguishable areas of neoplastic and non-neoplastic tissue (e.g., based on standard histopathological criteria), such as at the margin of a tumor, the level of expression may be assessed specifically in the area of neoplastic tissue, e.g., for purposes of classifying the tumor or other purposes described herein. In some embodiments a level measured in non-neoplastic tissue of the sample may be used as a reference level for purposes of comparison, e.g., as described herein.

In some embodiments a background level, which may reflect non-specific binding of a detection reagent, may be subtracted from a measured value of a gene product level.

In some embodiments multiple measurements are performed on a tumor sample and/or or multiple tumor samples from a tumor are assessed. In some embodiments the number of measurements performed on a sample or the number of samples assessed is between 2 and 10. In some embodiments an average value of expression level is used.

In some embodiments the level of a gene product is determined to be “increased” or “decreased” or “high” or “low” as compared with a reference level. A reference level may be a predetermined value, or range of values (e.g. from analysis of a set of samples) determined to correlate with increased sensitivity to OXPHOS inhibition or increased likelihood of sensitivity to an OXPHOS inhibitor. Any method herein that includes a step of assessing the level of gene expression may comprise a step of comparing the level of gene expression with a reference level. In some embodiments a reference level is an absolute level. In some embodiments a reference level is a relative level, such as a proportion of cells that exhibit weak or absent staining for a particular protein. In some embodiments a reference level is range of levels.

In some embodiments comparing a gene product level with a reference level may comprise determining a difference between the measured level and the reference level, e.g., by subtracting the reference level from the measured level or may comprise determining a ratio. A comparison may involve subjecting the results of one or more measurements to any appropriate statistical analysis in various embodiments.

In some embodiments expression data obtained from a panel of tumor reference samples are used to establish reference level(s) that represent increased or decreased expression or to establish reference level(s) that represent high or low expression levels. In some embodiments the reference samples are from cancers or cancer cell lines that are determined to be sensitive to glucose limitation. In some embodiments the reference samples are from cancers or cancer cell lines that are determined to be sensitive to OXPHOS inhibition. In some embodiments the reference samples are from cancers or cancer cell lines that are determined to be sensitive to biguanide(s), e.g., metformin. In some embodiments reference levels of expression that correlate, with OXPHOS inhibition sensitivity or biguanide sensitivity with at least a specified correlation coefficient (e.g., at least 80%, at least 90%, or more) are established. In some embodiments, a method may comprise determining a reference level. Reference samples may be of a particular tumor type, e.g., liver, breast, lung, pancreatic, kidney, etc., or a particular subtype, such as ER positive. ER negative, or triple negative breast tumors. In some embodiments a reference level is a level that has been determined using the same type of sample, comparable handling of the sample, same type of gene product (e.g., mRNA or protein), and same or equivalent detection technique as for the subject or tumor being tested.

In some embodiments archived tissue samples, which may be in the form of one or more tissue microarrays (TMA), are used. Tissue microarrays may be produced by obtaining small portions (e.g., disks) of tissue from various types of standard histologic sections (e.g., formalin-fixed paraffin-embedded (FFPE) samples) or from newly obtained samples and placing or embedding them in a regular arrangement (e.g., in mutually perpendicular rows and columns) on or in a substrate such as a paraffin block. A tissue microarray may comprise many, e.g., dozens or hundreds of samples (e.g., between about 50 and about 1000 samples), which can be analyzed in parallel and using uniform analysis conditions. See, e.g., Kononen J, et al., Tissue microarrays for high-throughput molecular profiling of tumor specimens. Nat Med 1998, 4:844-847; Equiluz, C., et al., Pathol Res Pract., 202(8):561-8, 2006. TMAs may be prepared using a hollow needle to remove tissue cores (e.g., as small as about 0.6 mm in diameter) from paraffin-embedded tissue samples. These tissue cores are then inserted in a paraffin block in an array pattern. Sections from such a block can be cut, e.g., using a microtome, mounted on a microscope slide, and then analyzed by any method of analysis, e.g., standard histological analysis methods such as IHC or FISH. Each microarray block can be cut into 100-500 sections, which can be subjected to independent tests.

In some embodiments cancers falling within the lower quartile of expression level of a gene of interest (i.e., the 25% of tumors having the lowest expression level) are classified as having a low expression level. In some embodiments cancers falling within the lower tenth of expression level of a gene of interest (i.e., the 10% of tumors having the lowest expression level) are classified as having a low expression level. In some embodiments the tumors are of a particular type or tissue of origin. The levels of expression that correlate with sensitivity, e.g., in in tumors of a particular type or tissue of origin may be used for classifying other tumors, e.g., other tumors of that type or tissue of origin. In some embodiments levels of expression that correlates with a specified correlation coefficient (e.g., at least 0.80, at least 0.85, at least 0.90, at least 0.925, at least 0.95, or more) with sensitivity in tumors or tumor cell lines in general or tumor or tumor cell lines of a particular type or tissue of origin are used. In some embodiments a correlation coefficient is a Pearson correlation coefficient. In some embodiments a correlation coefficient is a Spearman correlation coefficient.

A measured value or reference level may be semi-quantitative, qualitative, or approximate. For example, visual inspection (e.g., using microscopy) of a stained IHC sample can provide an assessment of the level of expression without necessarily counting cells or precisely quantifying the intensity of staining. In some embodiments one or more steps of a method described herein is performed at least in part by a machine, e.g., computer (e.g., is computer-assisted) or other apparatus (device) or by a system comprising one or more computers or devices. In some embodiments a computer is used in sample tracking, data acquisition, and/or data management. For example, in some embodiments a sample ID is entered into a database stored on a computer-readable medium in association with a measurement of expression. The sample ID may subsequently be used to retrieve a result of determining expression in the sample. In some embodiments, automated image analysis of a sample is performed using appropriate software, comprising computer-readable instructions to be executed by a computer processor. For example, a program such as ImageJ (Rasband, W. S., ImageJ, U. S. National Institutes of Health, Bethesda, Md., USA, http://imagej.nih.gov/ij/, 1997-2012; Schneider, C. A., et al., Nature Methods 9: 671-675, 2012; Abramoff, M. D., et al., Biophotonics International, 11(7): 36-42, 2004) or others having similar functionality may be used. In some embodiments, an automated imaging system is used. In some embodiments an automated image analysis system comprises a digital slide scanner. In some embodiments the scanner acquires an image of a slide (e.g., following IHC for detection of a gene product) and, optionally, stores or transmits data representing the image. Data may be transmitted to a suitable display device, e.g., a computer monitor or other screen. In some embodiments an image or data representing an image is added to a patient medical record.

In some embodiments a machine, e.g., an apparatus or system, is adapted, designed, or programmed to perform an assay for measuring expression of a gene listed in Table 1 or SLC2A3 or another gene listed in Table 4. In some embodiments an apparatus or system may include one or more instruments (e.g., a PCR machine), an automated cell or tissue staining apparatus, a device that produces, records, or stores images, and/or one or more computer processors. The apparatus or system may perform a process using parameters that have been selected for detection and/or quantification of a gene product of a gene listed in Table 1 or SLC2A3 or another gene listed in Table 4, e.g., in tumor samples. The apparatus or system may be adapted to perform the assay on multiple samples in parallel and/or may comprise appropriate software to provide an interpretation of the result. The apparatus or system may comprise appropriate input and output devices, e.g., a keyboard, display, printer, etc. In some embodiments a slide scanning device such as those available from Aperio Technologies (Vista, Calif.), e.g., the ScanScope AT, ScanScope CS, or ScanScope FL or is used.

In some embodiments an assessment of expression of a gene listed in Table 1 or SLC2A3 or another gene listed in Table 4 is used as a diagnostic test, which may be referred to as a “companion diagnostic”, to determine, e.g., whether a patient is a candidate for treatment with an OXPHOS inhibitor, e.g., a biguanide. In some embodiments a reagent or kit for performing such a diagnostic test may be packaged or otherwise supplied an OXPHOS inhibitor, e.g., a biguanide. In some embodiments an OXPHOS inhibitor, a biguanide, or pharmaceutical composition comprising such an agent may be approved by a government regulatory agency (such as the US FDA or government agencies having similar authority over the approval of therapeutic agents in other jurisdictions), e.g., allowed to be marketed, promoted, distributed, sold or otherwise provided commercially for treatment of humans or for veterinary purposes, with a recommendation or requirement that the subject is determined to be a candidate for treatment with the agent based at least in part on assessing the level of expression of a gene listed in Table 1 or SLC2A3 or another gene listed in Table 4 in a tumor of the subject to be treated. For example, the approval may be for an indication that includes a requirement that a tumor to be treated has a low level of such expression. Such a requirement or recommendation may be included in a package insert or label provided with the agent or composition In some embodiments a particular method for detection or measurement of a gene product or a specific detection reagent or specific kit comprising such reagent may be specified.

In certain embodiments any of the methods may comprise assigning a score to a sample (or to a tumor from which a sample was obtained) based at least in part on the level of expression measured in the sample. In some embodiments, a score is assigned using a scale of 0 to X, where 0 indicates that the sample is “negative” for the gene product (e.g., no to minimal detectable polypeptide, and X is a number that represents strong (high intensity) staining of the majority of cells. In some embodiments, a score is assigned using a scale of 0, 1, or 2, where 0 indicates that the sample is negative for expression (e.g., no or minimal detectable polypeptide), 1 is low to moderate level staining and 2 is strong (intense) staining of the majority of tumor cells. In some embodiments “no detectable expression” or “negative” means that the level detected, if any, is not noticeably or not significantly different to a background level.

In some embodiments a score is assigned based on assessing both the level of expression and the percentage of cells that exhibit expression. For example, a score can be assigned based on the percentage of cells that exhibit low expression and the extent to which expression level is decreased. It will be understood that if a tissue sample comprises areas of neoplastic tissue and areas of non-neoplastic tissue a score can be assigned based on expression in the neoplastic tissue. In some embodiments the non-neoplastic tissue may be used as a reference.

In some embodiments at least about 30%, 40%, 50%, 60%, 70%, 80%, 90%, or more tumor cells in a sample assessed express decreased levels of a gene listed in Table 1 or SLC2A3 or another gene listed in Table 4. A score can be obtained by evaluating one field or multiple fields in a cell or tissue sample. In some embodiments multiple samples from a tumor are evaluated. It will be appreciated that a score can be represented using numbers or using any suitable set of symbols or words instead of, or in combination with numbers. For example, scores can be represented as 0, 1, 2; negative, positive; negative, low, high; −, +, ++, +++; 1+, 2+, 3+, etc. In some embodiments, at least 10, 20, 50, 100, 200, 300, 400, 500, 1000 cells, or more, are assessed to evaluate expression in a sample or tumor and/or to assign a score to a sample or tumor. In some embodiments the number of cells is up to about 10⁴, 10⁵, 10⁶, 10⁷, 10⁸, or more. The number of cells may be selected as appropriate for the particular assay used and/or so as to achieve a particular degree of accuracy, repeatability, or reproducibility.

In some embodiments the number of categories in a useful scoring or classification system is least 2, e.g., 2, 3, or 4, or between 4 and 10, although the number of categories may be greater than 10 in some embodiments. In some embodiments a scoring or classification system is effective to divide a population of tumors or subjects into groups that differ in terms of a result or outcome such as response to a treatment or survival. A result or outcome may be assessed at a given time or over a given time period, e.g., 3 months, 6 months, 1 year, 2 years, 5 years, 10 years, 15 years, or 20 years from a relevant date such as the date of diagnosis or approximate date of diagnosis (e.g., within about 1 month of diagnosis) or a date after diagnosis, e.g., a date of initiating treatment. Various categories may be defined. For example, tumors may be classified as having low, intermediate, or high likelihood of sensitivity to OXPHOS inhibition or a biguanide, or a subject may be determined to have a low, intermediate, or high likelihood of experiencing a clinical response to OXPHOS inhibition or a biguanide. A variety of statistical methods may be used to correlate the likelihood of a particular outcome (e.g., sensitivity, resistance, response, lack of response, survival for at least a specified time period) with the relative or absolute level of expression. One of ordinary skill in the art will be able to select and perform appropriate statistical tests. Correlations may be calculated by standard methods, such as a chi-squared test, e.g., Pearson's chi-squared test. Such methods are well known in the art (see, e.g., Daniel, W. W., et al., Biostatistics: A Foundation for Analysis in the Health Sciences, 8th ed. (Wiley Series in Probability and Statistics), 2004 and/or Zar, J., Biostatistical Analysis, 5^{th} ed., Prentice Hall; 2009). Statistical analysis may be performed using appropriate software. Numerous computer programs suitable for performing statistical analysis are available. Examples, include, e.g., SAS, Stata, GraphPad Prism, and many others. R is a programming language and software environment useful for statistical computing and graphics that provides a wide variety of statistical and graphical techniques, including linear and nonlinear modeling, classical statistical tests, classification, clustering, and others.

One of ordinary skill in the art will appreciate that the terms “predicting”, “predicting the likelihood”, and like terms, as used herein, do not imply or require the ability to predict with 100% accuracy and do not imply or require the ability to provide a numerical value for a likelihood. Instead, such terms typically refer to forecast of an increased or a decreased probability that a result, outcome, event, etc., of interest (e.g., sensitivity of a tumor cell or tumor to OXPHOS inhibition or a biguanide) exists or will occur, e.g., when particular criteria or conditions exist, as compared with the probability that such result, outcome, or event, etc., exists or will occur when such criteria or conditions are not met. In some embodiments a numerical value may be provided, such as an absolute or relative likelihood. In some embodiments an increased likelihood is increased by at least 25%, 50%, 75%, 100%, 200% (2-fold), 300% (3-fold), 400% (4-fold), 500% (5-fold), or more. In some embodiments an increased likelihood is a likelihood of at least 20%, 30%, 40%, 50%, 60%, 70%, 80%, 90%, or more. It will also be understood that a method for predicting the likelihood of tumor cell or tumor sensitivity (or resistance) may comprise or be used together with one or more other methods. For example, assessment of expression may be used together with assessment of one or more additional genes, gene products, metabolites, or parameters. In some embodiments one or more such additional measurements may be combined with assessment of expression to increase the predictive value of the analysis (e.g., to provide a more conclusive determination of likelihood of sensitivity) in comparison to that obtained from measurement of a gene product alone. Thus a method of predicting likelihood can be a method useful to assist in predicting likelihood in combination with one or more other methods. The various components of a set of measurements may be assigned the same or similar weights or may be weighted differently.

In some embodiments, a level of a gene product (e.g., mRNA or polypeptide) of a gene listed in Table 1 of SCL3A2 or another gene listed in Table 4 is assessed and used together with levels of gene product(s) of one or more additional genes, e.g., for classifying a tumor cell, tumor cell line, or tumor according to predicted sensitivity to OXPHOS inhibition or biguanides. It will be understood that methods described herein of assessing expression, determining whether expression is increased or decreased, determining reference levels, etc., may be applied to assess expression of any gene of interest using appropriate detection reagents for gene products of such genes.

In certain embodiments the level of a mRNA or protein of interest is not assessed simply as a contributor to a cluster analysis, dendrogram, or heatmap based on gene expression profiling in which expression at least 10; 20; 50; 100; 500; 1,000, or more genes is assessed. In certain embodiments, if a level of a mRNA or protein of interest is measured as part of such a gene expression profile, the level of such mRNA or protein of interest is used in a manner that is distinct from the manner in which the expression of many or most other genes in the gene expression profile are used. For example, the level of such mRNA or protein of interest may be used independently of, e.g., without regard to, expression levels of most or all of the other genes or may be weighted more strongly than most or all other levels in analyzing or using the results.

In some embodiments the presence in a cancer or cancer cell line of one or more mutations in a gene, e.g., a mitochondrial gene, encoding an OXPHOS component (or other is indicative that the cancer or cancer cell line is sensitive to glucose limitation. In some embodiments the presence in a cancer or cancer cell line of one or more mutations associated mutations in a gene, e.g., a mitochondrial gene, encoding an OXPHOS component is indicative that the cancer or cancer cell line is sensitive to OXPHOS inhibition. In some embodiments the presence in a cancer or cancer cell line of one or more mutations in a gene, e.g., a mitochondrial gene, encoding an OXPHOS component is indicative that the cancer or cancer cell line is sensitive to biguanides, e.g., metformin. In some embodiments the mutation results in reduced amount and/or reduced functional activity of a protein encoded by the gene. In some embodiments the mutation is in a gene that encodes an OXPHOS component, e.g., a component of complex I, II, III, IV, or V. In some embodiments the mutation results in reduced OXPHOS capacity of mitochondria that harbor the mutation. In some embodiments a reduction in amount or functional activity is by at least 10%, 20%, 30%, 40%, 50%, 60%, 70%, 80%, 90%, or more, as compared to normal (i.e., in the absence of the mutation). In some embodiments a mutation is present in all mitochondria of a cell (homoplasmy). In some embodiments a mutation is present in some but not all mitochondria of a cell (heteroplasmy). In some embodiments a mutation is present in at least 10%, 20%, 30%, 40%, 50%, 60%, 70%, 80%, 90%, or more of the mitochondria of a cell. In some embodiments the mutation is present in at least some mitochondria in at least 10%, 20%, 30%, 40%, 50%, 60%, 70%, 80%, 90%, or more cells of a tumor (e.g., in a sample obtained from the tumor). In some embodiments a mutation is a deletion or insertion. In some embodiments a mutation results in an altered protein sequence. In some embodiments a mutation is a point mutation. In some embodiments a mutation results in a protein truncation. In some embodiments a mutation is a frameshift mutation. In some embodiments a mutation is in a gene that encodes a component of complex I, e.g., mitochondrial ND1 or ND5 or ND4. In some embodiments a mutation is an alteration (e.g., an A to G alteration) at position 161 of the coding region of the ND1 gene. In some embodiments a mutation is an alteration (e.g., an insertion, e.g., an insertion of an A between positions 89 and 90 of the coding region of the ND5 gene). In some embodiments a mutation is a frameshifting mutation in a PolyA tract located at mtDNA position 12418-12425. This mutation may have a prevalence approaching 7.5% and has been identified at least in the following cancers: lung, liver, colon, rectal, ovarian, and AML. In some embodiments the mutation is any of the mutations listed in Table 5 (see Example 9).

In some embodiments a method comprises sequencing DNA of a gene encoding an OXPHOS component in a glucose limitation sensitive cell line, identifying a mutation in such gene, and determining whether presence of the mutation correlates with glucose limitation sensitivity. In some embodiments sequencing comprises sequencing mtDNA.

In some embodiments the presence in a cancer or cancer cell line of one or more mutations associated with a human mitochondrial disorder (or other mutations in such genes that have not as yet been identified in human mitochondrial disorders) is indicative that the cancer or cancer cell line is sensitive to glucose limitation. In some embodiments a mutation associated with a human mitochondrial disorder results in reduced amount and/or reduced functional activity of a protein encoded by the gene harboring the mutation. In some embodiments the mutation is in a mitochondrial gene. In some embodiments the mutation results in reduced OXPHOS capacity. In some embodiments the mutation is in a gene that encodes an OXPHOS component, e.g., a component of complex I, II, III, IV, or V. In some embodiments the presence in a cancer or cancer cell line of one or more mutations associated with a human mitochondrial disorder (or other mutations in such genes that have not as yet been identified in human mitochondrial disorders) is indicative that the cancer or cancer cell line is sensitive to OXPHOS inhibition. In some embodiments the presence in a cancer or cancer cell line of one or more mutations associated with a human mitochondrial disorder (or other mutations in such genes that have not as yet been identified in human mitochondrial disorders) is indicative that the cancer or cancer cell line is sensitive to biguanides, e.g., metformin. A compendium of numerous human genes and genetic phenotypes that occur in humans, including many associated with mitochondrial diseases, is provided in McKusick V. A. (1998) Mendelian Inheritance in Man. A Catalog of Human Genes and Genetic Disorders, 12th Edn. The Johns Hopkins University Press, Baltimore. Md. and its online updated version Online Mendelian Inheritance in Man (OMIM), available at the National Center for Biotechnology Information (NCBI) website at http://www.ncbi.nlm.nih.gov/omim. In some embodiments a mitochondrial disorder, e.g., a mitochondrial disorder arising at least in part from a mutation in mtDNA is maternally inherited. In some embodiments a mitochondrial disorder is inherited in a Mendelian pattern. In some embodiments a mitochondrial disorder arises sporadically, i.e., it is not inherited from a parent. A mutation may be in the germ line or somatic. In some embodiments a mitochondrial disorder is caused at least in part by a mutation in a nuclear or mitochondrial gene that encodes a component of complex I, II, III, IV, or V. In some embodiments a mitochondrial disorder is caused at least in part by a mutation in a nuclear or mitochondrial gene that encodes an assembly factor for complex I, II, III, IV, or V. In some embodiments an assembly factor (typically a protein) is involved in transcription and/or translation of a subunit of complex I-V (e.g., a mitochondrion-encoded subunit), processing of a preprotein, membrane insertion, or cofactor biosynthesis or transport or incorporation. In general, a mutation, e.g., a mutation that causes a mitochondrial disease or other phenotype may comprise any type of alteration in DNA sequence, relative to a normal sequence, in various embodiments. In general, certain mutations may result in abnormal expression level and/or activity of a gene product. In some embodiments a mutation results in abnormal expression level and/or activity of a gene product that is a component of a metabolic pathway as compared with a level of expression or activity. In general, a mutation may affect any region of a gene. In some embodiments a mutation is in a region of a gene that is transcribed. In some embodiments a mutation results in an alteration in an encoded polypeptide sequence, as compared to a normal polypeptide sequence. In some embodiments a mutation is a nonsense mutation, missense mutation, frameshift mutation, or a mutation that impairs proper splicing (e.g., a splice site mutation). In some embodiments a mutation is in a regulatory region of a gene. In some embodiments a mutation results in abnormal expression of the gene containing the mutation. For example, a mutation may result in increased or decreased level of a gene product in at least some cells, as compared with a normal level of the gene product. In some embodiments, a mutation results in a deficiency of functional gene product. For example, a mutation may result in an alteration in an encoded gene product that causes the gene product to have reduced activity relative to a normal gene product or to interfere with activity of a normal gene product encoded by another allele of the gene in a diploid organism. A mutation in a regulatory region of a gene may result in a decreased synthesis of a gene product encoded by the gene. A normal nucleic acid (DNA, RNA) or polypeptide sequence may be, e.g., (i) a nucleic acid or polypeptide sequence in which the nucleotide or amino acid, respectively, present at each position in the sequence has a prevalence of at least 1% in a population or (ii) a nucleic acid or polypeptide sequence whose expression and activity do not differ detectably from that of the nucleic acid or polypeptide sequence of (i). A normal sequence may be, e.g., the most common sequence present in a population, a reference sequence (e.g., an NCBI RefSeq sequence, or a UniProt reference sequence), or a sequence in which the nucleotide or amino acid present at each position of the sequence is the most common nucleotide or amino acid present at that position in a population. In some embodiments a mutation has a prevalence of less than 0.5%, less than 0.1%, less than 0.05%, or less than 0.001% in a population. In some embodiments a mutation may result in an expression level or activity that lies outside a normal range for expression level or activity of a gene product, i.e., below the lower limit of normal or above the upper limit of normal. A normal range may be, e.g., a range that is accepted in the art as normal. In some embodiments a normal range may be defined as a range that would encompass at least 95% of values measured in a population. In some embodiments, a “population” may be the general population, e.g., of a city, state, country or other region. In some embodiments a population may consist of individuals without any known condition that directly affects the range being established. A normal range or normal sequence may be obtained by evaluating a representative sample of a population.

Mutations may be detected using any of a wide variety of methods known in the art. In some embodiments a hybridization-based method is used. In some embodiments a method based on PCR, e.g., real-time PCR, is used. Such methods include use of allele-specific competitive blocker PCR, blocker-PCR real-time genotyping with locked nucleic acids, restriction enzymes in conjunction with real-time PCR, and allele-specific kinetic PCR in conjunction with modified polymerases. Additional methods include ARMS-PCR, TaqMAMA, FLAG-PCR, and Allele-Specific PCR with a Blocking reagent (ASB-PCR). See, e.g., Morlan, J., et al., Mutation Detection by Real-Time PCR: A Simple, Robust and Highly Selective Method, PLoS ONE 4(2): e4584, doi:10.1371/journal.pone.0004584 and references therein for description of such methods. In some embodiments a mutation may be detected using allele-specific primer hybridization or allele-specific primer extension. Signal amplification assays include branched chain DNA assays and hybrid capture assays. Transcription based amplification and nucleic acid sequence based amplification (NASBA) may be used. In some embodiments allele-specific primer extension or allele-specific hybridization is used. Microarrays, e.g., oligonucleotide micorarrays, can be used, having probes for different alleles attached thereto. A microarray can be a solid phase or suspension array (e.g., a microsphere-based approach such as the Luminex platform).

In some embodiments sequencing is used to detect and/or identify a mutation. Sequencing, e.g., mtDNA sequencing, can be performed using any sequencing method in various embodiments. Examples of sequencing approaches include, e.g., chain termination sequencing (Sanger sequencing), 454 pyrosequencing, sequencing by synthesis (e.g., Illumina (Solexa) sequencing), sequencing by ligation (e.g., SOLiD sequencing), ion semiconductor sequencing, HeliScope single molecule sequencing, single molecule real time (SMRT) sequencing, nanopore DNA sequencing. In some embodiments high throughput sequencing is performed. In some embodiments high throughput sequencing (or next-generation sequencing) comprises any of a variety of technologies that parallelize the sequencing process, producing thousands, millions, or billions of short sequences at once. Such sequences may be matched against a reference sequence to, e.g., assemble a longer sequence, identify mutations, etc.

In some embodiments a method comprises determining the level of activation of 5′ adenosine monophosphate-activated protein kinase (AMPK). 5′ adenosine monophosphate-activated protein kinase (AMPK) is an enzyme that plays a number of important roles in cellular energy homeostasis in eukaryotic organisms. AMPK is activated under a variety of conditions that decrease ATP generation, such as nutrient starvation and hypoxia, as well as by metabolic poisons. AMPK regulates the activities of a number of key metabolic enzymes through phosphorylation, resulting in stimulation of various ATP-generating catabolic pathways and inhibition of various biosynthetic pathways that consume ATP, thereby helping protect cells from stresses that cause ATP depletion. AMPK is a heterotrimeric protein composed of α, β, and γ subunits. In mammals there are two genes that encode isoforms of the catalytic a subunit (α1 and α2), two genes that encode isoforms of the 3 subunit, and three genes that encode isoforms of the γ subunit (γ1, γ2, and γ3) isoforms. The a subunit contains the catalytic domain, whereas the β and γ subunits serve regulatory roles. The γ subunit includes four so-called cystathionine beta synthase (CBS) domains that allow AMPK to detect changes in the AMP:ATP and/or ADP:ATP ratio. The CBS domains form a site that binds AMP and two additional sites that competitively bind AMP, ADP, and ATP. Cellular energy status is sensed via the latter two sites. Binding of AMP causes conformational changes in AMPK that enhance its activation by promoting phosphorylation at a conserved threonine in the catalytic domain, inhibiting dephosphorylation of this residue, and allosteric activation. Phosphorylation of the a subunit within the catalytic domain (at a conserved threonine residue (Thr-172) by an upstream AMPK kinase (AMPKK) results in activation of AMPK. Binding of AMP to the γ subunit protects the activation loop from dephosphorylation by phosphatases such as PP2C, therefore leading to AMPK activation. The complex formed between LKB1 (STK 11), mouse protein 25 (MO25), and the pseudokinase STE-related adaptor protein (STRAD) has been identified as the major upstream kinase responsible for phosphorylation of AMPK at Thr-172. In some aspects, increased AMPK phosphorylation (indicative of AMPK activation) is indicative of a tumor or tumor cell line that is likely to be sensitive to glucose limitation, OXPHOS inhibition, and/or particular OXPHOS inhibitor(s), e.g., biguanides such as metformin.

In general, methods disclosed herein may be applied to any tumor cell, tumor cell line, tumor or sample comprising tumor cells. Various tumor types and tumor cell lines are mentioned herein. For example, in some embodiments a tumor is a solid tumor. In some embodiments a solid tumor is a liver, breast, gastrointestinal tract (e.g., stomach cancer, colon cancer, esophageal cancer, rectal cancer), cervical, ovarian, pancreatic, renal, prostate, esophageal, lung, or brain cancer (e.g., glioblastoma). In some embodiments a tumor is a hematological malignancy, e.g., a leukemia (e.g., AML), lymphoma, or myeloma.

In some embodiments, a tumor has detectably metastasized when assessed or treated. In some embodiments, a tumor has not detectably metastasized when assessed or treated. In some embodiments, a tumor is a recurrent tumor (i.e., a tumor that reappears after becoming undetectable) or a relapsed tumor (i.e., a tumor that has initially responded to therapy but then worsens). In some embodiments the tumor is resistant to one or more standard chemotherapy agents or regimens.

In some embodiments expression and/or presence or absence of a mutation (e.g., sequence) is assessed at a testing facility. A testing facility or individual may be qualified or accredited (e.g., by a national or international organization such as a government organization or a professional organization) to perform an assessment of expression and/or presence or absence of a mutation (e.g., sequence information) e.g., for purposes of tumor classification for treatment selection purposes. In some embodiments a testing facility is part of or affiliated with a health care facility. In some embodiments a testing facility is not part of or affiliated with a health care facility. It is contemplated that in some embodiments an assay of expression, activation, mutation status, or sequence may be performed at a testing facility that is remote from (e.g., at least 1 kilometer away from) the site where the sample is obtained from a subject. The testing facility may receive samples from multiple different health care providers. “Health care provider” refers to an individual (e.g., a physician or other health care worker) or an institution (e.g., a hospital, clinic, medical practice, or other health care facility) that provides health care services to individuals on a systematic or regular basis. Expression, activation, or and/or presence or absence of a mutation (e.g., sequence) may be assessed as part of a panel of molecular pathology tests performed for purposes of tumor classification, diagnosis, prognosis, or treatment selection.

In some embodiments a health care provider seeking to obtain an assessment of expression, activation, and/or presence or absence of a mutation (e.g., sequence) provides a sample (e.g., a tumor sample) to a testing facility with instructions to assess expression or sequence. In some embodiments providing a sample to a testing facility encompasses directly providing the sample (e.g., sending or transporting the sample), arranging for or directing or authorizing another individual or entity to send or transport, etc. Thus in some embodiments an assessment is obtained by a requestor, e.g., a health care provider, by requesting that such assessment be performed, e.g., by a testing facility. The term “requesting” in this context encompasses instructing, urging, demanding, directing, ordering, inducing, persuading, prompting, overseeing, arranging for, or otherwise causing another individual or entity to perform a method or step. In some embodiments a first individual or entity assists a second individual or entity in performing a step or method by, for example, providing: a sample, information about a sample, a detection reagent suitable for performing a step or method, a kit or detection device adapted to perform a step or method, or instructions for performing a method. The first individual or entity may or may not request that the method or step be performed. “Request” in this context is used interchangeably with “order”, “command”, “direct”, and like terms.

In some embodiments a sample is provided to a testing facility within no more than 1, 2, 3, 5, 7, 10, 14, 21, or 28 days after having been removed from a subject. The testing facility measures expression, activation, and/or presence or absence of a mutation (e.g., sequence) in the sample and provides a result. In some embodiments obtaining an assessment comprises entering an order for an assay of such expression into an electronic ordering system, e.g., of a health care facility. In some embodiments obtaining an assessment comprises receiving a result from a testing facility. In some embodiments obtaining an assessment comprises retrieving the result of an assessment from a database. In some embodiments a method of performing a diagnostic test comprises: (a) receiving a tumor sample obtained from a subject in need of treatment for a tumor; and (b) assessing expression, activation, and/or presence or absence of a mutation (e.g., sequence) in the tumor sample. In some embodiments the method comprises receiving a request to assess expression, activation, and/or presence or absence of a mutation (e.g., sequence) in the tumor sample or receiving a request to provide a result of such assessment in the tumor sample. In some embodiments the method further comprises providing a result of an assessment to a person or entity that provided the sample or made the request, such as a subject's health care provider. In some embodiments the result is provided by the testing facility within no more than 1, 2, 3, 5, 7, 10, 14, 21, or 28 days after having received the sample.

A result may be provided in any suitable format and/or using any suitable means. In some embodiments a result is provided in an electronic format; optionally a paper copy is provided instead of or in addition to an electronic format. In some embodiments a result is provided at least in part by entering the result into a computer, e.g., into a database, electronic medical record, laboratory information system (sometimes termed laboratory information management system), etc., wherein it may be accessed by or under direction of a requestor. In some embodiments a result may be provided via phone, voicemail, fax, text message, or email. In some embodiments a result is provided at least in part over a network, e.g., the Internet. In some embodiments a result comprises one or more numbers or scores representing an expression level, activation level, mutation status, and/or a narrative description. In some embodiments a result includes a classification of a tumor according to predicted sensitivity to OXPHOS inhibition or a particular OXPHOS inhibitor, e.g., a biguanide, e.g., metformin. In some embodiments a result indicates whether or not a tumor expresses appropriate characteristics such that a subject in need of treatment for the tumor is a candidate for treatment with an OXPHOS inhibitor, e.g., a biguanide, e.g., metformin. In some embodiments a result is provided together with additional information regarding a tumor or sample. Additional information may comprise, e.g., assessment of tumor grade, tumor stage, tumor type (e.g., cell type or tissue of origin) and/or results of assessing expression of one or more additional genes or activation or activity of a gene product. In some embodiments a result is provided in a report. In some embodiments additional information comprises results of a microscopic assessment, e.g., a pathology assessment.

In some embodiments a requestor (e.g., health care provider) treats a subject or selects a treatment for a subject based at least in part on the results of the assessment. In some embodiments the result indicates that the tumor has increased likelihood of sensitivity to glucose limitation, OXPHOS inhibition, or particular OXPHOS inhibitor(s), and the treatment used or selected is an OXPHOS inhibitor, e.g., a biguanide, e.g., metformin. In some embodiments the treatment further comprises an additional anti-cancer agent.

In some embodiments kits are provided. In some embodiments a kit comprises a detection reagent suitable for detecting expression level of a gene product of a gene listed in Table 1 or SLC2A3 or another gene listed in Table 4. In some embodiments a kit comprises a detection reagent suitable for detecting mutation status of a gene encoding an OXPHOS component, e.g., ND1 or ND5 or ND4. In some embodiments a kit comprises instructions for use of the kit and/or detection reagent to perform a method described herein. In some embodiments a kit comprises a control substance, e.g., gene product or a normal sequence.

### III. Identifying and Characterizing Agents

In some aspects, the present disclosure provides methods of testing an agent for its ability to inhibit the survival and/or proliferation of a tumor cell that is sensitive to glucose restriction. In some aspects, the present disclosure provides methods of testing an agent for its ability to inhibit the survival and/or proliferation of a tumor cell under conditions of glucose restriction. Based at least in part on the discoveries that tumor cells may vary with regard to their sensitivity to glucose restriction and that certain agents, such as biguanides, may exhibit synthetic interactions with glucose restriction, Applicants propose that conducting cell-based screens under conditions of glucose restriction will permit the identification of candidate chemotherapeutic agents that are effective under conditions of glucose restriction that exist within tumors in vivo, wherein the efficacy of such agents on at least some tumor cell types or subsets is at least in part dependent on such conditions. In some embodiments a method comprises identifying an agent that has a glucose-limitation dependent effect, e.g., glucose-limitation dependent inhibition of cell viability or proliferation. Without wishing to be bound by any theory, such agents may be overlooked or their effects may be underestimated in screens conducted under typical in vitro cell culture conditions such as standard glucose concentration. In some embodiments cancer cells are cultured in a Nutrostat. In some embodiments cancer cells are cultured under low glucose conditions.

In some aspects, the present disclosure relates to a nutrastatic culture system useful for mimicking tumor nutrient conditions. The use of the nutrastat to mimic low glucose conditions is exemplified herein, but the system may be used to analyze the effect of any nutrient condition on any one or more cell properties of interest (e.g., proliferation rate, oxygen consumption, metabolite profile, etc.) and/or to analyze the effect of any agent (e.g., a therapeutic agent or candidate therapeutic agent), optionally in combination with a nutrient condition of interest, on any such property.

In some aspects, the present disclosure provides the recognition that genes that are differentially required for proliferation under low glucose are suitable targets for identification of anti-tumor agents. In some embodiments inhibitors of such genes or their encoded gene products are useful as anti-tumor agents, e.g., for tumors that are sensitive to glucose limitation. In some aspects, the present disclosure provides methods of identifying a candidate anti-tumor agent, the methods comprising identifying an agent that inhibits expression or activity of a gene product of a gene listed in Table 1 or Table 4. In some embodiments the gene is CYC1 or UQCRC1. In some aspects, the present disclosure provides the recognition that genes that encode glucose transporters, e.g., SLC2A3, are suitable targets for identification of anti-tumor agents. In some aspects, the present disclosure provides methods of identifying a candidate anti-tumor agent, the methods comprising identifying an agent that inhibits expression or activity of a gene product of SLC2A3. In some embodiments an agent identified according to the methods is useful to treat a tumor that exhibits at least one indicator of sensitivity to glucose limitation. In some embodiments an agent identified according to the methods is useful to treat a tumor in combination with an OXPHOS inhibitor. In some embodiments the agent increases sensitivity of a tumor to glucose limitation. In some embodiments an agent identified according to the methods is useful to treat a tumor in combination with a biguanide. In some embodiments the agent increases sensitivity of a tumor to biguanides.

In some aspects, genes characterized in that low expression of the gene correlates with impaired glucose utilization are suitable targets for identification of anti-tumor agents. In some embodiments inhibitors of such genes or their encoded gene products are useful as anti-tumor agents, e.g., for tumors that have impaired glucose utilization, e.g., due to low expression or activity of one or more genes listed in Table 4. In some aspects, the present disclosure provides methods of identifying a candidate anti-tumor agent, the methods comprising identifying an agent that inhibits expression or activity of a gene product of a gene listed in Table 4. In some embodiments an agent identified according to the methods is useful to treat a tumor that exhibits at least one indicator of sensitivity to glucose limitation, such as low expression of any one or more of the afore-mentioned genes or low activity of a protein encoded by the gene (e.g., due to a mutation in the gene). In some embodiments, a tumor that has low but non-zero expression of a particular gene (or low but non-zero activity of the encoded protein) listed in Table 4 is treated with an agent that inhibits expression of that gene or that inhibits activity of a protein encoded by the gene. For example, a tumor with low expression of ENO1 may be treated with an ENO1 inhibitor; a tumor with low expression of GAPDH may be treated with a GAPDH inhibitor; a tumor with low expression of GPI may be treated with a GPI inhibitor, etc. In some embodiments an agent identified according to the methods is useful to treat a tumor in combination with an OXPHOS inhibitor. In some embodiments the agent increases sensitivity of a tumor to glucose limitation. In some embodiments an agent identified according to the methods is useful to treat a tumor in combination with a biguanide. In some embodiments the agent increases sensitivity of a tumor to biguanides.

An agent to be assessed or that is being assessed or has been assessed, e.g., with regard to its effect on gene expression, cell survival or proliferation or any other parameter of interest, may be referred to as a “test agent”. Any of a wide variety of agents may be used as test agents in various embodiments. For example, a test agent may be a small molecule, polypeptide, peptide, nucleic acid, oligonucleotide, lipid, carbohydrate, or hybrid molecule. Nucleic acids may be RNAi agents, e.g., siRNA or shRNA, or may be antisense oligonucleotides or may be cDNAs or portions thereof or other nucleic acids that can be expressed in cells, optionally encoding proteins. Agents can be obtained from natural sources or produced synthetically. Agents may be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. A library may comprise, e.g., between 100 and 500,000 compounds, or more. In some embodiments compounds are arrayed in multiwell plates. They may be dissolved in a solvent (e.g., DMSO) or provided in dry form, e.g., as a powder or solid. Collections of synthetic, semi-synthetic, and/or naturally occurring compounds may be tested. Compound libraries can comprise structurally related, structurally diverse, or structurally unrelated compounds. Compounds may be artificial (having a structure invented by man and not found in nature) or naturally occurring. In some embodiments a library comprises at least some compounds that have been identified as “hits” or “leads” in a drug discovery program and/or analogs thereof. A compound library may comprise natural products and/or compounds generated using non-directed or directed synthetic organic chemistry. A compound library may be a small molecule library. Other libraries of interest include peptide or peptoid libraries, cDNA libraries, oligonucleotide libraries, and RNAi libraries. A library may be focused (e.g., composed primarily of compounds having the same core structure, derived from the same precursor, or having at least one biochemical activity in common). Compound libraries are available from a number of commercial vendors such as Tocris BioScience, Nanosyn, BioFocus, and from government entities such as the U.S. National Institutes of Health (NIH). In some embodiments, a test agent which is an “approved human drug” may be tested. An “approved human drug” is an agent that has been approved for use in treating humans by a government regulatory agency such as the US Food and Drug Administration, European Medicines Evaluation Agency, or a similar agency responsible for evaluating at least the safety of therapeutic agents prior to allowing them to be marketed. A test agent may be, e.g., an antineoplastic, antibacterial, antiviral, antifungal, antiprotozoal, antiparasitic, antidepressant, antipsychotic, anesthetic, antianginal, antihypertensive, antiarrhythmic, antiinflammatory, analgesic, antithrombotic, antiemetic, immunomodulator, antidiabetic, lipid- or cholesterol-lowering (e.g., statin), anticonvulsant, anticoagulant, antianxiety, hypnotic (sleep-inducing), hormonal, or anti-hormonal drug, etc. Examples of approved drugs are found in, e.g., Goodman and Gilman's The Pharmacological Basis of Therapeutics, and/or Katzung, B., cited above. In some embodiments a test agent is a known anti-cancer agent. In some embodiments a test agent is not a known anti-cancer agent. In some embodiments a test agent is not an agent that is known to be present in detectable amounts in an ordinary cell culture medium, e.g., a cell culture medium ordinarily used for culturing tumor cells. In some embodiments, if a cell culture medium ingredient is used as a test agent, it is used at a concentration at least 5 times higher than that in which it is found in such ordinary cell culture medium.

An appropriate assay for an inhibitor of activity may be selected depending on the particular gene product of interest. In some embodiments the gene product has an enzymatic activity. An assay may comprise contacting the gene product with a substrate in the presence of a candidate agent and determining whether the candidate agent inhibits conversion of the substrate to a product. In some embodiments the substrate is detectably labeled. In some embodiments a method comprises identifying an agent that inhibits translocation of a glucose transporter, e.g., GLUT3, to the cell membrane. In some embodiments an assay described in US Pat. Pub. No. 20020052012 may be used, wherein the GLUT is GLUT3. In some embodiments a method of testing the ability of an agent to inhibit the survival and/or proliferation of a tumor cell comprises: (a) contacting one or more test cells with an agent that inhibits expression or activity of a gene product listed in Table 1 or SLC2A3 or another gene listed in Table 4; and (b) assessing the level of inhibition of the survival and/or proliferation of the one or more test cells by the agent. In some embodiments the method comprises (c) identifying the agent as a candidate anti-cancer agent if the test agent inhibits the survival and/or proliferation of the one or more test cells by the agent. In some embodiments the method comprises (c) comparing the level of inhibition of the survival and/or proliferation of the one or more test cells by the agent with the level of inhibition of the survival and/or proliferation of control cells not contacted with the agent and (d) identifying the agent as a candidate anti-cancer agent if the test agent inhibits the survival and/or proliferation of the one or more test cells by the agent as compared with survival and/or proliferation of the control cells. In some embodiments the test cells are cancer cells. In some embodiments the test cells are cancer cells that are sensitive to glucose limitation.

In some embodiments test cells and control cells are genetically matched, e.g., in that they originate from a single individual, cell or tissue sample, cell line, or cell, or from genetically identical (isogenic) or essentially genetically identical individuals (e.g., monozygotic twins, animals from an inbred strain), cell or tissue samples, cell lines, or cells. The term “essentially” is used in this context to encompass the possibility that cells may not be genetically identical even if they originate from a single cell, sample, or individual. For example, cells may acquire mutations in culture or in vivo and thus the genomic sequence of two cells derived from a single cell or individual may differ at one or more positions. In some embodiments, test cells and/or control cells are derived from isogenic or essentially isogenic and have undergone no more than 2, 3, 5, 10, 15, 20, 25, 30, 35, 40, 50, 60, 70, 80, 90, or 100 population doublings or passages following isolation as individual cell lines or cell populations before being used in a screen or assay to identify candidate anti-tumor agents.

In some embodiments test cells and/or control cells are genetically modified to cause them to express a gene at increased or decreased levels. Pairs of such test cells and control cells may be useful to identify or characterize an agent that binds to, acts on, or affects expression or activity of a gene product of such gene. Methods of producing genetically modified cells are well known in the art. For example in some embodiments test cells are generated from an initial cell population by introduction of a vector comprising a sequence that encodes a protein of interest, e.g., a glucose transporter, e.g., SLC2A3, so that the resulting cells express increased levels of the transporter as compared with cells that have not been so manipulated. One of skill in the art would know that due to the degeneracy of the genetic code, numerous different nucleic acid sequences would encode a desired polypeptide. In some embodiments test cells are caused to have reduced expression of a gene that encodes a protein of interest, e.g, a glucose transporter, by contacting them with an RNAi agent. In some embodiments cells are contacted with exogenous siRNA. In some embodiments a vector that comprises a template for transcription of a short hairpin RNA or antisense RNA targeted to a gene or transcript is introduced into cells, such that the resulting cells express an shRNA or antisense RNA that inhibits expression of the gene. A nucleic acid construct or vector may be introduced into cells by transfection, infection, or other methods known in the art. Cells may be contacted with an appropriate reagent (e.g., a transfection reagent) to promote uptake of a nucleic acid or vector by the cells. In some embodiments a genetic modification is stable such that it is inherited by descendants of the cell into which a vector or nucleic acid construct was introduced. A stable genetic modification usually comprises alteration of a cell's genomic DNA, such as integration of exogenous nucleic acid into the genome or deletion of genomic DNA. A nucleic acid construct or vector may comprise a selectable marker that facilitates identification and/or isolation of genetically modified cells and, if desired, establishment of a stable cell line. It will be understood that the term “genetically modified” refers to an original genetically modified cell or cell population and descendants thereof. Thus a genetically modified cell used in methods described herein may be a descendant of an original genetically modified cell.

In various embodiments the number of test agents is at least 10; 100; 1000; 10,000; 100,000; 250,000; 500,000 or more. In some embodiments test agents are tested in individual vessels, e.g., individual wells of a multiwell plate (sometimes referred to as microwell or microtiter plate or dish). In some embodiments a multiwell plate of use in performing an assay or culturing or testing cells or agents has 6, 12, 24, 96, 384, or 1536 wells. Cells can be contacted with one or more test agents for varying periods of time and/or at different concentrations. In certain embodiments cells are contacted with test agent(s) for between 1 hour and 20 days, e.g., for between 12 and 48 hours, between 48 hours and 5 days, e.g., about 3 days, between 5 days and 10 days, between 10 days and 20 days, or any intervening range or particular value. Cells can be contacted with a test agent during all or part of a culture period. Test agents can be added to culture media at the time of replenishing the media and/or between media changes. In some embodiments a compound is tested at 2, 3, 5, or more concentrations. Concentrations may range, for example, between about 10 nM and about 500 μM. For example, concentrations of about 100 nM, 1 μM, 10 μM, 100 μM, and 200 μM may be used.

In some embodiments, a high throughput screen (HTS) is performed. A high throughput screen can utilize cell-free or cell-based assays. High throughput screens often involve testing large numbers of compounds with high efficiency, e.g., in parallel. For example, tens or hundreds of thousands of compounds can be routinely screened in short periods of time, e.g., hours to days. Often such screening is performed in multiwell plates containing, at least 96 wells or other vessels in which multiple physically separated cavities or depressions are present in a substrate. High throughput screens often involve use of automation, e.g., for liquid handling, imaging, data acquisition and processing, etc. Certain general principles and techniques that may be applied in embodiments of a HTS of the present invention are described in Macarrón R & Hertzberg RP. Design and implementation of high-throughput screening assays. Methods Mol Biol., 565:1-32, 2009 and/or An WF & Tolliday NJ., Introduction: cell-based assays for high-throughput screening. Methods Mol Biol. 486:1-12, 2009, and/or references in either of these. Useful methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hν\ddot{ν}ser.

The term “hit” generally refers to an agent that achieves an effect of interest in a screen or assay, e.g., an agent that has at least a predetermined level of inhibitory effect on gene expression, protein activity, cell survival, proliferation, or other parameter of interest being measured in the screen or assay. Test agents that are identified as hits in a screen may be selected for further testing, development, or modification. In some embodiments a test agent is retested using the same assay or different assays. For example, a candidate anti-tumor agent may be tested against multiple different tumor cell lines or in an in vivo tumor model to determine its effect on tumor cell survival or proliferation, tumor growth, etc. Additional amounts of the test agent may be synthesized or otherwise obtained, if desired. Physical testing or computational approaches can be used to determine or predict one or more physicochemical, pharmacokinetic and/or pharmacodynamic properties of compounds identified in a screen. For example, solubility, absorption, distribution, metabolism, and excretion (ADME) parameters can be experimentally determined or predicted. Such information can be used, e.g., to select hits for further testing, development, or modification. For example, small molecules having characteristics typical of “drug-like” molecules can be selected and/or small molecules having one or more unfavorable characteristics can be avoided or modified to reduce or eliminated such unfavorable characteristic(s).

Additional compounds, e.g., analogs, that have a desired activity can be identified or designed based on compounds identified in a screen. In some embodiments structures of hit compounds are examined to identify a pharmacophore, which can be used to design additional compounds. An additional compound may, for example, have one or more altered, e.g., improved, physicochemical, pharmacokinetic (e.g., absorption, distribution, metabolism and/or excretion) and/or pharmacodynamic properties as compared with an initial hit or may have approximately the same properties but a different structure. For example, a compound may have higher affinity for the molecular target of interest, lower affinity for a nontarget molecule, greater solubility (e.g., increased aqueous solubility), increased stability, increased bioavailability, oral bioavailability, and/or reduced side effect(s), modified onset of therapeutic action and/or duration of effect. An improved property is generally a property that renders a compound more readily usable or more useful for one or more intended uses. Improvement can be accomplished through empirical modification of the hit structure (e.g., synthesizing compounds with related structures and testing them in cell-free or cell-based assays or in non-human animals) and/or using computational approaches. Such modification can make use of established principles of medicinal chemistry to predictably alter one or more properties.

In certain embodiments an agent identified or tested using a method described herein displays selective activity (e.g., inhibition of survival or proliferation, or other manifestation of toxicity) against test cells that are sensitive to glucose limitation, relative to its activity against control cells that are not sensitive to glucose limitation. For example, the IC₅₀ and/or IC₉₀ of an agent may be between about 2-fold and about 1000-fold lower, e.g., about 2, 5, 10, 20, 50, 100, 250, 500, or 1000-fold lower, for test cells versus control cells.

Data or results from testing an agent or performing a screen may be stored or electronically transmitted. Such information may be stored on a tangible medium, which may be a computer-readable medium, paper, etc. In some embodiments a method of identifying or testing an agent comprises storing and/or electronically transmitting information indicating that a test agent has one or more propert(ies) of interest or indicating that a test agent is a “hit” in a particular screen, or indicating the particular result achieved using a test agent. A list of hits from a screen may be generated and stored or transmitted. Hits may be ranked or divided into two or more groups based on activity, structural similarity, or other characteristics

Once a candidate anti-tumor agent is identified, additional agents, e.g., analogs, may be generated based on it, and may be tested for anti-tumor effect or other properties. An additional agent, may, for example, have increased cancer cell uptake, increased potency, increased stability, greater solubility, or any improved property. In some embodiments a labeled form of the agent is generated. The labeled agent may be used, e.g., to directly measure binding of an agent to its target.

In some embodiments various methods described in the present disclosure comprise measuring one or more characteristics of a cell or tumor such as cell survival or proliferation, glycolytic activity, expression level of one or more genes, activity of one or more gene products, or tumor size or growth rate. In some embodiments one or more cells, biological samples, or tumors are contacted with an agent or combination of agents and one or more characteristics such as cell survival or proliferation, glycolytic activity, expression level of one or more genes, activity of one or more gene products, or tumor size or growth rate is measured.

In some embodiments cells are maintained and/or contacted with one or more agents in vitro (in culture). Cultured cells can be maintained in a suitable cell culture vessel under appropriate conditions (e.g., appropriate temperature, gas composition, pressure, humidity) and in appropriate culture medium. Methods, culture media, and cell culture vessels (e.g., plates (dishes), wells, flasks, bottles, tubes, or other chambers) suitable for culturing cells are known to those of ordinary skill in the art. Typically the vessels contain a suitable tissue culture medium, and the test agent(s) are present in the tissue culture medium, e.g., test agent(s) are added to the culture medium before or after the medium is placed in the culture vessels. One of ordinary skill in the art can select a medium appropriate for culturing a particular cell type. In some embodiments a medium is a chemically defined medium. In some embodiments a medium is free or essentially free of serum or tissue extracts. In some embodiments serum or tissue extract is present. In some embodiments cells are non-adherent.

In some embodiments cells are adherent. Such cells may, for example, be cultured on a plastic or glass surface, which may in some embodiments be processed to render it suitable for mammalian cell culture. In some embodiments cells are cultured on or in a material comprising collagen, laminin, Matrigel®, or a synthetic polymer or other material that is intended to provide an environment that resembles in at least some respects the extracellular environment, e.g., extracellular matrix, found in certain tissues in vivo.

In some embodiments mammalian cells are used. In some embodiments mammalian cells are primate cells (human cells or non-human primate cells), rodent (e.g., mouse, rat, rabbit, hamster) cells, canine, feline, bovine, or other mammalian cells. In some embodiments avian cells are used. A cell may be a primary cell, immortalized cell, normal cell, abnormal cell, tumor cell, non-tumor cell, etc., in various embodiments. A cell may originate from a particular tissue or organ of interest or may be of a particular cell type. Primary cells may be freshly isolated from a subject or may have been passaged in culture a limited number of times, e.g., between 1-5 times or undergone a small number of population doublings in culture, e.g., 1-5 population doublings. In some embodiments a cell is a member of a population of cells, e.g., a member of a non-immortalized or immortalized cell line. In some embodiments, a “cell line” refers to a population of cells that has been maintained in culture for at least 10 passages or at least 10 population doublings. In some embodiments, a cell line is derived from a single cell. In some embodiments, a cell line is derived from multiple cells. In some embodiments, cells of a cell line are descended from a cell or cells originating from a single sample (e.g., a sample obtained from a tumor) or individual. A cell may be a member of a cell line that is capable of prolonged proliferation in culture, e.g., for longer than about 3 months (with passaging as appropriate) or longer than about 25 population doublings). A non-immortalized cell line may, for example, be capable of undergoing between about 20-80 population doublings in culture before senescence. In some embodiments, a cell line is capable of indefinite proliferation in culture (immortalized). An immortalized cell line has acquired an essentially unlimited life span, i.e., the cell line appears to be capable of proliferating essentially indefinitely. For purposes hereof, a cell line that has undergone or is capable of undergoing at least 100 population doublings in culture may be considered immortal. In some embodiments, cells are maintained in culture and may be passaged or allowed to double once or more following their isolation from a subject (e.g., between 2-5, 5-10, 10-20, 20-50, 50-100 times, or more) prior to use in a method disclosed herein. In some embodiments, cells have been passaged or permitted to double no more than 1, 2, 5, 10, 20, or 50 times following isolation from a subject prior to use in a method described herein. If desired, cells may be tested to confirm whether they are derived from a single individual or a particular cell line by any of a variety of methods known in the art such as DNA fingerprinting (e.g., short tandem repeat (STR) analysis) or single nucleotide polymorphism (SNP) analysis (which may be performed using, e.g., SNP arrays (e.g., SNP chips) or sequencing).

Numerous tumor cell lines and non-tumor cell lines are known in the art and may be used in various methods described herein. Cell lines can be generated using methods known in the art or obtained, e.g., from depositories or cell banks such as the American Type Culture Collection (ATCC), Coriell Cell Repositories, Deutsche Sammlung von Mikroorganismen und Zellkulturen (German Collection of Microorganisms and Cell Cultures; DSMZ), European Collection of Cell Cultures (ECACC), Japanese Collection of Research Bioresources (JCRB), RIKEN, Cell Bank Australia, etc. The paper and online catalogs of the afore-mentioned depositories and cell banks are incorporated herein by reference. Cells or cell lines may be of any cell type or tissue of origin in various embodiments. Tumor cells or tumor cell lines may be of any tumor type or tissue of origin in various embodiments. In some embodiments tumor cells, e.g., a tumor cell line, originates from a human tumor. In some embodiments tumor cells, e.g., a tumor cell line, originates from a tumor of a non-human animal. In some embodiments tumor cells originate from a naturally arising tumor (i.e., a tumor that was not intentionally induced or generated for, e.g., experimental purposes). In some embodiments a tumor cell line originates from a primary tumor. In some embodiments a tumor cell line originates from a metastatic tumor. In some embodiments a tumor cell line originates from a metastasis. In some embodiments a cell line has become spontaneously immortalized in cell culture. In some embodiments a tumor cell line is capable of giving rise to tumors when introduced into an immunocompromised host, e.g., an immunocompromised rodent such as an immunocompromised mouse.

In some embodiments tumor cells are experimentally produced tumor cells. Tumor cells can be produced by genetically modifying a non-tumor cell, e.g., a non-tumor somatic cell, e.g., by expressing or activating an oncogene in the non-tumor cell and/or inactivating or inhibiting expression of one or more tumor suppressor genes (TSG) or inhibiting activity of a gene product of a TSG. Certain experimentally produced tumor cells and exemplary methods of producing tumor cells are described in PCT/US2000/015008 (WO/2000/073420), in U.S. Ser. No. 10/767,018, in Elenbaas, et al., Genes and Development, 15(1):50-65, (2001); and/or Yang, J, et al, Cell 117, 927-939 (2004). In certain embodiments a non-immortal cell, e.g., a non-tumor cell, is immortalized by causing the cell to express telomerase catalytic subunit (e.g., human telomerase catalytic subunit; hTERT). In some embodiments a tumor cell is produced from a non-tumor cell by introducing one or more expression construct(s) or expression vector(s) comprising an oncogene into the cell or modifying an endogenous gene (proto-oncogene) by a targeted insertion into or near the gene or by deletion or replacement of a portion of the gene. For example, cells, e.g., non-tumor cells, can be immortalized with hTERT and transformed by expression of SV40 large T oncoprotein and oncogenic HRAS (e.g., H-rαsV12). In some embodiments a TSG is knocked out or functionally inactivated using gene targeting. For example, a portion of a TSG may be deleted or the TSG may be disrupted by an insertion. In some embodiments a TSG is inhibited by introducing into a cell one or more expression construct(s) or expression vector(s) encoding an inhibitory molecule (e.g., an RNAi agent such as a shRNA or a dominant negative or a negative regulator) that is capable of inhibiting the expression or activity of an expression product of a TSG. Oncogenes and/or TSG inhibitory molecules may be expressed under control of suitable regulatory elements, which may be constitutive or regulatable (e.g., inducible). In some embodiments tumor cells may be produced by expressing or activating multiple oncogenes and/or inhibiting or inactivating multiple TSGs, e.g., 1, 2, 3, 4, or more oncogenes and/or 1, 2, 3, 4, or more TSGs. Many combinations of oncogenes and/or TSGs whose expression/activation or inhibition/inactivation, respectively, can be used to induce tumors are known in the art. Suitable vectors and methods useful for producing genetically engineered tumor cells will be apparent to those of ordinary skill in the art.

The term “oncogene” encompasses nucleic acids that, when expressed, can increase the likelihood of or contribute to cancer initiation or progression. Normal cellular sequences (“proto-oncogenes”) can be activated to become oncogenes (sometimes termed “activated oncogenes”) by mutation and/or aberrant expression. In various embodiments an oncogene can comprise a complete coding sequence for a gene product or a portion that maintains at least in part the oncogenic potential of the complete sequence or a sequence that encodes a fusion protein. Oncogenic mutations can result, e.g., in altered (e.g., increased) protein activity, loss of proper regulation, or an alteration (e.g., an increase) in RNA or protein level. Aberrant expression may occur, e.g., due to chromosomal rearrangement resulting in juxtaposition to regulatory elements such as enhancers, epigenetic mechanisms, or due to amplification, and may result in an increased amount of proto-oncogene product or production in an inappropriate cell type. As known in the art, proto-oncogenes often encode proteins that control or participate in cell proliferation, differentiation, and/or apoptosis. These proteins include, e.g., various transcription factors, chromatin remodelers, growth factors, growth factor receptors, signal transducers, and apoptosis regulators. Oncogenes also include a variety of viral proteins, e.g., from viruses such as polyomaviruses (e.g., SV40 large T antigen) and papillomaviruses (e.g., human papilloma virus E6 and E7). A TSG may be any gene wherein a loss or reduction in function of an expression product of the gene can increase the likelihood of or contribute to cancer initiation or progression. Loss or reduction in function can occur, e.g., due to mutation or epigenetic mechanisms. Many TSGs encode proteins that normally function to restrain or negatively regulate cell proliferation and/or to promote apoptosis. In some embodiments an oncogene or TSG encodes a miRNA. Exemplary oncogenes include, e.g., MYC, SRC, FOS, JUN, MYB, RAS, RAF, ABL, ALK, AKT, TRK, BCL2, WNT, HER2/NEU, EGFR, MAPK, ERK, MDM2, CDK4, GLI1, GLI2, IGF2, TP53, etc. Exemplary TSGs include, e.g., RB, TP53, APC, NF1, BRCA1, BRCA2, PTEN, CDK inhibitory proteins (e.g., p16, p21), PTCH, WT1, etc. It will be understood that a number of these oncogene and TSG names encompass multiple family members and that many other TSGs are known.

Cells, e.g., tumor cells, may be maintained in a culture medium comprising an agent of interest. The effect of the agent on tumor cell viability, proliferation, tumor-initiating capacity, OXPHOS activity, or any other tumor cell property may be measured using any suitable method known in the art in various embodiments. In certain embodiments survival and/or proliferation of a cell or cell population may be determined by a cell counting assay (e.g., using visual inspection, automated image analysis, flow cytometer, etc.), a replication assay, a cell membrane integrity assay, a cellular ATP-based assay, a mitochondrial reductase activity assay, a BrdU, EdU, or H3-Thymidine incorporation assay, calcein staining, a DNA content assay using a nucleic acid dye, such as Hoechst Dye, DAPI, Actinomycin D, 7-aminoactinomycin D or propidium iodide, a cellular metabolism assay such as resazurin (sometimes known as AlamarBlue or by various other names), MTT, XTT, and CellTitre Glo, etc., a protein content assay such as SRB (sulforhodamine B) assay; nuclear fragmentation assays; cytoplasmic histone associated DNA fragmentation assay; PARP cleavage assay; TUNEL staining; or annexin staining. In some embodiments an assay may reflect two or more characteristics. For example, the CyQUANT® family of cell proliferation assays (Life Technologies) are based on both DNA content and membrane integrity. In some embodiments cell survival or proliferation is assessed by measuring expression of one or more genes that encode gene products that mediate cell survival or proliferation or cell death, e.g., genes that encode products that play roles in or regulate the cell cycle or cell death (e.g., apoptosis). Examples of such genes include, e.g., cyclin dependent kinases, cyclins, BAX/BCL2 family members, caspases, etc. One of ordinary skill in the art will be able to select appropriate genes to be used as indicators of cell survival or proliferation. It will be understood that in some embodiments an assay of cell survival and/or proliferation may determine cell number, e.g., number of living cells, and may not distinguish specifically between cell survival per se and cell proliferation, e.g., the assay result may reflect a combination of survival and proliferation. In some embodiments an assay able to specifically assess survival or proliferation or cell death (e.g., apoptosis or necrosis) may be used.

In some embodiments an agent or combination of agents is tested to determine whether it has an anti-tumor effect or to quantify an anti-tumor effect. In some embodiments an anti-tumor effect is inhibition of tumor cell survival or proliferation. It will be understood that inhibition of cell proliferation or survival by an agent or combination of agents may, or may not, be complete. For example, cell proliferation may, or may not, be decreased to a state of complete arrest for an effect to be considered one of inhibition or reduction of cell proliferation. In some embodiments, “inhibition” may comprise inhibiting proliferation of a cell that is in a non-proliferating state (e.g., a cell that is in the GO state, also referred to as “quiescent”) and/or inhibiting proliferation of a proliferating cell (e.g., a cell that is not quiescent). Similarly, inhibition of cell survival may refer to killing of a cell, or cells, such as by causing or contributing to necrosis or apoptosis, and/or the process of rendering a cell susceptible to death, e.g., causing or increasing the propensity of a cell to undergo apoptosis or necrosis. The inhibition may be at least about 10%, 15%, 20%, 25%, 30%, 35%, 40%, 45%, 50%, 55%, 60%, 65%, 70%, 75%, 80%, 85%, 90%, 95%, 99%, or 100% of a reference level (e.g., a control level). In some embodiments an anti-tumor effect is inhibition of the capacity of tumor cells to form colonies in suspension culture. In some embodiments an anti-tumor effect is inhibition of capacity of the one or more tumor cells to form colonies in a semi-solid medium such as soft agar or methylcellulose. In some embodiments an anti-tumor effect is inhibition of capacity of the one or more tumor cells to form tumor spheres in culture. In some embodiments an anti-tumor effect is inhibition of the capacity of the one or more tumor cells to form tumors in vivo.

In some embodiments sensitivity of a tumor cell, tumor cell line, or tumor to an agent or combination of agents, is assessed using an in vivo tumor model. An “in vivo” tumor model involves the use of one or more living non-human animals (“test animals”). For example, an in vivo tumor model may involve administration of an agent and/or introduction of tumor cells to one or more test animals. In some embodiments a test animal is a mouse, rat, or dog. Numerous in vivo tumor models are known in the art. By way of example, certain in vivo tumor models are described in U.S. Pat. No. 4,736,866; U.S. Ser. No. 10/990,993; PCT/US2004/028098 (WO/2005/020683); and/or PCT/US2008/085040 (WO/2009/070767). Introduction of one or more cells into a subject (e.g., by injection or implantation) may be referred to as “grafting”, and the introduced cell(s) may be referred to as a “graft”. In general, any tumor cells may be used in an in vivo tumor model in various embodiments. Tumor cells may be from a tumor cell line or tumor sample. In some embodiments tumor cells originate from a naturally arising tumor (i.e., a tumor that was not intentionally induced or generated for, e.g., experimental purposes). In some embodiments experimentally produced tumor cells may be used. The number of tumor cells introduced may range, e.g., from 1 to about 10, 10², 10³, 10⁴, 10⁵, 10⁶, 10⁷,10⁸, 10⁹, or more. In some embodiments the tumor cells are of the same species or inbred strain as the test animal. In some embodiments tumor cells may originate from the test animal. In some embodiments the tumor cells are of a different species than the test animal. For example, the tumor cells may be human cells. In some embodiments, a test animal is immunocompromised, e.g., in certain embodiments in which the tumor cells are from a different species to the test animal or originate from an immunologically incompatible strain of the same species as the test animal. For example, a test animal may be selected or genetically engineered to have a functionally deficient immune system or may subjected to radiation or an immunosuppressive agent or surgery such as removal of the thymus) so as to reduce immune system function. In some embodiments, a test animal is a SCID mouse, NOD mouse, NOD/SCID mouse, nude mouse, and/or Rag1 and/or Rag2 knockout mouse, or a rat having similar immune system dysfunction. Tumor cells may be introduced at an orthotopic or non-orthotopic location. In some embodiments tumor cells are introduced subcutaneously, under the renal capsule, or into the bloodstream. Non-tumor cells (e.g., fibroblasts, bone marrow derived cells), an extracellular matrix component or hydrogel (e.g., collagen or Matrigel®), or an agent that promotes tumor development or growth may be administered to the test animal prior to, together with, or separately from the tumor cells.

In some embodiments tumor cells are contacted with an agent prior to grafting (in vitro) and/or following grafting (by administering the agent to the test animal). The agent may be administered to the test animal at around the same time as the tumor cells, and/or at one or more subsequent times. The number, size, growth rate, metastasis, or other properties of resulting tumors (if any) may be assessed at one or more time points following grafting and, if desired, may be compared with a control in which tumor cells of the same type are grafted without contacting them with the agent or using a higher or lower concentration or dose of the agent.

In some embodiments a tumor arises due to neoplastic transformation that occurs in vivo, e.g., at least in part as a result of one or more mutations in a cell in a subject. In some embodiments a test animal is a tumor-prone animal. The test animal may, for example, be of a species or strain that naturally has a predisposition to develop tumors and/or may be a genetically modified tumor-prone animal. For example, in some embodiments the animal is a genetically engineered animal at least some of whose cells comprise, as a result of genetic modification, at least one activated oncogene and/or in which at least one tumor suppressor gene has been functionally inactivated. Standard methods of generating genetically modified animals, e.g., transgenic animals that comprises exogenous genes or animals that have an alteration to an endogenous gene, e.g., an insertion or an at least partial deletion or replacement (sometimes referred to as “knockout” or “knock-in” animal) can be used.

Any of a wide variety of methods and/or devices known in the art may be used to assess tumors in vivo. Tumor number, size, growth rate, or metastasis may, for example, be assessed using various imaging modalities, e.g., 1, 2, or 3-dimensional imaging (e.g., using X-ray, CT scan, ultrasound, or magnetic resonance imaging, etc.) and/or functional imaging (e.g., PET scan) may be used to detect or assess lesions (local or metastatic), e.g., to measure anatomical tumor burden, detect new lesions (e.g., metastases), etc. In some embodiments PET scanning with the glucose analog fluorine-18 (F-18) fluorodeoxyglucose (FDG) as a tracer is used. As known in the art, FDG is taken up and phosphorylated by glucose-using cells. FDG remains trapped in cells that take it up until it decays, which results in intense radiolabeling of tissues with high glucose uptake, such as the brain, the liver, and certain cancers. In some embodiments tumor(s) may be removed from the body (e.g., at necropsy) and assessed (e.g., tumors may be counted, weighed, and/or size (e.g., dimensions) measured). In some embodiments the size and/or number of tumors may be determined non-invasively. For example, in certain tumor models, tumor cells that are fluorescently labeled (e.g., by expressing a fluorescent protein such as GFP) can be monitored by various tumor-imaging techniques or instruments, e.g., non-invasive fluorescence methods such as two-photon microscopy. The size of a tumor implanted or developing subcutaneously can be monitored and measured underneath the skin. In certain embodiments a tumor is considered sensitive to an agent if the growth rate or size (e.g., estimated volume or weight) of the tumor is reduced by at least 50%, 60%, 70%, 80%, 90%, 95%, or more, by treatment at a dose (or series of doses) that are tolerated by a subject. In certain embodiments a tumor is rendered undetectable. In some embodiments recurrence is prevented for at least a period of time. In some embodiments a reduction in tumor growth rate or size or prevention of recurrence is maintained at least while treatment is continued. In some embodiments such reduction or prevention of recurrence is maintained for at least about 3, 4, 6, 8, 12, 16, 24, 36, 44, 52 weeks, or more, e.g., at least about 15, 18, 24 months, 3-5 years, or more. In some embodiments sufficient tumor cells may be eradicated so that the tumor does not recur after cessation of treatment when assessed at least about 3, 4, 6, 8, 12, 16, 24, 36, 44, 52 weeks, or more, e.g., at least about 15, 18, 24 months, 3-5 years, or more, after cessation of treatment.

In some embodiments, treatment sensitivity of a tumor in a human subject may be evaluated at least in part using objective criteria such as the original or revised Response Evaluation Criteria In Solid Tumors (RECIST), a guideline that can be used to objectively determine when or whether cancer patients improve (“respond”), remain about the same (“stable disease”), or worsen (“progressive disease”) based on anatomical tumor burden (e.g., measured using physical examination and/or imaging techniques such as those mentioned above). A response may be either a “complete response” or a “partial response”. The original RECIST guideline is described in Therasse P, et al. J Natl Cancer Inst (2000) 92:205-16. A revised RECIST guideline (Version 1.1) is described in Eisenhauer, E., et al., Eur J Cancer. (2009) 45(2):228-47). In the case of brain tumors, response assessment (e.g., in high-grade gliomas such as glioblastoma) can use the Macdonald criteria (Macdonald D, et al. (1990) Response criteria for phase II studies of supratentorial malignant glioma. J Clin Oncol 8:1277-1280), e.g., as extrapolated to magnetic resonance imaging (MRI) (Rees J (2003) Advances in magnetic resonance imaging of brain tumours. Curr Opin Neurol 16:643-650). An updated version of the Macdonald criteria may be used (Wen, P Y, et al., J Clin Oncol. (2010) 28(11): 1963-72). In the case of lymphomas or leukemias, response criteria known in the art can be used (see, e.g., Cheson B D, et al. Revised response criteria for malignant lymphoma. J Clin Oncol 2007; 10:579-86). It will be appreciated that the guidelines and criteria mentioned herein for assessing tumor sensitivity are merely exemplary. Modified or updated versions thereof or other reasonable criteria (e.g., as determined by a person of ordinary skill in the art) may be used. Clinical assessment of symptoms or signs associated with tumor presence, stage, regression, progression, or recurrence may be used. In certain embodiments criteria based on anatomic tumor burden should reasonably correlate with a clinically meaningful benefit such as increased survival (e.g., increased progression-free survival, increased cancer-specific survival, or increased overall survival) or at least improved quality of life such as reduction in one or more symptoms. In some embodiments a response lasts for at least 2, 3, 4, 5, 6, 8, 12 months, or more. In some embodiments tumor response or recurrence may be assessed at least in part by testing a sample comprising a body fluid such as blood for the presence of tumor cells and/or for the presence or level or change in level of one or more substances (e.g., microRNA, protein) produced or secreted by tumor cells. For example, prostate specific antigen (PSA) and carcinoembryonic antigen (CEA) are two such markers. The extracellular domain of HER2 can be shed from the surface of tumor cells and enter the circulation. A normal level or a reduction in level over time of one or more substances derived from tumor cells may indicate a response or maintenance of remission. An abnormally high level or an increase in level over time may indicate progression or recurrence.

In some embodiments, treatment sensitivity of a tumor in a subject, e.g., a human subject, is assessed by evaluating survival, e.g., 3 month or 6 month survival, or 1, 2, 5, or 10 year survival. In some embodiments, overall survival is assessed. In some embodiments disease-specific survival (i.e., survival considering only mortality due to cancer) is assessed. In some embodiments, progression-free survival is assessed. In some embodiments, a tumor is considered sensitive to a compound if treatment with the compound results in an increased survival relative to predicted survival in the absence of treatment. In some embodiments, a tumor is considered sensitive to a compound if adding the compound to a cancer treatment regimen results in an increased survival relative to predicted survival using the same cancer regimen but without the compound. In some embodiments, a tumor is considered sensitive to a an agent if using the agent in place of a different agent in a standard or experimental cancer treatment regimen results in an increased response, e.g., increased survival, relative to predicted survival using the standard or experimental cancer treatment regimen.

In some embodiments, a difference between two or more measurements or between two or more groups of samples or subjects is statistically significant as determined using an appropriate statistical test or analytical method. One of ordinary skill in the art will be able to select an appropriate statistical test or analytical method for evaluating statistical significance.

In some embodiments, a difference between two or more measurements or between two or more groups of subjects would be considered clinically meaningful or clinically significant by one of ordinary skill in the art. In some embodiments statistically significant refers to a P-value of less than 0.05, e.g., less than 0.025, e.g., less than 0.01, e.g., less than 0.005. In some embodiments a P-value is a two-tailed P-value.

In some embodiments of any aspect or embodiment in the present disclosure relating to cells, a population of cells, cell sample, or similar terms, the number of cells is between 10 and 10¹³ cells. In some embodiments the number of cells may be at least about 10, 10⁴, 10⁵, 10⁶, 10⁷, 10⁸, 10⁹, 10¹⁰, 10¹¹, 10¹² cells, or more. In some embodiments, the number of cells is between 10⁵ and 10¹² cells, e.g., at least 10⁶, 10⁷, 10⁸, 10⁹, 10¹⁰, 10¹¹, up to about 10¹²or about 10¹³. In some embodiments a screen is performed using multiple populations of cells and/or is repeated multiple times. In some embodiments, the number of cells is between 10⁵ and 10¹² cells, e.g., at least 10⁶, 10⁷, 10⁸, 10⁹, 10¹⁰, 10¹¹, up to about 10¹². In some embodiments smaller numbers of cells are of use, e.g., between 1-10⁴ cells. In some embodiments a population of cells is contained in an individual vessel, e.g., a culture vessel such as a culture plate, flask, or well. In some embodiments a population of cells is contained in multiple vessels. In some embodiments two or more cell populations are pooled to form a larger population.

In some embodiments, one or more compound(s) with a desired IC₅₀ or IC₉₀ is identified. In some embodiments, an IC₅₀ and/or IC₉₀ is no greater than 100 mg/ml, e.g., no greater than 10 mg/ml, e.g., no greater than 1.0 mg/ml, e.g., no greater than 100 μg/ml, e.g., no greater than 10 μg/ml, e.g., no greater than 5 μg/ml or no greater than 1 μg/ml. In some embodiments, an IC₅₀ and/or IC₉₀ is less than or equal to 500 μM. In some embodiments, an IC₅₀ and/or IC₉₀ is less than or equal to 100 μM. In some embodiments, an IC₅₀ and/or is IC₉₀ less than or equal to 10 μM. In some embodiments, an IC₅₀ and/or IC₉₀ is in the nanomolar range, i.e., less than or equal to 1 μM. In some embodiments, an IC₅₀ and/or IC₉₀ between 10 nM and 100 nM, between 100 nM and 500 nM, or between 500 nM and 1 μM. In some embodiments a dose response curve is obtained at one or more time points. For example, cells may be exposed to a range of different concentrations, and cell survival or proliferation may be assessed at one or more time points thereafter. An IC₅₀ and/or IC₉₀ may be obtained from a dose response curve using a regression model, e.g., a nonlinear regression model.

In some embodiments a screen is performed to identify a candidate OXPHOS inhibitor. In some embodiments such a screen comprises identifying an agent that binds to an OXPHOS component. An agent identified as a candidate inhibitor of OXPHOS may be further tested to more directly determine its effect on glycolysis or OXPHOS, e.g., by measuring OCR, ECAR, or a ratio thereof, optionally in the presence of an OXPHOS inhibitor. In some embodiments a candidate modulator of glycolysis is tested to confirm its effect on glycolysis by measuring one or more indicators of glycolysis such as ECAR or OCR ECAR. In some embodiments a candidate OXPHOS modulator is tested to confirm its effect on OXPHOS by measuring one or more indicators of OXPHOS such as OCR. In some embodiments OCR may be measured in the presence and in the absence of an OXPHOS inhibitor to determine the proportion of OCR due to OXPHOS. In some embodiments one or more indicators of glycolysis or OXPHOS is measured using an extracellular flux analyzer such as the XF24 or XF96 Extracellular Flux Analyzer (Seahorse Bioscience, Billerica, Mass.). In some embodiments one or more such measurements is performed in the presence of a known glycolysis inhibitor or a known inhibitor of mitochondrial respiration such as rotenone to specifically identify the contribution of glycolysis or mitochondrial respiration to a measured value, e.g., OCR. In some embodiments cell viability is measured in a parallel experiment with substantially identically processed cells using a method that does not rely on ATP production as an indicator of cell viability. For example, calcein AM staining may be used. In some embodiments the rate of oxygen consumption may be determined using Clark electrodes or the rate of extracellular acidification may be determined using a microphysiometer or by measuring lactate concentration. Lactate concentration may be determined using an assay in which lactate is oxidized by lactate dehydrogenase to generate a product which interacts with a probe to produce a color (e.g., using a kit available from BioVision Inc., Milpitas, Calif., USA or Abcam Inc, Cambridge, Mass., USA) or by monitoring NADH production in a mixture that contains, in addition to lactic dehydrogenase and NAD⁺, hydrazine, and glycine buffer, pH 9.2. Absorbance due to formation of NADH can be detected at 340 nm using a spectrophotometer.

In some embodiments a screen is performed to identify a candidate inhibitor of ENO1, GAPDH, GPI, HK1, PKM, SLC2A1, SLC2A3, TPI1 ALDOA, PFKP, or PGK1. In some embodiments of any aspect herein, cells are cultured or measurement of OCR, ECAR, or cell survival or proliferation or any other parameter of interest is performed under conditions in which oxygen is present at levels equal to or greater than typical physiological levels. In some embodiments of any aspect herein, cells are cultured or measurement of OCR, ECAR, or cell survival or proliferation or any other parameter of interest is performed under conditions in which glucose is limited (e.g., at or below about 1 mM). In some embodiments conditions such as those typically used in mammalian tissue culture, such as in a culture chamber controlled to have a gas composition with about a 5% CO₂ level and an oxygen level approximately that of atmospheric oxygen levels (21%) are used. In some embodiments conditions in which oxygen level is between about 1% and about 2%, about 2% and about 5%, about 5% to about 10%, or about 10% to about 20% are used.

It will be understood that screens or assays to identify or test modulators of a particular polypeptide may make use of variants of the particular polypeptide. For example, functional variants may be used. In some embodiments a functional variant may comprise a heterologous polypeptide portion, such as an epitope tag or fluorescent protein, which may facilitate detection or isolation.

In some embodiments a computer-aided computational approach sometimes referred to as “virtual screening” is used in the identification of candidate inhibitors. Structures of compounds may be screened for ability to bind to a region (e.g., a “pocket”) of a target molecule that is accessible to the compound. The region may be a known or potential active site or any region accessible to the compound, e.g., a concave region on the surface or a cleft or the pore of a transporter. A variety of docking and pharmacophore-based algorithms are known in the art, and computer programs implementing such algorithms are available. Commonly used programs include Gold, Dock, Glide, FlexX, Fred, and LigandFit (including the most recent releases thereof). See, e.g., Ghosh, S., et al., Current Opinion in Chemical Biology, 10(3): 194-2-2, 2006; McInnes C., Current Opinion in Chemical Biology; 11(5): 494-502, 2007, and references in either of the foregoing articles, which are incorporated herein by reference. In some embodiments a virtual screening algorithm may involve two major phases: searching (also called “docking”) and scoring. During the first phase, the program automatically generates a set of candidate complexes of two molecules (test compound and target molecule) and determines the energy of interaction of the candidate complexes. The scoring phase assigns scores to the candidate complexes and selects a structure that displays favorable interactions based at least in part on the energy. To perform virtual screening, this process may be repeated with a large number of test compounds to identify those that, for example, display the most favorable interactions with the target. In some embodiments, low-energy binding modes of a small molecule within an active site or possible active site are identified. Variations may include the use of rigid or flexible docking algorithms and/or including the potential binding of water molecules.

Numerous small molecule structures are available and can be used for virtual screening. A collection of compound structures may sometimes referred to as a “virtual library”. For example, ZINC is a publicly available database containing structures of millions of commercially available compounds that can be used for virtual screening (http://zinc.docking.org/; Shoichet, J. Chem. Inf. Model., 45(1):177-82, 2005). A database containing about 250,000 small molecule structures is available on the National Cancer Institute (U.S.) website (at http://129.43.27.140/ncidb2/). In some embodiments multiple small molecules may be screened, e.g., up to 50,000; 100,000; 250,000; 500,000, or up to 1 million, 2 million, 5 million, 10 million, or more. Compounds can be scored and, optionally, ranked by their potential to bind to a target. Compounds identified in virtual screens can be tested in cell-free or cell-based assays or in animal models to confirm their ability to inhibit activity of a target molecule and/or to assess their effect on survival or proliferation of tumor cells in vitro or in vivo.

Computational approaches can be used to predict one or more physico-chemical, pharmacokinetic and/or pharmacodynamic properties of compounds identified in physical or virtual screens. For example, absorption, distribution, metabolism, and excretion (ADME) parameters can be predicted. Such information can be used, e.g., to select hits for further testing or modification. For example, small molecules having characteristics typical of “drug-like” molecules can be selected and/or small molecules having one or more undesired characteristics can be avoided.

In some embodiments any of the method may comprise testing a candidate agent, in a tumor model. A tumor model may comprise cultured tumor cells or may be an in vivo model. Examples of tumor models are described herein. Ere

In some embodiments, a tumor that is sensitive to glucose limitation is treated with a GLUT inhibitor. As used herein, a “GLUT inhibitor” is an agent that inhibits SLC2A1 or SLC2A3 expression or activity. In some embodiments a GLUT inhibitor selectively inhibits GLUT1, GLUT3, or both, as compared with inhibition of at least one other glucose transporter, preferably as compared with inhibition of multiple other glucose transporters. A selective GLUT inhibitor inhibits its target(s) (e.g., GLUT1 and/or GLUT3) with a lower IC50 than nontarget glucose transporters. In some embodiments a GLUT inhibitor is a small molecule or polypeptide (e.g., an antibody) that binds to the GLUT1 or GLUT3 transporter and blocks the ability of the transporter to transport glucose. Exemplary antibodies that bind to GLUT1 or GLUT3 are described in the Examples. It would be appreciated that a non-human antibody may be used to generate a chimeric or humanized antibody, or a fully human antibody may be used. In some embodiments a GLUT inhibitor is a glucose analog such as 2-deoxyglucose. In some embodiment a GLUT inhibitor is a flavonoid such as phloretin, genestein, or silybin/silibinin.). In some embodiments the GLUT inhibitor is an siRNA that inhibits expression of SLC2A1 or SLC2A3. In some embodiments the tumor is identified as being sensitive to low glucose as described herein, e.g., by assessing mitochondrial DNA for mutations, by measuring expression of one or more genes listed in Table 1 or Table 4. e.g., by assessing expression of genes constituting a gene expression signature indicative of low glucose utilization.

### IV. Combination Therapy

In some embodiments an OXPHOS inhibitor or agent that inhibits expression or activity of a gene product of a gene listed in Table 1 or SCL3A2 or another gene listed in Table 4 is used to treat a subject in need of treatment for a tumor in combination with any one or more additional anti-cancer therapeutic modalities (e.g., chemotherapeutic drugs, surgery, radiotherapy (e.g., γ-radiation, neutron beam radiotherapy, electron beam radiotherapy, proton therapy, brachytherapy, and systemic radioactive isotopes), endocrine therapy, immunotherapy, biologic response modifiers (e.g., interferons, interleukins), hyperthermia (e.g., radiofrequency ablation or other methods of delivering heat such as using lasers, high intensity focused ultrasound or microwaves), cryotherapy, etc.) or combinations thereof, useful for treating a subject in need of treatment for a tumor. In some embodiments a biguanide is used in combination with an agent that inhibits expression of a gene listed in Table 1 or Table 4 or inhibits activity of a gene product encoded by a gene listed in Table 1 or Table 4. In some embodiments a biguanide is used in combination with a GLUT inhibitor.

Agents used in combination may be administered in the same composition or separately in various embodiments. When they are administered separately, two or more agents may be given simultaneously or sequentially (in any order). If administered separately, the time interval between administration of the agents can vary. Agents or non-pharmacological therapies used in combination can be administered or used in any temporal relation to each other such that they produce a beneficial effect in at least some subjects. In some embodiments a beneficial effect produced by a combination is at least as great as, or greater than, that which would be achieved by each therapy individually. In some embodiments, administration of first and second agents is performed such that (i) a dose of the second agent is administered before more than 90% of the most recently administered dose of the first agent has been metabolized to an inactive form or excreted from the body; or (ii) doses of the first and second agents are administered at least once within 8 weeks of each other (e.g., within 1, 2, 4, or 7 days, or within 2, 3, 4, 5, 6, 7, or 8 weeks of each other); (iii) the therapies are administered at least once during overlapping time periods (e.g., by continuous or intermittent infusion); or (iv) any combination of the foregoing. In some embodiments agents may be administered individually at substantially the same time (e.g., within less than 1, 2, 5, or 10 minutes of one another). In some embodiments agents may be administered individually within less than 3 hours, e.g., less than 1 hour. In some embodiments agents may be administered by the same route of administration. In some embodiments agents may be administered by different routes of administration. It will be understood that any of the afore-mentioned time frames pertaining to combination therapy may apply to agents and/or to non-pharmacological therapies such as hyperthermia, externally administered radiotherapy, etc.

A “regimen” or “treatment protocol” refers to a selection of one or more agent(s), dose level(s), and optionally other aspects(s) that describe the manner in which therapy is administered to a subject, such as dosing interval, route of administration, rate and duration of a bolus administration or infusion, appropriate parameters for administering radiation, etc. Many cancer chemotherapy regimens include combinations of drugs that have different cytotoxic or cytostatic mechanisms and/or that typically result in different dose-limiting adverse effects. For example, an agent that acts on DNA (e.g., alkylating agent) and an anti-microtubule agent are a common combination found in many chemotherapy regimens.

For purposes herein a regimen that has been tested in a clinical trial, e.g., a regimen that has been shown to be acceptable in terms of safety and, in some embodiments, showing at least some evidence of efficacy, will be referred to as a “standard regimen” and an agent used in such a regimen may be referred to as a “standard chemotherapy agent”. In some embodiments a standard regimen or standard chemotherapy agent is a regimen or chemotherapy agent that is used in clinical practice in oncology. In some embodiments pharmaceutical agents used in a standard regimen are all approved drugs. See, e.g., DeVita, supra for examples of standard regimens. It will be understood that different standard regiments may be selected as appropriate based on factors such as tumor type, tumor grade, tumor stage, concomitant illnesses, concomitant illnesses, general condition of the patient, etc.

In some embodiments an OXPHOS inhibitor is added to a standard regimen or substituted for one or more of the agents typically used in a standard regimen. In some embodiments a biguanide is added to a standard regimen or substituted for one or more of the agents typically used in a standard regimen. Non-limiting examples of cancer chemotherapeutic agents that may be used include, e.g., alkylating and alkylating-like agents such as nitrogen mustards (e.g., chlorambucil, chlormethine, cyclophosphamide, ifosfamide, and melphalan), nitrosoureas (e.g., carmustine, fotemustine, lomustine, streptozocin); platinum agents (e.g., alkylating-like agents such as carboplatin, cisplatin, oxaliplatin, BBR3464, satraplatin), busulfan, dacarbazine, procarbazine, temozolomide, thioTEPA, treosulfan, and uramustine; antimetabolites such as folic acids (e.g., aminopterin, methotrexate, pemetrexed, raltitrexed); purines such as cladribine, clofarabine, fludarabine, mercaptopurine, pentostatin, thioguanine; pyrimidines such as capecitabine, cytarabine, fluorouracil, floxuridine, gemcitabine; spindle poisons/mitotic inhibitors such as taxanes (e.g., docetaxel, paclitaxel), vincas (e.g., vinblastine, vincristine, vindesine, and vinorelbine), epothilones; cytotoxic/anti-tumor antibiotics such anthracyclines (e.g., daunorubicin, doxorubicin, epirubicin, idarubicin, mitoxantrone, pixantrone, and valrubicin), compounds naturally produced by various species of Streptomyces (e.g., actinomycin, bleomycin, mitomycin, plicamycin) and hydroxyurea; topoisomerase inhibitors such as camptotheca (e.g., camptothecin, topotecan, irinotecan) and podophyllums (e.g., etoposide, teniposide); monoclonal antibodies for cancer therapy such as anti-receptor tyrosine kinases (e.g., cetuximab, panitumumab, trastuzumab), anti-CD20 (e.g., rituximab and tositumomab), and others for example alemtuzumab, aevacizumab, gemtuzumab; photosensitizers such as aminolevulinic acid, methyl aminolevulinate, porfimer sodium, and verteporfin; tyrosine and/or serine/threonine kinase inhibitors, e.g., inhibitors of Abl, Kit, insulin receptor family member(s), VEGF receptor family member(s), EGF receptor family member(s), PDGF receptor family member(s), FGF receptor family member(s), mTOR, Raf kinase family, phosphatidyl inositol (PI) kinases such as PI3 kinase, PI kinase-like kinase family members, cyclin dependent kinase (CDK) family members, Aurora kinase family members (e.g., kinase inhibitors that are on the market or have shown efficacy in at least one phase III trial in tumors, such as cediranib, crizotinib, dasatinib, erlotinib, gefitinib, imatinib, lapatinib, nilotinib, sorafenib, sunitinib, vandetanib), growth factor receptor antagonists, and others such as retinoids (e.g., alitretinoin and tretinoin), altretamine, amsacrine, anagrelide, arsenic trioxide, asparaginase (e.g., pegasparagase), bexarotene, bortezomib, denileukin diftitox, estramustine, ixabepilone, masoprocol, mitotane, and testolactone, Hsp90 inhibitors, proteasome inhibitors (e.g, bortezomib), angiogenesis inhibitors, e.g., anti-vascular endothelial growth factor agents such as bevacizumab (Avastin) or VEGF receptor antagonists or soluble VEGF receptor domain (e.g., VEGF-Trap), matrix metalloproteinase inhibitors, various pro-apoptotic agents (e.g., apoptosis inducers), Ras inhibitors, anti-inflammatory agents, cancer vaccines, or other immunomodulating therapies, RNAi agents targeted to oncogenes, etc. It will be understood that the preceding classification is non-limiting. A number of anti-tumor agents have multiple activities or mechanisms of action and could be classified in multiple categories or classes or have additional mechanisms of action or targets. In certain embodiments an OXPHOS inhibitor is administered in combination with an angiogenesis inhibitor. In certain embodiments a biguanide is administered in combination with an angiogenesis inhibitor. Such combination therapy may maintain glucose limitation sensitivity of a tumor by inhibiting angiogenesis that would otherwise result in new blood vessel growth to supply the tumor.

### V. Pharmaceutical Compositions and Methods of Treatment

Agents and compositions disclosed herein or identified as disclosed herein may be administered to a subject, e.g., a subject in need of treatment of cancer, by any suitable route such as by intravenous, intraarterial, oral, intranasal, subcutaneous, intramuscular, intraosseus, intrasternal, intraperitoneal, intrathecal, intratracheal, intraocular, sublingual, vaginal, rectal, dermal, or pulmonary administration. Administration of a compound of composition may thus comprise introducing a compound or composition into or onto the body by any suitable route. Depending upon the type of condition (e.g., cancer) to be treated, agents may, for example, be introduced into the vascular system, inhaled, ingested, etc. Thus, a variety of administration modes, or routes, are available. The particular mode selected will, in various embodiments, generally depend on one or more factors such as the particular cancer being treated, the dosage required for therapeutic efficacy, and agents (if any) used in combination. The methods, generally speaking, may be practiced using any mode of administration that is medically or veterinarily acceptable, meaning any mode that produces acceptable levels of efficacy without causing clinically unacceptable (e.g., medically or veterinarily unacceptable) adverse effects. The term “parenteral” includes intravenous, intraarterial, intramuscular, intraperitoneal, subcutaneous, intraosseus, and intrasternal injection, or infusion techniques. In some embodiments a method comprises dispensing a compound or composition for administration to a subject as described herein. In some embodiments administration takes place in a health care setting such as a hospital, clinic, or physician's office. In some embodiments administration comprises self-administration.

It will be understood that in some embodiments administration of an agent or composition may be performed for one or more purposes in addition to or instead of for treatment purposes. For example, in some embodiments a detection reagent is administered for purposes of in vivo detection of expression or activity of a target molecule. In some embodiments an agent or composition is administered for diagnosis or monitoring or for testing the agent or composition.

In some embodiments a route or location of administration is selected based at least in part on the location of a tumor. For example, an agent or composition may be administered locally, e.g., to or near a tissue or organ harboring or suspected of harboring a tumor or from which a tumor has been removed. Local delivery may increase the anti-tumor effect by locally increasing the concentration of the agent at the tumor site as compared with the concentration that would be achieved using other delivery approaches, may reduce metabolism or clearance as compared with systemic administration, or may reduce the incidence or severity of side effects as compared with systemic administration. In some embodiments administration near a tissue or organ harboring or suspected of harboring a tumor or from which a tumor has been removed comprises administration within up to 5 cm, 10 cm, 15 cm, 20 cm, or 25 cm from the edge or margin of the tumor or organ.

In some embodiments, a method comprises administering an agent locally by administering it directly into the arterial blood supply of a tumor in a subject. The agent or composition may be administered into the artery using standard methods known in the art. In some embodiments the agent or composition is administered using a catheter. Insertion of the catheter may be guided or observed by imaging, e.g., fluoroscopy, or other suitable methods known in the art.

In some embodiments treating a subject in need of treatment for a tumor comprises administering one or more agents that reduce one or more side effects resulting from treatment of the tumor. For example, the one or more agents may control nausea or promote elimination or detoxification of substances released as a result of tumor lysis.

In some embodiments, inhaled medications are of use. Such administration allows direct delivery to the lung, e.g., for treatment of lung cancer, although it could be used to achieve systemic delivery in certain embodiments. In some embodiments, intrathecal administration may be used, e.g., in a subject with a tumor of the central nervous system, e.g., a brain tumor.

In some embodiments an agent or composition is administered prior to, during, and/or following ablation, radiation, or surgical removal. Treatment prior to ablation, radiation, or surgery may be performed at least in part to reduce the size of the tumor and render it more amenable to ablation, radiation, or surgical therapy. Treatment during or after ablation, radiation, or surgery may be performed at least in part to eliminate residual tumor cells and/or to reduce the likelihood of recurrence.

Suitable preparations, e.g., substantially pure preparations, of an active agent (e.g., an OXPHOS inhibitor, biguanide, etc.) may be combined with one or more pharmaceutically acceptable carriers or excipients, etc., to produce an appropriate pharmaceutical composition. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds is well-known in the art (see, for example, “Remington's Pharmaceutical Sciences”, E. W. Martin, 19th Ed., 1995, Mack Publishing Co.: Easton, Pa., and more recent editions or versions thereof, such as Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types).

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate. fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic. Pharmaceutical compositions and agents for use in such compositions may be manufactured under conditions that meet standards or criteria prescribed by a regulatory agency such as the US FDA (or similar agency in another jurisdiction) having authority over the manufacturing, sale, and/or use of therapeutic agents. For example, such compositions and agents may be manufactured according to Good Manufacturing Practices (GMP) and/or subjected to quality control procedures appropriate for pharmaceutical agents to be administered to humans.

For oral administration, agents can be formulated by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art.

Formulations for oral delivery may incorporate agents to improve stability in the gastrointestinal tract and/or to enhance absorption.

For administration by inhalation, pharmaceutical compositions may be delivered in the form of an aerosol spray from a pressured container or dispenser which contains a suitable propellant, e.g., a gas such as carbon dioxide, a fluorocarbon, or a nebulizer. Liquid or dry aerosol (e.g., dry powders, large porous particles, etc.) can be used. The disclosure contemplates delivery of compositions using a nasal spray or other forms of nasal administration. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers.

For topical applications, pharmaceutical compositions may be formulated in a suitable ointment, lotion, gel, or cream containing the active components suspended or dissolved in one or more pharmaceutically acceptable carriers suitable for use in such composition.

For local delivery to the eye, pharmaceutical compositions may be formulated as solutions or micronized suspensions in isotonic, pH adjusted sterile saline, e.g., for use in eye drops, or in an ointment. In some embodiments intraocular administration is used. Routes of intraocular administration include, e.g., intravitreal injection, retrobulbar injection, peribulbar injection, subretinal, sub-Tenon injection, and subconjunctival injection.

Pharmaceutical compositions may be formulated for transmucosal or transdermal delivery. For transmucosal or transdermal administration, penetrants appropriate to the barrier to be permeated may be used in the formulation. Such penetrants are generally known in the art. Pharmaceutical compositions may be formulated as suppositories (e.g., with conventional suppository bases such as cocoa butter and other glycerides) or as retention enemas for rectal delivery.

In some embodiments, a pharmaceutical composition includes one or more agents intended to protect the active agent(s) against rapid elimination from the body, such as a controlled release formulation, implants (e.g., macroscopic implants such as discs, wafers, etc.), microencapsulated delivery system, etc. Compounds may be encapsulated or incorporated into particles, e.g., microparticles or nanoparticles. Biocompatible polymers, e.g., biodegradable biocompatible polymers, can be used, e.g., in the controlled release formulations, implants, or particles. A polymer may be a naturally occurring or artificial polymer. Depending on the particular polymer, it may be synthesized or obtained from naturally occurring sources. An agent may be released from a polymer by diffusion, degradation or erosion of the polymer matrix, or combinations thereof. A polymer or combination of polymers, or delivery format (e.g., particles, macroscopic implant) may be selected based at least in part on the time period over which release of an agent is desired. A time period may range, e.g., from a few hours (e.g., 3-6 hours) to a year or more. In some embodiments a time period ranges from 1-2 weeks up to 3-6 months, or between 6-12 months. After such time period release of the agent may be undetectable or may be below therapeutically useful or desired levels. A polymer may be a homopolymer, copolymer (including block copolymers), straight, branched-chain, or cross-linked. Various polymers of use in drug delivery are described in Jones, D., Pharmaceutical Applications of Polymers for Drug Delivery, ISBN 1-85957-479-3, ChemTec Publishing, 2004. Useful polymers include, but are not limited to, poly-lactic acid (PLA), poly-glycolic acid (PGA), poly-lactide-co-glycolide (PLGA), poly(phosphazine), poly(phosphate ester), polycaprolactones, polyanhydrides, ethylene vinyl acetate, polyorthoesters, polyethers, and poly(beta amino esters). Other polymers useful in various embodiments include polyamides, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, poly(butyric acid), poly(valeric acid), and poly(lactide-cocaprolactone). Peptides, polypeptides, proteins such as collagen or albumin, polysaccharides such as sucrose, chitosan, dextran, alginate, hyaluronic acid (or derivatives of any of these) and dendrimers are of use in certain embodiments. Methods for preparation of such will be apparent to those skilled in the art. Additional polymers include cellulose derivatives such as, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxymethylcellulose, carboxylethyl cellulose, cellulose triacetate, cellulose sulphate sodium salt, polycarbamates or polyureas, cross-linked poly(vinyl acetate) and the like, ethylene-vinyl ester copolymers such as ethylene-vinyl acetate (EVA) copolymer, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer, or mixtures thereof. Chemical derivatives of the afore-mentioned polymers, e.g., substitutions, additions of chemical groups, for example, alkyl, alkylene, hydroxylations, oxidations, and other modifications routinely made by those skilled in the art can be used. A particle, implant, or formulation may be composed of a single polymer or multiple polymers. A particle or implant may be homogeneous or non-homogeneous in composition. In some embodiments a particle comprises a core and at least one shell or coating layer, wherein, in some embodiments, the composition of the core differs from that of the shell or coating layer. A therapeutic agent or label may be physically associated with a particle, formulation, or implant in a variety of different ways. For example, agents may be encapsulated, attached to a surface, dispersed homogeneously or nonhomogeneously in a matrix, etc. Methods for preparation of such formulations, implants, or particles will be apparent to those skilled in the art. Liposomes or other lipid-containing particles can be used as pharmaceutically acceptable carriers in certain embodiments. In some embodiments a controlled release formulation, implant, or particles may be introduced or positioned within a tumor, near a tumor or its blood supply, in or near a region from which a tumor was removed, at or near a site of known or potential metastasis (e.g., a site to which a tumor is prone to metastasize), etc. Microparticles and nanoparticles can have a range of dimensions. In some embodiments a microparticle has a diameter between 100 nm and 100 μm. In some embodiments a microparticle has a diameter between 100 nm and 1 μm, between 1 μm and 20 μm, or between 1 μm and 10 μm. In some embodiments a microparticle has a diameter between 100 nm and 250 nm, between 250 nm and 500 nm, between 500 nm and 750 nm, or between 750 nm and 1 μm. In some embodiments a nanoparticle has a diameter between 10 nm and 100 nm, e.g., between 10 nm and 20 nm, between 20 nm and 50 nm, or between 50 nm and 100 nm. In some embodiments particles are substantially uniform in size or shape. In some embodiments particles are substantially spherical. In some embodiments a particle population has an average diameter falling within any of the afore-mentioned size ranges. In some embodiments a particle population consists of between about 20% and about 100% particles falling within any of the afore-mentioned size ranges or a subrange thereof, e.g. about 40%, 40%, 50%, 60%, 70%, 80%, 90%, 95%, etc. In the case of non-spherical particles, the longest straight dimension between two points on the surface of the particle rather than the diameter may be used as a measure of particle size. Such dimension may have any of the length ranges mentioned above. In some embodiments a particle comprises a detectable label or detection reagent or has a detectable label or detection reagent attached thereto. In some embodiments a particle is magnetic, e.g., to facilitate removal or separation of the particle from a composition that comprises the particle and one or more additional components.

Forms of polymeric matrix that may contain and/or be used to deliver an agent include films, coatings, gels (e.g., hydrogels), which may be implanted or applied to an implant or indwelling device such as a stent or catheter.

In general, the size, shape, and/or composition of a polymeric material, matrix, or formulation may be appropriately selected to result in release in therapeutically useful amounts over a useful time period, in the tissue into the polymeric material, matrix, or formulation is implanted or administered.

In some embodiments a tautomeric, enantiomeric, diastereoisomeric, epimeric forms or a solvate of any of the agents described herein, e.g., an OXPHOS inhibitor, biguanide, etc., may be used. In some embodiments, a pharmaceutically acceptable salt, ester, salt of such ester, active metabolite, prodrug, or any adduct or derivative of a compound, e.g., an OXPHOS inhibitor, biguanide, etc., which upon administration to a subject in need thereof is capable of providing the compound, directly or indirectly, is used. In some embodiments a pharmaceutically acceptable salt, ester, salt of such ester, active metabolite, prodrug, or adduct or derivative may be formulated and, in general, used for the same purpose(s) as such compound.

The term “pharmaceutically acceptable salt” refers to those salts which are, within the scope of sound medical judgment, suitable for use in contact with the tissues of humans and/or lower animals without undue toxicity, irritation, allergic response and the like, and which are commensurate with a reasonable benefit/risk ratio. A wide variety of appropriate pharmaceutically acceptable salts are well known in the art. Pharmaceutically acceptable salts include, but are not limited to, those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate., stearate, succinate, sulfate, tartrate, thiocyanate, p-toluenesulfonate, undecanoate, valerate salts, and the like. In some embodiments cases, a compound may contain one or more acidic functional groups and, thus, be capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary, tertiary, or quaternary amine. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Representative organic amines useful for the formation of base addition salts include ethylamine, diethylamine, ethylenediamine, ethanolamine, diethanolamine, piperazine and the like.

A therapeutically effective dose of an active agent in a pharmaceutical composition may be within a range of about 1 μg/kg to about 500 mg/kg body weight, about 0.001 mg/kg to about 100 mg/kg, about 0.001 mg/kg to about 10 mg/kg, about 0.01 mg/kg to about 25 mg/kg, about 0.1 mg/kg to about 20 mg/kg body weight, about 1 mg/kg to about 10 mg/kg, about 1 mg/kg to about 3 mg/kg, about 3 mg/kg to about 5 mg/kg, about 5 mg/kg to about 10 mg/kg. In some embodiments doses of agents described herein may range, e.g., from about 10 μg to about 10,000 mg, e.g., from about 100 μg to about 5,000 mg, e.g., from about 0.1 mg to about 1000 mg once or more per day, week, month, or other time interval, in various embodiments. In some embodiments a dose is expressed in terms of mg/m² body surface area. Body surface area may be estimated using standard methods. In some embodiments a single dose is administered while in other embodiments multiple doses are administered. Those of ordinary skill in the art will appreciate that appropriate doses in any particular circumstance depend upon the potency of the agent(s) utilized, and may optionally be tailored to the particular recipient. The specific dose level for a subject may depend upon a variety of factors including the activity of the specific agent(s) employed, severity of the disease or disorder, the age, body weight, general health of the subject, etc.

In certain embodiments an agent may be used at the maximum tolerated dose or a sub-therapeutic dose or any dose there between, e.g., the lowest dose effective to achieve a therapeutic effect. Maximum tolerated dose (MTD) refers to the highest dose of a pharmacological or radiological treatment that can be administered without unacceptable toxicity, that is, the highest dose that has an acceptable risk/benefit ratio, according to sound medical judgment. In general, the ordinarily skilled practitioner can select a dose that has a reasonable risk/benefit ratio according to sound medical judgment. A MTD may, for example, be established in a population of subjects in a clinical trial. In certain embodiments an agent is administered in an amount that is lower than the MTD, e.g., the agent is administered in an amount that is about 10%, 20%, 30%, 40%, 50%, 60%, 70%, 80%, or 90% of the MTD.

It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with an appropriate pharmaceutically acceptable carrier. In some embodiments a pharmaceutically acceptable unit dosage form contains a predetermined amount of an agent, e.g., an OXPHOS inhibitor, such amount being appropriate to treat a subject in need of treatment for a cancer. In some embodiments a pharmaceutically acceptable unit dosage form contains a predetermined amount of a biguanide, such amount being appropriate to treat a subject in need of treatment for a cancer.

It will be understood that a therapeutic regimen may include administration of multiple unit dosage forms over a period of time. In some embodiments, a subject is treated for between 1-7 days. In some embodiments a subject is treated for between 7-14 days. In some embodiments a subject is treated for between 14-28 days. In other embodiments, a longer course of therapy is administered, e.g., over between about 4 and about 10 weeks. In some embodiments multiple courses of therapy are administered. In some embodiments, treatment may be continued indefinitely. For example, a subject at risk of cancer recurrence may be treated for any period during which such risk exists. A subject may receive one or more doses a day, or may receive doses every other day or less frequently, within a treatment period. Treatment courses may be intermittent.

In some embodiments, an agent is provided in a pharmaceutical pack or kit comprising one or more containers (e.g., vials, ampoules, bottles) containing the agent and, optionally, one or more other pharmaceutically acceptable ingredients. Optionally associated with such container(s) can be a notice in the form prescribed by a governmental agency regulating the manufacture, use or sale of pharmaceutical products, which notice reflects approval by the agency of manufacture, use or sale for human administration. The notice may describe, e.g., doses, routes and/or methods of administration, approved indications (e.g., cancers that the agent or pharmaceutical composition has been approved for use in treating), mechanism of action, or other information of use to a medical practitioner and/or patient. In some embodiments the notice specifies that the agent is to be used for treating tumors that have increased likelihood of sensitivity to the agent (or agents of its class) or equivalent language. In some embodiments a particular test for assessing expression, activation, mutation status of a tumor is suggested or specified, e.g., as part of an indication. Different ingredients may be supplied in solid (e.g., lyophilized) or liquid form. Each ingredient will generally be suitable as aliquoted in its respective container or provided in a concentrated form. Kits may also include media for the reconstitution of lyophilized ingredients. The individual containers of the kit are preferably maintained in close confinement for commercial sale.

One of ordinary skill in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure provides embodiments in which exactly one member of the group is present in, employed in, or otherwise relevant to a given product or process. The present disclosure also provides embodiments in which more than one, or all of the group members are present in, employed in, or otherwise relevant to a given product or process. Furthermore, it is to be understood that the present disclosure provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects described herein where appropriate. It is also contemplated that any of the embodiments or aspects or teachings can be freely combined with one or more other such embodiments or aspects whenever appropriate and regardless of where such embodiment(s), aspect(s), or teaching(s) appear in the present disclosure. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect can be explicitly excluded from the claims, regardless of whether the specific exclusion is recited in the specification. For example, any one or more agents, disorders, subjects, or combinations thereof, can be excluded.

Where the claims or description relate to a product (e.g., a composition of matter), it should be understood that methods of making or using the product according to any of the methods disclosed herein, and methods of using the product for any one or more of the purposes disclosed herein, are encompassed by the present disclosure, where applicable, unless otherwise indicated or unless it would be evident to one of ordinary skill in the art that a contradiction or inconsistency would arise. Where the claims or description relate to a method, it should be understood that product(s), e.g., compositions of matter, device(s), or system(s), useful for performing one or more steps of the method are encompassed by the present disclosure, where applicable, unless otherwise indicated or unless it would be evident to one of ordinary skill in the art that a contradiction or inconsistency would arise.

Where ranges are given herein, embodiments are provided in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, embodiments that relate analogously to any intervening value or range defined by any two values in the series are provided, and that the lowest value may be taken as a minimum and the greatest value may be taken as a maximum. Where a phrase such as “at least”, “up to”, “no more than”, or similar phrases, precedes a series of numbers herein, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated where applicable. A reasonable lower or upper limit may be selected or determined by one of ordinary skill in the art based, e.g., on factors such as convenience, cost, time, effort, availability (e.g., of samples, agents, or reagents), statistical considerations, etc. In some embodiments an upper or lower limit differs by a factor of 2, 3, 5, or 10, from a particular value. Numerical values, as used herein, include values expressed as percentages. For each embodiment in which a numerical value is prefaced by “about” or “approximately”, embodiments in which the exact value is recited are provided. For each embodiment in which a numerical value is not prefaced by “about” or “approximately”, embodiments in which the value is prefaced by “about” or “approximately” are provided. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. In some embodiments a method may be performed by an individual or entity. In some embodiments steps of a method may be performed by two or more individuals or entities such that a method is collectively performed. In some embodiments a method may be performed at least in part by requesting or authorizing another individual or entity to perform one, more than one, or all steps of a method. In some embodiments a method comprises requesting two or more entities or individuals to each perform at least one step of a method. In some embodiments performance of two or more steps is coordinated so that a method is collectively performed. Individuals or entities performing different step(s) may or may not interact. In some embodiments a request is fulfilled, e.g., a method or step is performed, in exchange for a fee or other consideration and/or pursuant to an agreement between a requestor and an individual or entity performing the method or step. It should also be understood that unless otherwise indicated or evident from the context, any product or composition described herein may be considered “isolated”. It should also be understood that, where applicable, unless otherwise indicated or evident from the context, any method or step of a method that may be amenable to being performed mentally or as a mental step or using a writing implement such as a pen or pencil, and a surface suitable for writing on, such as paper, may be expressly indicated as being performed at least in part, substantially, or entirely, by a machine, e.g., a computer, device (apparatus), or system, which may, in some embodiments, be specially adapted or designed to be capable of performing such method or step or a portion thereof.

Section headings used herein are not to be construed as limiting in any way. It is expressly contemplated that subject matter presented under any section heading may be applicable to any aspect or embodiment described herein.

Embodiments or aspects herein may be directed to any agent, composition, article, kit, and/or method described herein. It is contemplated that any one or more embodiments or aspects can be freely combined with any one or more other embodiments or aspects whenever appropriate. For example, any combination of two or more agents, compositions, articles, kits, and/or methods that are not mutually inconsistent, is provided. It will be understood that any description or exemplification of a term anywhere herein may be applied wherever such term appears herein (e.g., in any aspect or embodiment in which such term is relevant) unless indicated or clearly evident otherwise.

### EXAMPLES

### Overview of Certain of the Examples, and Certain Materials and Methods

The cancer cell response to low glucose is not well documented due to the difficulty in maintaining constant glucose levels with standard methods, as cells rapidly deplete glucose from the media. Therefore, we developed a continuous flow cell culture system (Nutrostat) enabling cell culture at controlled nutrient levels, and performed pooled shRNA loss-of-function genetic screens of 2,719 metabolic genes at low or standard glucose concentrations. We identified a subset of genes involved in mitochondrial oxidative phosphorylation (OXPHOS) that are differentially required for proliferation under low glucose. We also simultaneously determined the glucose-dependent growth properties of 30 cancer cell lines. Those cell lines most sensitive to glucose limitation are universally incapable of inducing OXPHOS upon glucose restriction principally due to either dysfunctional mitochondria or poor glucose import. Together, these data demonstrate that specific OXPHOS components are of major importance for mitochondrial function at the glucose concentrations present in the tumor microenvironment, and will inform the design of future chemotherapeutics targeting the mitochondria.

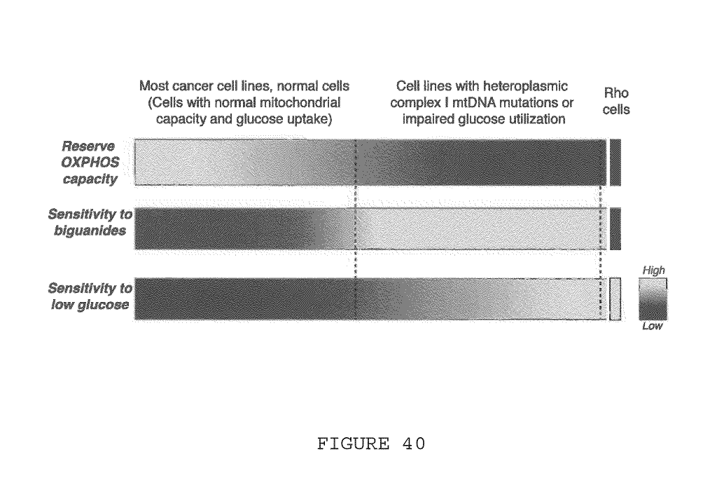

As described herein, we find that cancer cells exhibit diverse responses to glucose limitation and identify defects in glucose utilization and mitochondrial function as major determinants of low glucose sensitivity (FIG. 40). These biomarkers may pinpoint cancer cells likely to respond to OXPHOS inhibition alone under tumor-relevant glucose concentrations. Such a targeted strategy may be better tolerated than previously proposed approaches of combining inhibition of OXPHOS and glycolysis²¹⁻²³. Moreover, our findings underscore the importance of considering glucose concentrations when evaluating the sensitivity of cancer cells to biguanides or other OXPHOS inhibitors. The methods described here should be valuable for studying the responses of cancer cells to tumour-relevant concentrations of other highly consumed nutrients, such as amino acids²⁴, and to additional compounds that target metabolism.

Certain materials and methods that may be used in multiple examples are described below. It will be understood that certain other methods described in particular examples below are employed in multiple examples.

Cell Lines and Reagents:

Cell lines were obtained from the Broad Institute Cancer Cell Line Encyclopedia with the exceptions of HL-60, Daudi, HuT 78, MC116, Raji, and U-937, which were kindly provided by Robert Weinberg (Whitehead Institute, Cambridge, Mass., USA), KMS-26 and KMS-27 which were purchased from the JCRB Cell Bank, Immortalized B lines 1 and 2 which were provided by Dr. Christoph Klein (Carl Hannover Medical School, Germany), and Cal-62 which was provided by James A. Fagin (Memorial Sloan-Kettering Cancer Center, New York, N.Y., USA). To normalize for media specific effects on cell metabolism, all cell lines were grown in RPMI base medium containing 10% heat inactivated fetal bovine serum, 2 mM glutamine, penicillin, and streptomycin. The NDI1 antibody is a kind gift of Takao Yagi (The Scripps Research Institute, La Jolla, Calif., USA). Additional antibodies used are: Actin (I-19, Santa Cruz), Glut3 (ab15311, Abcam), RPS6 (Cell Signaling), CYC1 (Sigma) and UQCRC1 (H00007384-B01P, Novus).

Cell lines are from the following cancer origins. PANC1 (Pancreas), NCI-H838 (Lung), NCI-H596 (Lung), NCI-H1792 (Lung), A549 (Lung), NU-DHL-1 (Lymphoma), BxPC3 (Pancreas), Cal-62 (Thyroid), HCC-1438 (Lung), HCC-827 (Lung), L-363 (Plasma Cell Leukemia), MOLP-8 (Multiple Myeloma), LP-1 (Multiple Myeloma). Additional cell lines and their tissue origins are listed in Supplementary Table 1. One cell line (SNU-1) was randomly selected for authentication by STR profiling, and cell lines were authenticated by mtDNA sequencing (NCI-H82, Jurkat, NU-DHL-1, U-937, BxPC3, Cal-62, HCC-1438, HCC-827, Raji, MC116, KMS-26, NCI-H929, NCI-H2171).

Cell Proliferation Assays—Cell Counting:

Cells were plated in triplicate in 24 well plates at 5-20 thousand cells per well in 2 mL RPMI base media under the conditions described in each experiment (i.e. varying glucose concentration or phenformin treatment). After four days, the entire contents of the well was resuspended and counted (suspension cells) or trypsinized, resuspended and counted (adherent cells) using a Beckman Z2 Coulter Counter with a size selection setting of 8-30 um. The increase in cell number compared to the initially plated sample was calculated and all values were normalized to their control in 10 mM glucose unless otherwise indicated.

Cell Proliferation Assays—ATP-Based Measurements:

Cells were plated in replicates of five in 96 well plates at 0.5-1 thousand cells per well in 200 uL RPMI base media under the conditions described in each experiment, and a separate group of 5 wells was also plated for each cell line with no treatment for an initial time point. After 5 hours (untreated cells for initial time point) or after 3 days (with varying treatment conditions), 40 uL of Cell Titer Glo reagent (Promega) was added to each well, mixed briefly, and the luminescence read on a Luminometer (Molecular Devices). For wells with treatments causing an increase in luminescence, the fold change in luminescence relative to the initial luminescence was computed and this fold change for each condition was normalized to untreated wells (no effect=1). For wells with treatments causing a decrease in luminescence, the fold decrease in luminescence relative to the initial luminescence was computed (no viable cells present=−1)

Lentiviral shRNAs:

Lentiviral shRNAs were obtained from the The RNAi Consortium (TRC) collection of the Broad Institute. The TRC#s for the shRNAs used are below. For each gene, the order of the TRC numbers matches the order of the shRNAs as numbered elsewhere. The TRC website is: http://www.broadinstitute.org/rnai/trc/lib

| | |
|---|---|
| CYC1 | (TRCN0000064606, TRCN0000064603, TRCN0000064605), |
| UQCRC1 | (TRCN0000233157, TRCN0000046484, TRCN0000046487) |
| NDUFA7 | (TRCN0000026423, TRCN0000026454) |
| NDUFB1 | (TRCN0000027148, TRCN0000027173) |
| COX5A | (TRCN0000045961, TRCN0000045960) |
| UQCRH | (TRCN0000046528, TRCN0000046530) |
| UQCRFS1 | (TRCN0000046522, TRCN0000046519) |
| NDUFB10 | (TRCN0000026589, TRCN0000026579) |
| UQCR11 | (TRCN0000046465, TRCN0000046467) |
| NDUFA11 | (TRCN0000221374, TRCN0000221376) |
| NDUFV1 | (TRCN0000221380, TRCN0000221378) |
| PKM | (TRCN0000037612, TRCN0000195405) |
| RFP | (TRCN0000072203) |

Statistics and Animal Models:

Most experiments described below were repeated at least three times. T-tests were heteroscedastic to allow for unequal variance and distributions assumed to follow a Student's t distribution, and these assumptions are not contradicted by the data. No samples or animals were excluded from analysis, and sample size estimates were not used. Animals were randomly assigned into a treatment group with the constraint that the starting tumor burden in the treatment and control groups were similar. Studies were not conducted blind. The following abbreviations may be used in the Examples and/or elsewhere herein: UMP: Uridine Monophosphate; CMP: Cytidine Monophosphate; GMP: Guanosine Monophosphate; AMP: Adenosine Monophosphate; CDP: Cytidine Diphosphate; UDP: Uridine Diphosphate; GDP: Guanosine Diphosphate; NAD+/NADH: Nicotinaminde Adenine Dinucleotide (oxidized and reduced forms); NADP: Nicotinaminde Adenine Dinucleotide Phosphate; ADP: Adenosine Diphosphate; IMP: Inosine Monophosphate; 5-HIAA: 5-Hydroxyindoleacetic acid; 2-HG: 2-hydroxyglutarate; cAMP: cyclic AMP; Fruc: Fluctose; Glu: Glucose; Gal: Galactose; F P: Fructose 1-phosphate; F6P: Fructose 6-phosphate; G1P: Glucose 1-phosphate; G6P: Glucose 6-phosphate; PEP: Phosphoenolpyruvate; 3-PGA: 3-phosphoglycerate; F16DP: Fructose 1,6-diphosphate; F26DP: Fructose 2,6-diphosphate; G16DP: Glucose 1,6-diphosphate; Py: Pyruvate; Mal: Malate; FCCP: Carbonyl cyanide 4-(trifluoromethoxy)phenylhydrazone; TMPD: N,N,N′,N′-Tetramethyl-p-Phenylenediamine; CE: ceramide; DAG: Diacylglycerol. Fatty acids may additionally have annotations indicating the number of carbons and number of unsaturated linkages separated by a colon (e.g. 18:2).

### Example 1

### Development of a System for Studying Effects of Glucose Concentration

We are interested in better understanding which metabolic genes are required for cancer relevant processes such as proliferation, survival, and cell state, in the context of environment and genotype, and why. Among the environmental factors of interest to us is nutrient availability. In the case of many tumors (e.g., many solid tumors), the tumor microenvironment is likely to be nutrient poor. A viability threshold exists in that tumor cells located more than about 100-200 microns from a microvessel have reduced viability.

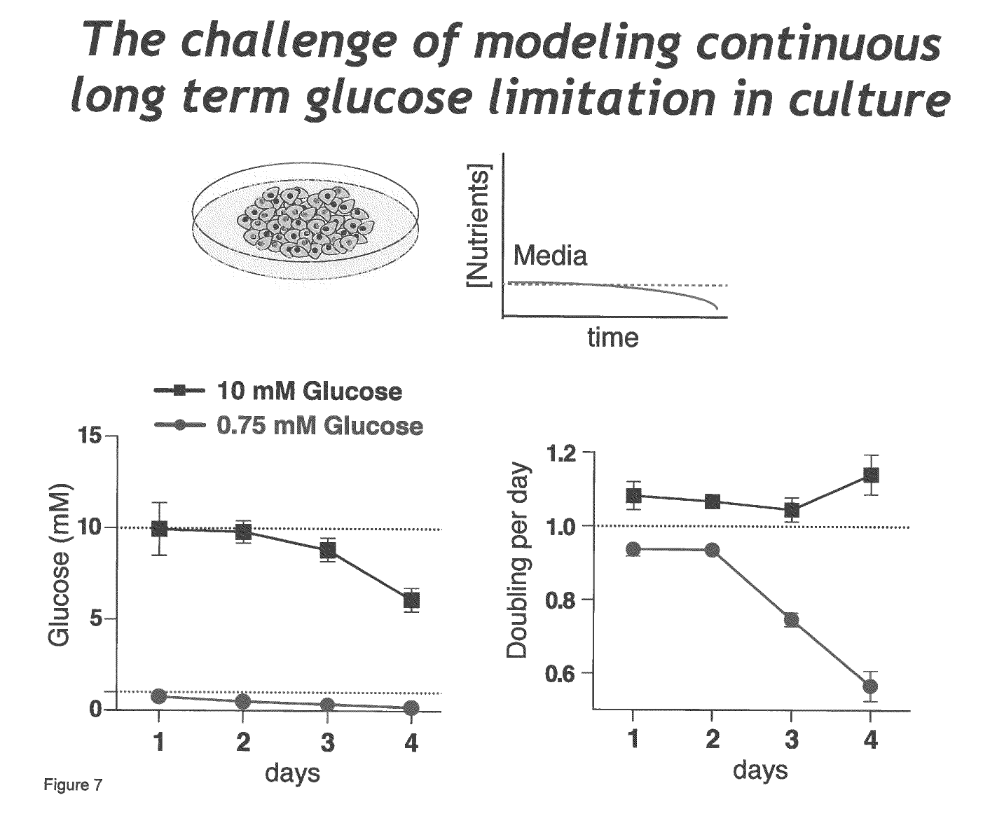

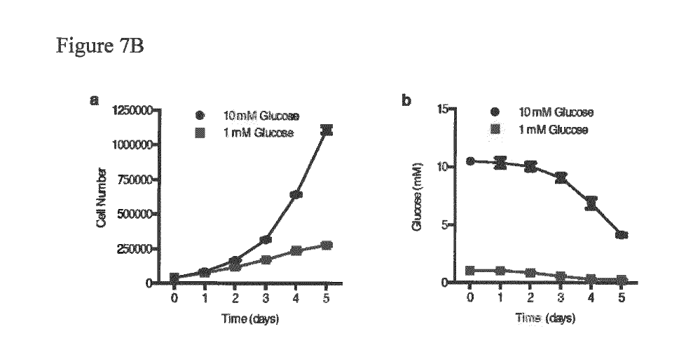

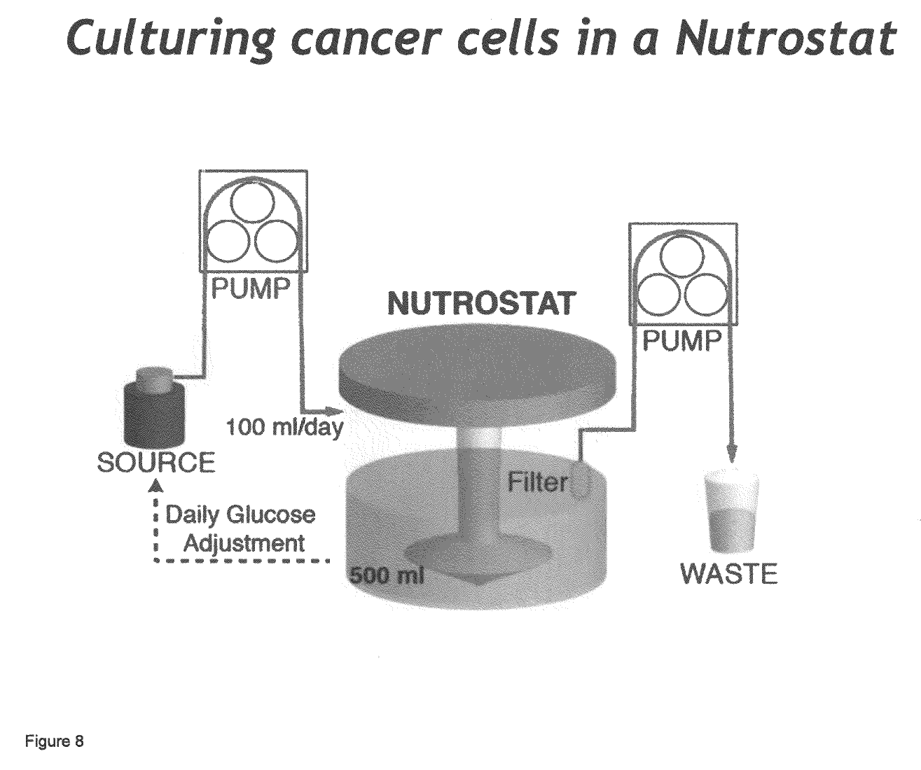

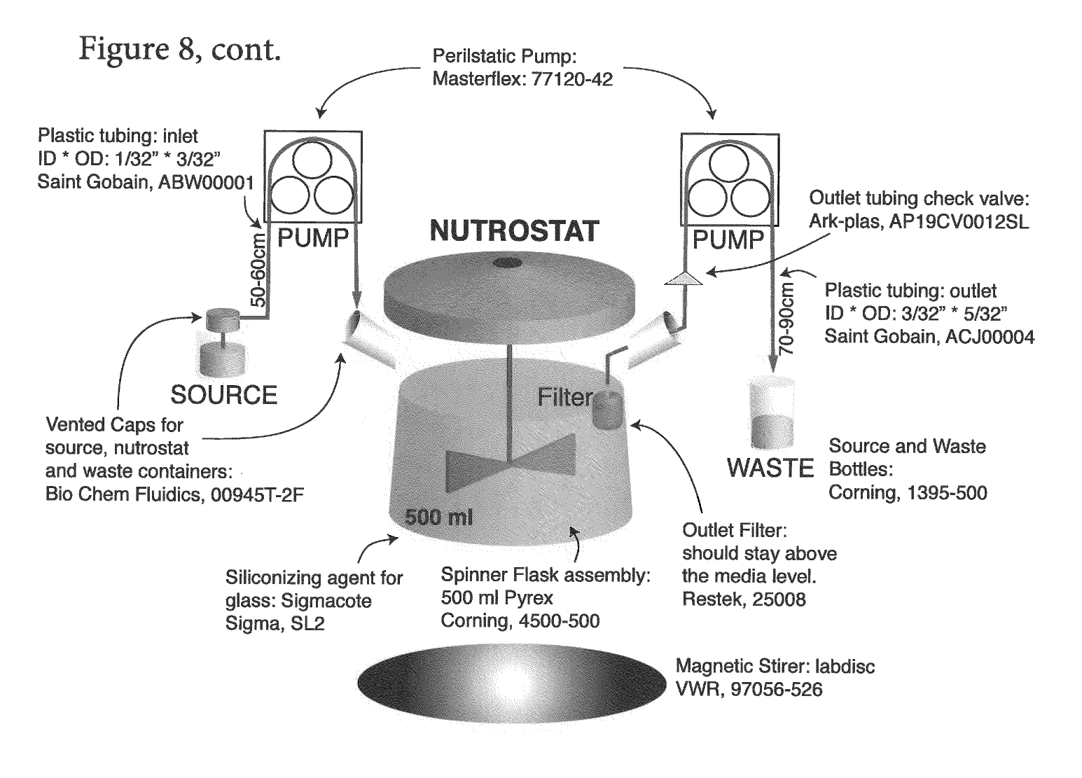

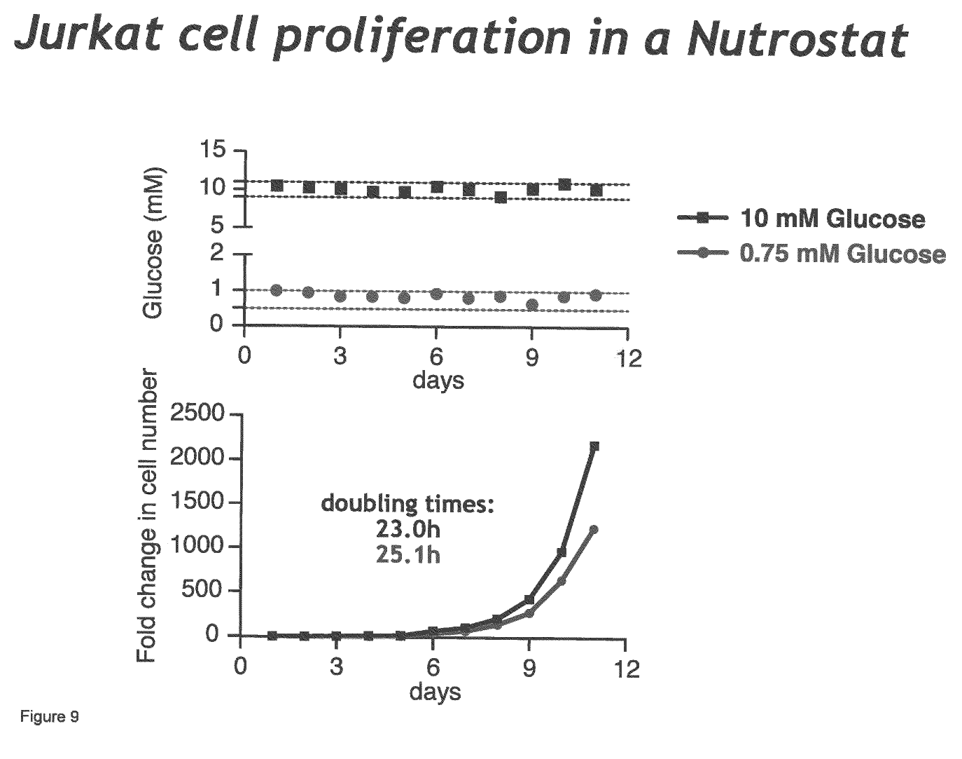

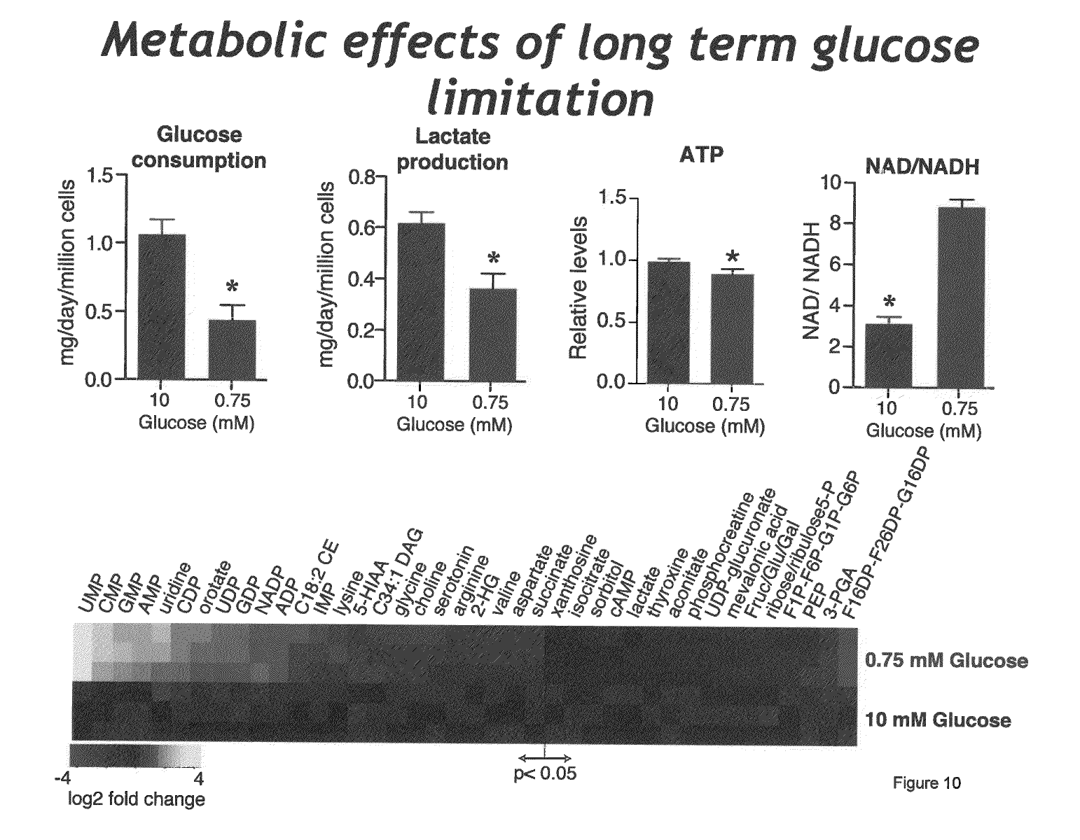

There are a number of challenges to modeling continuous long term nutrient limitation (or excess) in culture. When cells are grown in a standard cell culture system, nutrient concentrations in the medium drop over time as cells utilize the nutrients. The rate of change in nutrient concentrations varies depending on factor such as the number of cells and their proliferation rate. Effects arising due to concentrations of specific nutrients cannot be readily distinguished. Studying the effects of nutrient limitation over a significant time period is particularly challenging in part because a drop in the level of an essential nutrient may rapidly result in loss of cell viability. For example, we found that Jurkat cells grown in culture medium containing an initial glucose concentration of 0.75 mM exhibited a rapid decrease in proliferation rate once the glucose concentration fell below about 0.05 mM glucose (FIG. 7). In order to facilitate studies on the effect of nutrient levels on tumor cell processes, we designed a system for growing cells in culture under conditions in which the concentration of one or more selected nutrients is held constant. A schematic diagram of our system, termed a Nutrostat, is shown in FIG. 8, where the selected nutrient is glucose. Our system maintains an approximately constant concentration of glucose and, by continuously adding fresh media to the culture chamber and removing media from it also avoids rapid changes in concentration of nutrients and metabolic byproducts that may result from replacing the medium at intervals. As shown in FIG. 9, the Nutrostat successfully maintains cells at a constant glucose concentration for prolonged periods. This system allows the detailed analysis of effects of long term glucose limitation. Various changes arising under low glucose conditions are detailed in FIG. 10. Despite having a small effect on Jurkat cell proliferation, long term culture in low glucose caused profound metabolic changes: rates of glucose consumption and lactate production decreased as did levels of intermediates in the upper glycolysis and pentose-phosphate pathways. The NAD/NADH ratio went up while the energy charge strongly decreased, as revealed by a substantial increase in nucleoside monophosphates and a drop in ATP levels. As described further below, we used the system to (i) identify genes that are differentially required upon culture in low glucose versus high glucose medium; and (ii) identify cancer cell lines that exhibited differential sensitivity to low versus high glucose concentrations. Briefly, we found that cancer cell lines exhibit diverse responses to glucose limitation. A subset of lines (˜15%) have limited spare respiratory capacity through diverse defects. These defects define lines and tumors that are more sensitive to OXPHOS inhibitors. In particular, we found that deficiencies in glucose utilization or Complex I underlie sensitivity of cells to low glucose sensitivity of cancer cells.

Nutrostat Design

Equipment used in constructing the Nutrostat (FIG. 8): peristaltic pumps with accompanying tubing (Masterflex, manufacturer number 77120-42), 500 mL spinner flasks (Corning, product #4500-500), 9 position stirplate (Bellco Glass, manufacturer number 7785-D9005) or Lab Disk magnetic stirrer (VWR #97056-526), Tygon tubing (Saint Gobain Performance Plastics, manufacturer number ACJ00004 (outlet, 3/32“×5/32”) and ABW00001 (inlet, 1/32“× 3/32”), Outlet filter (Restek, catalog number 25008), vented caps for source and waste containers (Bio Chem Fluidics, catalog number 00945T-2F), and outlet tubing check valve (Ark-plas, catalog number AP19CV0012SL) to prevent backflow. Spinner flasks were siliconized before each use using Sigmacote (Sigma #SL2) according to the manufacturer's method, and autoclaved. Outlet filter was cleaned prior to use by passing phosphate buffered saline and then 70% ethanol through the filter in both the forward and reverse directions. Plastic tubing was replaced prior to each experiment and was cut to 50-60 cm pieces and threaded through the caps for the source or waste vessel, over the peristaltic pump, and through the caps on the spinner flask. The outlet tubing was cut ˜5 cm from the spinner flask to allow for the introduction of the check valve and prevent back-flow of media. Tubing was adjusted to the following heights: source vessel, bottom; spinner flask inlet, 3 cm from cap (above media level); spinner flask outlet+filter, empirically adjusted so that the volume of media in the vessel is maintained at 500 mL; waste vessel, 2 cm from the cap. The entire assembled setup was autoclaved prior to use. Flow rate of the inlet peristaltic pump was adjusted empirically to 100 mL per day using phosphate buffered saline before the introduction of culture media, and the flow rate of the waste pump was set to safely exceed 100 mL per day to prevent media accumulation in the vessel. Some escape of cells from the vessel and accumulation in the waste vessel was normal. Media was sampled directly from the vessel by pipette. The mass of glucose consumed by the Nutrostat over time was modeled by the following equation:

Gₙᵤₜᵣₒₛₜₐₜ(t)=∫₀^{t}N₀*Q_{glucose}*2^{t/a} dt

Where N₀ is the starting cell number, Q_{glucose} is the consumption rate of glucose (g/cell/day), a is the doubling time of the cell line (days), and t is time (days). The values for N₀, Q_{glucose} and a were empirically determined before the start of the experiment. The Nutrostat glucose consumption was calculated in hourly increments and balanced by the amount of glucose leaving or entering the chamber such that Gₙᵤₜᵣₒₛₜₐₜ over the one hour time interval=([Gluc]_{source}*Vᵢₙ)−([Gluc]ₙᵤₜᵣₒₛₜₐₜ*Vₒᵤₜ) where [Gluc]ₙᵤₜᵣₒₛₜₐₜ is the Nutrostat glucose concentration, [Gluc]_{source} is the source media glucose concentration, Vₒᵤₜ is the volume of media leaving the chamber, and Vᵢₙ is the volume of media entering the chamber (Vₒᵤₜ=Vᵢₙ=0.1 L/day). The [Gluc]_{source} was adjusted daily so that the [Gluc]ₙᵤₜᵣₒₛₜₐₜ predicted by the model remained between the desired glucose concentration boundaries, and adherence of the actual glucose concentration in the Nutrostat to the model was periodically evaluated by measuring the glucose concentration of media samples using a glucose oxidase assay (Fisher Scientific, catalog number TR-15221).

Metabolite Profiling:

For metabolite concentration measurements, 10 million Jurkat cells were cultured in Nutrostats for 2 weeks before metabolite extraction. Cells were rapidly washed three times with cold PBS, and metabolites were extracted by the addition of 80% ice-cold methanol. Endogenous metabolite profiles were obtained using LC-MS as described²⁶. Metabolite levels (n=3 biological replicates) were normalized to cell number.

Lactate and NAD(H) Measurements:

Lactate was measured as previously described²⁵ using the same medium that was used for glucose consumption measurements (above). NAD(H) was measured using the Fluoro NAD kit (Cell Technology FLNADH 100-2) according to the manufacturer's protocol.

### Example 2

### Identification of Genes that are Differentially Essential for Proliferation in Low Glucose

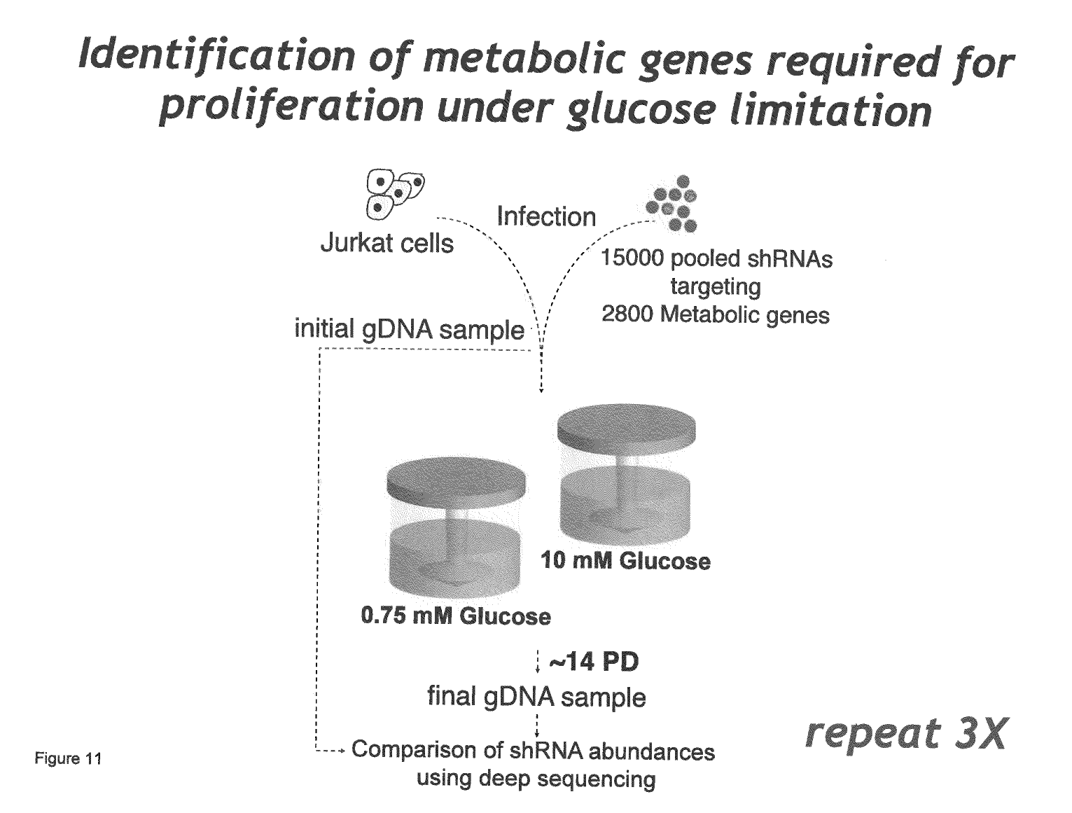

We undertook a loss of function genetic screen for genes that affect the sensitivity of cancer cells to glucose restriction. A schematic diagram of the screen is presented in FIG. 11. Jurkat cells were cultured in RPMI medium at standard culture conditions for this cell type (˜10 mM glucose, 2 mM glutamine). RPMI media with 2 mM glutamine was used for this and all other experiments described herein unless otherwise indicated. Different glucose concentrations were used as indicated, and various substances were included in the media in some experiments as indicated.

The cells were infected with a pool of lentiviruses harboring about 15,000 shRNAs targeted to about 2800 metabolic genes (Possemato, R. et al. Functional genomics reveal that the serine synthesis pathway is essential in breast cancer. Nature 476, 346-350, (2011)), more specifically, 2,752 transporters and metabolic enzymes. Lentiviral plasmids encoding ˜15,000 shRNAs targeting these genes (median of 5 shRNAs per gene) as well as 30 non-targeting control shRNAs were obtained and combined to generate a single plasmid pool, the composition of which is described in Supplementary Table 2. Plasmid pools were used to generate lentivirus-containing supernatants and target cell lines were infected in 2 ug/mL polybrene as described²⁵. Specifically, the titer of lentiviral supernatants was determined by infecting targets cells at several concentrations, counting the number of drug resistant infected cells after 3 days of selection. 30 million target cells were infected at an MOI of ˜0.5 to ensure that most cells contained only a single viral integrant and ensure proper library complexity. Infected cells were selected with 0.5 ug/mL puromycin for 3 days. An initial sample of cells was harvested and genomic DNA (gDNA) was obtained. Remaining cells were then cultured in a Nutrostat (described in Example 1) under conditions of either 0.75 mM or 10 mM glucose. Cells were inoculated in Nutrostats at ˜15M cells per 500 mL culture. Glucose concentrations were measured daily and adjusted as described above. Cultures were split back once to maintain a cell density of less than 500K cells/mL. Genomic DNA was harvested from each of the two cell populations after about 14 population doublings. Samples were processed as described²⁵ except that two rounds of PCR were used and the primers used to amplify shRNA inserts and perform deep sequencing (Illumina) are as provided below.

Primers for amplifying shRNAs encoded in genomic
DNA:
First Round of PCR (15 cycles):
5′ primer:
AATGGACTATCATATGCTTACCGTAACTTGAAAGTATTTCG
3′ primer:
CTTTAGTTTGTATGTCTGTTGCTATTATGTCTACTATTCTTTCCC
Second Round of PCR:
Barcoded Forward Primer (‘N’s indicate location
of sample-specific barcode sequence):
AATGATACGGCGACCACCGAGAAAGTATTTCGATTTCTTGGCTTTA
TATATCTTGTGGA NNNN ACGA
Common Reverse Primer:
CAAGCAGAAGACGGCATACGAGCTCTTCCGATCTTGTGGATGAATA
CTGCCATTTGTCTCGAGGTC
Illurnina Sequencing Primer:
GAGAAAGTATTTCGATTTCTTGGCTTTATATATCTTGTGGA

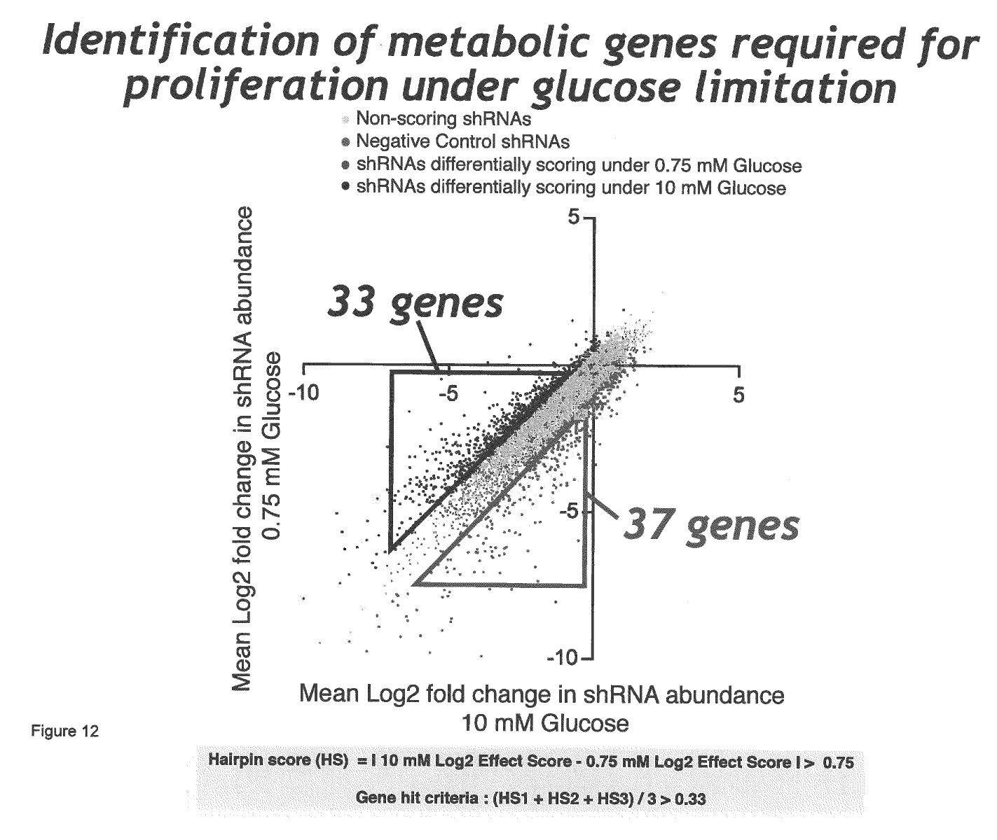

Deep sequencing was used to determine the abundance of each shRNA in each cell population. shRNAs present at fewer than 100 reads in the initial post-infection sample were eliminated from further analysis. Because the lentiviral pool contained shRNA expression vectors pLKO.1 and pLKO.005, to eliminate any backbone-specific amplification bias, the abundance measurements of shRNAs in the pLKO.005 vector were normalized such that the distribution of shRNA abundances in the pLKO.005 vector matched the distribution of shRNA abundances in the pLKO.1 vector in each sample. Abundance of each shRNA in the low and high glucose populations was determined relative to the abundance of that shRNA in the genomic DNA from the initial sample (obtained before Nutrostat culture). The enrichment or depletion of each shRNA in the low versus high glucose populations as compared to the initial population was then determined. For example, an shRNA might be depleted 8-fold in the 0.75 mM condition, but only 2-fold in the 10 mM condition. This would reflect a small requirement in the 10 mM condition for the gene product encoded by the gene inhibited by that shRNA, but a much larger requirement for that gene product in the 0.75 mM condition. Each shRNA hairpin was assigned a score as follows: Hairpin score (HS)=|10 mM Log 2 Effect Score−0.75 mM Log 2 Effect Score|>0.75. Thus, individual shRNAs were identified as differentially scoring in high glucose versus low glucose using a Log 2 fold change cutoff of −0.75 (high glucose versus low glucose). Hairpin scores that were not >0.75 were disregarded. The screen was performed 3 times. Gene hit criteria were: (HS1+HS2+HS3)/3>0.33. In other words, for each comparison, genes were considered hits if >33% of the shRNAs targeting that gene scored when averaging across all replicates. These data are depicted for each shRNA in FIG. 12 and a summary of hits is presented.

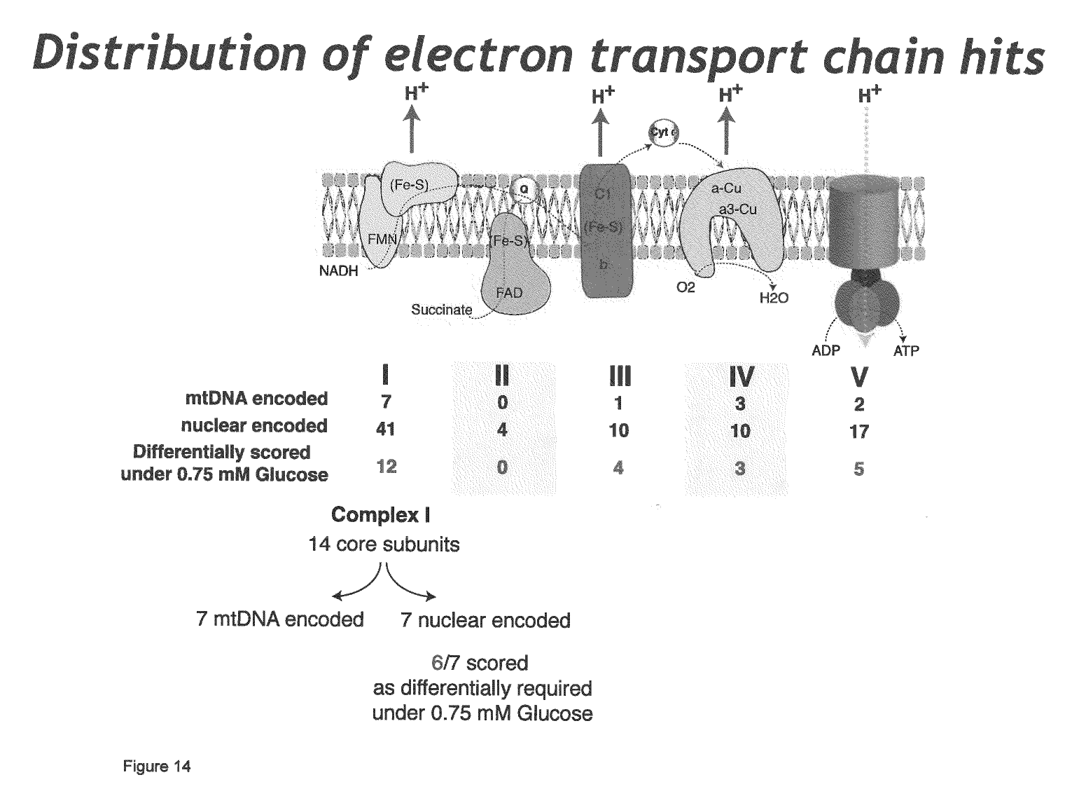

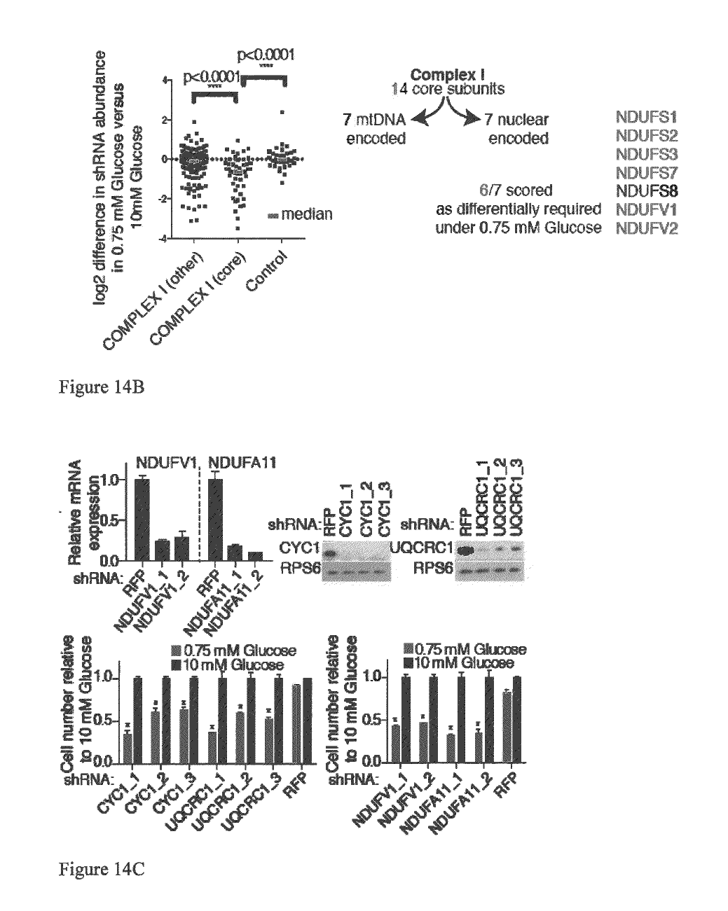

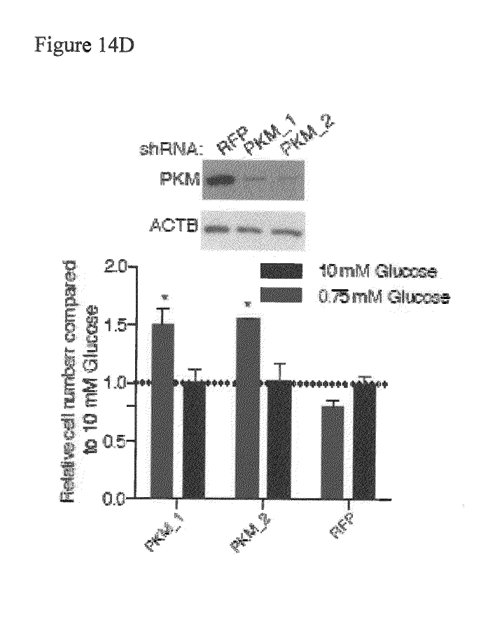

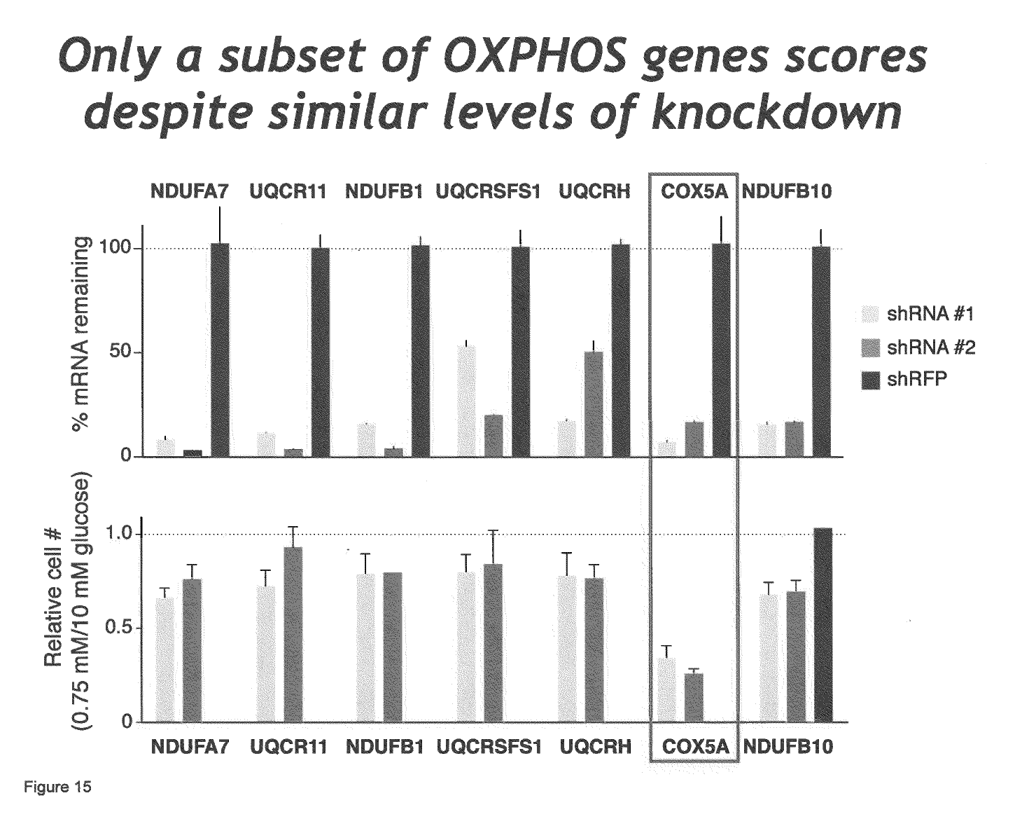

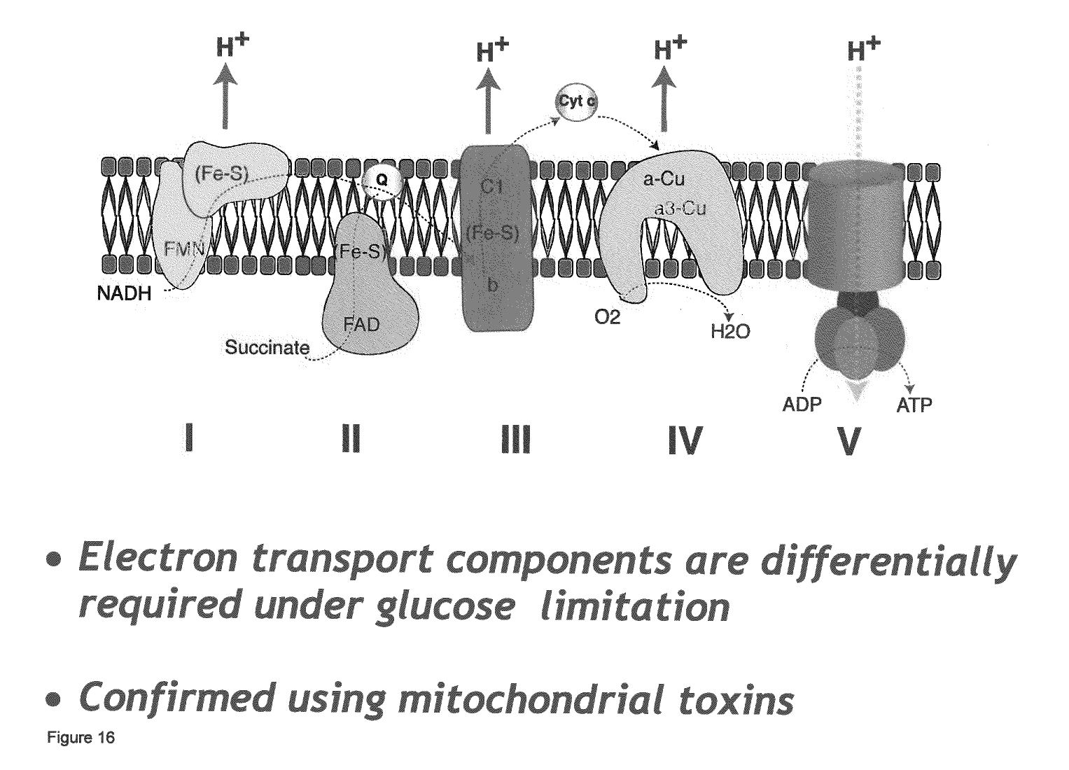

ShRNAs that scored as hits and were relatively less abundant in cells grown at 0.75 mM than in cells that were cultured in 10 mM glucose were considered to score “positive” in the screen as corresponding to genes that are differentially essential for proliferation in low glucose (i.e., loss of expression of the genes targeted by these ShRNAs reduced the ability of cells to proliferate in low glucose as compared with their ability to proliferate under standard (high) glucose conditions). These genes are listed in the right column of FIG. 13 and in Table 1. The screen identified a number of components of mitochondrial oxidative phosphorylation complexes I, III, IV, and V as being differentially essential for proliferation in low glucose. For example, six of seven nuclear encoded Complex I components (conserved between mammals and bacteria⁹), scored as differentially required for proliferation in low glucose (FIG. 14(A)), a significant enrichment compared to non-core subunits (p<0.0012, FIG. 14(B)). (Although not indicated on the figure, PISD and ACAD9 are also genes that are involved in mitochondrial OXPHOS. ACAD9 has recently been showed to be a member of Complex I.) As indicated on FIG. 13, PISD and ACAD9 also scored, as did SLC2A1, the gene encoding the GLUT1 glucose transporter. Only a subset of OXPHOS genes scored despite similar levels of knockdown (exemplified in FIG. 15). Pathways scoring as preferentially required in low glucose are Complex I (p<9.3×10⁻⁴⁹), III (p<6.6×10⁻²⁰), IV (p<8.3×10⁻¹⁰) and V (p<5.6×10⁻¹⁹). The top 1-2 genes scoring by “% shRNAs scoring” from Complex I, III and IV were further validated and are reported in FIG. 14(C) (Complex I genes NDUFV1 and NDUFA11, and Complex III genes CYC1 and UQCRC1). Complex I was followed up, e.g., as described below, e.g., Example, 9 and Example 12 using a specific inhibitor (phenformin). The differential requirement for various electron transport chain components under low glucose conditions was also confirmed using mitochondrial toxins (Example 4). ShRNAs that scored as hits and were relatively less abundant in cells cultured at 10 mM than in cells cultured at 0.75 mM glucose were considered to score “positive” in the screen as corresponding to genes that are differentially essential for proliferation in standard (high) glucose (i.e., loss of expression of the genes targeted by these ShRNAs reduced the ability of cells to proliferate in high glucose as compared with their ability to proliferate in low glucose). These genes are listed in the left column of FIG. 13 and in Table 2. See also FIG. 14(D). The screen identified a number of genes encoding proteins involved in glycolysis as being differentially required for proliferation in high glucose. These glycolytic genes may be required for optimum utilization of glucose in high glucose conditions and may become less important for optimal growth under lower glucose conditions.

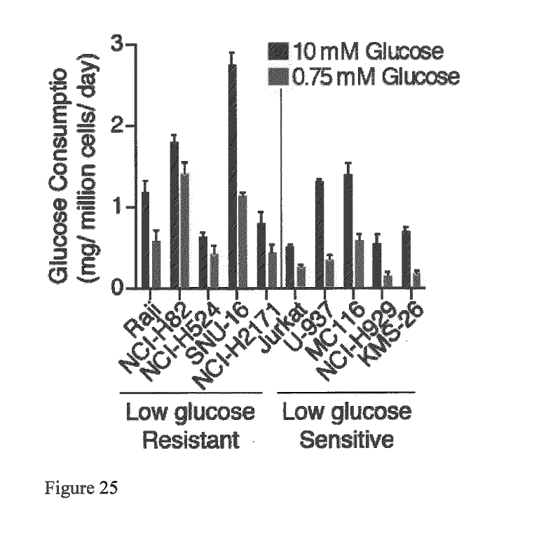

Overall, we identified 28 and 36 genes whose suppression preferentially inhibited cell proliferation in high or low glucose, respectively (FIG. 13). Genes selectively required in 10 mM glucose were enriched for glycolytic genes (GAPDH, ALDOA, PKM, ENO1; p<8.6×10⁻⁷). Genes selectively required under 0.75 mM glucose consisted almost exclusively of the nuclear-encoded components of the mitochondrial oxidative phosphorylation (OXPHOS) complexes I, III, IV and V (FIG. 12. FIG. 14). Two genes required for OXPHOS function, ACAD9 and PISD^{9,10}, also scored, as did SLC2A1, the gene encoding the GLUT1 glucose transporter. Short-term individual assays validated that efficient suppression of top scoring OXPHOS genes selectively decreased proliferation under low glucose, while hairpins targeting non-scoring OXPHOS genes did so to a significantly lesser extent (FIG. 25). Thus, a screen of metabolic genes pinpointed OXPHOS as the key metabolic process required for optimal proliferation of cancer cells under glucose limitation. Alternative methods for identifying hits from RNAi based screens were employed using the GENE-E program⁸ (Broad Institute). Gene scores and P values were calculated using the Kolmogorov-Smirnov method, using the weighted sum of the top two scoring hairpins, or using the Second best scoring hairpin. These alternative methods all identify highly significant numbers of Complex I, III, IV and V genes as being differentially essential in 0.75 mM glucose.

### Example 3

### Cancer Cell Lines Exhibit Diverse Responses to Glucose Limitation

We designed a system to mark individual cell lines with stable DNA barcodes so that multiple cell lines could be cultured together under the same conditions. We constructed a lentiviral plasmid library that consisted of 90 DNA barcodes. Upon stable integration, this barcode introduces a unique, identifiable, and heritable mark into the genome that permits tracking the proliferation of individual cancer cell lines among a mixed group. To mark individual cell lines with DNA barcodes, a unique seven base pair sequence was transduced into cells using lentiviruses produced from a pLKO.1P vector into which the following sequence was cloned utilizing the following primers, which had been annealed and ligated to an AgeI and EcoRI restriction enzyme cut vector:

Sequence inserted (‘N’s indicate location of cell-specific barcode sequence):

TTTTAGCACTGCCNNNNNNNCTCGCGGGCCGCAGGTCCAT
Primers:
TOP:
CCGGTTTTTAGCATCGCCNNNNNNNCTCGCGGCCGCAGGTCCATG
BOTTOM:
AATTCATGGACCTGCGGCCGCGAGNNNNNNNGGCGATGCTAAAAA

The sequence of individual lentiviral vectors was determined by Sanger sequencing and vectors containing unique sequences were chosen for transduction into cell lines. Each cell line was infected with three barcodes in separate infections so that the proliferation of each cell line could be measured three times independently in a single experiment. Proliferation assays of the individually barcoded cell lines verified that the barcodes did not affect cell proliferation in short term assays. To perform the cell competition assays, all of the barcoded cell lines were mixed in equal proportion with bias for slower proliferating cell lines being over-represented in the initial population. The Nutrostats were inoculated with 5M pooled cells at 10 mM or 0.75 mM glucose concentrations and the proliferation and glucose consumption of the culture carefully monitored to adjust for any time dependent changes in the per cell glucose consumption rate. After 15 population doublings, cells were harvested for genomic DNA isolation and processed for deep sequencing as described above. Barcode abundance was determined in the starting population or after 15 population doublings, and the fold change in barcode abundance relative to the abundance of Jurkat cell line barcodes was calculated. Based on the number of population doublings of the entire culture and the known doubling time of the Jurkat cell line, the doubling time (hours) of each cell line in the mixture was calculated according to the following formula:

293 hours/(Log₂ FC_{cell line}−Log₂ FC_{Jurkat}+PD_{Jurkat})

where 293 hours is the duration of the experiment, Log₂ FC_{cell line} is the Log₂ Fold change in abundance of barcode for the given cell line in the final sample compared to the initial, Log₂ FC_{Jurkat} is the Log₂ fold change for the Jurkat cell line, and PD_{Jurkat} is the empirically determined number of population doublings that the Jurkat cell line underwent during 293 hours (i.e. 12.2 doublings in 10 mM glucose and 11.3 doublings in 0.75 mM glucose conditions).

Using this system, we grew a mixed panel of individually barcoded cancer cell lines at low glucose levels and simultaneously identified the growth abilities upon glucose limitation. This system allowed us to stratify precisely the relative sensitivities of these cell lines to high versus low glucose. The system could also be used in a similar manner with other nutrients or substances (e.g., toxins, established or potential chemotherapeutic agents).

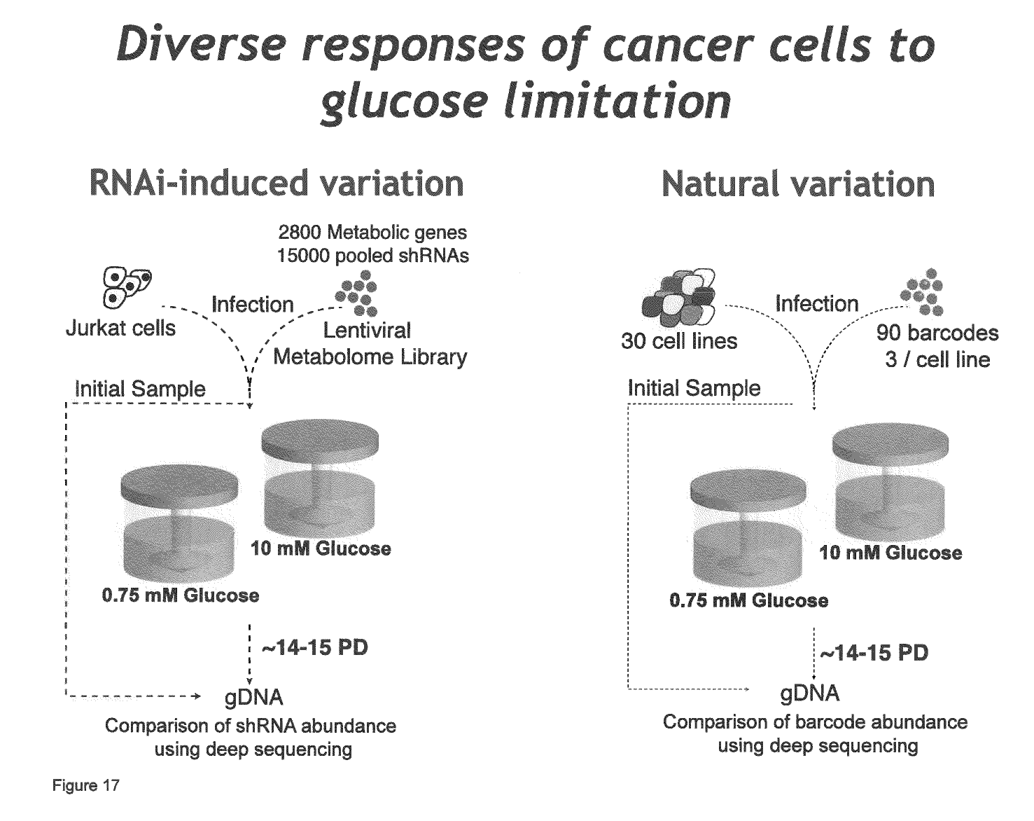

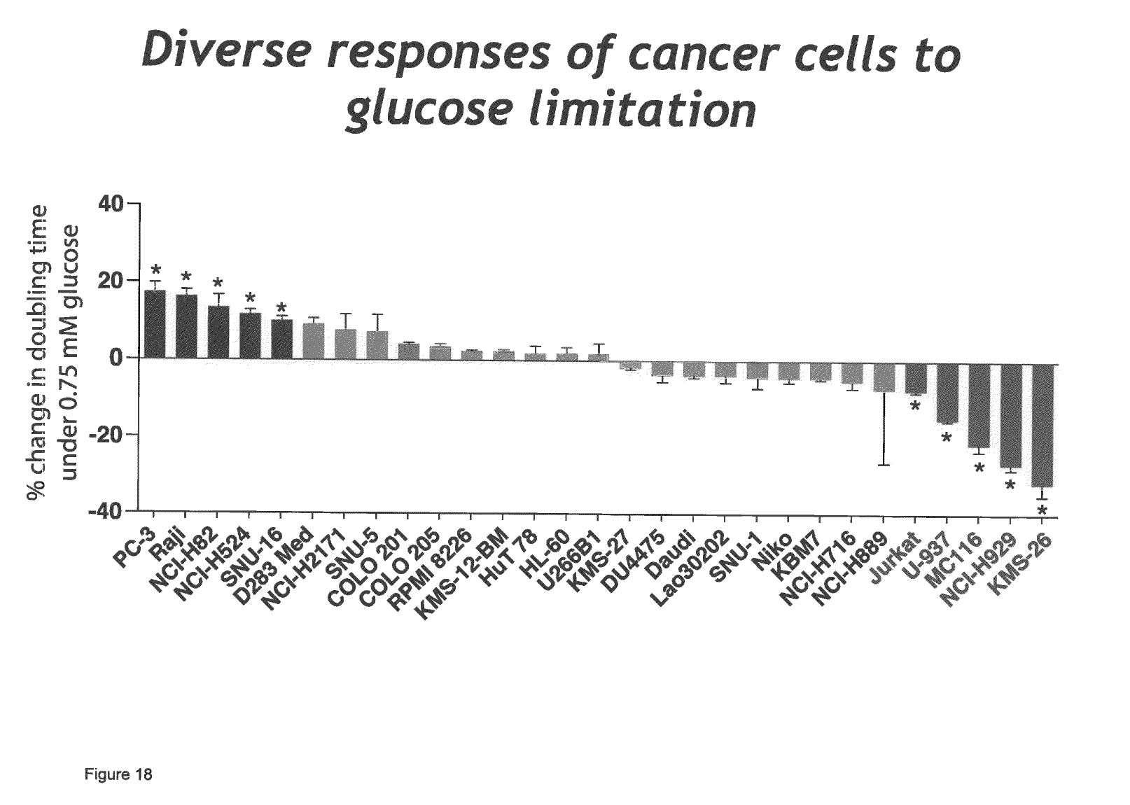

A schematic diagram of our approach to stratify the relative sensitivities of cell lines to high versus low glucose is shown in FIG. 17. We cultured an equal number of cells from each of about 30 different cancer cell lines of diverse cancer types together in a Nutrostat under either 0.75 mM glucose or 10 mM glucose conditions for 14-15 population doublings. We then harvested genomic DNA from the cells from each culture and determined abundance of each barcode using deep sequencing. This allowed us to order the cell lines according to their ability to proliferate under the different culture conditions. We calculated the % doubling time of each cell line under 0.75 mM glucose conditions and ordered them accordingly. Results are shown on FIG. 18. Some cell lines (e.g., PC-3, Raji, NCI-H82, NCI-H524, SNU-16) exhibited an increased ability to proliferate in 0.75 mM glucose as compared with 10 mM glucose. These cell lines (and others whose doubling time did not decrease in 0.75 mM glucose) were deemed resistant to glucose limitation. Other cell lines (e.g., Jurkat, U-937, MC116, NCI-H929. KMS-26) exhibited a decreased ability to proliferate in 0.75 mM glucose as compared with 10 mM glucose. These cell lines (and others whose doubling time decreased in 0.75 mM glucose) were deemed sensitive to glucose limitation.

### Example 4

### Studies with Mitochondrial Toxins Confirm Importance of OXPHOS Components for Growth Under Glucose Limitation

To confirm the shRNA experiments described above (Example 2) showing that mitochondrial components are essential for growth in low glucose, we treated cells in high and low glucose with various mitochondrial toxins targeting these same components. The result was similar to that using the shRNAs, namely that inhibition of these mitochondrial complexes was more toxic under low glucose specifically in glucose limitation sensitive cancer cell lines identified in Example 3.

### Example 5

### Expression Levels of CYC1 and UQCRC1 in Tumor Cells Predicts Sensitivity to Glucose Limitation

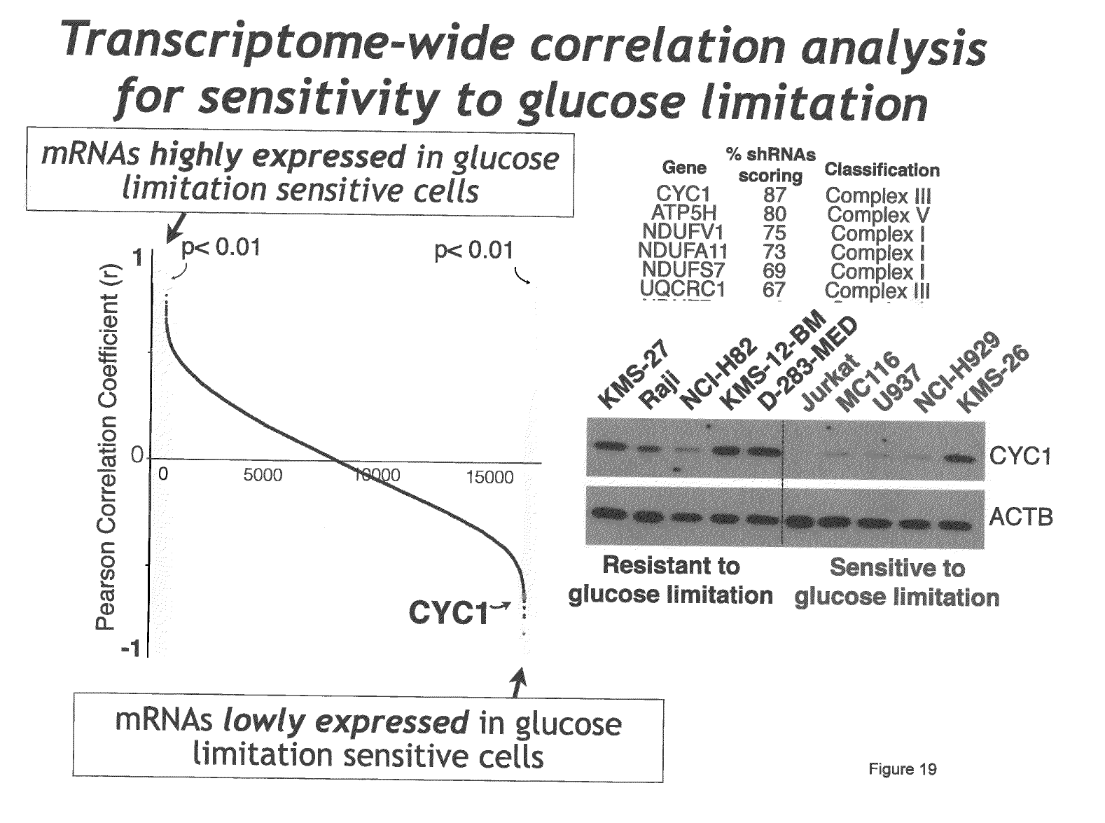

We performed transcriptome-wide correlation analysis for sensitivity to glucose limitation using publicly available steady state gene expression data for the various cell lines (e.g., glucose limitation sensitive or glucose limitation resistant). This allowed us to identify mRNAs that are highly expressed or expressed at low levels in glucose limitation sensitive cells (FIG. 19). We identified two mitochondrial genes, CYC1 and UQCRC1, as being strongly associated with sensitivity to low glucose. Expression of these genes was absent or very low in most glucose restriction sensitive cell lines as compared with expression in cell lines that were resistant to glucose limitation. The absent or low expression of CYC1 in cell lines that were sensitive to glucose restriction was confirmed at the protein level by Western blot (FIG. 19). CYC1 and UQCRC1 were among the strongest scoring genes in the screen described in Example 2, suggesting a causal relationship between reduced expression of CYC1 and UQCRC1 and glucose sensitivity. Exemplary results of correlation analysis across 25 cell lines are presented in Table 3.

TABLE 3
Correlation of Sensitivity to Low Glucose with
Gene Expression Across 25 Cell Lines

| GENE | PEARSON CORRELATION (“R”) |
|---|---|
| RHOV | 0.803 |
| ARMC7 | 0.726 |
| ADAMTS16 | 0.715 |
| CPSF3L | 0.683 |
| PUF60 | 0.678 |
| SSPN | 0.677 |
| C20orf27 | 0.652 |
| DVL1 | 0.650 |
| OTUB1 | 0.647 |
| MRPL49 | 0.643 |
| SLC25A39 | 0.641 |
| DNAJC11 | 0.641 |
| MUC3A | 0.636 |
| CYC1 | 0.632 |
| MFN2 | 0.623 |
| RIN2 | 0.622 |
| NUP85 | 0.621 |
| PAX7 | 0.620 |
| B4GALT2 | 0.619 |
| SLC27A4 | 0.618 |
| UQCRC1 | 0.618 |
| SMPDL3B | 0.616 |
| ZPBP | 0.614 |
| CYHR1 | 0.613 |
| MPZL1 | 0.613 |

### Example 6

### Basis for Cancer Cell Sensitivity to Glucose Limitation

We explored the possibility that variations in mitochondrial DNA (mtDNA) amount or mitochondrial mass could explain the differential sensitivity of cell lines to glucose limitation. MitoTracker® Green was used to measure mitochondrial mass. 2×10⁵ cells were incubated directly with 50 nM Mitotracker Green FM (Invitrogen M7514) in RPMI for 40 minutes at 37° C. Cells were then centrifuged at 4,000 rpm for 5 minutes at 4° C. and the overlying media removed. Cells were kept on ice, washed once with ice-cold PBS, and resuspended in ice-cold PBS with 7-AAD (Invitrogen A1310) for FACS analysis of live cells. The mean Mitotracker Green fluorescence intensity was used as a measure of relative mitochondrial mass. For copy number, total DNA was isolated using the QIAamp DNA Minikit and real-time PCR was used to estimate relative differences in mtDNA copy number between different cell lines. Alu repeat elements were used as controls. Primers used were:

ND1_F/R:
CCCTAAAACCCGCCACATCT/GAGCGATGGTGAGAGCTAAGGT
ND2_F/R:
TGTTGGTTATACCCTTCCCGTACTA/CCTGCAAAGATGGTAGAGTAGATGA
Alu_F/R:
CTTGCAGTGAGCCGAGATT/GAGACGGAGTCTCGCTCTGTC

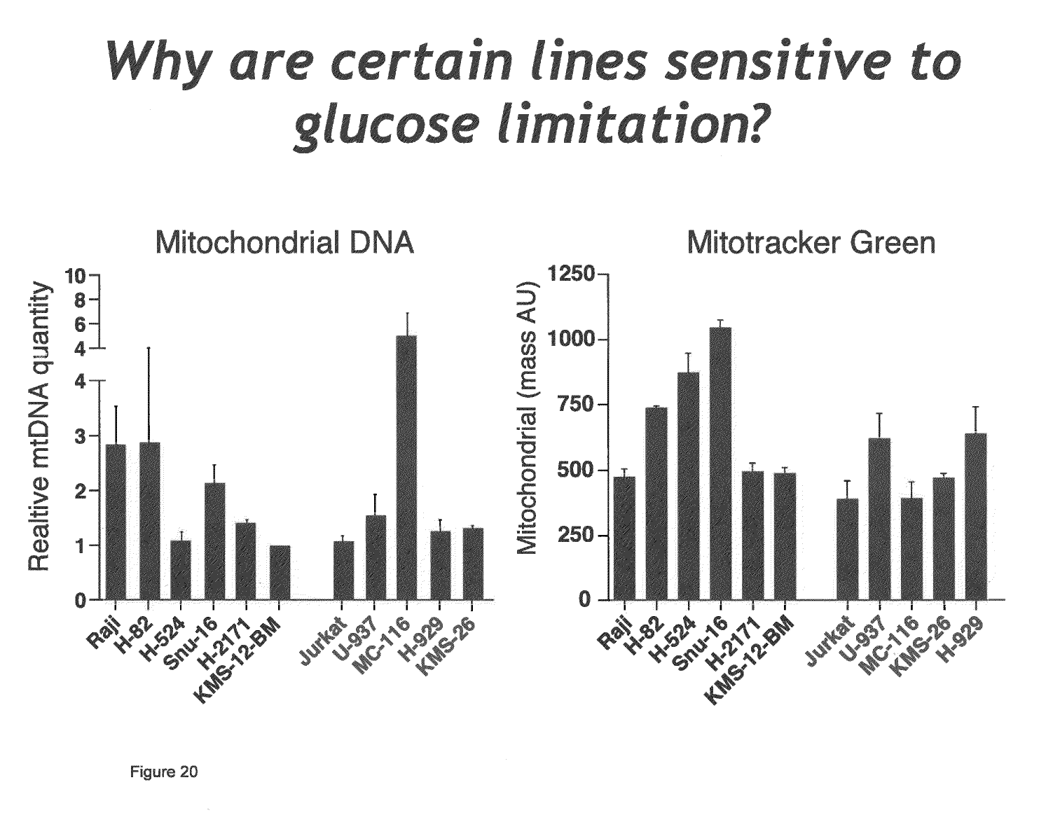

Based on the results obtained (FIG. 20), it appears that variations in mtDNA amount or mitochondrial mass do not correlate with the glucose limitation sensitive phenotype.

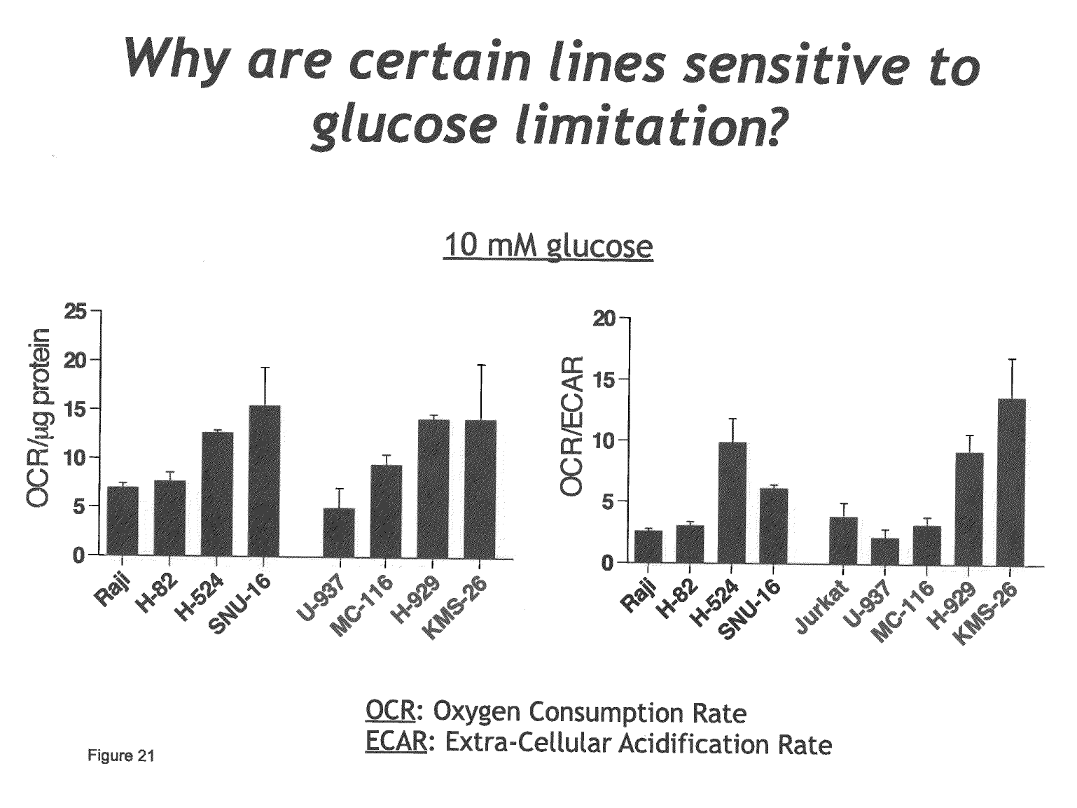

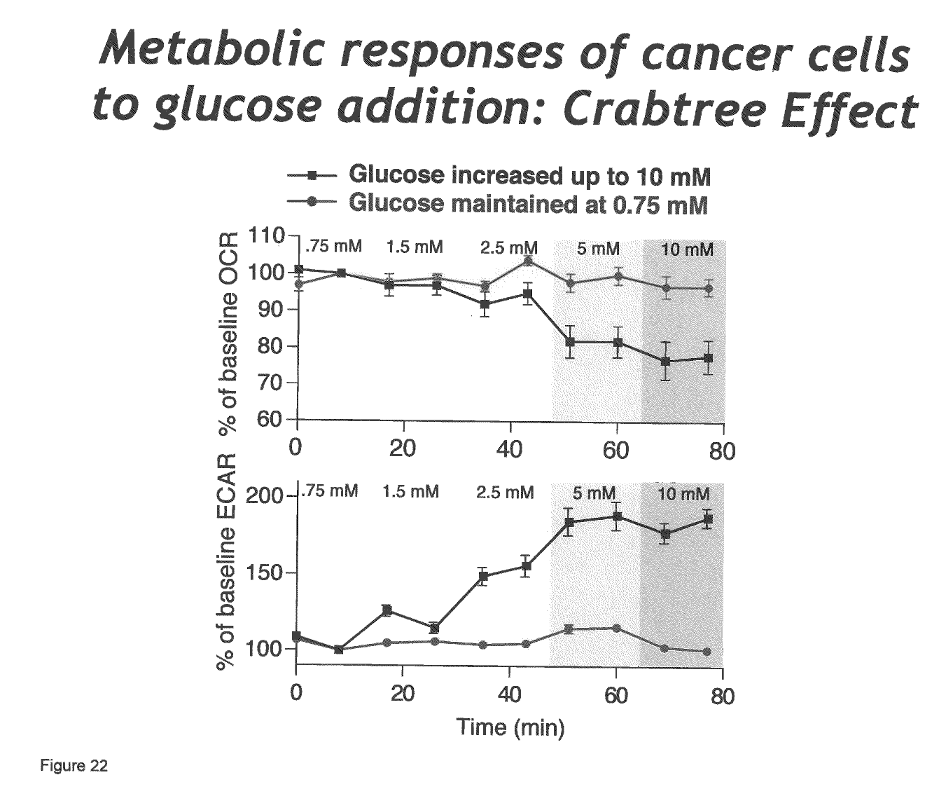

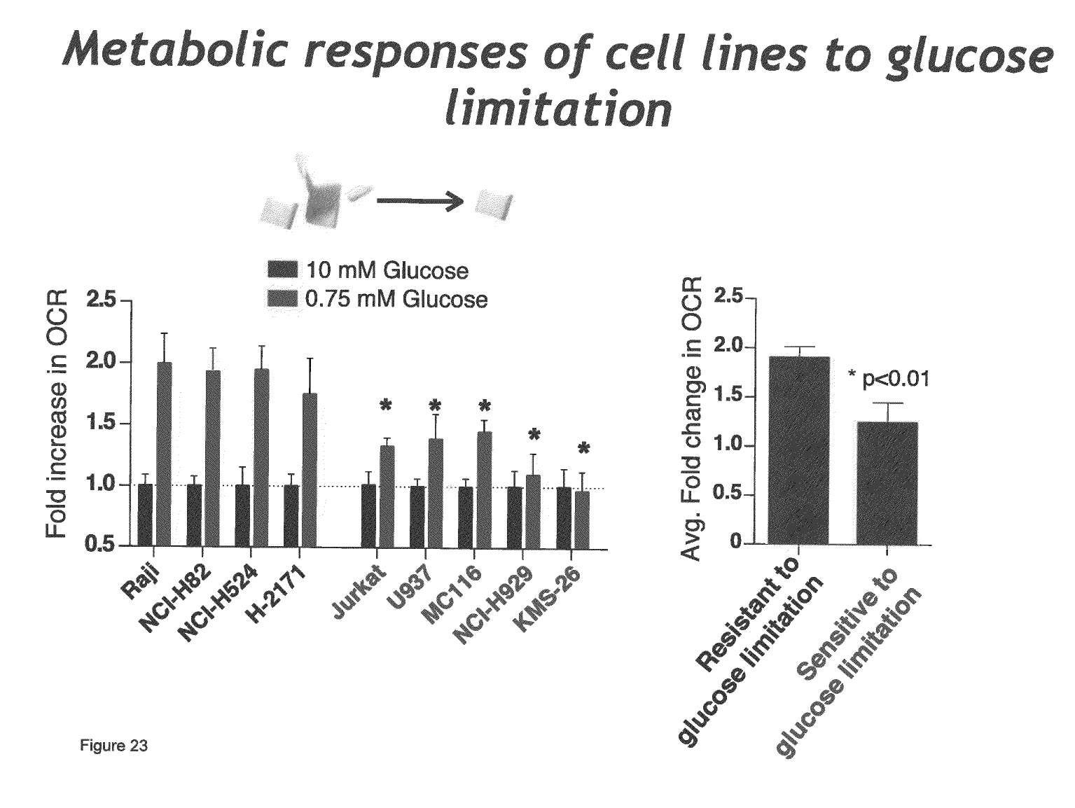

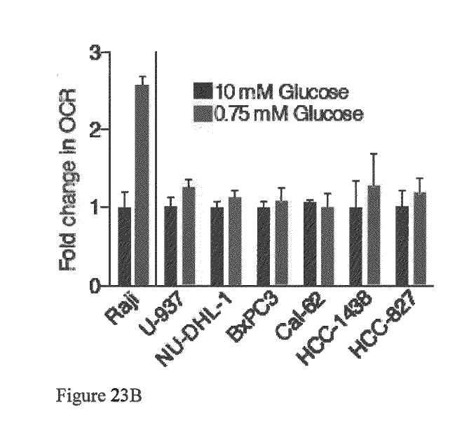

We measured the oxygen consumption rate (OCR) and extracellular acidification rate (ECAR) using an X24 Extracellular Flux Analyzer (Seahorse Bioscience, Billerica, Mass.) and determined the OCR/ECAR ratio for glucose limitation sensitive cell lines and glucose limitation resistant cell lines at 10 mM glucose and did not find significant differences between the two groups (FIG. 21). We found that the Crabtree effect (a phenomenon that has been well established in yeast whereby additional glucose increases glycolysis and suppresses oxidative phosphorylation) is operative in Jurkat cells at 10 mM glucose (FIG. 22). We examined the change in OCR upon shifting cells from 10 mM glucose to 0.75 mM glucose. As shown in FIG. 23, on average the glucose limitation sensitive cell lines showed a reduced ability to increase their OCR upon transfer to low glucose as compared with glucose limitation resistant cell lines. The finding that low glucose sensitive cell lines upregulated OCR less than the resistant ones was further confirmed using additional cell lines (FIG. 23(B)).

Oxygen consumption of intact or permeabilized cells (Example 8) was measured using an XF24 Extracellular Flux Analyzer (Seahorse Bioscience) as follows: For suspension cells, Seahorse plates were coated with Cell TAK (BD, 0.02 mg/ml in 0.1 M NaHO3) for 20 minutes to increase adherence of suspension cells. 250,000 cells then were attached to the plate by centrifugation at 2200 rpm without brakes for 5 min. For adherent cells, 40,000 to 80,000 cells were plated the night before the experiments. RPMI 8226 (US biological #9011) was used as the assay media for all experiments with the indicated glucose concentrations in the presence of 2 mM Glutamine without serum. For spare respiratory capacity measurements, increasing FCCP concentrations (0.1, 0.5 and 2 uM) were used in order to assess maximum OCR of each cell line. For basal oxygen consumption measurements, cell number or protein concentration was used for normalization.

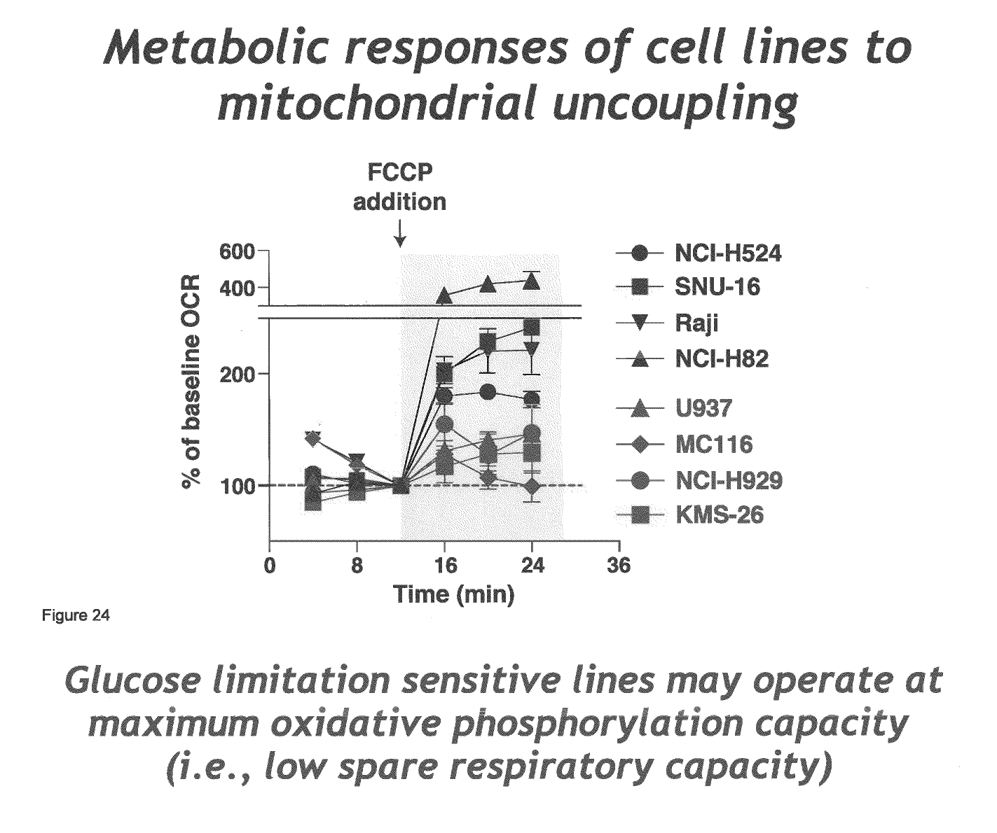

Metabolic responses of cell lines to mitochondrial uncoupling were analyzed by treating cells from a number of glucose limitation sensitive or resistant cell lines with the mitochondrial uncoupling agent FCCP and measuring OCR. Results are presented in FIG. 24. Glucose limitation sensitive cells exhibited only a modest increase their OCR in response to mitochondrial coupling, in contrast to the results observed in glucose limitation resistant cell lines, which generally exhibited a much greater increase in OCR. The results suggest that glucose limitation sensitive lines may operate at maximum oxidative phosphorylation capacity (i.e., low spare respiratory capacity). Thus, they may rely relatively more on glycolysis for their energy needs and may be more greatly affected by limited glucose availability than cell lines that have higher spare respiratory capacity.

### Example 7

### Defective Glucose Uptake in Certain Cancer Cell Lines can Account for Sensitivity to Glucose Limitation

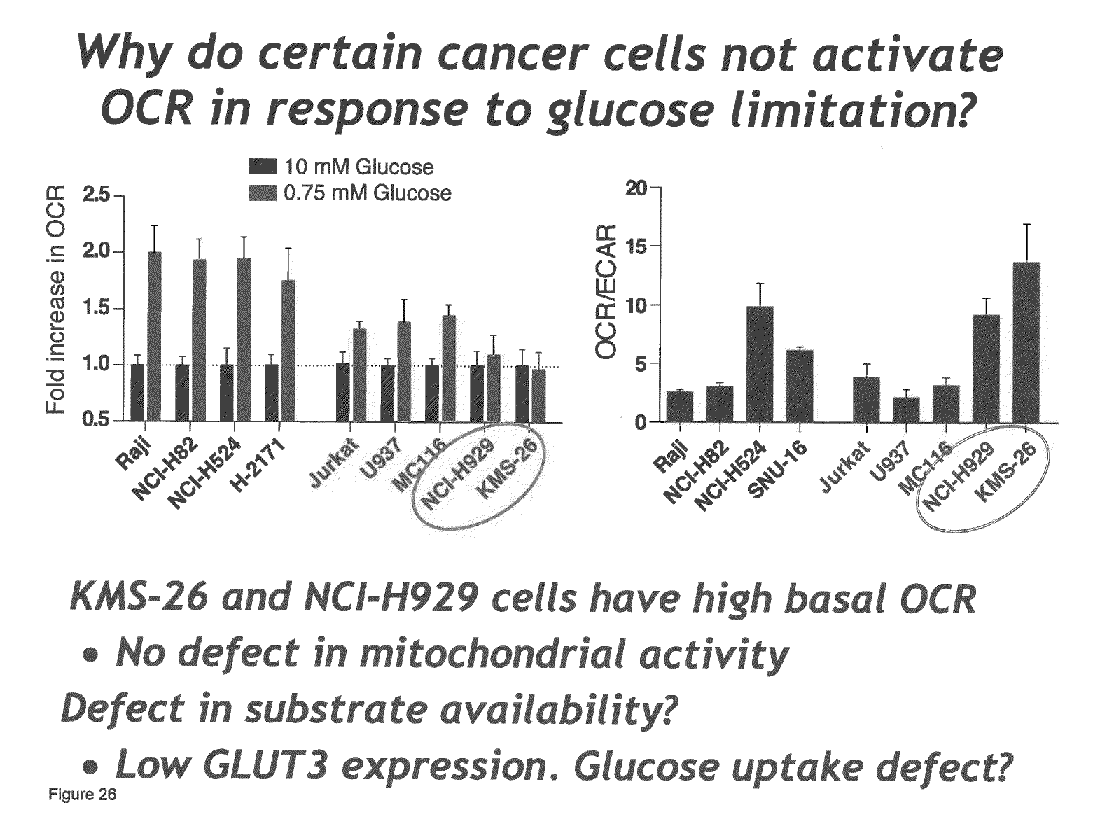

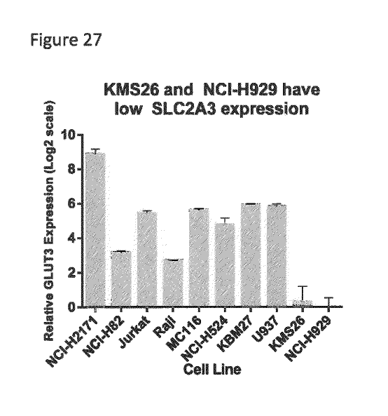

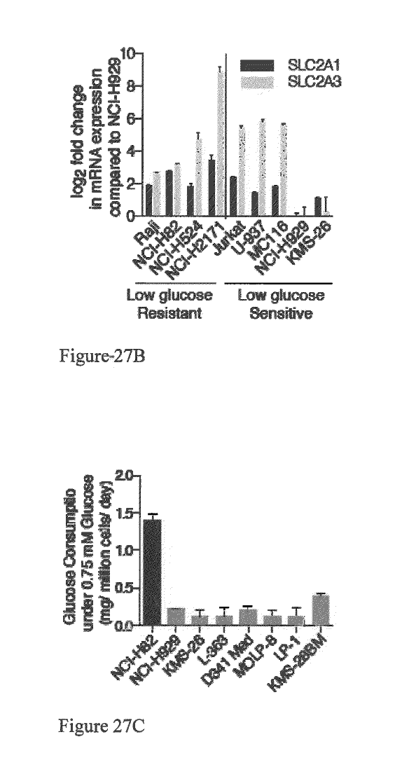

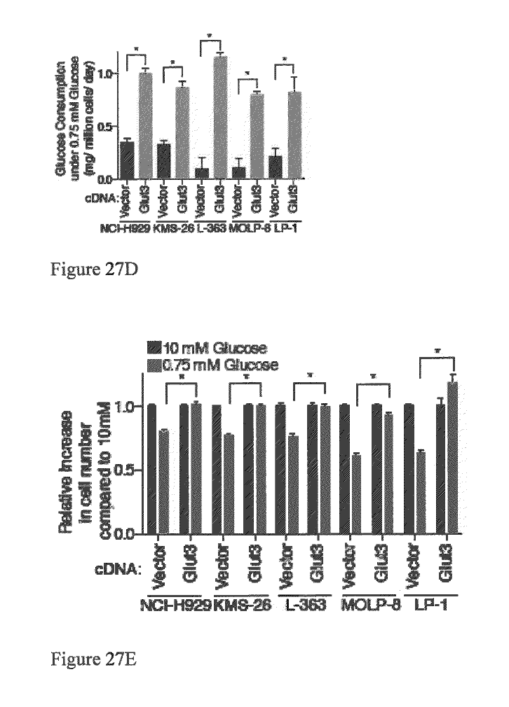

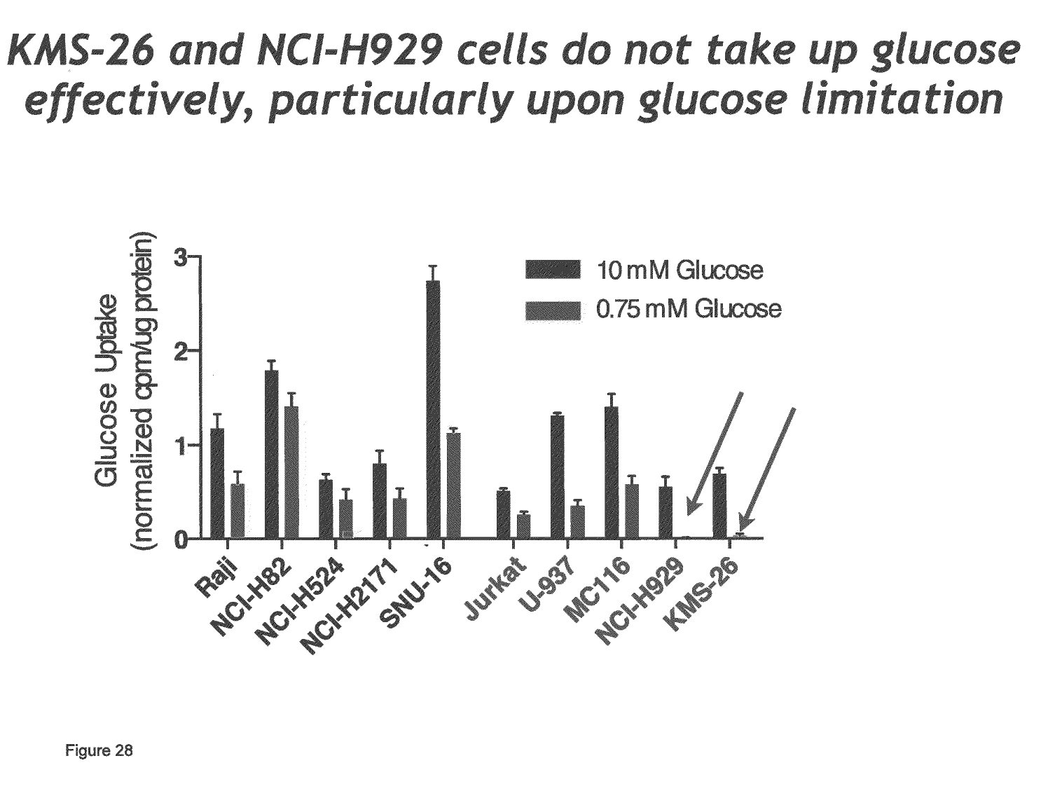

We sought to determine why certain cancer cells do not activate OCR in response to glucose limitation. We considered substrate availability as a potential cause. We found that KMS26 and NCI-H929 cells, both of which were identified as sensitive to glucose limitation, have high basal OCR at 10 mM glucose and did not exhibit a defect in mitochondrial activity (FIG. 26). We found, however, that these cell lines exhibited low expression of GLUT3 (solute carrier family 2, facilitated glucose transporter member 3 (SLC2A3). FIG. 27 shows expression of SLC2A3 in various cancer cell lines relative to expression in NCI-H929 cells. This finding suggested to us that these cell lines may have a defect in glucose uptake. We confirmed that KMS26 and NCI-H929 cells have defective glucose consumption (FIG. 25) and do not take up glucose effectively, particularly upon glucose limitation (FIG. 28). The low expression of the GLUT3 and GLUT1 glucose transporters in low glucose sensitive cell lines was verified in additional cell lines by qPCR (FIG. 27(B)), Real-time qPCR was performed as follows: RNA was isolated using the RNeasy Kit (Qiagen) according to the manufacturer's protocol. RNA was spectrophotometrically quantified and equal amounts were used for cDNA synthesis with the Superscript II RT Kit (Invitrogen). To isolate genomic and mitochondrial DNA we used the Blood and Tissue Kit (Qiagen). qRT-PCR or qPCR analysis of gene expression or copy number was performed on a ABI Real Time PCR System (Applied Biosystems) with the SYBR green Mastermix (Applied Biosystems). All primers were designed using the Primer3 software and aligned to the human reference genomes using blast to verify their specificity. The primers used for GLUT3 and GLUT1 are as follows GLUT1_F/R: tcgtcggcatcctcatcgcc/ccggttctcctcgttgcggt; GLUT3_F/R: ttgctcttcccctccgctgc/accgtgtgcctgcccttcaa. Results were normalized to RPL0 levels.

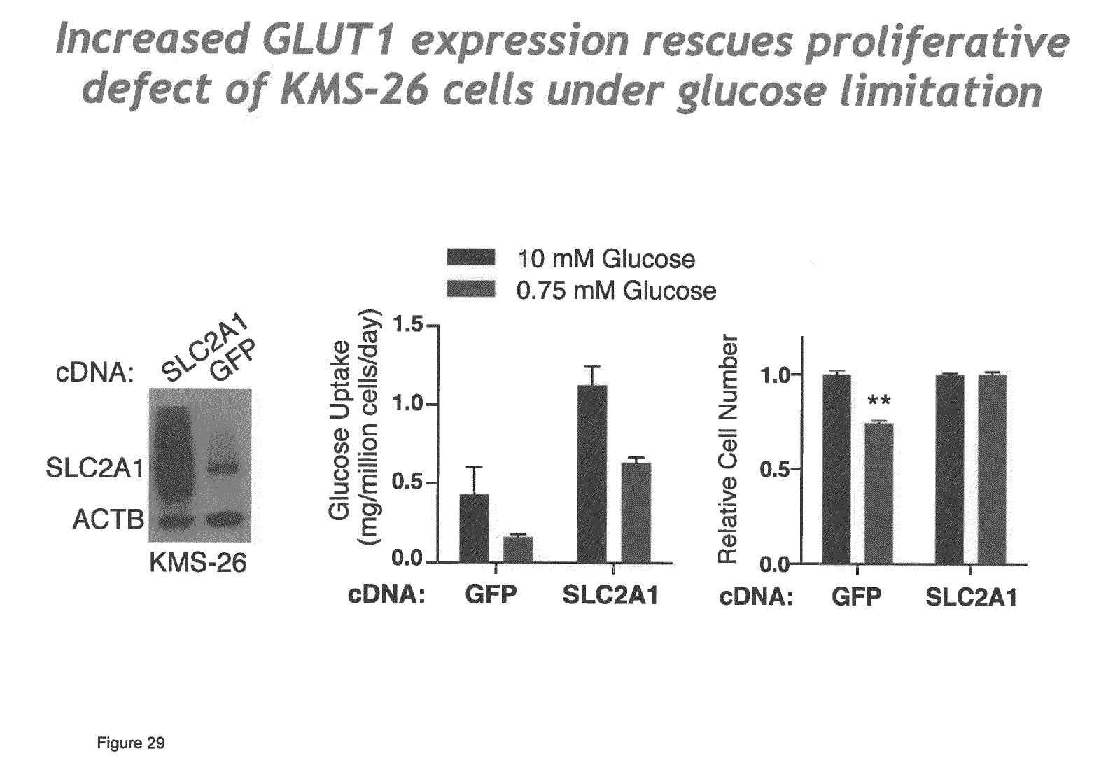

We expressed the glucose transporter GLUT1 (SLC2A) in KMS26 cells to determine whether increased glucose transporter expression could rescue the proliferative defect of KMS26 cells under glucose limitation. FIG. 29 (left side) is a Western blot showing greatly increased expression of SLC2A1 following introduction of SLC2A1 cDNA into KMS26 cells relative to KMS-26 cells into which a cDNA encoding GFP was introduced as a control. Increased GLUT I expression significantly increased glucose uptake under both low (0.75 mM) and high (10 mM) glucose conditions, as shown in the plot to the left of the Western blot in FIG. 29. As shown in the plot on the right side of FIG. 29, we found that increased GLUT expression does indeed rescue the proliferative defect that KMS26 cells exhibit under glucose limitation.

We expressed GLUT3 (SLC2A3) in KMS26 cells using a retroviral vector to determine whether increased expression of this glucose transporter could rescue the proliferative defect of KMS-26 cells under glucose limitation. The retroviral SLC2A3 vector was generated by cloning into the BamHI and EcoRI sites of the pMXS-ires-blast vector a cDNA insert generated by PCR from a cDNA from Open Biosystems (cat # MHS1010-7429646) using the primers below, followed by standard cloning techniques:

SLC2A3 Bam HI F:
GCA TGG ATC CAC CAT GGG CAC ACA GAA GGT
CAC
SLC2A3 Mfel R:
GCA TCA ATT GTT AGA CAT TGG TGG TGG TCT CC

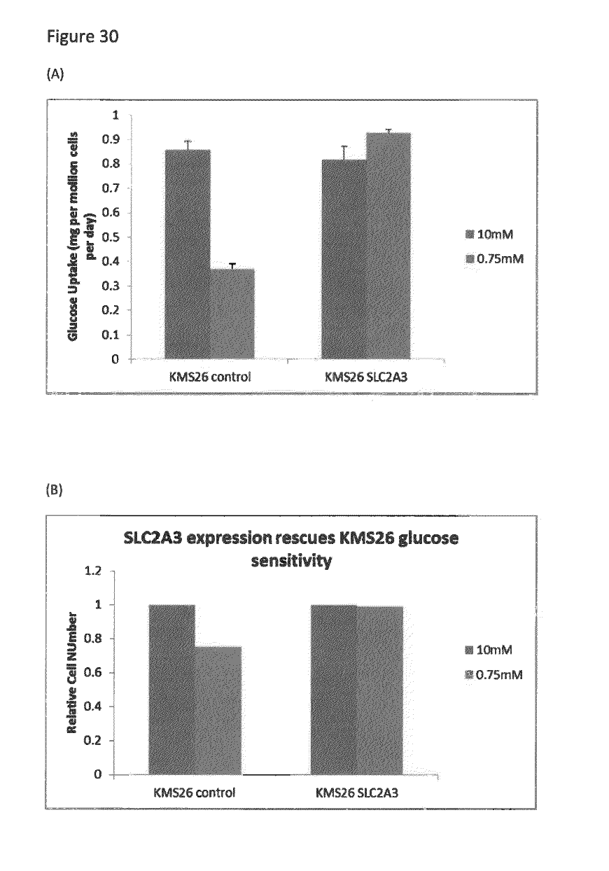

Increased GLUT3 expression significantly increased glucose uptake under low (0.75 mM) glucose conditions, as shown in FIG. 30(A). As shown in FIG. 30(B), we found that increased GLUT3 expression does indeed rescue the proliferative defect that KMS26 cells exhibit under glucose limitation.

We found that the shRNAs designed to inhibit GLUT3 that were present in the shRNA pool tested in the screen described in Example 2 were actually poor inhibitors of GLUT3 expression, which may explain why they did not score as hits in the screen.

Thus, we conclude that defective glucose transport due, for example, to reduced or absent expression of one or more glucose transporters, can result in the failure of certain cancer cells to activate OCR in response to glucose limitation and at least partly account for the sensitivity to glucose limitation of such cancer cells. Expression of the glucose transporter SLC2A1 or SLC2A3 increases glucose import and prevents these cells from being sensitive to glucose limitation, as shown in FIGS. 29 and 30. In particular. SLC2A3 is of interest in that expression is variable in cancer cell lines and because SLC2A3 is a high affinity (low Km) transporter of glucose compared to SLC2A1, and the cell lines in question (KMS26 and NCI-H929) exhibit particularly low levels of SLC2A3 (see below). We propose that cell lines which do not express SLC2A3 are unable to take up glucose upon glucose limitation and therefore are sensitive to OXPHOS inhibition, e.g., by compounds that inhibit OXPHOS (such as metformin) under these conditions. Cancers that have a high percentage (>20%) of cell lines exhibiting low SLC2A3 expression include, e.g., prostate, esophagus, breast, stomach, lung, and pancreas. Measurement of SLC2A3 expression in such cancers, e.g., by measuring SLC2A3 mRNA or protein in a sample obtained from the cancer) can be used to predict sensitivity to OXPHOS inhibition and identify patients with cancer who would benefit from treatment with OXPHOS inhibitors.

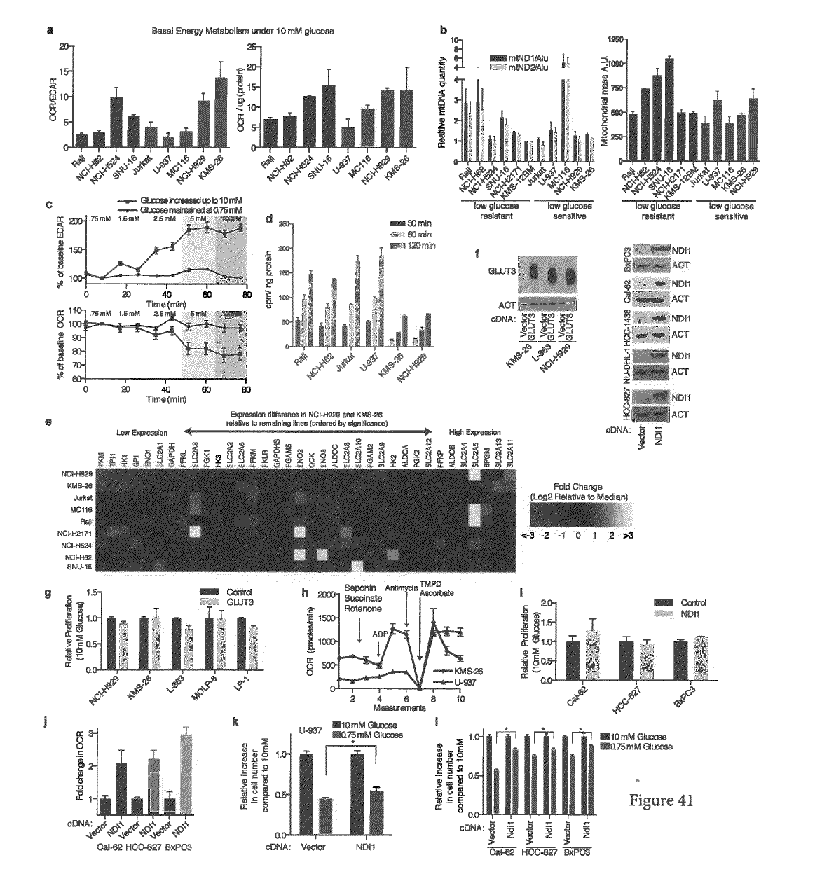

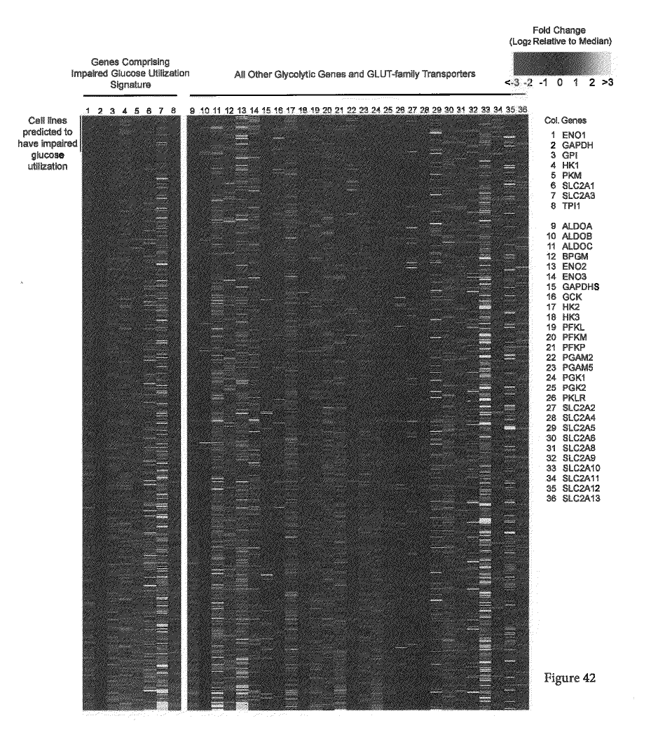

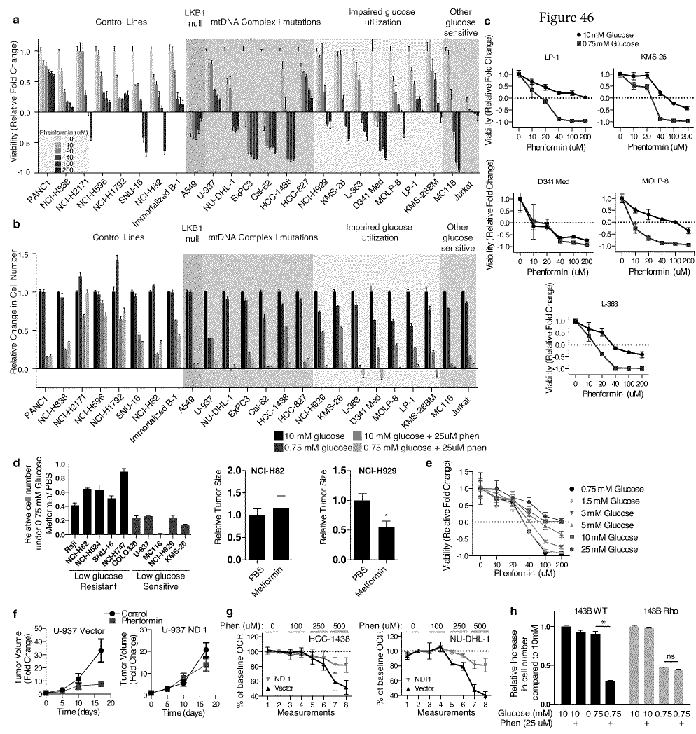

Analysis of publicly available gene expression data confirmed that these cell lines have low expression of the GLUT3 and GLUT1 glucose transporters, as well as lower levels of several glycolytic enzymes (FIG. 41e). Low expression levels of these genes constitute a gene expression signature indicative of low glucose utilization. The genes identified as comprising the impaired glucose utilization gene expression signature were ENO1, GAPDH, GPI, HK 1 PKM, SLC2A1, SLC2A3, and TPI1. Using gene expression data for 967 cell lines¹² we identified additional lines with this expression signature as described in the following paragraph (bioinformatics analysis) and obtained five of them (FIG. 42 and Table 6). In low glucose media, the five lines (LP-1, L-363, MOLP-8, D341 Med, KMS-28BM) had the predicted defect in glucose consumption and proliferation, like NCI-H929 and KMS-26 cells (FIG. 27(C) and FIG. 46b). In all cell lines tested (KMS-26, NCI-H929, L-363, LP-1, MOLP-8), GLUT3 over-expression was sufficient to rescue these phenotypes (FIGS. 27(D) and 27(E), FIG. 41f), while not substantially affecting proliferation in high glucose (FIG. 41g), arguing that a glucose utilization defect can account for why the proliferation of certain cancer cells is sensitive to low glucose. Low expression of ALDOA, PFKP, and PGK1 was also found to correlate with impaired glucose utilization.

The bioinformatic identification of cell lines with impaired glucose utilization described above was performed as follows: Gene expression data for all glycolytic genes and glucose transporters was compared between glucose utilization deficient cell lines (KMS-26 and NCI-H929) and all of the other cell lines, and those genes whose expression was significantly lower in the glucose utilization deficient lines were selected (SLC2A1, HK1, GAPDH, ENO1, GPI, TPI1, and PKM). SLC2A3 was also included as its expression was found to be significantly altered using qPCR. Log₂ transformed expression data for these eight genes was extracted for all 967 cell lines from the Cancer Cell Line Encyclopedia. For each cell line, we computed the difference between the expression level of each gene and the median expression level in all cell lines. These values were summed across all eight genes, and the cell lines were ranked in order of gene expression from lowest to highest (Table 6). Those cell lines included KMS-26 and NCI-H929, and from the other thirty cell lines with the lowest expression level of these genes, readily available lines were chosen.

Measurements of glucose consumption and uptake were performed as follows: Cells were plated in 10 mM or 0.75 mM glucose media at 5-20K cells per mL in 24 well plates in 1 mL media in replicates of four. Media was harvested after four days of culture and the number of cells counted. Harvested media was assayed by a glucose oxidase assay and the absorbance at 500 nm determined of assay buffer plus spent media, media from control wells containing no cells, or media containing no glucose, allowing the concentration of glucose in the spent media to be calculated according to Beer's Law. The mass of glucose consumed was normalized to the average number of cells present in the well, which was calculated by integrating the number of cells present during the course of the experiment over four days assuming simple exponential growth of the cells during the course of the experiment from the measured starting to final number of cells. For glucose import, cells were incubated in 0.75 mM glucose media overnight. The following day, Tritium-labeled 2-DG (5 μCi/mL, Moravek) in RPMI was added to 300,000 cells in fresh 0.75 mM glucose media. The import was stopped after 30, 60 and 120 min by the addition of cold HBSS containing the Glucose transporter inhibitor Cytochalasin B. The cells were next washed once with ice-cold HBSS and lysed in 400 μl RIPA buffer with 1% SDS. Radioactive counts were determined by a scintillation counter and scintillation reads were normalized to the total protein concentration of each sample.

### Example 8

### Glucose Limitation Sensitive Cell Line U937 has Defective Complex I and Complex II Activity

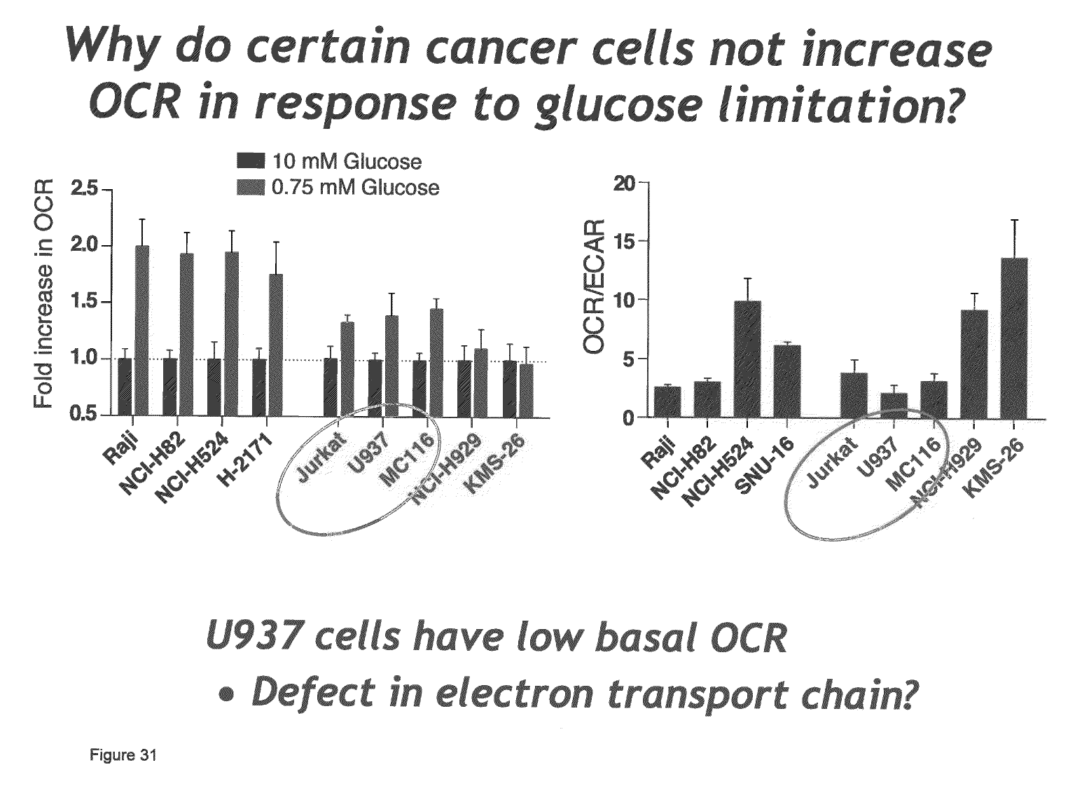

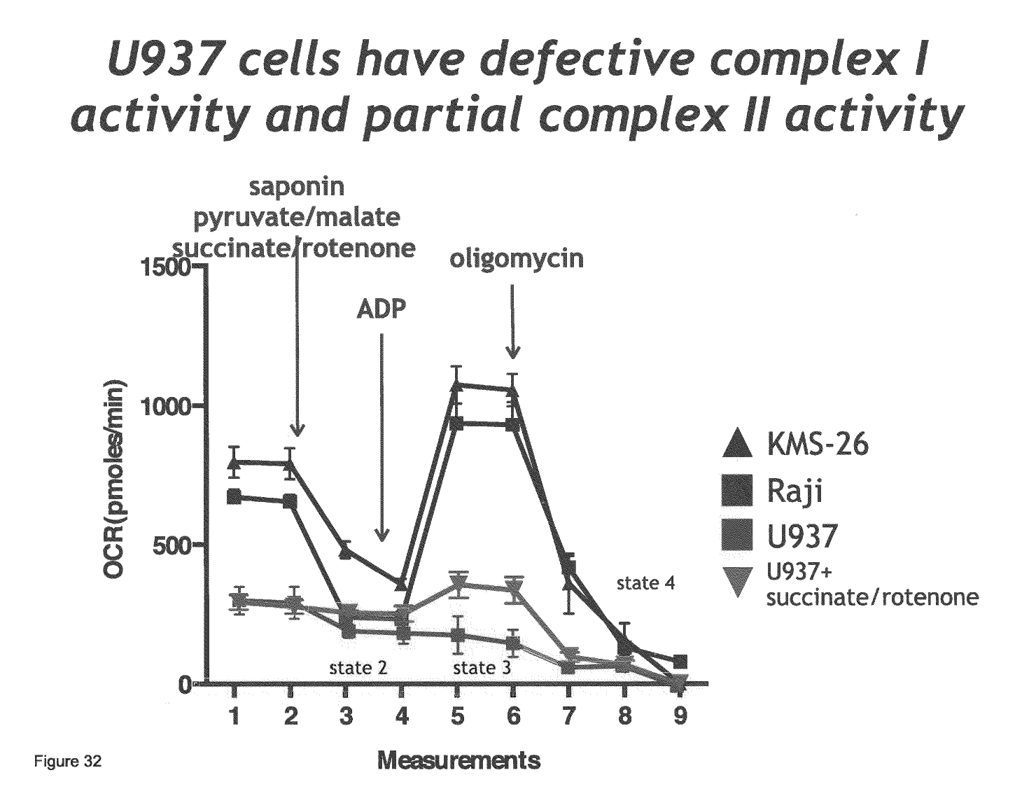

We sought to uncover the reason why certain cell lines that are capable of effective glucose uptake, such as U937, failed to increase OCR in response to glucose limitation (FIG. 31). We considered mitochondrial dysfunction as a potential cause. FIG. 32 presents data showing oxygen consumption of three cell lines upon addition of various drugs in an assay that is designed to directly test the functionality of the mitochondria. These assays were performed as described previously²⁷. Briefly, cells were re-suspended and plated cells (300,000 cells in 500 μl per well) in MAS-1 buffer (70 mM Sucrose, 220 mM Mannitol, 10 mM KH₂PO₄, 5 mM MgCl₂, 2 mM HEPES, 1 mM EGTA, 0.2% FA free BSA, pH 7.2). Saponin (50 μg/ml), methyl pyruvate/malate (10 mM/5 mM) for functional assessment of complex I, Succinate (5 mM)/Rotenone (0.5 uM) and Antimycin (1 uM) for functional assessment of complex II and III, TMPD/Ascorbate (10 mM/50 mM) for functional assessment of complex IV, and 4 mM ADP was added to permeabilized cells to activate respiration in the mitochondria. We used the complex V inhibitor oligomycin (0.5 μM) to measure oxygen consumption in the absence of oxidative phosphorylation. All compounds were diluted in the assay buffer and injected into the wells sequentially as indicated for each experiment. For the black lines and the blue line shown in FIG. 31, saponin and pyruvate and malate are added first. This permeabilizes the cell and allows direct access to the mitochondrial. Because there are no substrates for the mitochondria to do OXPHOS, oxygen consumption drops at this point. Next, ADP is added. After addition of ADP, complex V (the last complex in the OXPHOS chain) is able to run and oxygen consumption increases. This reflects the health and functionality of complexes I, III, IV and V. As shown, U937 cells (a cell line that was identified above as sensitive to glucose limitation) are deficient in one or more of these components, while KMS26 and Raji cells (cell lines that were identified above as resistant to glucose limitation) that were included as controls are not. Next oligomycin is added, terminating OXPHOS. This is a control to show that the OXPHOS induced by addition of ADP is due to the action of the electron transport chain.

The red line reflects these experiments repeated as above for U937 cells with succinate and rotenone added at the first step instead of pyruvate and malate. These molecules allow for assessment of complex II activity in contrast to above where complex I was directly assessed. U937 cells have some, but very little complex II activity. We found that U-937 cells had a profound defect in utilizing substrates for Complexes I (pyruvate and malate) and II (succinate), but not Complex IV (TMPD and ascorbate).

### Example 9

### U937 Cells have Mutations in Complex I Components that May Predict Sensitivity to Glucose Limitation and Metformin Sensitivity

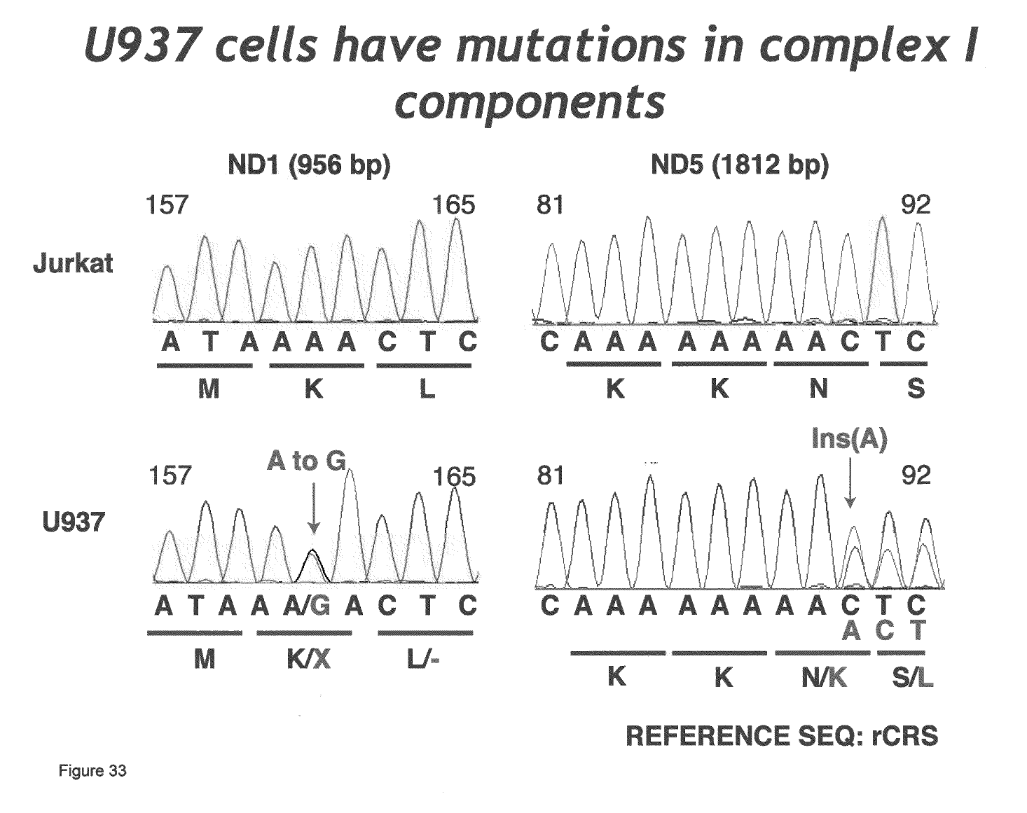

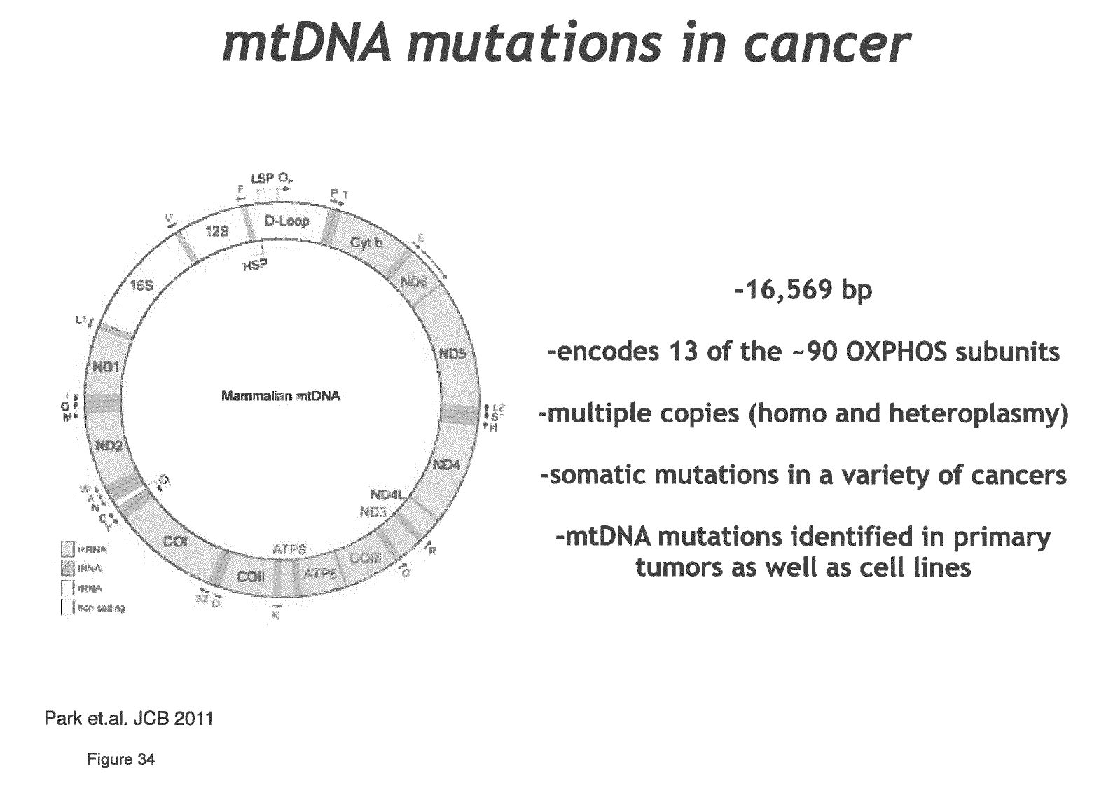

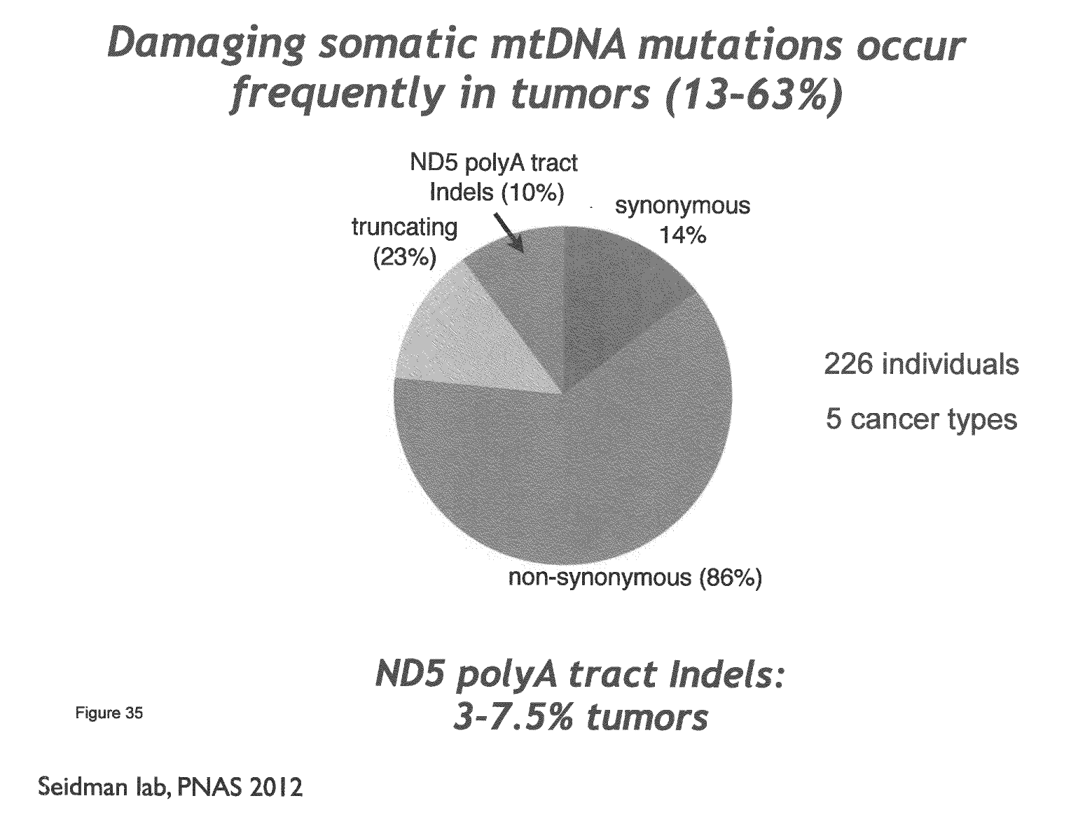

We sequenced a number of mitochondrial genes to determine whether mutations in mtDNA might underlie the defective Complex I activity of U937 cells. As noted above, in permeabilized cell mitochondrial function assays, U-937 cells had a profound defect in utilizing substrates for Complexes I (pyruvate and malate) and II (succinate), but not Complex IV (TMPD and ascorbate). This cell line is sensitive to glucose limitation and metformin, we believe, due to a mutation in several key mitochondrial genes. mtDNA sequencing identified two particular mutations (FIG. 33), which are expected to compromise Complex I function, namely heteroplasmic truncating mutations in ND1 and ND5. One of these mutations is a frameshifting mutation at the end of a polyA tract (a string of 8 consecutive As) located at mtDNA position 12418-12425. This mutation has been identified in other studies and may have a prevalence approaching 7.5% (FIG. 35). This particular mutation has been identified in the following cancers: Lung, Liver, Colon, Rectal, Ovarian, and AML (from our data and data described in Larman, T A, et al., Spectrum of somatic mitochondrial mutations in five cancers, PNAS (2012), 109(35): 14087-14091). Cancers and cancer cell lines with mutations in this location or other mtDNA mutations may also be sensitive to glucose limitation and to OXPHOS inhibitors such as biguanides. This includes, for example, the pancreatic cancer cell lines BxPC3, which has a mutation at G9804A in the gene CO3, a mutation found in patients with LHON, a human mitochondrial deficiency disorder. We obtained this cell line and found it to be sensitive to phenformin. Other cell lines have been identified which carry mutations in mtDNA encoded genes, which have been found in human patients to cause mitochondrial diseases (diseases characterized by mitochondrial dysfunction, e.g., decreased OXPHOS capacity). These mutations may also predict sensitivity to OXPHOS inhibitors, e.g., biguanides, e.g., metformin. Listed below are other mtDNA genes which harbor mutations in cancer in common with patient syndromes:

### MT-RNR1

### MT-ND1

### MT-N D2

### MT-ND3

### MT-ND4

### MT-ND5

### MT-N D6

### MT-CYB

### MT-CO1

### MT-CO3

### MT-ATP6

Cancers and cancer cell lines harboring one or more mutations associated with a human mitochondrial disorder (or other mutations in such genes that have not as yet been identified in human mitochondrial disorders) may predict sensitivity to glucose limitation and to OXPHOS inhibitors.

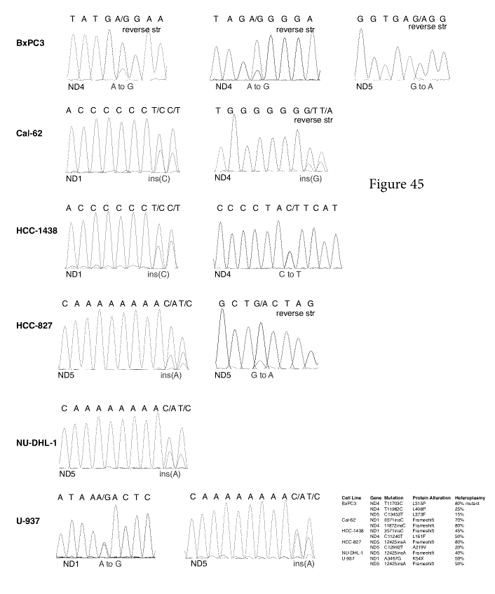

We used available cancer genome resequencing data and information from the literature^{12,13} to identify additional cell lines with mtDNA mutations in Complex I subunits and obtained five, including two with the same ND5 mutation as U-937 cells (Table 5; FIG. 45).

Hybrid capture genome resequencing data of 912 cell lines from the Broad Institute Cancer Cell Line Encyclopedia (data kindly provided by Dr. Levi Garraway (DFCI/Broad)) were mined for spurious mtDNA reads, which were aligned to the Revised Cambridge Reference Sequence. Sufficient data were obtained to reach an average of 5× coverage in 504 cell lines. Cell lines with frameshifting insertions or deletions in Complex I subunits were identified from the data, and the presence of the predicted mutations confirmed by Sanger sequencing using the primers listed below in PCR followed by sequencing reactions. The degree of heteroplasmy was estimated based upon the ratio of the area under the curves of the wild type allele to the mutant allele from Sanger sequence traces. Common variants were identified and filtered out by comparison to a database of such variants (MITOMAP: www.mitomap.org) and by the presence of these variants in >1% of the other cell lines in the CCLE set.

Primers for sequencing of mtDNA encoded Complex I genes:

ND1:
MT-ND1 F
GGT TTG TTA AGA TGG CAG AGC CC
MT-ND1 R
GAT GGG TTC GAT TCT CAT AGT CCT AG
ND2:
MT-ND2 F
TAA GGT CAG CTA AAT AAG CTA TCG GGC
MT-ND2 R
CTT AGC TGT TAC AGA AAT TAA GTA TTG CAA C
ND3, ND4L and 5′ end of ND4:
MT-ND3/4 F
TTG ATG AGG GTC TTA CTC TTT TAG TAT AAA T
MT-ND3/4 R
GAT AAG TGG CGT TGG CTT GCC AT
3′ end of ND4:
MT-ND4 F
CCT TTT CCT CCG ACC CCC TAA CA
MT-ND4 R
TAG CAG TTC. TTG TGA GCT TTC TCG GT
5′ end of ND5:
MT-ND5 F
AAC ATG GCT TTC TCA ACT TTT AAA GGA TAA C
MT-ND5 R
CGT TTG TGT ATG ATA TGT TTG CGG TTT C
ND6 and 3′ end of ND5:
MT-ND 5/6 F
ACT TCA ACC TCC CTC ACC ATT GG
MT-ND 5/6 R
TCA TTG GTG TTC TTG TAG TTG AAA TAC AAC

Like U-937, the additional lines (BxPC3, Cal-62, HCC-1438, HCC-827, NU-DHL-1) weakly boosted OCR in low glucose media (FIG. 23(B)) and had a proliferation defect in this condition (FIG. 46b). To ask if these phenotypes are caused by Complex I dysfunction, we expressed the S. cerevisiae NDI1 gene, which catalyzes electron transfer from NADH to ubiquinone without proton translocation^{5,14}. This ubiquinone oxidoreductase allows bypass of Complex I function. The retroviral ND1 vector was generated by cloning into the EcoRI and XhoI sites of the pMXS-ires-blast vector a cDNA insert generated by PCR from a yeast genomic library using the primers below, followed by standard cloning techniques:

Ndi1 EcoRI F:
ATGAATTCCATCACATCATCGAATTAC
Ndi1 XhoI R:
ATCTCGAGAAAAGGGCATGTTAATTTCATCTATAAT

NDI1 expression significantly increased the basal OCR of the Complex I defective cells (Cal-62, HCC-827, BxPC3, U-937) and partly rescued their proliferation defect in low glucose, while not substantially affecting proliferation in high glucose (FIG. 41f,i-l).

Table 5 lists certain mutations that were identified in mitochondrial genes in various cell lines that are sensitive to glucose limitation.

TABLE 5
Selected Mutations in Mitochondrial Genes Encoding OXPHOS
Complex I Components

| Cell Line | Gene | Mutation | Protein Alteration | Heteroplasmy |
|---|---|---|---|---|
| BxPC3 | ND4 | T11703C | L315P | 80% mutant |
| | ND4 | T11982C | L408P | 25% |
| | ND5 | C13453T | L373F | 15% |
| Cal-62 | ND1 | 3571insC | Frameshift | 70% |
| | ND4 | 11872insC | Frameshift | 80% |
| HCC-1438 | ND1 | 3571insC | Frameshift | 45% |
| | ND4 | C11240T | L161F | 50% |
| HCC-827 | ND5 | 12425insA | Frameshift | 80% |
| | ND5 | C12992T | A219V | 20% |
| NU-DHL-1 | ND5 | 12425insA | Frameshift | 40% |
| U-937 | ND1 | A3467G | K54X | 50% |
| | ND5 | 12425insA | Frameshift | 50% |

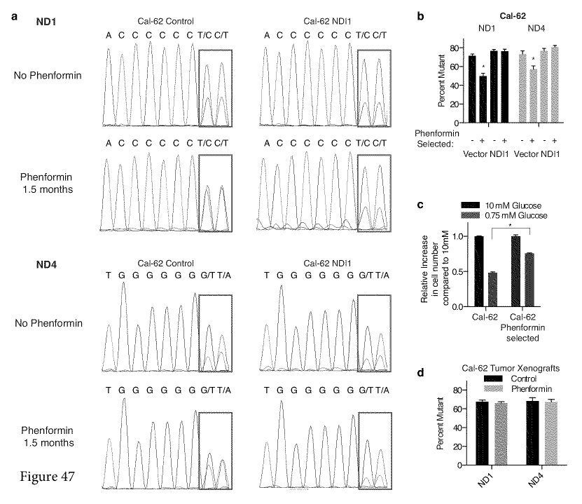

In an alternative approach, Cal-62 cells were selected at a concentration of phenformin that permitted half-maximal growth compared to the unselected line (approximately 5 uM for 2 weeks, 10 uM for 1.5 weeks, 15 uM for 1.5 weeks, and 20 uM for 1 week). Cells were split 1:10 when nearing confluence. After selection, cells were removed from phenformin for at least 3 days before starting proliferation assays. The ratio of wild type to mutant mtDNA was calculated by summing the 11 Sanger Sequencing peak height measurements per nucleotide position for the wild type and mutant allele allowing for the percent mutant calculated. These values were averaged over three nucleotide positions for which the base in the wild type and mutant sequence differs. Culture of Cal-62 cells for 1.5 months in the presence of a Complex I inhibitor (phenformin) yielded a population of cells with significantly enriched wild-type mtDNA content and a corresponding decrease in sensitivity to low glucose, changes not observed in cells expressing NDI1 (FIG. 47).

These data identify defective glucose utilization and mitochondrial dysfunction as two distinct mechanisms for conferring sensitivity to glucose limitation on cancer cell lines. Other sensitizing mechanisms may also exist as MC116 cells are sensitive to glucose limitation but do not appear to have either of these defects.

### Example 10

### Glucose Limitation-Sensitive Cell Lines are Sensitive to OXPHOS Inhibition

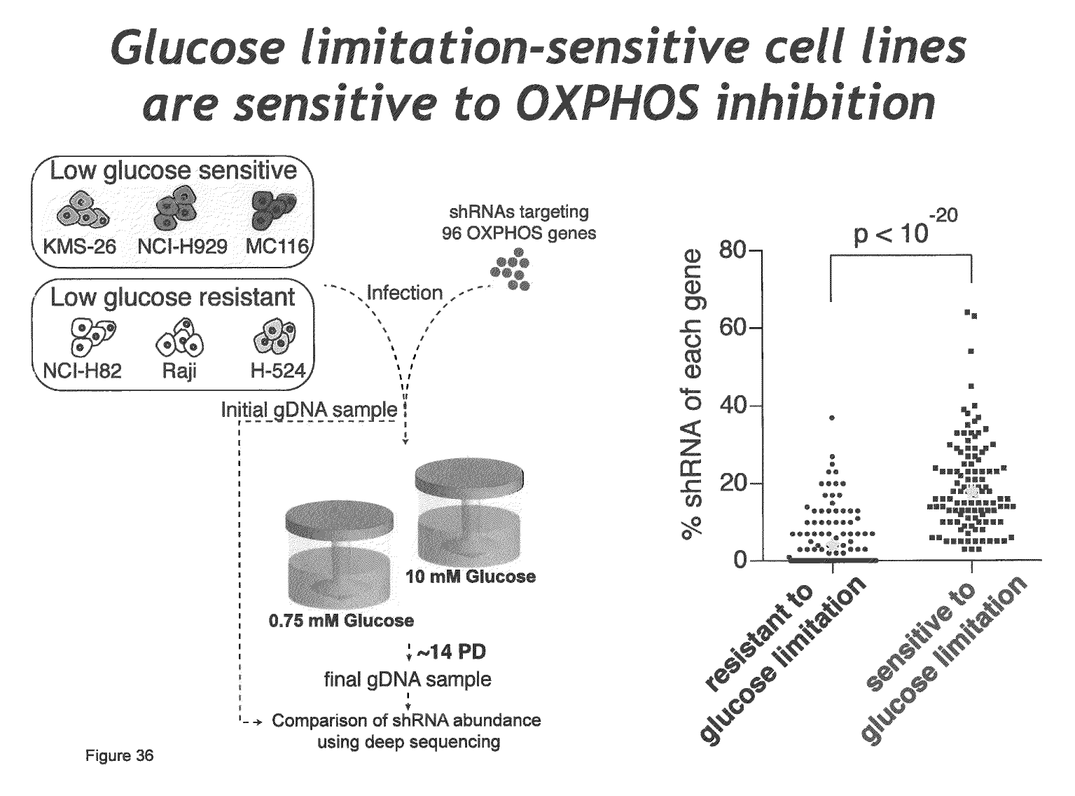

We utilized the Nutrostat system to analyze the differential requirement for various genes encoding OXPHOS components in glucose limitation sensitive and glucose limitation resistant cancer cell lines. A schematic diagram of our experiment designed to examine sensitivity of glucose limitation sensitive and glucose limitation resistant cell lines to inhibition of OXPHOS brought about by shRNA-mediated inhibition of various genes encoding OXPHOS components is shown in FIG. 36. FIG. 36 shows data from screens done on the 6 cell lines indicated using only the focused pool targeting genes related to oxidative phosphorylation. The chart on the right in FIG. 36 shows that the resistant cell lines are largely resistant to suppression of the genes upon glucose restriction, whereas the sensitive cell lines are largely sensitive to suppression of these genes under glucose limitation. These data expand on the Jurkat screen to demonstrate that the data obtained here are relevant to a larger set of cell lines and that the resistant cell lines are more resistant to inhibition of the mitochondria than the sensitive cell lines. Each point corresponds to a single gene in the pool and y-axis is the percentage of hairpins targeting that gene which score in the screen. In summary, results showed that glucose limitation-sensitive cell lines are sensitive to OXPHOS inhibition brought about by loss of expression of OXPHOS genes targeted by ShRNAs.

### Example 11

### Glucose Limitation-sensitive Cell Lines are Sensitive to Metformin

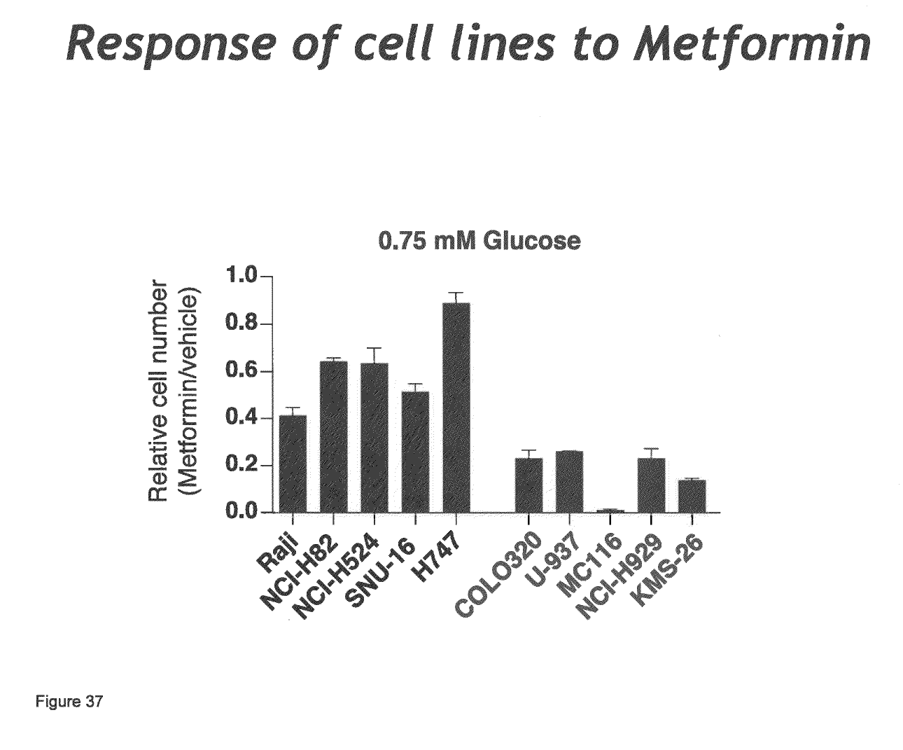

We tested the sensitivity of a panel of glucose limitation sensitive and glucose limitation resistant tumor cell lines to treatment with metformin, a compound that inhibits OXPHOS at least in part by inhibiting complex 1. Cells were exposed to 2 mM metformin for 3 days. As shown in FIG. 37, under low glucose conditions the glucose limitation sensitive cell lines were markedly more sensitive to metformin than glucose limitation resistant cell lines.

### Example 12

### Cell Lines with mtDNA-Encoded Complex I Mutations or Impaired Glucose Limitation are Sensitive to Phenformin

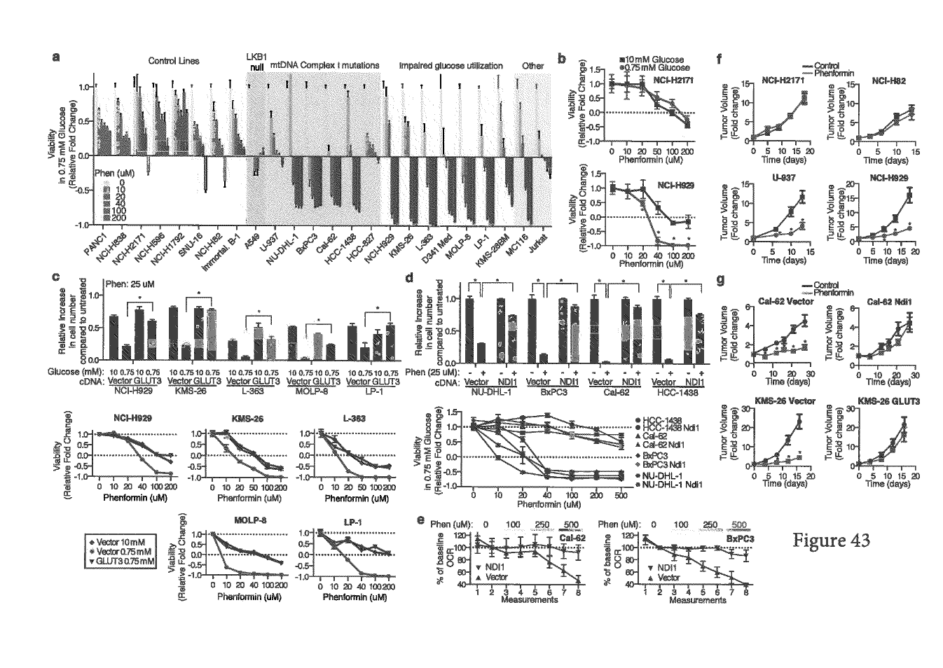

We examined the sensitivity of low glucose sensitive cells lines to the potent biguanide phenformin. In low glucose media, cell lines with mtDNA-encoded Complex I mutations (U-937, BxPC3, Cal-62, HCC-1438, HCC-827, NU-DHL-1) or impaired glucose utilization (NCI-H929, KMS-26, LP-1, L-363, MOLP-8, D341 Med, KMS-28BM) were 5-20 fold more sensitive to phenformin compared to control cancer cell lines or an immortalized B cell line (FIG. 43a), and similar results were obtained with metformin or when using direct cell counting as a readout (FIG. 46a,b,d). The low glucose sensitive cell lines, particularly those with impaired glucose utilization, tended to be more sensitive to phenformin in 0.75 than 10 mM glucose, but substantial sensitivity persisted at 1.5-3.0 mM glucose (FIG. 43b, FIG. 46c,e). Importantly, in cells with impaired glucose utilization, GLUT3 over-expression almost completely rescued the phenformin sensitivity specific to the low glucose condition, such that GLUT3-expressing cells in 0.75 mM glucose and control cells in 10 mM glucose were similarly affected by phenformin (FIG. 43c). Likewise, in cells with mutations in Complex 1, NDI1 expression almost completely rescued the effects of phenformin on proliferation (FIG. 43d) and oxygen consumption (FIG. 43e, FIG. 46g). We found that cells lacking mtDNA (143B Rho) are insensitive to phenformin but sensitive to low glucose (FIG. 46h), suggesting that phenformin sensitivity may be restricted to cells with the intermediate levels of mitochondrial dysfunction typically seen in cancer cells with mitochondrial dysfunction rather than cells with complete loss of mitochondrial function.

### Example 13

### Low Expression Levels of CYC1 and UQCRC1 in Tumor Cells Predicts Sensitivity to Biguanides Under Glucose Restriction

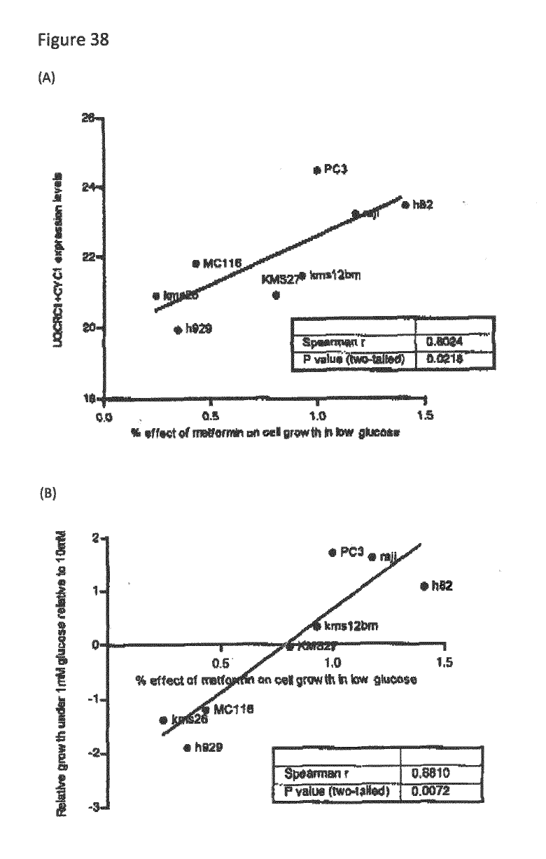

We tested the hypothesis that expression of CYC1 and UQCRC1 could predict sensitivity to metformin under conditions in which glucose is limiting. Using a panel of 8 cell lines with varying expression of CYC1 and UQCRVC1, we demonstrated that expression of these genes correlated with sensitivity to metformin under low glucose (FIG. 38(A)). 1 mM glucose was used as a low glucose condition in this experiment. Furthermore, the sensitivity of cell lines to low glucose itself also correlated with the combined sensitivity to metformin and low glucose, suggesting a synthetic lethal interaction between these two states (FIG. 38(B)).

### Example 14

### In Vivo Effects of Metformin on Tumors Derived from Glucose Limitation Sensitive or Glucose Resistant Cancer Cell Lines

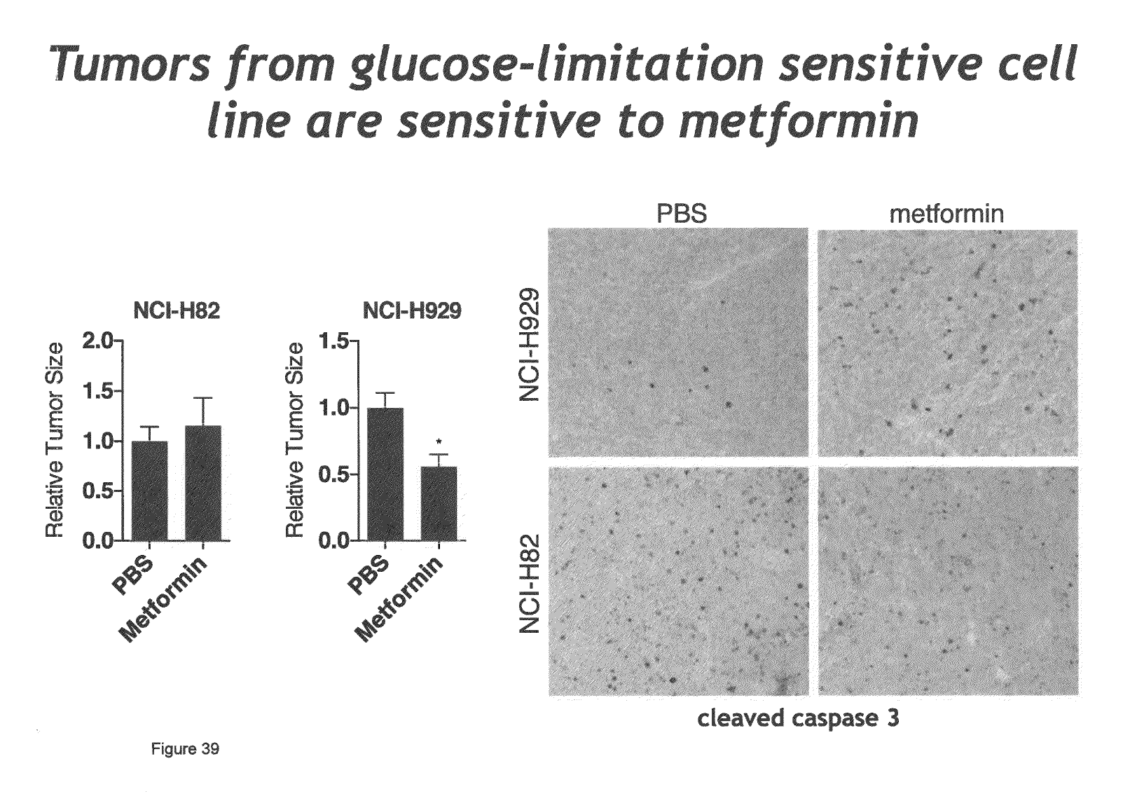

In vivo data was obtained initially using one cell line that is resistant to glucose limitation (NCI-H82) and one cell line that is sensitive to glucose limitation (NCI-H929). Mice harboring established tumors derived from these cell lines were treated with metformin (400 mg/kg) or vehicle (PBS) for close to 3 weeks and tumor size was measured. The tumor sizes were averaged for all tumors in that particular group at the end. The data demonstrate that metformin has a differential effect on the sensitive cell line, as predicted by our in vitro results (FIG. 39, plots). Tumors from the sensitive line are on average half the size in mice treated with metformin than in mice treated with vehicle. There is also an increase in cleaved caspase 3 (a marker of apoptosis) only in tumors from the sensitive line (FIG. 39, micrographs). These results confirm that glucose limitation sensitivity correlates with sensitivity to metformin and that glucose limitation experienced by tumors in vivo is accurately modeled by concentrations of ˜0.75 mM glucose in vitro.

### Example 15

### GLUT3 Over-Expression Increases Tumor Xenograft Growth and Cell Proliferation in Low Glucose Media

We performed a competitive proliferation assay comparing the growth of KMS-26 cells overexpressing GLUT3 with the growth of vector-infected KMS-26 cells under low glucose conditions. To perform the competitive proliferation assay, KMS-26 cells with vector control and GLUT3 overexpression were mixed in equal amounts and an initial mixed sample was collected. Mixed cells were then cultured in different glucose concentrations in vitro and additionally injected subcutaneously to NOD-SCID mice. After 2.5 weeks, genomic DNA was isolated from initial sample, cells cultured in different glucose concentrations in vitro, and tumors grown in mice. Using a 5′ common primer targeting the vector (AGTAGACGGCATCGCAGCTTGGATA) and 3′ primers targeting the vector (GGCGGAATTTACGTAGCGGCC) or GLUT3 (GAGCCGATTGTAGCAACTGTGATGG), the abundance of the integrated viruses were determined and the relative abundance of KMS-26 Vector and KMS-26 GLUT3 cells inferred.

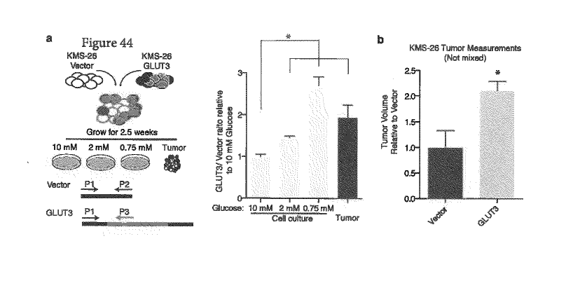

Consistent with the results described above indicating that a glucose utilization defect can account for why the proliferation of certain cancer cells is sensitive to low glucose, over-expression of GLUT3 provided a growth advantage to KMS-26 cells compared to vector infected controls grown under 0.75-2.0 mM glucose in culture and in tumor xenografts (FIG. 44).

### Example 16

### In Vivo Effects of Phenformin on Tumors Derived from Cancer Cell Lines with mtDNA Mutations

Further in vivo experiments were conducted using additional tumor cell lines. Xenografts were initiated with 2-5 million cells per injection site implanted subcutaneously into the right and left flanks of 5-8 week old male NOD.CB 17 Scid/J mice (Jackson Labs). Once tumours were palpable in all animals (>50 mm³ volume by caliper measurements), mice were assigned randomly into biguanide treated or untreated groups and caliper measurements were taken every 3-4 days until tumour burden approached the limits set by institutional guidelines. Tumour volume was assessed according to the formula ½*W*W*L or 4/3*3.14*W/2*L/2*D/2 for large tumors. Phenformin was delivered in drinking water as described previously¹⁵ at 1.7 mg/ml concentration with 5 mg/ml sucralose (Splenda), and metformin was delivered by daily IP injection (300 mg/kg). All experiments involving mice were carried out with approval from the Committee for Animal Care at MIT and under supervision of the Department of Comparative Medicine at MIT.

Consistent with the findings described above and with the low glucose environment of tumors^{1,2,7}, phenformin inhibited the growth of mouse tumour xenografts derived from cancer cells with mtDNA mutations (Cal-62, BxPC3, U-937) or poor glucose consumption (KMS-26, NCI-H929), but not from cells lacking these defects (NCI-H2171 and NCI-H82) (FIG. 43f, g). The effects of phenformin on tumour xenograft growth were rescued in mtDNA mutant cells by the introduction of NDI1, and in KMS-26 cells by the over-expression of GLUT3 (FIG. 43g, FIG. 46f), demonstrating that the effect of phenformin on these xenografts has a cell autonomous component. Thus, the glucose utilization gene signature described herein and mutations in mtDNA-encoded Complex I subunits may serve as biomarkers for identifying tumours that are particularly sensitive to biguanide (e.g., phenformin) treatment. The prevalence of truncating mutations in mtDNA-encoded OXPHOS components is reported to be as high as 16%¹⁶, and we detect the low glucose import gene expression signature in at least 5% of cell lines profiled (many lines with the signature are derived from multiple myelomas and small cell lung cancer), suggesting that a significant proportion of tumors may be particularly sensitive to biguanide treatment.

### Example 17

### AMPK Pathway Activation in Glucose Limitation Sensitive Cell Lines

We examined the ability of glucose limitation sensitive and resistant cell lines to activate the AMPK pathway and found that glucose limitation resistant cells line activate the AMPK pathway upon glucose restriction, while the sensitive cells do not. A phosphorylation-state specific antibody (recognizing AMPK alpha subunit phosphorylated on Thr172) was used to measure AMPK activation. We found that basal AMPK phosphorylation is much higher in the sensitive cell lines, suggesting that AMPK phosphorylation may be used to predict sensitivity to glucose limitation, OXPHOS inhibition, and biguanides.

### REFERENCES

1 Hirayama, A. et al. Quantitative metabolome profiling of colon and stomach cancer microenvironment by capillary electrophoresis time-of-flight mass spectrometry. Cancer Res 69, 4918-4925 (2009).
2 Gullino, P. M., Grantham, F. H. & Courtney, A. H. Glucose consumption by transplanted tumors in vivo. Cancer Res 27, 1031-1040 (1967).
3 Owen, M. R., Doran, E. & Halestrap, A. P. Evidence that metformin exerts its antidiabetic effects through inhibition of complex I of the mitochondrial respiratory chain. Biochem J 348 Pt 3, 607-614 (2000).
4 El-Mir, M. Y. et al. Dimethylbiguanide inhibits cell respiration via an indirect effect targeted on the respiratory chain complex I. J Biol Chem 275, 223-228 (2000).
Seo, B. B., Matsuno-Yagi, A. & Yagi, T. Modulation of oxidative phosphorylation of human kidney 293 cells by transfection with the internal rotenone-insensitive NADH-quinone oxidoreductase (NDI1) gene of Saccharomyces cerevisiae. Biochim Biophys Acta 1412, 56-65 (1999).
6 Cairns, R. A., Harris, I. S. & Mak, T. W. Regulation of cancer cell metabolism. Nat Rev Cancer 11, 85-95 (2011).
7 Urasaki, Y., Heath, L. & Xu, C. W. Coupling of glucose deprivation with impaired histone H2B monoubiquitination in tumors. PLoS One 7, e36775 (2012).
8 Luo, B. et al. Highly parallel identification of essential genes in cancer cells. Proc Natl Acad Sci USA 105, 20380-20385 (2008).
9 Tasseva, G. et al. Phosphatidylethanolamine deficiency in Mammalian mitochondria impairs oxidative phosphorylation and alters mitochondrial morphology. J Biol Chem 288, 4158-4173 (2013).
10 Haack, T. B. et al. Exome sequencing identifies ACAD9 mutations as a cause of complex I deficiency. Nat Genet 42, 1131-1134 (2010).
11 Crabtree, H. G. Observations on the carbohydrate metabolism of tumours. Biochem J 23, 536-545 (1929).
12 Barretina, J. et al. The Cancer Cell Line Encyclopedia enables predictive modelling of anticancer drug sensitivity. Nature 483, 603-607 (2012).
13 Jones, J. B. et al. Detection of mitochondrial DNA mutations in pancreatic cancer offers a “mass”-ive advantage over detection of nuclear DNA mutations. Cancer Res 61, 1299-1304 (2001).
14 Santidrian, A. F. et al. Mitochondrial complex I activity and NAD+/NADH balance regulate breast cancer progression. J Clin Invest 123, 1068-1081 (2013).
15 Shackelford, D. B. et al. LKB1 inactivation dictates therapeutic response of non-small cell lung cancer to the metabolism drug phenformin. Cancer Cell 23, 143-158 (2013).
16 Larman, T. C. et al. Spectrum of somatic mitochondrial mutations in five cancers. Proc Natl Acad Sci USA 109, 14087-14091 (2012).
17 Evans, J. M., Donnelly, L. A., Emslie-Smith, A. M., Alessi, D. R. & Morris, A. D.

Metformin and reduced risk of cancer in diabetic patients. BMJ 330, 1304-1305 (2005).
18 Decensi, A. et al. Metformin and cancer risk in diabetic patients: a systematic review and meta-analysis. Cancer Prev Res (Phila) 3, 1451-1461 (2010).
19 Birsoy, K., Sabatini, D. M. & Possemato, R. Untuning the tumor metabolic machine: Targeting cancer metabolism: a bedside lesson. Nat Med 18, 1022-1023 (2012).
20 Pollak, M. Metformin and pancreatic cancer: a clue requiring investigation. Clin Cancer Res 18, 2723-2725 (2012).
21 Hall, A. et al. Dysfunctional oxidative phosphorylation makes malignant melanoma cells addicted to glycolysis driven by the (V600E)BRAF oncogene. Oncotarget 4, 584-599 (2013).
22 Ben Sahra, I., Tanti, J. F. & Bost, F. The combination of metformin and 2 deoxyglucose inhibits autophagy and induces AMPK-dependent apoptosis in prostate cancer cells. Autophagy 6 (2010).
23 Javeshghani, S. et al. Carbon source and myc expression influence the antiproliferative actions of metformin. Cancer Res 72, 6257-6267 (2012).
24 Jain, M. et al. Metabolite profiling identifies a key role for glycine in rapid cancer cell proliferation. Science 336, 1040-1044 (2012).
25 Possemato, R. et al. Functional genomics reveal that the serine synthesis pathway is essential in breast cancer. Nature 476, 346-350 (2011).
26 Birsoy, K. et al. MCT 1-mediated transport of a toxic molecule is an effective strategy for targeting glycolytic tumors. Nat Genet 45, 104-108 (2013).
27 Clerc, P. & Polster, B. M. Investigation of mitochondrial dysfunction by sequential microplate-based respiration measurements from intact and permeabilized neurons. PLoS One 7, e34465 (2012).

TABLE 6

| Cell Line | SUM |
|---|---|
| LP1_HAEMATOPOIETIC_AND_LYMPHOID_TISSUE | 446 |
| NCIH1092_LUNG | 726 |
| NCIH1618_LUNG | 776 |
| D341MED_CENTRAL_NERVOUS_SYSTEM | 961 |
| NCIH660_PROSTATE | 997 |
| NCIH1105_LUNG | 1003 |
| OPM2_HAEMATOPOIETIC_AND_LYMPHOID_TISSUE | 1011 |
| L363_HAEMATOPOIETIC_AND_LYMPHOID_TISSUE | 1041 |
| NCIH929_HAEMATOPOIETIC_AND_LYMPHOID_TISSUE | 1043 |
| SKMM2_HAEMATOPOIETIC_AND_LYMPHOID_TISSUE | 1082 |
| HEP3B217_LIVER | 1108 |
| KHM1B_HAEMATOPOIETIC_AND_LYMPHOID_TISSUE | 1123 |
| NCIH1436_LUNG | 1160 |
| EB2_HAEMATOPOIETIC_AND_LYMPHOID_TISSUE | 1181 |
| KMS26_HAEMATOPOIETIC_AND_LYMPHOID_TISSUE | 1286 |
| SNU175_LARGE_INTESTINE | 1299 |
| CA46_HAEMATOPOIETIC_AND_LYMPHOID_TISSUE | 1319 |
| MOLP8_HAEMATOPOIETIC_AND_LYMPHOID_TISSUE | 1334 |
| CORL311_LUNG | 1378 |
| NCIH2196_LUNG | 1388 |
| MOLP2_HAEMATOPOIETIC_AND_LYMPHOID_TISSUE | 1419 |
| T47D_BREAST | 1427 |
| DMS79_LUNG | 1427 |
| EJM_HAEMATOPOIETIC_AND_LYMPHOID_TISSUE | 1431 |
| NCIH2066_LUNG | 1485 |
| ECC12_STOMACH | 1529 |
| THP1_HAEMATOPOIETIC_AND_LYMPHOID_TISSUE | 1540 |
| NCIH508_LARGE_INTESTINE | 1554 |
| MHHCALL4_HAEMATOPOIETIC_AND_LYMPHOID_TISSUE | 1555 |
| EB1_HAEMATOPOIETIC_AND_LYMPHOID_TISSUE | 1556 |
| NCIH1184_LUNG | 1568 |
| DKMG_CENTRAL_NERVOUS_SYSTEM | 1588 |
| KPNYN_AUTONOMIC_GANGLIA | 1595 |
| NCIH1581_LUNG | 1601 |
| NCIH209_LUNG | 1608 |
| NCIH1876_LUNG | 1620 |
| HCC38_BREAST | 1646 |
| NCIH1963_LUNG | 1647 |
| MDAMB134VI_BREAST | 1651 |
| SW403_LARGE_INTESTINE | 1660 |
| MHHCALL3_HAEMATOPOIETIC_AND_LYMPHOID_TISSUE | 1691 |
| JHOM2B_OVARY | 1700 |
| GRANTA519_HAEMATOPOIETIC_AND_LYMPHOID_TISSUE | 1716 |
| KMBC2_URINARY_TRACT | 1719 |
| NCIH661_LUNG | 1731 |
| SNU761_LIVER | 1757 |
| JURKA_HAEMATOPOIETIC_AND_LYMPHOID_TISSUE | 1766 |
| MHHES1_BONE | 1768 |
| SNU520_STOMACH | 1776 |
| OVKATE_OVARY | 1784 |
| KMS28BM_HAEMATOPOIETIC_AND_LYMPHOID_TISSUE | 1784 |
| DMS153_LUNG | 1795 |
| BICR56_UPPER_AERODIGESTIVE_TRACT | 1797 |
| SNU1079_BILIARY_TRACT | 1807 |
| TOLEDO_HAEMATOPOIETIC_AND_LYMPHOID_TISSUE | 1824 |
| NCIH1930_LUNG | 1827 |
| KMS21BM_HAEMATOPOIETIC_AND_LYMPHOID_TISSUE | 1838 |
| JM1_HAEMATOPOIETIC_AND_LYMPHOID_TISSUE | 1853 |
| U266B1_HAEMATOPOIETIC_AND_LYMPHOID_TISSUE | 1854 |
| NH6_AUTONOMIC_GANGLIA | 1858 |
| NUGC4_STOMACH | 1859 |
| PECAPJ15_UPPER_AERODIGESTIVE_TRACT | 1866 |
| NALM6_HAEMATOPOIETIC_AND_LYMPHOID_TISSUE | 1896 |
| DAUDI_HAEMATOPOIETIC_AND_LYMPHOID_TISSUE | 1897 |
| HCC1500_BREAST | 1916 |
| LS513_LARGE_INTESTINE | 1928 |
| RPMI8402_HAEMATOPOIETIC_AND_LYMPHOID_TISSUE | 1939 |
| CHP126_AUTONOMIC_GANGLIA | 1950 |
| COLO699_LUNG | 1962 |
| D283MED_CENTRAL_NERVOUS_SYSTEM | 1964 |
| PF382_HAEMATOPOIETIC_AND_LYMPHOID_TISSUE | 1998 |
| JHH7_LIVER | 2016 |
| ISTMES1_PLEURA | 2017 |
| SKNDZ_AUTONOMIC_GANGLIA | 2018 |
| OE33_OESOPHAGUS | 2020 |
| CAPAN1_PANCREAS | 2021 |
| NCIH1693_LUNG | 2034 |
| RKO_LARGE_INTESTINE | 2035 |
| MEC1_HAEMATOPOIETIC_AND_LYMPHOID_TISSUE | 2041 |
| PATU8988S_PANCREAS | 2047 |
| ASPC1_PANCREAS | 2048 |
| SCLC21H_LUNG | 2066 |
| NCIH69_LUNG | 2075 |
| OAW28_OVARY | 2091 |
| SUPB15_HAEMATOPOIETIC_AND_LYMPHOID_TISSUE | 2101 |
| EFO21_OVARY | 2107 |
| ECC10_STOMACH | 2107 |
| BICR22_UPPER_AERODIGESTIVE_TRACT | 2117 |
| NCIH889_LUNG | 2126 |
| NCIH2227_LUNG | 2127 |
| GSS_STOMACH | 2130 |
| KMS27_HAEMATOPOIETIC_AND_LYMPHOID_TISSUE | 2147 |
| KARPAS620_HAEMATOPOIETIC_AND_LYMPHOID_TISSUE | 2149 |
| NCIH2029_LUNG | 2180 |
| NCIH1836_LUNG | 2182 |
| MFE280_ENDOMETRIUM | 2184 |
| KASUMI1_HAEMATOPOIETIC_AND_LYMPHOID_TISSUE | 2188 |
| NCIH1694_LUNG | 2189 |
| KM12_LARGE_INTESTINE | 2206 |
| TE5_OESOPHAGUS | 2209 |
| SKNMC_BONE | 2213 |
| NCIH2110_LUNG | 2229 |
| CMLT1_HAEMATOPOIETIC_AND_LYMPHOID_TISSUE | 2230 |
| NCIH2172_LUNG | 2240 |
| SNU449_LIVER | 2259 |
| NALM1_HAEMATOPOIETIC_AND_LYMPHOID_TISSUE | 2259 |
| HCC202_BREAST | 2264 |
| NCIH2141_LUNG | 2269 |
| PK59_PANCREAS | 2280 |
| NCIH2081_LUNG | 2283 |
| KMM1_HAEMATOPOIETIC_AND_LYMPHOID_TISSUE | 2298 |
| COV434_OVARY | 2322 |
| HCC56_LARGE_INTESTINE | 2332 |
| HCC1599_BREAST | 2342 |
| DMS454_LUNG | 2343 |
| KPNSI9S_AUTONOMIC_GANGLIA | 2347 |
| SKNFI_AUTONOMIC_GANGLIA | 2347 |
| UT7_HAEMATOPOIETIC_AND_LYMPHOID_TISSUE | 2350 |
| SNU216_STOMACH | 2371 |
| OCILY3_HAEMATOPOIETIC_AND_LYMPHOID_TISSUE | 2375 |
| RERFLCKI_LUNG | 2386 |
| OVMANA_OVARY | 2393 |
| SHP77_LUNG | 2397 |
| EFE184_ENDOMETRIUM | 2400 |
| MOLT13_HAEMATOPOIETIC_AND_LYMPHOID_TISSUE | 2400 |
| CAL148_BREAST | 2404 |
| CAOV4_OVARY | 2404 |
| BICR31_UPPER_AERODIGESTIVE_TRACT | 2420 |
| KCIMOH1_PANCREAS | 2430 |
| RCHACV_HAEMATOPOIETIC_AND_LYMPHOID_TISSUE | 2437 |
| JVM3_HAEMATOPOIETIC_AND_LYMPHOID_TISSUE | 2445 |
| SUIT2_PANCREAS | 2446 |
| HS172T_URINARY_TRACT | 2446 |
| JHOS4_OVARY | 2450 |
| NCIH841_LUNG | 2450 |
| NCIH522_LUNG | 2452 |
| HDQP1_BREAST | 2453 |
| NCIH1048_LUNG | 2457 |
| HCC2157_BREAST | 2463 |
| ECGI10_OESOPHAGUS | 2469 |
| TC71_BONE | 2480 |
| MOLT4_HAEMATOPOIETIC_AND_LYMPHOID_TISSUE | 2486 |
| RCM1_LARGE_INTESTINE | 2495 |
| NCIH196_LUNG | 2500 |
| DMS114_LUNG | 2506 |
| SKCO1_LARGE_INTESTINE | 2508 |
| SNU626_CENTRAL_NERVOUS_SYSTEM | 2511 |
| GSU_STOMACH | 2514 |
| CHP212_AUTONOMIC_GANGLIA | 2515 |
| HUH7_LIVER | 2518 |
| TE617T_SOFT_TISSUE | 2521 |
| MDAMB157_BREAST | 2522 |
| HS604T_HAEMATOPOIETIC_AND_LYMPHOID_TISSUE | 2527 |
| KE39_STOMACH | 2538 |
| AZ521_STOMACH | 2538 |
| UACC812_BREAST | 2554 |
| OCIM1_HAEMATOPOIETIC_AND_LYMPHOID_TISSUE | 2559 |
| KASUMI6_HAEMATOPOIETIC_AND_LYMPHOID_TISSUE | 2566 |
| NCIH596_LUNG | 2568 |
| EFM19_BREAST | 2573 |
| NCIH146_LUNG | 2574 |
| TT_THYROID | 2585 |
| SNU16_STOMACH | 2602 |
| SIMA_AUTONOMIC_GANGLIA | 2608 |
| PFEIFFER_HAEMATOPOIETIC_AND_LYMPHOID_TISSUE | 2613 |
| SBC5_LUNG | 2616 |
| CHAGOK1_LUNG | 2618 |
| HCC1187_BREAST | 2621 |
| KPNRTBM1_AUTONOMIC_GANGLIA | 2626 |
| KMS12BM_HAEMATOPOIETIC_AND_LYMPHOID_TISSUE | 2627 |
| SHSY5Y_AUTONOMIC_GANGLIA | 2636 |
| HUPT4_PANCREAS | 2637 |
| CAMA1_BREAST | 2641 |
| TE125T_SOFT_TISSUE | 2641 |
| KMS34_HAEMATOPOIETIC_AND_LYMPHOID_TISSUE | 2646 |
| SKM1_HAEMATOPOIETIC_AND_LYMPHOID_TISSUE | 2666 |
| LAMA84_HAEMATOPOIETIC_AND_LYMPHOID_TISSUE | 2668 |
| A4FUK_HAEMATOPOIETIC_AND_LYMPHOID_TISSUE | 2671 |
| RS411_HAEMATOPOIETIC_AND_LYMPHOID_TISSUE | 2681 |
| HCC1143_BREAST | 2690 |
| G401_SOFT_TISSUE | 2696 |
| MHHCALL2_HAEMATOPOIETIC_AND_LYMPHOID_TISSUE | 2696 |
| HCC1806_BREAST | 2701 |
| ALEXANDERCELLS_LIVER | 2707 |
| ACCMESO1_PLEURA | 2711 |
| WSUDLCL2_HAEMATOPOIETIC_AND_LYMPHOID_TISSUE | 2720 |
| KMS20_HAEMATOPOIETIC_AND_LYMPHOID_TISSUE | 2720 |
| SW1116_LARGE_INTESTINE | 2724 |
| BL70_HAEMATOPOIETIC_AND_LYMPHOID_TISSUE | 2727 |
| HUTU80_SMALL_INTESTINE | 2732 |
| REH_HAEMATOPOIETIC_AND_LYMPHOID_TISSUE | 2738 |
| SKNBE2_AUTONOMIC_GANGLIA | 2748 |
| 697_HAEMATOPOIETIC_AND_LYMPHOID_TISSUE | 2751 |
| KMS11_HAEMATOPOIETIC_AND_LYMPHOID_TISSUE | 2754 |
| KASUMI2_HAEMATOPOIETIC_AND_LYMPHOID_TISSUE | 2759 |
| HEC6_ENDOMETRIUM | 2768 |
| GDM1_HAEMATOPOIETIC_AND_LYMPHOID_TISSUE | 2775 |
| TEN_ENDOMETRIUM | 2779 |
| GP2D_LARGE_INTESTINE | 2785 |
| REC1_HAEMATOPOIETIC_AND_LYMPHOID_TISSUE | 2790 |
| OCUM1_STOMACH | 2794 |
| NCO2_HAEMATOPOIETIC_AND_LYMPHOID_TISSUE | 2806 |
| JHOS2_OVARY | 2811 |
| SNU81_LARGE_INTESTINE | 2814 |
| PANC1_PANCREAS | 2818 |
| CPCN_LUNG | 2823 |
| HEC151_ENDOMETRIUM | 2826 |
| PANC0213_PANCREAS | 2827 |
| JHH5_LIVER | 2828 |
| QGP1_PANCREAS | 2833 |
| SU8686_PANCREAS | 2835 |
| TO175T_HAEMATOPOIETIC_AND_LYMPHOID_TISSUE | 2835 |
| MFE319_ENDOMETRIUM | 2836 |
| NALM19_HAEMATOPOIETIC_AND_LYMPHOID_TISSUE | 2837 |
| JURLMK1_HAEMATOPOIETIC_AND_LYMPHOID_TISSUE | 2841 |
| TCCPAN2_PANCREAS | 2845 |
| BCP1_HAEMATOPOIETIC_AND_LYMPHOID_TISSUE | 2846 |
| KS1_CENTRAL_NERVOUS_SYSTEM | 2864 |
| ALLSIL_HAEMATOPOIETIC_AND_LYMPHOID_TISSUE | 2867 |
| CHL1_SKIN | 2871 |
| NCIH510_LUNG | 2876 |
| SNU182_LIVER | 2883 |
| JHH6_LIVER | 2887 |
| HS751T_HAEMATOPOIETIC_AND_LYMPHOID_TISSUE | 2892 |
| MC116_HAEMATOPOIETIC_AND_LYMPHOID_TISSUE | 2893 |
| CORL47_LUNG | 2896 |
| DOHH2_HAEMATOPOIETIC_AND_LYMPHOID_TISSUE | 2896 |
| KYSE30_OESOPHAGUS | 2897 |
| CFPAC1_PANCREAS | 2898 |
| HEC251_ENDOMETRIUM | 2907 |
| ZR7530_BREAST | 2912 |
| ONS76_CENTRAL_NERVOUS_SYSTEM | 2919 |
| SNU245_BILIARY_TRACT | 2921 |
| HPAFII_PANCREAS | 2924 |
| BT483_BREAST | 2932 |
| SNUC1_LARGE_INTESTINE | 2936 |
| COV644_OVARY | 2938 |
| BV173_HAEMATOPOIETIC_AND_LYMPHOID_TISSUE | 2948 |
| HPAC_PANCREAS | 2958 |
| HUH28_BILIARY_TRACT | 2962 |
| KYM1_SOFT_TISSUE | 2967 |
| SNU398_LIVER | 2974 |
| NCIH747_LARGE_INTESTINE | 2977 |
| MM1S_HAEMATOPOIETIC_AND_LYMPHOID_TISSUE | 2979 |
| CORL24_LUNG | 2980 |
| CAL54_KIDNEY | 2980 |
| HUPT3_PANCREAS | 2985 |
| HT55_LARGE_INTESTINE | 2986 |
| GCIY_STOMACH | 2990 |
| NCIH211_LUNG | 2993 |
| KP3_PANCREAS | 2997 |
| CALU3_LUNG | 2998 |
| LC1SQSF_LUNG | 3001 |
| CORL88_LUNG | 3009 |
| SNU407_LARGE_INTESTINE | 3011 |
| CAL51_BREAST | 3011 |
| PEER_HAEMATOPOIETIC_AND_LYMPHOID_TISSUE | 3016 |
| CIM_SKIN | 3022 |
| FU97_STOMACH | 3028 |
| HEC59_ENDOMETRIUM | 3029 |
| SNUC2A_LARGE_INTESTINE | 3030 |
| KG1_HAEMATOPOIETIC_AND_LYMPHOID_TISSUE | 3035 |
| HCC1419_BREAST | 3038 |
| COLO320_LARGE_INTESTINE | 3046 |
| RH41_SOFT_TISSUE | 3047 |
| OVCAR8_OVARY | 3048 |
| CORL23_LUNG | 3050 |
| EN_ENDOMETRIUM | 3062 |
| OUMS27_BONE | 3064 |
| KYSE150_OESOPHAGUS | 3066 |
| RMGI_OVARY | 3069 |
| NCIH1781_LUNG | 3071 |
| HS611T_HAEMATOPOIETIC_AND_LYMPHOID_TISSUE | 3072 |
| ISHIKAWAHERAKLIO02ER_ENDOMETRIUM | 3077 |
| HS839T_SKIN | 3087 |
| TE14_OESOPHAGUS | 3090 |
| OUMS23_LARGE_INTESTINE | 3091 |
| NCIH2106_LUNG | 3092 |
| F36P_HAEMATOPOIETIC_AND_LYMPHOID_TISSUE | 3092 |
| CADOES1_BONE | 3095 |
| GMS10_CENTRAL_NERVOUS_SYSTEM | 3099 |
| HSC4_UPPER_AERODIGESTIVE_TRACT | 3106 |
| HCC366_LUNG | 3106 |
| SNU620_STOMACH | 3114 |
| DB_HAEMATOPOIETIC_AND_LYMPHOID_TISSUE | 3115 |
| P3HR1_HAEMATOPOIETIC_AND_LYMPHOID_TISSUE | 3134 |
| EOL1_HAEMATOPOIETIC_AND_LYMPHOID_TISSUE | 3134 |
| KYSE450_OESOPHAGUS | 3136 |
| OCIAML2_HAEMATOPOIETIC_AND_LYMPHOID_TISSUE | 3136 |
| SNUC4_LARGE_INTESTINE | 3138 |
| LS411N_LARGE_INTESTINE | 3152 |
| VMCUB1_URINARY_TRACT | 3153 |
| TOV112D_OVARY | 3160 |
| JHH2_LIVER | 3161 |
| NCIH2126_LUNG | 3162 |
| COLO205_LARGE_INTESTINE | 3169 |
| JVM2_HAEMATOPOIETIC_AND_LYMPHOID_TISSUE | 3171 |
| KU812_HAEMATOPOIETIC_AND_LYMPHOID_TISSUE | 3174 |
| MDAMB453_BREAST | 3177 |
| COLO792_SKIN | 3179 |
| EHEB_HAEMATOPOIETIC_AND_LYMPHOID_TISSUE | 3180 |
| SNU213_PANCREAS | 3183 |
| SCC9_UPPER_AERODIGESTIVE_TRACT | 3185 |
| 8MGBA_CENTRAL_NERVOUS_SYSTEM | 3189 |
| MDAMB175VII_BREAST | 3190 |
| NCIH854_LUNG | 3192 |
| MPP89_PLEURA | 3192 |
| JK1_HAEMATOPOIETIC_AND_LYMPHOID_TISSUE | 3192 |
| TE159T_SOFT_TISSUE | 3197 |
| MHHNB11_AUTONOMIC_GANGLIA | 3198 |
| SET2_HAEMATOPOIETIC_AND_LYMPHOID_TISSUE | 3198 |
| JHH1_LIVER | 3199 |
| BT474_BREAST | 3201 |
| HS729_SOFT_TISSUE | 3201 |
| SW1990_PANCREAS | 3208 |
| HUH6_LIVER | 3211 |
| MUTZ5_HAEMATOPOIETIC_AND_LYMPHOID_TISSUE | 3212 |
| BEN_LUNG | 3213 |
| NCIH838_LUNG | 3215 |
| CCK81_LARGE_INTESTINE | 3221 |
| BHY_UPPER_AERODIGESTIVE_TRACT | 3221 |
| HCC1937_BREAST | 3228 |
| CORL51_LUNG | 3228 |
| MDAPCA2B_PROSTATE | 3232 |
| HLE_LIVER | 3240 |
| MDAMB435S_SKIN | 3243 |
| PLCPRF5_LIVER | 3245 |
| ZR751_BREAST | 3247 |
| P12ICHIKAWA_HAEMATOPOIETIC_AND_LYMPHOID_TISSUE | 3261 |
| WM793_SKIN | 3263 |
| ST486_HAEMATOPOIETIC_AND_LYMPHOID_TISSUE | 3265 |
| HS870T_BONE | 3271 |
| MOLM13_HAEMATOPOIETIC_AND_LYMPHOID_TISSUE | 3290 |
| NB4_HAEMATOPOIETIC_AND_LYMPHOID_TISSUE | 3290 |
| IM95_STOMACH | 3292 |
| RDES_BONE | 3292 |
| SUDHL6_HAEMATOPOIETIC_AND_LYMPHOID_TISSUE | 3300 |
| SNGM_ENDOMETRIUM | 3300 |
| SNU719_STOMACH | 3302 |
| MDAMB415_BREAST | 3304 |
| NOMO1_HAEMATOPOIETIC_AND_LYMPHOID_TISSUE | 3304 |
| OVISE_OVARY | 3307 |
| LS180_LARGE_INTESTINE | 3309 |
| M07E_HAEMATOPOIETIC_AND_LYMPHOID_TISSUE | 3312 |
| HCC44_LUNG | 3316 |
| NCIH1568_LUNG | 3316 |
| TT_OESOPHAGUS | 3319 |
| KNS60_CENTRAL_NERVOUS_SYSTEM | 3319 |
| NCIH2286_LUNG | 3322 |
| SKHEP1_LIVER | 3323 |
| CL34_LARGE_INTESTINE | 3329 |
| GA10_HAEMATOPOIETIC_AND_LYMPHOID_TISSUE | 3334 |
| SKES1_BONE | 3338 |
| HEC1A_ENDOMETRIUM | 3340 |
| MOLT16_HAEMATOPOIETIC_AND_LYMPHOID_TISSUE | 3343 |
| IMR32_AUTONOMIC_GANGLIA | 3356 |
| HMCB_SKIN | 3366 |
| 22RV1_PROSTATE | 3371 |
| HUG1N_STOMACH | 3374 |
| DV90_LUNG | 3374 |
| JHUEM3_ENDOMETRIUM | 3374 |
| EM2_HAEMATOPOIETIC_AND_LYMPHOID_TISSUE | 3381 |
| NCIH1155_LUNG | 3386 |
| WM983B_SKIN | 3392 |
| PCM6_HAEMATOPOIETIC_AND_LYMPHOID_TISSUE | 3394 |
| RERFGC1B_STOMACH | 3396 |
| HEC108_ENDOMETRIUM | 3397 |
| SNU5_STOMACH | 3412 |
| HS600T_SKIN | 3412 |
| SNU1196_BILIARY_TRACT | 3413 |
| SW1573_LUNG | 3413 |
| SKOV3_OVARY | 3421 |
| HCC1569_BREAST | 3422 |
| SUPT1_HAEMATOPOIETIC_AND_LYMPHOID_TISSUE | 3425 |
| VCAP_PROSTATE | 3426 |
| NCIH1666_LUNG | 3437 |
| NCIH1734_LUNG | 3447 |
| HT115_LARGE_INTESTINE | 3453 |
| HSC2_UPPER_AERODIGESTIVE_TRACT | 3455 |
| MKN1_STOMACH | 3455 |
| SW48_LARGE_INTESTINE | 3458 |
| HDMYZ_HAEMATOPOIETIC_AND_LYMPHOID_TISSUE | 3458 |
| SNU283_LARGE_INTESTINE | 3459 |
| HL60_HAEMATOPOIETIC_AND_LYMPHOID_TISSUE | 3463 |
| SNU119_OVARY | 3468 |
| NCIH810_LUNG | 3477 |
| SEM_HAEMATOPOIETIC_AND_LYMPHOID_TISSUE | 3478 |
| RERFLCAI_LUNG | 3479 |
| G402_SOFT_TISSUE | 3481 |
| SW948_LARGE_INTESTINE | 3482 |
| NCIH446_LUNG | 3482 |
| YAPC_PANCREAS | 3483 |
| SW837_LARGE_INTESTINE | 3483 |
| HS737T_BONE | 3490 |
| SW780_URINARY_TRACT | 3495 |
| SCC15_UPPER_AERODIGESTIVE_TRACT | 3501 |
| KELLY_AUTONOMIC_GANGLIA | 3507 |
| CL14_LARGE_INTESTINE | 3509 |
| SNU8_OVARY | 3516 |
| SKNAS_AUTONOMIC_GANGLIA | 3521 |
| EVSAT_BREAST | 3525 |
| DU4475_BREAST | 3525 |
| HS895T_SKIN | 3526 |
| LCLC97TM1_LUNG | 3536 |
| LC1F_LUNG | 3539 |
| NCIH1437_LUNG | 3555 |
| DAOY_CENTRAL_NERVOUS_SYSTEM | 3555 |
| T173_BONE | 3555 |
| KO52_HAEMATOPOIETIC_AND_LYMPHOID_TISSUE | 3556 |
| COLO668_LUNG | 3561 |
| LOUNH91_LUNG | 3564 |
| MDAMB468_BREAST | 3572 |
| LU65_LUNG | 3573 |
| OV56_OVARY | 3579 |
| NCIH2342_LUNG | 3584 |
| KARPAS422_HAEMATOPOIETIC_AND_LYMPHOID_TISSUE | 3595 |
| PANC1005_PANCREAS | 3596 |
| M059K_CENTRAL_NERVOUS_SYSTEM | 3597 |
| P31FUJ_HAEMATOPOIETIC_AND_LYMPHOID_TISSUE | 3612 |
| KATOIII_STOMACH | 3615 |
| NCIH526_LUNG | 3621 |
| TE6_OESOPHAGUS | 3624 |
| HCC2218_BREAST | 3625 |
| DETROIT562_UPPER_AERODIGESTIVE_TRACT | 3629 |
| EFM192A_BREAST | 3637 |
| WM1799_SKIN | 3637 |
| OC314_OVARY | 3643 |
| ABC1_LUNG | 3644 |
| A704_KIDNEY | 3645 |
| KMRC20_KIDNEY | 3646 |
| PL45_PANCREAS | 3654 |
| KMH2_HAEMATOPOIETIC_AND_LYMPHOID_TISSUE | 3655 |
| HT_HAEMATOPOIETIC_AND_LYMPHOID_TISSUE | 3660 |
| PECAPJ34CLONEC12_UPPER_AERODIGESTIVE_TRACT | 3678 |
| A673_BONE | 3683 |
| TUHR14TKB_KIDNEY | 3684 |
| JHH4_LIVER | 3690 |
| C3A_LIVER | 3692 |
| SUPM2_HAEMATOPOIETIC_AND_LYMPHOID_TISSUE | 3699 |
| HS863T_BONE | 3712 |
| RT4_URINARY_TRACT | 3714 |
| NUGC3_STOMACH | 3715 |
| NCCSTCK140_STOMACH | 3716 |
| KNS81_CENTRAL_NERVOUS_SYSTEM | 3718 |
| HCC2935_LUNG | 3729 |
| COV318_OVARY | 3732 |
| HS616T_HAEMATOPOIETIC_AND_LYMPHOID_TISSUE | 3738 |
| HUT102_HAEMATOPOIETIC_AND_LYMPHOID_TISSUE | 3739 |
| KP4_PANCREAS | 3740 |
| U937_HAEMATOPOIETIC_AND_LYMPHOID_TISSUE | 3743 |
| MONOMAC6_HAEMATOPOIETIC_AND_LYMPHOID_TISSUE | 3757 |
| TCCSUP_URINARY_TRACT | 3760 |
| 2313287_STOMACH | 3765 |
| CAPAN2_PANCREAS | 3766 |
| COLO680N_OESOPHAGUS | 3770 |
| TGBC11TKB_STOMACH | 3771 |
| NCIH226_LUNG | 3787 |
| A172_CENTRAL_NERVOUS_SYSTEM | 3787 |
| KURAMOCHI_OVARY | 3790 |
| KMS18_HAEMATOPOIETIC_AND_LYMPHOID_TISSUE | 3793 |
| MCF7_BREAST | 3804 |
| CAL62_THYROID | 3807 |
| COV362_OVARY | 3815 |
| G292CLONEA141B1_BONE | 3816 |
| HS229T_LUNG | 3816 |
| NCIH292_LUNG | 3820 |
| A253_SALIVARY_GLAND | 3822 |
| SUDHL1_HAEMATOPOIETIC_AND_LYMPHOID_TISSUE | 3825 |
| SJRH30_SOFT_TISSUE | 3829 |
| HS343T_BREAST | 3830 |
| LK2_LUNG | 3835 |
| NCIH82_LUNG | 3835 |
| COV504_OVARY | 3841 |
| CAS1_CENTRAL_NERVOUS_SYSTEM | 3842 |
| SNU899_UPPER_AERODIGESTIVE_TRACT | 3849 |
| SJSA1_BONE | 3851 |
| YD8_UPPER_AERODIGESTIVE_TRACT | 3852 |
| MEWO_SKIN | 3855 |
| BCPAP_THYROID | 3855 |
| HARA_LUNG | 3860 |
| SNU1076_UPPER_AERODIGESTIVE_TRACT | 3862 |
| CAL12T_LUNG | 3864 |
| WM88_SKIN | 3866 |
| A204_SOFT_TISSUE | 3874 |
| MALME3M_SKIN | 3875 |
| DMS53_LUNG | 3876 |
| YMB1_BREAST | 3876 |
| HT1197_URINARY_TRACT | 3881 |
| HS852T_SKIN | 3882 |
| HS739T_BREAST | 3884 |
| CAL33_UPPER_AERODIGESTIVE_TRACT | 3885 |
| HOS_BONE | 3888 |
| KE37_HAEMATOPOIETIC_AND_LYMPHOID_TISSUE | 3900 |
| HS934T_SKIN | 3900 |
| HMC18_BREAST | 3904 |
| RPMI8226_HAEMATOPOIETIC_AND_LYMPHOID_TISSUE | 3924 |
| LNCAPCLONEFGC_PROSTATE | 3925 |
| A3KAW_HAEMATOPOIETIC_AND_LYMPHOID_TISSUE | 3931 |
| HEPG2_LIVER | 3931 |
| BL41_HAEMATOPOIETIC_AND_LYMPHOID_TISSUE | 3937 |
| JHUEM2_ENDOMETRIUM | 3937 |
| UACC893_BREAST | 3938 |
| DANG_PANCREAS | 3942 |
| NIHOVCAR3_OVARY | 3957 |
| HS819T_BONE | 3958 |
| RI1_HAEMATOPOIETIC_AND_LYMPHOID_TISSUE | 3961 |
| BICR6_UPPER_AERODIGESTIVE_TRACT | 3963 |
| ESS1_ENDOMETRIUM | 3964 |
| RERFLCAD1_LUNG | 3965 |
| COLO783_SKIN | 3966 |
| PATU8902_PANCREAS | 3977 |
| HLC1_LUNG | 3980 |
| HS274T_BREAST | 3983 |
| MFE296_ENDOMETRIUM | 3990 |
| JEKO1_HAEMATOPOIETIC_AND_LYMPHOID_TISSUE | 3994 |
| KE97_HAEMATOPOIETIC_AND_LYMPHOID_TISSUE | 4006 |
| AM38_CENTRAL_NERVOUS_SYSTEM | 4006 |
| OV90_OVARY | 4008 |
| OCIAML5_HAEMATOPOIETIC_AND_LYMPHOID_TISSUE | 4013 |
| PATU8988T_PANCREAS | 4015 |
| SNU61_LARGE_INTESTINE | 4019 |
| 769P_KIDNEY | 4020 |
| HS698T_LARGE_INTESTINE | 4027 |
| NCIH2405_LUNG | 4028 |
| PANC0203_PANCREAS | 4033 |
| FTC238_THYROID | 4040 |
| CW2_LARGE_INTESTINE | 4046 |
| HS675T_LARGE_INTESTINE | 4046 |
| DMS273_LUNG | 4047 |
| SKMEL2_SKIN | 4047 |
| NCIH1385_LUNG | 4047 |
| SCABER_URINARY_TRACT | 4052 |
| SNU1040_LARGE_INTESTINE | 4053 |
| HS940T_SKIN | 4063 |
| AML193_HAEMATOPOIETIC_AND_LYMPHOID_TISSUE | 4066 |
| HUNS1_HAEMATOPOIETIC_AND_LYMPHOID_TISSUE | 4074 |
| HCC4006_LUNG | 4079 |
| COLO704_OVARY | 4082 |
| NCIH2347_LUNG | 4082 |
| HCC70_BREAST | 4083 |
| SNU1197_LARGE_INTESTINE | 4085 |
| HUH1_LIVER | 4091 |
| NCIH1623_LUNG | 4093 |
| BFTC905_URINARY_TRACT | 4095 |
| OVK18_OVARY | 4096 |
| TE9_OESOPHAGUS | 4098 |
| LU99_LUNG | 4098 |
| MKN74_STOMACH | 4105 |
| BT549_BREAST | 4109 |
| HCC78_LUNG | 4111 |
| KG1C_CENTRAL_NERVOUS_SYSTEM | 4118 |
| MINO_HAEMATOPOIETIC_AND_LYMPHOID_TISSUE | 4123 |
| HUT78_HAEMATOPOIETIC_AND_LYMPHOID_TISSUE | 4128 |
| RL_HAEMATOPOIETIC_AND_LYMPHOID_TISSUE | 4133 |
| TALL1_HAEMATOPOIETIC_AND_LYMPHOID_TISSUE | 4137 |
| KMRC3_KIDNEY | 4139 |
| OVSAHO_OVARY | 4144 |
| LI7_LIVER | 4144 |
| HS618T_LUNG | 4144 |
| HH_HAEMATOPOIETIC_AND_LYMPHOID_TISSUE | 4150 |
| NCIH1838_LUNG | 4154 |
| SNU308_BILIARY_TRACT | 4155 |
| COLO678_LARGE_INTESTINE | 4155 |
| RT11284_URINARY_TRACT | 4159 |
| COLO741_SKIN | 4159 |
| NCIH23_LUNG | 4159 |
| BHT101_THYROID | 4161 |
| KYSE180_OESOPHAGUS | 4164 |
| NCIH1373_LUNG | 4166 |
| NCIH524_LUNG | 4168 |
| RKN_SOFT_TISSUE | 4168 |
| SCC4_UPPER_AERODIGESTIVE_TRACT | 4172 |
| SKMEL5_SKIN | 4177 |
| HGC27_STOMACH | 4184 |
| SNU324_PANCREAS | 4191 |
| NCIH1648_LUNG | 4198 |
| PL21_HAEMATOPOIETIC_AND_LYMPHOID_TISSUE | 4206 |
| TYKNU_OVARY | 4209 |
| OE19_OESOPHAGUS | 4210 |
| SW1417_LARGE_INTESTINE | 4214 |
| NCIH1573_LUNG | 4218 |
| YH13_CENTRAL_NERVOUS_SYSTEM | 4219 |
| HT1376_URINARY_TRACT | 4219 |
| MSTO211H_PLEURA | 4224 |
| NCIH1944_LUNG | 4226 |
| NCIH1915_LUNG | 4233 |
| KPL1_BREAST | 4234 |
| KARPAS299_HAEMATOPOIETIC_AND_LYMPHOID_TISSUE | 4235 |
| RPMI7951_SKIN | 4243 |
| CMK115_HAEMATOPOIETIC_AND_LYMPHOID_TISSUE | 4246 |
| MONOMAC1_HAEMATOPOIETIC_AND_LYMPHOID_TISSUE | 4255 |
| HEC1B_ENDOMETRIUM | 4256 |
| HS281T_BREAST | 4260 |
| MORCPR_LUNG | 4261 |
| PANC0813_PANCREAS | 4267 |
| CORL279_LUNG | 4272 |
| HCC33_LUNG | 4278 |
| HS706T_BONE | 4282 |
| PK45H_PANCREAS | 4283 |
| LS123_LARGE_INTESTINE | 4301 |
| SW1463_LARGE_INTESTINE | 4303 |
| MEG01_HAEMATOPOIETIC_AND_LYMPHOID_TISSUE | 4309 |
| AMO1_HAEMATOPOIETIC_AND_LYMPHOID_TISSUE | 4318 |
| J82_URINARY_TRACT | 4321 |
| AN3CA_ENDOMETRIUM | 4322 |
| NCIH1355_LUNG | 4329 |
| JHUEM1_ENDOMETRIUM | 4330 |
| K562_HAEMATOPOIETIC_AND_LYMPHOID_TISSUE | 4330 |
| SUDHL8_HAEMATOPOIETIC_AND_LYMPHOID_TISSUE | 4333 |
| YKG1_CENTRAL_NERVOUS_SYSTEM | 4337 |
| JHOC5_OVARY | 4339 |
| PANC0327_PANCREAS | 4340 |
| SKNSH_AUTONOMIC_GANGLIA | 4340 |
| LOVO_LARGE_INTESTINE | 4341 |
| PANC0504_PANCREAS | 4353 |
| NCIH1703_LUNG | 4354 |
| HEL_HAEMATOPOIETIC_AND_LYMPHOID_TISSUE | 4356 |
| MG63_BONE | 4363 |
| SNU1077_ENDOMETRIUM | 4364 |
| GAMG_CENTRAL_NERVOUS_SYSTEM | 4365 |
| JMSU1_URINARY_TRACT | 4367 |
| T3M4_PANCREAS | 4369 |
| NCIH2052_PLEURA | 4370 |
| NCIH2085_LUNG | 4377 |
| ISTMES2_PLEURA | 4379 |
| NB1_AUTONOMIC_GANGLIA | 4381 |
| SNU201_CENTRAL_NERVOUS_SYSTEM | 4385 |
| SCC25_UPPER_AERODIGESTIVE_TRACT | 4391 |
| BDCM_HAEMATOPOIETIC_AND_LYMPHOID_TISSUE | 4404 |
| PECAPJ41CLONED2_UPPER_AERODIGESTIVE_TRACT | 4406 |
| UMUC1_URINARY_TRACT | 4407 |
| RL952_ENDOMETRIUM | 4415 |
| MCAS_OVARY | 4418 |
| SNU410_PANCREAS | 4423 |
| RERFLCAD2_LUNG | 4425 |
| HCC1171_LUNG | 4441 |
| A2780_OVARY | 4454 |
| NCIH1341_LUNG | 4474 |
| L1236_HAEMATOPOIETIC_AND_LYMPHOID_TISSUE | 4480 |
| SKMEL3_SKIN | 4487 |
| HLFA_LUNG | 4487 |
| COLO775_HAEMATOPOIETIC_AND_LYMPHOID_TISSUE | 4489 |
| NCIN87_STOMACH | 4490 |
| C2BBE1_LARGE_INTESTINE | 4495 |
| LN229_CENTRAL_NERVOUS_SYSTEM | 4496 |
| HEC50B_ENDOMETRIUM | 4500 |
| ES2_OVARY | 4501 |
| RD_SOFT_TISSUE | 4501 |
| HS606T_BREAST | 4515 |
| MOTN1_HAEMATOPOIETIC_AND_LYMPHOID_TISSUE | 4515 |
| OC316_OVARY | 4516 |
| NCIH1435_LUNG | 4518 |
| SNU685_ENDOMETRIUM | 4520 |
| LXF289_LUNG | 4521 |
| MEC2_HAEMATOPOIETIC_AND_LYMPHOID_TISSUE | 4523 |
| SNU478_BILIARY_TRACT | 4525 |
| HCC1954_BREAST | 4530 |
| GI1_CENTRAL_NERVOUS_SYSTEM | 4530 |
| G361_SKIN | 4534 |
| HS683_CENTRAL_NERVOUS_SYSTEM | 4537 |
| JIMT1_BREAST | 4539 |
| CAKI1_KIDNEY | 4545 |
| EPLC272H_LUNG | 4552 |
| MOLM16_HAEMATOPOIETIC_AND_LYMPHOID_TISSUE | 4555 |
| NCIH1651_LUNG | 4559 |
| HS766T_PANCREAS | 4573 |
| CAL120_BREAST | 4574 |
| HS840T_UPPER_AERODIGESTIVE_TRACT | 4578 |
| MV411_HAEMATOPOIETIC_AND_LYMPHOID_TISSUE | 4591 |
| SR786_HAEMATOPOIETIC_AND_LYMPHOID_TISSUE | 4594 |
| EBC1_LUNG | 4597 |
| OVCAR4_OVARY | 4598 |
| SQ1_LUNG | 4600 |
| CMK86_HAEMATOPOIETIC_AND_LYMPHOID_TISSUE | 4602 |
| NCIH716_LARGE_INTESTINE | 4609 |
| PC3_PROSTATE | 4611 |
| NAMALWA_HAEMATOPOIETIC_AND_LYMPHOID_TISSUE | 4617 |
| SNU46_UPPER_AERODIGESTIVE_TRACT | 4627 |
| CL40_LARGE_INTESTINE | 4630 |
| KNS42_CENTRAL_NERVOUS_SYSTEM | 4632 |
| CAL851_BREAST | 4634 |
| SW480_LARGE_INTESTINE | 4638 |
| OCILY10_HAEMATOPOIETIC_AND_LYMPHOID_TISSUE | 4643 |
| SW1353_BONE | 4651 |
| VMRCLCP_LUNG | 4655 |
| PSN1_PANCREAS | 4670 |
| KCL22_HAEMATOPOIETIC_AND_LYMPHOID_TISSUE | 4670 |
| OAW42_OVARY | 4678 |
| LS1034_LARGE_INTESTINE | 4680 |
| KYSE410_OESOPHAGUS | 4682 |
| BT20_BREAST | 4693 |
| COLO818_SKIN | 4704 |
| HS821T_BONE | 4710 |
| KNS62_LUNG | 4719 |
| SNU475_LIVER | 4721 |
| SW1710_URINARY_TRACT | 4722 |
| NCIH727_LUNG | 4729 |
| NCIH1869_LUNG | 4731 |
| WM115_SKIN | 4731 |
| HS822T_BONE | 4738 |
| HS688AT_SKIN | 4740 |
| CAL27_UPPER_AERODIGESTIVE_TRACT | 4744 |
| KYSE510_OESOPHAGUS | 4745 |
| L428_HAEMATOPOIETIC_AND_LYMPHOID_TISSUE | 4746 |
| NCIH520_LUNG | 4747 |
| AGS_STOMACH | 4747 |
| TE1_OESOPHAGUS | 4759 |
| CORL95_LUNG | 4766 |
| HCC2279_LUNG | 4768 |
| MDAMB436_BREAST | 4769 |
| KYSE140_OESOPHAGUS | 4773 |
| 5637_URINARY_TRACT | 4783 |
| YD38_UPPER_AERODIGESTIVE_TRACT | 4783 |
| ACHN_KIDNEY | 4794 |
| TE4_OESOPHAGUS | 4799 |
| HCT116_LARGE_INTESTINE | 4800 |
| SNUC5_LARGE_INTESTINE | 4800 |
| MJ_HAEMATOPOIETIC_AND_LYMPHOID_TISSUE | 4801 |
| MDST8_LARGE_INTESTINE | 4811 |
| NCIH358_LUNG | 4815 |
| KYSE520_OESOPHAGUS | 4815 |
| HEYA8_OVARY | 4842 |
| KU1919_URINARY_TRACT | 4857 |
| KMRC1_KIDNEY | 4858 |
| VMRCRCZ_KIDNEY | 4859 |
| SKLMS1_SOFT_TISSUE | 4859 |
| TE15_OESOPHAGUS | 4862 |
| VMRCRCW_KIDNEY | 4862 |
| HCC1428_BREAST | 4863 |
| VMRCLCD_LUNG | 4868 |
| SKMES1_LUNG | 4873 |
| UMUC3_URINARY_TRACT | 4876 |
| HCC95_LUNG | 4877 |
| L540_HAEMATOPOIETIC_AND_LYMPHOID_TISSUE | 4878 |
| GOS3_CENTRAL_NERVOUS_SYSTEM | 4881 |
| SW900_LUNG | 4887 |
| NUGC2_STOMACH | 4888 |
| UACC257_SKIN | 4889 |
| SNU1214_UPPER_AERODIGESTIVE_TRACT | 4890 |
| KYSE270_OESOPHAGUS | 4899 |
| SUPT11_HAEMATOPOIETIC_AND_LYMPHOID_TISSUE | 4900 |
| T84_LARGE_INTESTINE | 4904 |
| A498_KIDNEY | 4906 |
| 143B_BONE | 4910 |
| NUDUL1_HAEMATOPOIETIC_AND_LYMPHOID_TISSUE | 4914 |
| RERFLCSQ1_LUNG | 4915 |
| SKMEL1_SKIN | 4916 |
| WM2664_SKIN | 4923 |
| SF295_CENTRAL_NERVOUS_SYSTEM | 4923 |
| SH10TC_STOMACH | 4926 |
| HS742T_BREAST | 4928 |
| KYSE70_OESOPHAGUS | 4930 |
| ONCODG1_OVARY | 4933 |
| COLO829_SKIN | 4934 |
| MELHO_SKIN | 4937 |
| SNU423_LIVER | 4941 |
| 1321N1_CENTRAL_NERVOUS_SYSTEM | 4946 |
| HS695T_SKIN | 4952 |
| HS936T_SKIN | 4961 |
| HS888T_BONE | 4962 |
| NCIH2291_LUNG | 4970 |
| HS571T_OVARY | 4971 |
| FADU_UPPER_AERODIGESTIVE_TRACT | 4973 |
| PANC0403_PANCREAS | 4976 |
| SNU1272_KIDNEY | 4978 |
| NCIH322_LUNG | 4986 |
| NCIH1755_LUNG | 4987 |
| HS939T_SKIN | 4987 |
| PECAPJ49_UPPER_AERODIGESTIVE_TRACT | 4994 |
| GCT_SOFT_TISSUE | 4997 |
| HDLM2_HAEMATOPOIETIC_AND_LYMPHOID_TISSUE | 4997 |
| BICR18_UPPER_AERODIGESTIVE_TRACT | 5011 |
| KP2_PANCREAS | 5013 |
| CORL105_LUNG | 5017 |
| NMCG1_CENTRAL_NERVOUS_SYSTEM | 5018 |
| TE441T_SOFT_TISSUE | 5025 |
| NCIH2170_LUNG | 5032 |
| HUCCT1_BILIARY_TRACT | 5033 |
| MDAMB361_BREAST | 5035 |
| SIGM5_HAEMATOPOIETIC_AND_LYMPHOID_TISSUE | 5035 |
| NCIH2228_LUNG | 5038 |
| MKN45_STOMACH | 5052 |
| LCL103H_LUNG | 5054 |
| HLF_LIVER | 5057 |
| SKMEL31_SKIN | 5064 |
| TF1_HAEMATOPOIETIC_AND_LYMPHOID_TISSUE | 5077 |
| HEL9217_HAEMATOPOIETIC_AND_LYMPHOID_TISSUE | 5084 |
| OV7_OVARY | 5086 |
| CI1_HAEMATOPOIETIC_AND_LYMPHOID_TISSUE | 5088 |
| ME1_HAEMATOPOIETIC_AND_LYMPHOID_TISSUE | 5095 |
| U2OS_BONE | 5097 |
| NCIH650_LUNG | 5107 |
| NCIH441_LUNG | 5108 |
| L33_PANCREAS | 5110 |
| RAJI_HAEMATOPOIETIC_AND_LYMPHOID_TISSUE | 5111 |
| LOUCY_HAEMATOPOIETIC_AND_LYMPHOID_TISSUE | 5113 |
| RERFLCMS_LUNG | 5123 |
| TE8_OESOPHAGUS | 5126 |
| HT29_LARGE_INTESTINE | 5127 |
| NCIH1395_LUNG | 5138 |
| HS944T_SKIN | 5141 |
| AU565_BREAST | 5143 |
| KOPN8_HAEMATOPOIETIC_AND_LYMPHOID_TISSUE | 5149 |
| DU145_PROSTATE | 5152 |
| NCIH1299_LUNG | 5159 |
| IGR1_SKIN | 5161 |
| SNU738_CENTRAL_NERVOUS_SYSTEM | 5170 |
| SNU1_STOMACH | 5173 |
| MKN7_STOMACH | 5173 |
| 647V_URINARY_TRACT | 5174 |
| BICR16_UPPER_AERODIGESTIVE_TRACT | 5185 |
| YD15_SALIVARY_GLAND | 5188 |
| SUDHL5_HAEMATOPOIETIC_AND_LYMPHOID_TISSUE | 5194 |
| PC14_LUNG | 5195 |
| TE10_OESOPHAGUS | 5199 |
| 42MGBA_CENTRAL_NERVOUS_SYSTEM | 5203 |
| PK1_PANCREAS | 5211 |
| COLO800_SKIN | 5217 |
| SUDHL4_HAEMATOPOIETIC_AND_LYMPHOID_TISSUE | 5225 |
| COLO684_ENDOMETRIUM | 5227 |
| RMUGS_OVARY | 5231 |
| HSC3_UPPER_AERODIGESTIVE_TRACT | 5236 |
| KMRC2_KIDNEY | 5236 |
| CGTHW1_THYROID | 5236 |
| CALU1_LUNG | 5238 |
| NCIH2087_LUNG | 5242 |
| COLO849_SKIN | 5246 |
| HEC265_ENDOMETRIUM | 5265 |
| COLO679_SKIN | 5268 |
| LOXIMVI_SKIN | 5284 |
| NCIH460_LUNG | 5285 |
| NCIH2122_LUNG | 5289 |
| BC3C_URINARY_TRACT | 5289 |
| IPC298_SKIN | 5294 |
| BFTC909_KIDNEY | 5302 |
| YD10B_UPPER_AERODIGESTIVE_TRACT | 5308 |
| SNU503_LARGE_INTESTINE | 5312 |
| CCFSTTG1_CENTRAL_NERVOUS_SYSTEM | 5314 |
| NCIH1975_LUNG | 5314 |
| DEL_HAEMATOPOIETIC_AND_LYMPHOID_TISSUE | 5318 |
| NCIH2023_LUNG | 5323 |
| SNU489_CENTRAL_NERVOUS_SYSTEM | 5325 |
| SNU878_LIVER | 5332 |
| NCIH2452_PLEURA | 5334 |
| NCIH3255_LUNG | 5347 |
| HCC1395_BREAST | 5362 |
| HS294T_SKIN | 5367 |
| FUOV1_OVARY | 5373 |
| NCIH1792_LUNG | 5376 |
| SKUT1_SOFT_TISSUE | 5378 |
| SNU601_STOMACH | 5386 |
| SKMEL30_SKIN | 5394 |
| SUPHD1_HAEMATOPOIETIC_AND_LYMPHOID_TISSUE | 5407 |
| JL1_PLEURA | 5413 |
| KLE_ENDOMETRIUM | 5414 |
| T24_URINARY_TRACT | 5440 |
| MOGGCCM_CENTRAL_NERVOUS_SYSTEM | 5451 |
| SNU387_LIVER | 5451 |
| NCIH2171_LUNG | 5451 |
| SW620_LARGE_INTESTINE | 5452 |
| SNU886_LIVER | 5456 |
| CALU6_LUNG | 5457 |
| SNU668_STOMACH | 5459 |
| SKBR3_BREAST | 5463 |
| A101D_SKIN | 5464 |
| K029AX_SKIN | 5475 |
| MIAPACA2_PANCREAS | 5482 |
| TE11_OESOPHAGUS | 5493 |
| SKMEL28_SKIN | 5507 |
| CAL29_URINARY_TRACT | 5524 |
| NCIH1793_LUNG | 5524 |
| SW1271_LUNG | 5525 |
| SUDHL10_HAEMATOPOIETIC_AND_LYMPHOID_TISSUE | 5535 |
| SKLU1_LUNG | 5570 |
| HCT15_LARGE_INTESTINE | 5573 |
| CL11_LARGE_INTESTINE | 5580 |
| HT1080_SOFT_TISSUE | 5582 |
| SW579_THYROID | 5584 |
| A549_LUNG | 5592 |
| KALS1_CENTRAL_NERVOUS_SYSTEM | 5592 |
| NCIH2009_LUNG | 5594 |
| MESSA_SOFT_TISSUE | 5596 |
| IGR37_SKIN | 5603 |
| SNB19_CENTRAL_NERVOUS_SYSTEM | 5631 |
| DND41_HAEMATOPOIETIC_AND_LYMPHOID_TISSUE | 5634 |
| OSRC2_KIDNEY | 5636 |
| U118MG_CENTRAL_NERVOUS_SYSTEM | 5640 |
| BECKER_CENTRAL_NERVOUS_SYSTEM | 5643 |
| U251MG_CENTRAL_NERVOUS_SYSTEM | 5657 |
| CAL78_BONE | 5666 |
| BXPC3_PANCREAS | 5669 |
| KYO1_HAEMATOPOIETIC_AND_LYMPHOID_TISSUE | 5676 |
| HS578T_BREAST | 5678 |
| MOLM6_HAEMATOPOIETIC_AND_LYMPHOID_TISSUE | 5678 |
| DM3_PLEURA | 5686 |
| DLD1_LARGE_INTESTINE | 5689 |
| NCIH2030_LUNG | 5693 |
| LUDLU1_LUNG | 5698 |
| U138MG_CENTRAL_NERVOUS_SYSTEM | 5701 |
| IGROV1_OVARY | 5727 |
| RCC10RGB_KIDNEY | 5728 |
| T98G_CENTRAL_NERVOUS_SYSTEM | 5731 |
| 786O_KIDNEY | 5743 |
| HT144_SKIN | 5746 |
| MOGGUVW_CENTRAL_NERVOUS_SYSTEM | 5749 |
| ML1_THYROID | 5749 |
| NCIH1339_LUNG | 5780 |
| MDAMB231_BREAST | 5781 |
| 8505C_THYROID | 5794 |
| SW1088_CENTRAL_NERVOUS_SYSTEM | 5795 |
| HS746T_STOMACH | 5815 |
| NCIH647_LUNG | 5821 |
| CAOV3_OVARY | 5824 |
| NCIH28_PLEURA | 5837 |
| RT112_URINARY_TRACT | 5866 |
| TOV21G_OVARY | 5893 |
| SW1783_CENTRAL_NERVOUS_SYSTEM | 5896 |
| NCIH1650_LUNG | 5900 |
| 8305C_THYROID | 5948 |
| NCIH1563_LUNG | 5970 |
| FTC133_THYROID | 5973 |
| SNU840_OVARY | 5983 |
| A375_SKIN | 5989 |
| TT2609C02_THYROID | 6002 |
| HCC827_LUNG | 6014 |
| CMK_HAEMATOPOIETIC_AND_LYMPHOID_TISSUE | 6082 |
| HPBALL_HAEMATOPOIETIC_AND_LYMPHOID_TISSUE | 6117 |
| TM31_CENTRAL_NERVOUS_SYSTEM | 6123 |
| IGR39_SKIN | 6130 |
| MELJUSO_SKIN | 6148 |
| OVTOKO_OVARY | 6189 |
| SF126_CENTRAL_NERVOUS_SYSTEM | 6196 |
| KIJK_HAEMATOPOIETIC_AND_LYMPHOID_TISSUE | 6196 |
| TUHR4TKB_KIDNEY | 6228 |
| SNU1105_CENTRAL_NERVOUS_SYSTEM | 6242 |
| SNU349_KIDNEY | 6288 |
| HCC15_LUNG | 6291 |
| KLM1_PANCREAS | 6293 |
| OCIAML3_HAEMATOPOIETIC_AND_LYMPOID_TISSUE | 6297 |
| CAKI2_KIDNEY | 6316 |
| SH4_SKAN | 6321 |
| HTK_HAEMATOPOIETIC_AND_LYMPHOID_TISSUE | 6322 |
| RVH421_SKIN | 6335 |
| A2058_SKIN | 6341 |
| LN18_CENTRAL_NERVOUS_SYSTEM | 6347 |
| 59M_OVARY | 6370 |
| EFO27_OVARY | 6396 |
| UACC62_SKIN | 6411 |
| GRM_SKIN | 6420 |
| RH30_SOFT_TISSUE | 6440 |
| 639V_URINARY_TRACT | 6452 |
| H4_CENTRAL_NERVOUS_SYSTEM | 6504 |
| GB1_CENTRAL_NERVOUS_SYSTEM | 6533 |
| HCC1195_LUNG | 6537 |
| NCIH2444_LUNG | 6554 |
| U87MG_CENTRAL_NERVOUS_SYSTEM | 6612 |
| LMSU_STOMACH | 6654 |
| NUDHL1_HAEMATOPOIETIC_AND_LYMPHOID_TISSUE | 6658 |
| IALM_LUNG | 6710 |
| SKMEL24_SKIN | 6749 |
| C32_SKIN | 6771 |
| OCILY19_HAEMATOPOIETIC_AND_LYMPHOID_TISSUE | 6782 |
| SNU466_CENTRAL_NERVOUS_SYSTEM | 6916 |
| S117_SOFT_TISSUE | 6933 |
| DBTRG05MG_CENTRAL_NERVOUS_SYSTEM | 7062 |
| JHOM1_OVARY | 7113 |
| TUHR10TKB_KIDNEY | 7248 |

1-12. (canceled)

12A. (canceled)

13. The method of claim 109, wherein the biguanide is metformin.

14. The method of claim 109, wherein the method comprises: (a) measuring the level of at least one indicator of sensitivity to glucose restriction in the tumor cell, tumor cell line, or tumor, or in a sample obtained therefrom; (b) comparing the level of the at least one indicator of sensitivity to glucose restriction with a reference level selected to indicate sensitivity or resistance to glucose restriction; and (c) using result(s) of the comparison to (i) classify the tumor cell, tumor cell line, or tumor according to predicted sensitivity to glucose restriction, predicted sensitivity to OXPHOS inhibition, and/or predicted sensitivity to biguanides, (ii) generate a prediction of the likelihood of sensitivity to glucose restriction, likelihood of sensitivity to OXPHOS inhibition, and/or the likelihood of sensitivity to biguanides, or (iii) identify the tumor cell, tumor cell line, or tumor as having an increased likelihood of sensitivity to glucose restriction, as having an increased likelihood of sensitivity to OXPHOS inhibition, and/or as having an increased likelihood of sensitivity to biguanides.

15. The method of claim 109, wherein the at least one indicator of sensitivity to glucose restriction comprises the level of expression of one or more genes listed in Table 1, wherein decreased expression of the one or more genes is indicative of increased sensitivity to glucose restriction.

16. (canceled)

17. The method of claim 15, wherein assessing the level of expression of a gene comprises measuring the level of a gene product encoded by the gene in the tumor cell, tumor cell line, or tumor, or in a sample obtained from the tumor cell, tumor cell line, or tumor.

18-68. (canceled)

69. A method of testing the ability of an agent to selectively inhibit the survival and/or proliferation of tumor cells under conditions of restriction of a selected nutrient, the method comprising (a) contacting test cells with an agent under conditions of restriction of a selected nutrient; (b) measuring the level of inhibition of the survival and/or proliferation of the test cells by the agent; and (c) comparing the level of inhibition of the survival and/or proliferation of the test cells by the agent under conditions of restriction of the selected nutrient with the level of inhibition of the survival and/or proliferation of comparable test cells by the agent under conditions in which the selected nutrient is not restricted, wherein the agent is identified as a candidate agent that selectively inhibits the survival and/or proliferation of tumor cells under conditions of restriction of the selected nutrient if the extent to which the agent inhibits the survival and/or proliferation of the test cells under conditions of restriction of the selected nutrient is greater than the extent to which the agent inhibits the survival and/or proliferation of comparable test cells under conditions of glucose excess.

70. The method of claim 69, wherein the test cells are cultured under conditions in which nutrients other than the selected nutrient are in excess and the concentration of the selected nutrient is maintained at an approximately constant low concentration.

71-74. (canceled)

75. The method of claim 69, further comprising (d) identifying the agent as a candidate anti-tumor agent if the agent inhibits survival and/or proliferation of the cells that are more sensitive to restriction of the selected nutrient to a greater extent than that to which it inhibits survival and/or proliferation of the cells that are less sensitive to restriction of the selected nutrient.

76. The method of claim 75, further comprising administering an agent identified as a candidate anti-tumor agent to an animal that serves as a tumor model and assessing the effect of the agent on tumor formation, development, or growth.

77-108. (canceled)

109. A method of inhibiting survival or proliferation of a tumor cell comprising: determining that the tumor cell or a tumor or tumor cell line from which the tumor cell arose exhibits at least one indicator of sensitivity to glucose restriction; and contacting the tumor cell with a biguanide.

110. A method of inhibiting growth or progression of a tumor comprising: determining that the tumor exhibits at least one indicator of sensitivity to glucose restriction; and contacting the tumor with a biguanide.

111. The method of claim 109, wherein the at least one indicator of sensitivity to glucose restriction comprises ability to take up glucose.

112. The method of claim 109, wherein the at least one indicator of sensitivity to glucose restriction comprises low expression of SLC2A3.

113. The method of claim 109, wherein the at least one indicator of sensitivity to glucose restriction comprises a defect in OXPHOS.

114. The method of claim 109, wherein the at least one indicator of sensitivity to glucose restriction comprises a mutation in a gene encoding an OXPHOS component.

115. The method of claim 109, wherein the at least one indicator of sensitivity to glucose restriction comprises a mutation in a gene encoding ND1 or ND5.

116. The method of claim 110, wherein the method comprises: (a) measuring the level of at least one indicator of sensitivity to glucose restriction in the tumor cell, tumor cell line, or tumor, or in a sample obtained therefrom; (b) comparing the level of the at least one indicator of sensitivity to glucose restriction with a reference level selected to indicate sensitivity or resistance to glucose restriction; and (c) using result(s) of the comparison to (i) classify the tumor cell, tumor cell line, or tumor according to predicted sensitivity to glucose restriction, predicted sensitivity to OXPHOS inhibition, and/or predicted sensitivity to biguanides, (ii) generate a prediction of the likelihood of sensitivity to glucose restriction, likelihood of sensitivity to OXPHOS inhibition, and/or the likelihood of sensitivity to biguanides, or (iii) identify the tumor cell, tumor cell line, or tumor as having an increased likelihood of sensitivity to glucose restriction, as having an increased likelihood of sensitivity to OXPHOS inhibition, and/or as having an increased likelihood of sensitivity to biguanides.

117. The method of claim 110 wherein the at least one indicator of sensitivity to glucose restriction comprises the level of expression of one or more genes listed in Table 1, wherein decreased expression of the one or more genes is indicative of increased sensitivity to glucose restriction.

118. The method of claim 110 wherein the at least one indicator of sensitivity to glucose restriction comprises low expression of SLC2A3.

119. The method of claim 110, wherein the at least one indicator of sensitivity to glucose restriction comprises a defect in OXPHOS.

120. The method of claim 110, wherein the at least one indicator of sensitivity to glucose restriction comprises a mutation in a gene encoding an OXPHOS component.
